(19) United States
(12) Patent Application Publication
(10) Pub. No.: US 2025/0268919 A1
(43) Pub. Date: Aug. 28, 2025

(54) COMPOUNDS FOR THE TREATMENT OF CANCER

(71) Applicant: Amneal Pharmaceuticals LLC, Bridgewater, NJ (US)

(72) Inventors: Parva Yogeshchandra PUROHIT, Ahmedabad (IN); Vishalgiri Gunvantgiri GOSWAMI, Ahmedabad (IN); Chirag Chimanlal MEHTA, Ahmedabad (IN); Mahesh Angadrao BARMADE, Nilanga (IN); Ganesh Vishwanath SANGLE, Dusarbid (IN); Vishal Bharatbhai UNADKAT, Ahmedabad (IN); Ketan Gordhanbhai VARIYA, Surat (IN); Heta Nishil PANDYA, Ahmedabad (IN)

(21) Appl. No.: 18/717,086

(22) PCT Filed: May 26, 2022
(86) PCT No.: PCT/IB2022/054955

§ 371 Date: Jun. 6, 2024

(30) Foreign Application Priority Data
Dec. 6, 2021 (IN) 202121056647

(51) Int. Cl. A61K 31/58; A61P 35/00; C07J 31/00; C07J 41/00; C07J 43/00

(52) U.S. Cl. CPC A61K 31/58; A61P 35/00; C07J 31/006; C07J 41/0044; C07J 43/003

(57) ABSTRACT

The present invention relates to novel compounds or pharmaceutically acceptable salts thereof, useful for the treatment of cancer. The present disclosure also relates to pharmaceutical composition of the novel compound and method of treating benign or malignant disease of the breast or reproductive tract, prostate cancer, or endometrial cancer using the same.

### FIELD OF INVENTION

The present invention relates to novel compounds and process for the preparation thereof. The present disclosure also relates to pharmaceutical composition of novel compounds and method of treating breast cancer using the same.

### BACKGROUND OF THE INVENTION

Cancer is one of the major causes of concern of death throughout the world. Breast cancer is one of the most common types of cancer in women in the United States. Once breast cancer forms, cancer cells can spread to other parts of the body (metastasize), making it life-threatening. Breast cancer can spread by growing into nearby tissues in the breast. It can also spread when the cancer cells get into and travel through the blood or lymph systems.

There are treatments available for breast cancer such as hormonal therapy, chemotherapy, monoclonal antibodies, chemotherapy. Breast cancers is identified by the presence of estrogen receptors (ER+) and progesterone receptors (PR+) on their surface (sometimes referred to together as hormone receptors). These ER+cancers can be treated with drugs that either block the receptors, e.g. tamoxifen, or block the production of estrogen with an aromatase inhibitor such as anastrozole or letrozole. Fulvestrant is an estrogen receptor antagonist for the treatment of hormone receptor positive metastatic breast cancer in postmenopausal women with disease progression following anti-estrogen therapy.

Another class of compound such as CDK (cyclin-dependent kinase) inhibitor such as palbociclib, ribociclib, abemaciclib is also prescribed in breast cancer. They are for use in postmenopausal women with breast cancer that is estrogen receptor positive and HER2 negative.

However, there is still an unmet need for the treatment of breast cancer. Accordingly, an object of the present invention is to provide a novel compound or a salt thereof useful for the treatment of the breast cancer.

### SUMMARY OF THE INVENTION

In one embodiment, the present invention relates to compound of formula I:

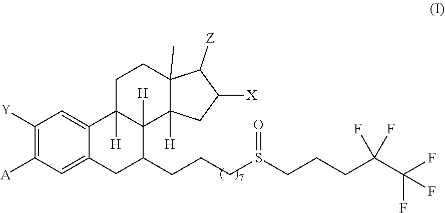

wherein;
X is halogen or hydrogen;
Y is alkyl, —O-alkyl or hydrogen;
Z is selected from ═O, ═N—OH, —O-alkyl, —O-haloalkyl, —NH—OH or —O-alkyl-OH wherein the bond between the Z substituent attached to carbon can be single or double bond depend on the substituent selected;
A is selected from

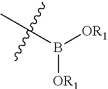

or —O—R₂, wherein R1 is selected from hydrogen or alkyl; R₂ is selected independently from group consisting of
i)

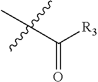

wherein R₃ is selected from NH₂, NHR₄, or NR₅R₆; R₄ is selected from group comprising optionally substituted alkyl, alkenyl, alkynyl, cycloalkyl, heterocycloalkyl, aryl, or heteroaryl; R₅ and R₆ are selected independently from group comprising optionally substituted alkyl, aryl, heteroaryl, heterocycloalkyl, cycloalkyl; or R₅ and R₆ are taken together along with the nitrogen to which they are attached to form a three to seven membered heterocyclic ring and may optionally be substituted at a substitutable position with one or more R₇ radicals, wherein the one or more R₇ radicals are independently selected at each occurrence from the group consisting of optionally substituted alkyl, alkylcarbonyl, formyl, halo, haloalkyl, alkylphosphate, phosphate, oxo, cyano, nitro, amino, hydroxyalkylamino, alkoxy, alkoxycarbonyl, carboxyalkyl, hydroxyalkyl, alkenyl, alkynyl, alkylthio, cycloalkyl, aryl, heteroaryl, aralkyl, heterocycloalkyl, alkylthioalkyl, arylcarbonyl, aralkylcarbonyl, alkoxyalkyl;
ii)

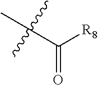

wherein R₈ is selected from group comprising optionally substituted alkyl, alkenyl, alkynyl, cycloalkyl, aryl, heteroaryl; or —CR₉R₁₀; where R₉ and R₁₀ are taken together along with the carbon to which they are attached to form a three to seven membered saturated, partially unsaturated or unsaturated heterocyclic ring in which up to 4 carbon atoms are replaced by heteroatoms chosen from the group consisting of O, S or N and may optionally be substituted at a substitutable position with one or more R₁₁ radicals, wherein one or more R₁₁ radicals are independently selected at each occurrence from the group consisting of optionally substituted alkyl, cycloalkyl, heterocycloalkyl, aryl, heteroaryl, alkylcarbonyl, formyl, halo, alkylphosphate, phosphate, cyano, nitro, alkoxy, amino, alkoxycarbonyl, alkenyl, alkynyl, alkylthio, arylcarbonyl;
iii)

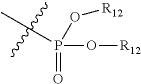

wherein each R₁₂ is selected independently from hydrogen; optionally substituted optionally substituted alkyl, aryl, acyl, heterocycloalkyl or heteroaryl;
iv)

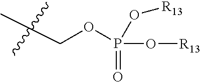

wherein each R₁₃ is selected independently from hydrogen; optionally substituted alkyl, cycloalkyl, heterocycloalkyl, aryl, or heteroaryl;
v) amino acids, wherein the amino acid is linked via ester linkage at the point of attachment;
vi)

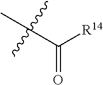

wherein R¹⁴ is

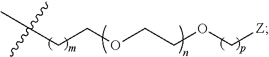

wherein m and p are independently selected from 0 to 5, n refers to degree of polymerization and is selected from 1 to 250 and Z is optionally substituted alkyl or cycloalkyl;
vii)

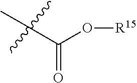

wherein R¹⁵ is selected from group comprising of optionally substituted alkyl, alkenyl, alkynyl, cycloalkyl, heterocycloalkyl, aryl and heteroaryl;
viii)

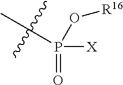

wherein R¹⁶ is selected from group comprising of hydrogen; optionally substituted alkyl, aryl, acyl, heteroaryl; and X is optionally substituted heterocycloalkyl;
ix)

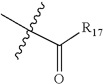

wherein R₁₇ is selected from optionally substituted bicyclic compounds, spirocyclic compounds or bridged bicyclic compounds; pharmaceutically acceptable salts, enantiomers, diastereomers, racemates, or solvates thereof.

In one embodiment, the present invention relates to compound of formula (I-A):

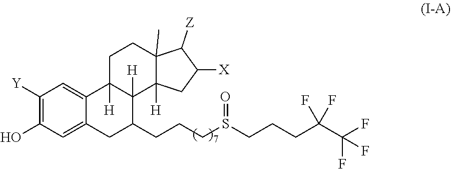

wherein;
X is halogen or hydrogen;
Y is alkyl, —O-alkyl or hydrogen;
Z is selected from ═O, ═N—OH, —O-alkyl, O-haloalkyl, —NH—OH or —O-alkyl-OH wherein the bond between the Z substituent attached to carbon can be single or double bond depend on the substituent selected; pharmaceutically acceptable salts, enantiomers, diastereomers, racemates, or solvates thereof.

In one embodiment, the present invention relates to compound of formula A:

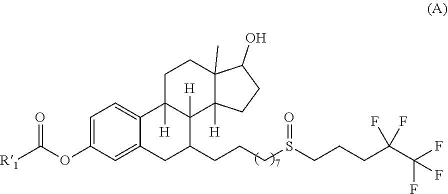

wherein,
R′₁ is selected from optionally substituted heterocycloalkyl, heteroaryl, and

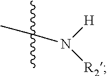

wherein one or more substitution on optionally substituted heterocycloalkyl, heteroaryl is selected from alkyl, haloalkyl, hydroxy, amino, alkylamino, dialkylamino, halo, and oxo group;
R′₂ is selected from group comprising optionally substituted heterocycloalkyl, heteroaryl and aryl group, wherein one or more substitution on heterocycloalkyl, heteroaryl and aryl is selected from alkyl, haloalkyl, hydroxy, amino, alkylamino, dialkylamino, unsubstituted heterocycloalkyl, substituted heterocycloalkyl, halo, and oxo group, wherein one or more substitution on substituted heterocycloalkyl is selected from alkyl, haloalkyl, hydroxy, amino, alkylamino, dialkylamino, halo, and oxo group;
enantiomers, diastereomers, racemates, pharmaceutically acceptable salts or solvates thereof.

In another embodiment, the present invention relates to compound of formula B:

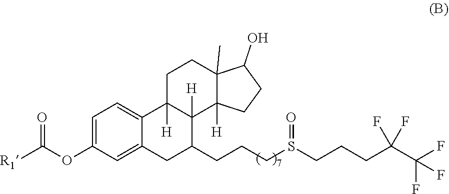

wherein;
R′₁ is selected from

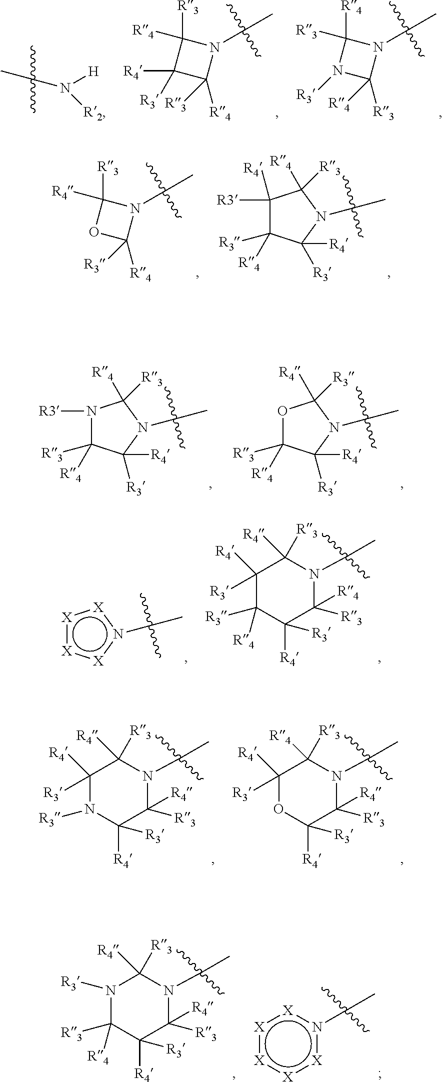

wherein R′₂ is selected from group comprising optionally substituted heterocycloalkyl, heteroaryl and aryl group,
wherein one or more substitution on heterocycloalkyl, heteroaryl and aryl is selected from alkyl, haloalkyl, hydroxy, amino, alkylamino, dialkylamino, unsubstituted heterocycloalkyl, substituted heterocycloalkyl, halo, and oxo group,
wherein one or more substitution on the substituted heterocycloalkyl is selected from alkyl, haloalkyl, hydroxy, amino, alkylamino, dialkylamino, halo, and oxo group;
[symbol] represents the aromatic ring;
X is each independently selected from N, O, S,

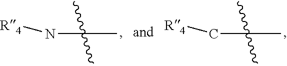

wherein R″₃, R″₄, R₃′, R₄′ is each independently selected from hydrogen, alkyl, haloalkyl, hydroxy, amino, alkylamino, dialkylamino, heterocycloalkyl, substituted heterocycloalkyl, and halo group, wherein the substituted heterocycloalkyl is substituted by one or more substitution is selected from alkyl, haloalkyl, hydroxy, amino, alkylamino, dialkylamino, halo, and oxo group; or each R″₃ and R₃′ or R″₄, and R₄′ or R″₃ and R″₄, or R₃′ and R₄′ are taken together along with the atom to which they are attached to form a three to seven membered saturated, partially unsaturated or unsaturated ring which may contain one or more heteroatoms selected from N, O and S, and the ring may optionally be substituted at a substitutable position with one or more R′₅ radicals, wherein the one or more R′₅ radicals are independently selected from hydrogen, alkyl, haloalkyl, hydroxy, amino, alkylamino, dialkylamino, halo, and oxo group; or each R″₃ and R₃′ or R″₄, and R₄′ are taken together along with the atom to which they are attached to form double bond; or each R″₃ and R″₄, or R₃′ and R₄′ are taken together form oxo( [symbol] ) bond;
enantiomers, diastereomers, racemates, pharmaceutically acceptable salts or solvates thereof.

In another embodiment, the present invention relates to compound of formula C:

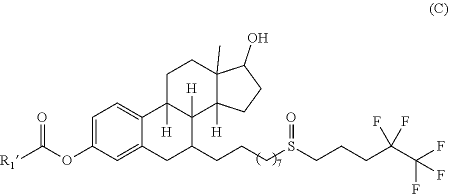

wherein;
R′₁ is selected from

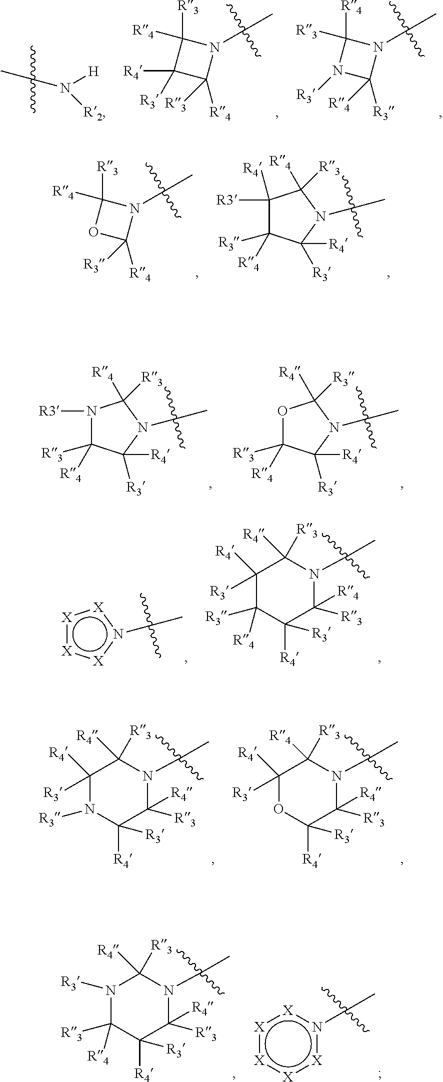

R′₂ is selected from,

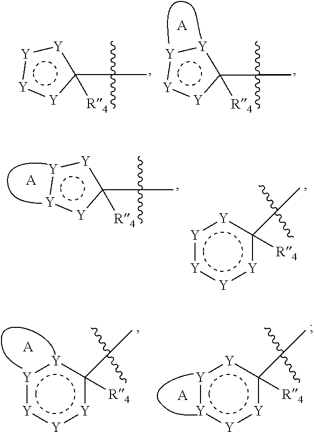

[symbol] represents the aromatic ring;
[symbol] represents the saturation, partial unsaturation or the complete unsaturation in the ring;
X is each independently selected from N, O, S,

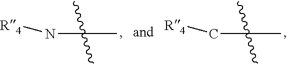

Y is selected from C,

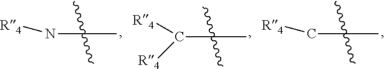

N, O, and S;
fused ring A represents an optionally substituted saturated, unsaturated, or aromatic four, five, six, or seven member ring having zero or more heteroatoms in the ring, the remaining atoms of the ring being carbon with each heteroatom being independently selected from nitrogen, oxygen and sulfur, wherein ring A is optionally substituted with one or more R″₆ radicals independently selected from hydrogen, alkyl, haloalkyl, hydroxy, amino, alkylamino, dialkylamino, unsubstituted heterocycloalkyl, substituted heterocycloalkyl, halo, and oxo group, wherein the one or more substitution on substituted heterocycloalkyl is selected from alkyl, haloalkyl, hydroxy, amino, alkylamino, dialkylamino, halo, and oxo group;
R″₃, R″₄, R₃′, R₄′ each independently selected from H, alkyl, haloalkyl, hydroxy, amino, alkylamino, dialkylamino, heterocycloalkyl, substituted heterocycloalkyl, and halo group, wherein the substituted heterocycloalkyl is substituted by one or more substitution is selected from alkyl, haloalkyl, hydroxy, amino, alkylamino, dialkylamino, halo, and oxo group; or each R″₃ and R₃′ or R″₄, and R₄′ or R″₃ and R″₄, or R₃′ and R₄′ are taken together along with the atom to which they are attached to form a three to seven membered saturated, partially unsaturated or unsaturated ring which may contain one or more heteroatoms selected from N, O and S, and the ring may optionally be substituted at a substitutable position with one or more R′₅ radicals, wherein the one or more R′₅ radicals are independently selected from hydrogen, alkyl, haloalkyl, hydroxy, amino, alkylamino, dialkylamino, halo, and oxo group; or each R″₃ and R₃′ or R″₄, and R₄′ are taken together along with the atom to which they are attached to form double bond; or each R″₃ and R″₄, or R₃′ and R₄′ are taken together form oxo( [symbol] ) bond;
enantiomers, diastereomers, racemates, pharmaceutically acceptable salts or solvates thereof.

In another embodiment, the present invention relates to pharmaceutical composition comprising novel compound; pharmaceutically acceptable salts, enantiomers, diastereomers, racemates, or solvates thereof; and pharmaceutically acceptable excipients.

The pharmaceutical compositions of the present disclosure can be in any form known to those of skill in the art. The pharmaceutical compositions of this invention may be administered orally, parenterally, by inhalation spray, topically, rectally, nasally, buccally or via an implanted reservoir, preferably oral administration, or administration by injection.

In another embodiment, the invention provides use of compounds of the present invention in the preparation of a pharmaceutical formulation as describe hereinabove, for the treatment of a benign or malignant disease of the breast or reproductive tract.

### BRIEF DESCRIPTION OF THE FIGURE

FIG. 1: Mean±SD plasma concentration—time profile following single oral administration of compound 121 to Female CD-1 mice.

FIG. 2: Cytotoxic effect of compound 121 on the human Breast Cancer MCF-7 cells: Graph was plotted with concentration on X axis and percentage viability on Y axis using Graph Pad Prism-9. Each point on the curve corresponding to the test concentration represents mean+/−SEM of three replicates.

FIG. 3. Effect of compound 121 on Uterus Weights (Wet): Data is represented as Mean±SEM n=3-8, *p<0.0001, E2 Vs. Treatment One Way ANOVA followed by Dunnett's multiple comparison test. Mean tumor volume±SD of vehicle and test compounds. PO; per oral, E2; 17α-ethynyl estradiol.

FIG. 4. Effect of compound 121 on MCF7 tumor bearing nude mice. Mean tumor volume±SD of vehicle and test compounds. Comparisons of tumor volume measurements is made by two-way ANOVA.

FIG. 5: Mean plasma concentration—time profile following single oral administration of Compound 121 and Fulvestrant to Cynomolgus monkeys.

FIG. 6: Cytotoxic effect of Compound Ia on the human Breast Cancer MCF-7 cells: Graph was plotted with concentration on X axis and percentage viability on Y axis using Graph Pad Prism-9. Each point on the curve corresponding to the test concentration represents mean+/−SEM of three replicates.

FIG. 7: Cytotoxic effect of Compound I-bh on the human Breast Cancer MCF-7 cells: Graph was plotted with concentration on X axis and percentage viability on Y axis using Graph Pad Prism-9. Each point on the curve corresponding to the test concentration represents mean+/−SEM of three replicates.

FIG. 8:. Effect of Compound I-bh on ER alpha expression in human breast cancer MCF-7 cell lines: N=2 per group, Data shown as Mean±SEM. ###p<0.001 vs. Vehicle, ***p<0.001 vs. Vehicle. One Way ANOVA, Dunnett's Post Test, Percentage shown is % inhibition of ER alpha expression in hMCF7 cell lines by treatment compared to their respective Vehicle.

FIG. 9: Effect of Compound I-a on ER alpha expression in human breast cancer MCF-7 cell lines: N=2 per group. Data shown as Mean±SEM. ###p<0.001 vs. Vehicle ***p<0.001, **p<0.01, *p<0.05 vs. Vehicle One Way ANOVA, Dunnett's Post Test. Percentage shown is % inhibition of ER alpha expression in hMCF7 cell lines by treatment compared to their respective Vehicle.

FIG. 10: Mean time vs plasma concentration of compound I-a after oral administration of compound I-a in rats (10 mg/Kg).

FIG. 11: Mean time vs plasma concentration of compound I-bh after oral administration of compound I-bh in rats (10 mg/Kg).

FIG. 12. Effect of Compound I-a on MCF7 tumor bearing nude mice. Mean tumor volume±SD of vehicle and test compounds. Comparisons of tumor volume measurements is made by two-way ANOVA.

### DETAILED DESCRIPTION OF THE INVENTION

The terms “a” and “an” do not denote a limitation of quantity, but rather denote the presence of at least one of the referenced items.

The term “about” as used herein, when referring to a measurable value is meant to encompass variations of ±10%, preferably ±5%, more preferably ±1% and still more preferably ±0.1% from the specified value.

The term “cancer” refers to conditions including solid cancers, lymphomas and leukemias. Examples of different types of cancer include, but are not limited to, breast cancer, lung cancer, ovarian cancer, prostate cancer, endometrial cancer, colorectal cancer, liver cancer, renal cancer, bladder cancer, thyroid cancer, pleural cancer, pancreatic cancer, uterine cancer, cervical cancer, testicular cancer, anal cancer, bile duct cancer, gastrointestinal carcinoid tumors, esophageal cancer, gall bladder cancer, appendix cancer, small intestine cancer, stomach cancer, cancer of the central nervous system, skin cancer, choriocarcinoma, head and neck cancer, blood cancer, osteogenic sarcoma, fibrosarcoma, neuroblastoma, glioma, melanoma, B-cell lymphoma, non-Hodgkin's lymphoma, Burkitt's lymphoma, small cell lymphoma, large cell lymphoma, monocytic leukemia, myelogenous leukemia, acute lymphocytic leukemia, acute myelocytic leukemia, and multiple myeloma.

As used herein, the term “alkyl” refers to a straight or branched, saturated, aliphatic radical having from 1 to about 10 carbon atoms, for example, methyl, ethyl, propyl, isopropyl, n-butyl, i-butyl, t-butyl and the like.

As used herein, the term “haloalkyl” refers to an alkyl group, as defined above, containing at least 1 carbon atoms substituted with at least one halo group. As used herein “haloalkyl” groups useful in the present invention include, but are not limited to, methyl, ethyl, n-propyl, isopropyl, isobutyl and n-butyl substituted independently with one or more halo groups, e.g., fluoro, chloro, bromo and iodo. Example of haloalkyl includes but are not limited to —CH₂Cl, —CHCl₂, —CCl₃, —CH₂F, —CHF₂, —CF₃ and the like.

The term “alkenyl” refers to a straight chain or branched hydrocarbon having from 2 to 10 carbon atoms with at least one carbon-carbon double bond. Alkenyl groups can have any suitable number of double bonds, including, but not limited to 1, 2, 3, 4, 5 or more. In one embodiment, alkenyl groups include ethenyl (—CH═CH₂), 2-propenyl (allyl, —CH₂—CH═CH₂) and the like.

The term “alkynyl” refers an alkynyl groups having from 2 to 10 carbon atoms and having at least 1-2 sites of alkynyl unsaturation, alkynyl groups include ethynyl (—C≡CH), propargyl (—CH₂—C≡CH), 1-butenyl, 2-butenyl, isobutenyl, butadienyl and the like.

The term “cycloalkyl” refers a saturated carbocyclic group having from 3 to 10 carbon atoms having a single ring (e.g., cyclohexyl) or multiple condensed rings (e.g., norbornyl). cycloalkyl include cyclopropyl, cyclobutyl, cyclpentyl, cyclohexyl, cycloheptyl, norbornyl and the like.

The term “heterocyclic ring” refers organic compounds that contain a ring structure containing atoms in addition to carbon, such as sulphur, oxygen or nitrogen, as part of the ring. There can be more than one hetero atom substitution in the ring structure selected from sulphur, oxygen or nitrogen. The ring can be saturated, partially saturated or unsaturated ring structure which includes “heterocycloalkyl” and “heteroaryl”.

The term “heterocycloalkyl” refers a C₃-C₁₀ cycloalkyl group according to the definition above, in which up to 4 carbon atoms are replaced by heteroatoms chosen from the group consisting of O, S or N. In one embodiment heterocycloalkyl can be saturated, partially saturated or unsaturated ring structure. For example saturated heterocycloalkyl are aziridinyl, oxiranyl, azetidinyl, oxetanyl, pyrrolidinyl, tetrahydrofuranyl, tetrahydro-thienyl, pyrazolidinyl, imidazolidinyl, oxazolidinyl, isoxazolidinyl, thiazolidinyl, piperidinyl, tetrahydropyranyl, tetrahydrothiopyranyl, piperazinyl, morpholinyl, thiomorpholinyl, 1,1-dioxo-thiomorpholin-4-yl, azepanyl, diazepanyl, homopiperazinyl, or oxazepanyl.

The term “aryl” refers a cyclic aromatic hydrocarbon radical consisting of one or more fused rings containing 6-14 carbon atoms in which at least one ring is aromatic in nature, for example phenyl, naphthyl, 1,2,3,4-tetrahydronaphthalenyl, indanyl and the like.

The term “heteroaryl” refer to monocyclic or fused bicyclic rings containing 5-14 atoms, in which at least one ring is aromatic in nature, and which contains at least one heteroatom, selected from N, O or S., for example pyridyl, pyrrolyl, furyl, thienyl, imidazolyl, oxazolyl, isoxazolyl, thiazolyl, isothiazolyl, pyrazolyl, 1,2,3-triazolyl, 1,2,4-triazolyl, benzothienyl, benzotriazolyl, isobenzothienyl, indolyl, isoindolyl, benzimidazolyl, imidazo[1,2-a]pyridyl, benzothiazolyl, benzoxazolyl, quinolizinyl, quinazolinyl, benzoquinolyl and the like.

The term “bicyclic” refers to compound that features two joined rings. A bicyclic compound can be carbocyclic or heterocyclic. Both the rings further can be aliphatic or aromatic or a combination of aliphatic and aromatic.

The term “spirocyclic” refers to compound where two rings share only one single atom, the spiro atom, and which is usually a quaternary carbon. A spirocyclic compound can be carbocyclic or heterocyclic.

The term “bridged bicyclic” refers to the compound with two rings which shares three or more atoms, wherein the two bridgehead atoms is separated by a bridge containing at least one atom. A bridged bicyclic compound can be carbocyclic or heterocyclic.

As used herein “fused/condensed” refers a compound where two rings share two adjacent atoms. In other words, the rings share one covalent bond.

The term “halogen” refers chlorine, iodine, fluorine and bromine.

As used herein, the term “leaving group” or “L” or “L₁” can be defined as part of a substrate that cleaved by the action of a nucleophile. Examples of leaving groups include, but are not limited to: halogen (F, Cl, Br, and I), tosylate, mesylate, triflate, acetate, hydroxyl, camphorsulfonate, aryloxide, and aryloxide and the like.

The term “alkoxy” refers to the group —O-alkyl.

The term “alkoxyalkyloxy” refers to the group (alkyl-O-alky-O—).

The term “alkoxycarbonyloxy” refers to the group (alkyl-O—CO—O—).

The term “phosphate” refers to —O—PO(OH)₂.

The term “phosphonyl” refers to —PO₃H₂.

The term “alkylphosphate” refers to (-alkyl-O— PO(OH)₂).

The term “hydroxyalkylamino” refer to (—NH-alkyl-OH).

The term “halo” or “halogen” refers to F, Cl, Br, and I. The term “Oxo” refers to ═O.

The term “completely unsaturated” as used here in refers to aromatic group

The term “amino acid” as used herein refers to two stereoisomeric forms, called “D” and “L.” The D and L form of any amino acid have identical physical properties and chemical reactivities, but rotate the plane of plane-polarized light equally but in opposite directions and react at different rates with asymmetric reagents. All naturally occurring amino acids in proteins are in the L form. Amino acid comprises lysine, valine, tryptophan, phenylalanine, methionine, leucine, threonine, isoleucine, arginine, histidine, tyrosine, carnitine, serine, glutamine, aspartic acid, proline, glycine, cysteine, alanine, glutamic acid. Amino acid may be present as either “D” or “L” enantiomer.

The term “—C(O)-amino acid” refers to the amino acid attached to carbonyl group via amide or ester linkage: more preferably via amide linkage.

The term “optionally” means the subsequently described event or circumstance can or cannot occur, and that the description includes instances where the event or circumstance occurs and instances where it does not.

The term “pharmaceutically acceptable carrier” of “pharmaceutically acceptable excipients” refers to a non-toxic carrier that may be administered to a patient, together with a compound of this invention, and which does not destroy the pharmacological activity thereof.

The term “pharmaceutically acceptable excipient” as used herein includes vehicles, adjuvants, or diluents or other auxiliary substances, such as those conventional in the art, which are readily available to the public. For example, pharmaceutically acceptable excipients include pH adjusting and buffering agents, tonicity adjusting agents, stabilizers, wetting agents and the like.

As used herein, the term “salt” refers to an acid or base salt of a compound of the invention. Salts of basic compounds are salts formed with mineral acids, organic carboxylic acids, organic sulfonic acids, and the like. Examples of suitable acids include hydrochloric, hydrobromic, sulfuric, nitric, perchloric, fumaric, maleic, phosphoric, glycolic, lactic, salicylic, succinic, toluene-p-sulfonic, tartaric, acetic, citric, methanesulfonic, formic, benzoic, malonic, naphthalene-2-sulfonic and benzenesulfonic acids. Salts of acidic compounds are formed with bases, namely cationic species such as alkali and alkaline earth metal cations e.g., sodium, lithium, potassium, calcium, and magnesium ions as well as ammonium cations e.g., ammonium, trimethylammonium and diethylammonium. Salts of acidic compounds are formed with organic bases, namely tromethamine and meglumine.

The term “subject” includes any subject, generally a rodent, non-rodent, mammal (e.g., human, canine, feline, equine, bovine, ungulate, rabbit etc.), adult or child. The subject used may be healthy or in which treatment for a disorder is desired. The terms “subject” and “patient” may be used interchangeably herein.

The compounds of this invention contain one or more asymmetric carbon atoms and thus occur as racemate and racemic mixtures, single enantiomers, diastereomeric mixtures and individual diastereomers. All such isomeric forms of these compounds are expressly included in the present invention. Each stereo genic carbon may be of the R or S configuration.

As used herein, the term “deprotecting agents” includes, but are not limited to, hydrogen and palladium on carbon (H₂, Pd/C), ammonium formate and palladium on carbon (HCOONH₄, Pd/C), hydrogen and palladium hydroxide on carbon (H₂, Pd(OH)₂/C), combination of Pd/C and Pd(OH)₂/C and acid such as hydrochloride acid, hydrobromic acid and the like.

For purposes of the present invention, the term “substituted” shall be understood to include adding or replacing one or more atoms contained within a functional group or compound with one or more different atoms.

Unless otherwise constrained by the definition of the individual substituent, the above set out groups, like “alkyl”, “alkenyl”, “alkynyl”, “heterocyclic ring”, “cycloalkyl”, “heterocycloalkyl”, “aryl”, “bicyclic” “spirocyclic”, “bridged bicyclic” and “heteroaryl” etc. groups can optionally be substituted with one or more substituents selected from the group consisting of radicals such as “C1-C8-alkyl”, “C2-C8-alkenyl”, “C2-C8-alkynyl”, “cycloalkyl”, “heterocycloalkyl”, “aryl”, “heteroaryl”, “amino”, “alkylamino”, “dialkylamino” “acyl”, “acyloxy”, “acylamino”, “aminocarbonyl”, “alkoxycarbonyl”, “alkoxyalkyloxy”, “alkoxycarbonyloxy”, “carbamate,” “sulfinyl”, “sulfonyl”, “alkoxy”, “sulfanyl”, “halogen”, “carboxy”, “haloalkyl”, “cyano”, “hydroxy”, “mercapto”, “nitro”, “phosphate”, “oxo”, “alkylphosphate” and the like. The one or more substituents is selected from 1 to 10 in number; more preferably from 1 to 5 in number. In one embodiments, radical is further optionally substituted with 1 to 3 substituents selected from C1-C8-alkyl”, “C2-C8-alkenyl”, “C2-C8-alkynyl”, “cycloalkyl”, “heterocycloalkyl”, “aryl”, “heteroaryl”, “amino”, “alkylamino”, “dialkylamino” “acyl”, “acyloxy”, “acylamino”, “aminocarbonyl”, “alkoxycarbonyl”, “alkoxyalkyloxy”, “alkoxycarbonyloxy”, “carbamate,” “sulfinyl”, “sulfonyl”, “alkoxy”, “sulfanyl”, “halogen”, “carboxy”, “haloalkyl”, “cyano”, “hydroxy”, “mercapto”, “nitro”, “phosphate”, “oxo”, “alkylphosphate” and the like.

The term “Oxidising agent” as used herein includes but not limited to reagents such as Pyridinium dichromate (PDC), Pyridinium chlorochromate (PCC), Dess-Martin periodinane (DMP), 2-Iodoxybenzoic acid, Tetrapropylammonium perruthenate (TPAP) 2,2,6,6-tetramethylpiperidine 1-oxyl (TEMPO), Sodium dichromate, Phosphorus pentachloride, Phosphorus trichloride, Hydrogen peroxide, tert-butyl hydroperoxide (TBHP), Iron (III) nitrate nonahydrate, sodium hypochlorite, sulfur trioxide pyridine complex, chromium (VI) oxide, ammonium cerium(IV) nitrate, manganese (IV) oxide, manganese (II) nitrate tetrahydrate, rhodium(III) chloride hydrate, dipyridine chromium trioxide, barium ferrate (VI), Barium permanganate, potassium permanganate, ruthenium (IV) oxide, cerium(IV) ammonium sulfate, The examples of name reactions of oxidations includes but not limiting to Swern oxidation, Jones oxidation, Oppenauer oxidation, pfitzer-Moffatt oxidation, Parikh-Doering oxidation, Albright-Goldman oxidation, Corey-Kim oxidation, and collins reagent, the like or mixtures thereof.

Base used in the present invention can be inorganic and organic base. The examples of organic base includes but not limiting to amines such as diisopropylethylamine, triethylamine, pyridine, 1,8-diazabicyclo[5.4.0]undec-7-ene (DBU), imidazole, N,N-dimethyl aniline, N,N-dimethyl amino pyridine (DMAP), 1,5-diazabicyclo[4.3.0]non-5-ene (DBN) and the like or mixtures thereof. The examples of inorganic base includes but not limiting to alkali or alkaline earth metal carbonate, bicarbonate, hydroxide or phosphate such as potassium carbonate, sodium carbonate, lithium carbonate, sodium bicarbonate, potassium bicarbonate, lithium bicarbonate, sodium hydroxide, potassium hydroxide, lithium hydroxide, potassium phosphate, sodium phosphate; hydride such as sodium hydride, lithium hydride or potassium hydride; alkoxide such as sodium or potassium methoxide or ethoxide, tertiary butoxide and the like or mixtures thereof.

As used herein, the term “solvent” refers to the solvents include, but are not limited to, nitriles such as acetonitrile, propionitrile, butyronitrile, isobutyronitrile and the like; ethers such as dioxane, diethyl ether, diisopropylether, tetrahydrofuran, dimethoxyethane and the like; hydrocarbon such as toluene, xylene, hexane, heptane, cyclohexane and the like; chlorinated hydrocarbon such as methylene chloride, ethylene dichloride, carbon tetra chloride, chloroform, chlorobenzene and the like; polar aprotic solvents such as N,N-dimethylformamide (DMF), dimethyl acetamide (DMAc), dimethyl sulfoxide (DMSO) and the like or mixtures thereof.

The novel compounds of the present invention can be used in conventional solid or liquid pharmaceutical forms, for example as tablets, capsules, powders, granules, solutions, injectables, or sprays. The novel compounds of the present invention for this purpose be processed with conventional pharmaceutical excipients such as binders, bulking agents, preservatives, disintegrants, flow regulators, plasticizers, wetting agents, dispersants, emulsifiers, solvents, release-slowing agents, antioxidants and/or propellant gases.

In one embodiment, the compounds of this invention are administered in combination therapies with other agents, they may be administered sequentially or concurrently to the patient. Alternatively, pharmaceutical compositions according to this invention may be comprised of a combination of a compound of this invention and another therapeutic agent.

In one embodiment, the present invention relates to compound of formula I:

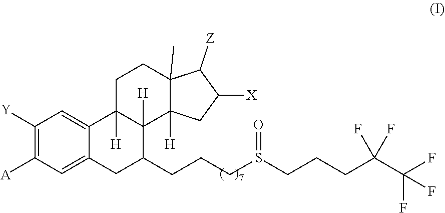

wherein;
X is halogen or hydrogen;
Y is alkyl, —O-alkyl or hydrogen;
Z is selected from ═O, ═N—OH, —O-alkyl, —O-haloalkyl, —NH—OH or —O-alkyl-OH wherein the bond between the Z substituent attached to carbon can be single or double bond depend on the substituent selected;
A is selected from

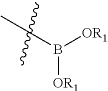

or —O—R₂, wherein R₁ is selected from hydrogen or alkyl; R₂ is selected independently from group consisting of
i)

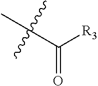

wherein R₃ is selected from NH₂, NHR₄, or NR₅R₆; R₄ is selected from group comprising optionally substituted alkyl, alkenyl, alkynyl, cycloalkyl, heterocycloalkyl, aryl, or heteroaryl; R₅ and R₆ are selected independently from group comprising optionally substituted alkyl, aryl, heteroaryl, heterocycloalkyl, cycloalkyl; or R₅ and R₆ are taken together along with the nitrogen to which they are attached to form a three to seven membered heterocyclic ring and may optionally be substituted at a substitutable position with one or more R₇ radicals, wherein the one or more R₇ radicals are independently selected at each occurrence from the group consisting of optionally substituted alkyl, alkylcarbonyl, formyl, halo, haloalkyl, alkylphosphate, phosphate, oxo, cyano, nitro, amino, hydroxyalkylamino, alkoxy, alkoxycarbonyl, carboxyalkyl, hydroxyalkyl, alkenyl, alkynyl, alkylthio, cycloalkyl, aryl, heteroaryl, aralkyl, heterocycloalkyl, alkylthioalkyl, arylcarbonyl, aralkylcarbonyl, alkoxyalkyl;
ii)

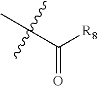

wherein R₈ is selected from group comprising optionally substituted alkyl, alkenyl, alkynyl, cycloalkyl, aryl, heteroaryl; or —CR₉R₁₀; where R₉ and R₁₀ are taken together along with the carbon to which they are attached to form a three to seven membered saturated, partially unsaturated or unsaturated heterocyclic ring in which up to 4 carbon atoms are replaced by heteroatoms chosen from the group consisting of O, S or N and may optionally be substituted at a substitutable position with one or more R₁₁ radicals, wherein one or more R₁₁ radicals are independently selected at each occurrence from the group consisting of optionally substituted alkyl, cycloalkyl, heterocycloalkyl, aryl, heteroaryl, alkylcarbonyl, formyl, halo, alkylphosphate, phosphate, cyano, nitro, alkoxy, amino, alkoxycarbonyl, alkenyl, alkynyl, alkylthio, arylcarbonyl;
iii)

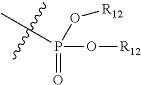

wherein each R₁₂ is selected independently from hydrogen; optionally substituted optionally substituted alkyl, aryl, acyl, heterocycloalkyl or heteroaryl;
iv)

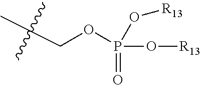

wherein each R₁₃ is selected independently from hydrogen; optionally substituted alkyl, cycloalkyl, heterocycloalkyl, aryl, or heteroaryl;
v) amino acids, wherein the amino acid is linked via ester linkage at the point of attachment;
vi)

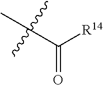

wherein R¹⁴ is n

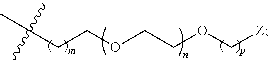

wherein m and p are independently selected from 0 to 5, n refers to degree of polymerization and is selected from 1 to 250 and Z is optionally substituted alkyl or cycloalkyl;
vii)

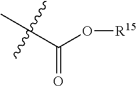

wherein R¹⁵ is selected from group comprising of optionally substituted alkyl, alkenyl, alkynyl, cycloalkyl, heterocycloalkyl, aryl and heteroaryl;
viii)

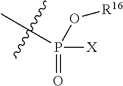

wherein R¹⁶ is selected from group comprising of hydrogen; optionally substituted alkyl, aryl, acyl, heteroaryl; and X is optionally substituted heterocycloalkyl;
ix)

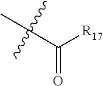

wherein R₁₇ is selected from optionally substituted bicyclic compounds, spirocyclic compounds or bridged bicyclic compounds; pharmaceutically acceptable salts, enantiomers, diastereomers, racemates, or solvates thereof.

In one embodiment, the present invention relates to compound of formula I:

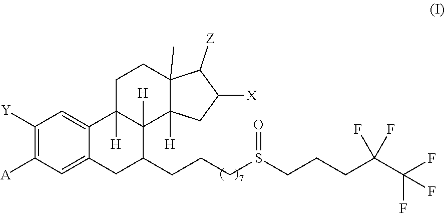

wherein;
X is halogen or hydrogen;
Y is alkyl, —O-alkyl or hydrogen;
Z is selected from ═O, ═N—OH, —O-alkyl, —O-haloalkyl, —NH—OH or —O-alkyl-OH wherein the bond between the Z substituent attached to carbon can be single or double bond depend on the substituent selected;
A is selected as —O—R₂, wherein R₂ is selected independently from group consisting of
i)

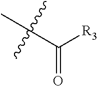

wherein R₃ is selected from NH₂, NHR₄, or NR₅R₆; R₄ is selected from group comprising optionally substituted alkyl, alkenyl, alkynyl, cycloalkyl, heterocycloalkyl, aryl, or heteroaryl; R₅ and R₆ are selected independently from group comprising optionally substituted alkyl, aryl, heteroaryl, heterocycloalkyl, cycloalkyl; or R₅ and R₆ are taken together along with the nitrogen to which they are attached to form a three to seven membered heterocyclic ring and may optionally be substituted at a substitutable position with one or more R₇ radicals, wherein the one or more R₇ radicals are independently selected at each occurrence from the group consisting of optionally substituted alkyl, alkylcarbonyl, formyl, halo, haloalkyl, alkylphosphate, phosphate, oxo, cyano, nitro, amino, hydroxyalkylamino, alkoxy, alkoxycarbonyl, carboxyalkyl, hydroxyalkyl, alkenyl, alkynyl, alkylthio, cycloalkyl, aryl, heteroaryl, aralkyl, heterocycloalkyl, alkylthioalkyl, arylcarbonyl, aralkylcarbonyl, alkoxyalkyl;
ii)

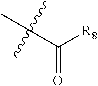

wherein R₈ is selected from group comprising optionally substituted alkyl, alkenyl, alkynyl, cycloalkyl, aryl, heteroaryl; or —CR₉R₁₀; where R₉ and R₁₀ are taken together along with the carbon to which they are attached to form a three to seven membered saturated, partially unsaturated or unsaturated heterocyclic ring in which up to 4 carbon atoms are replaced by heteroatoms chosen from the group consisting of O, S or N and may optionally be substituted at a substitutable position with one or more R₁₁ radicals, wherein one or more R₁₁ radicals are independently selected at each occurrence from the group consisting of optionally substituted alkyl, cycloalkyl, heterocycloalkyl, aryl, heteroaryl, alkylcarbonyl, formyl, halo, alkylphosphate, phosphate, cyano, nitro, alkoxy, amino, alkoxycarbonyl, alkenyl, alkynyl, alkylthio, arylcarbonyl;
iii)

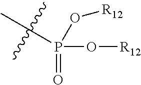

wherein each R₁₂ is selected independently from hydrogen; optionally substituted optionally substituted alkyl, aryl, acyl, heterocycloalkyl or heteroaryl;
iv)

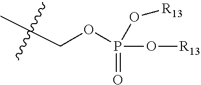

wherein each R₁₃ is selected independently from hydrogen; optionally substituted alkyl, cycloalkyl, heterocycloalkyl, aryl, or heteroaryl;
v) amino acids, wherein the amino acid is linked via ester linkage at the point of attachment;
vi)

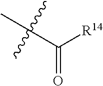

wherein R¹⁴ is

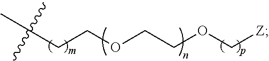

wherein m and p are independently selected from 0 to 5, n refers to degree of polymerization and is selected from 1 to 250 and Z is optionally substituted alkyl or cycloalkyl;
vii)

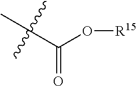

wherein R¹⁵ is selected from group comprising of optionally substituted alkyl, alkenyl, alkynyl, cycloalkyl, heterocycloalkyl, aryl and heteroaryl;
viii)

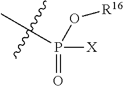

wherein R¹⁶ is selected from group comprising of hydrogen; optionally substituted alkyl, aryl, acyl, heteroaryl; and X is optionally substituted heterocycloalkyl;
ix)

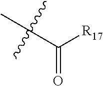

wherein R₁₇ is selected from optionally substituted bicyclic compounds, spirocyclic compounds or bridged bicyclic compounds; pharmaceutically acceptable salts, enantiomers, diastereomers, racemates, or solvates thereof.

In one embodiment, the present invention relates to compound of formula (I-A):

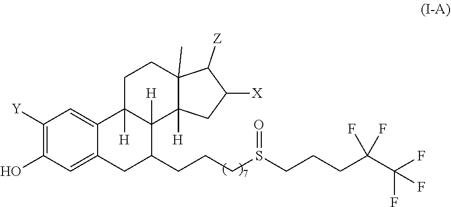

wherein;
X is halogen or hydrogen;
Y is alkyl, —O-alkyl or hydrogen;
Z is selected from ═O, ═N—OH, —O-alkyl, O-haloalkyl, —NH—OH or —O-alkyl-OH wherein the bond between the Z substituent attached to carbon can be single or double bond depend on the substituent selected; pharmaceutically acceptable salts, enantiomers, diastereomers, racemates, or solvates thereof.

In one embodiment of the present invention, bicyclic compounds can be selected from group consisting of

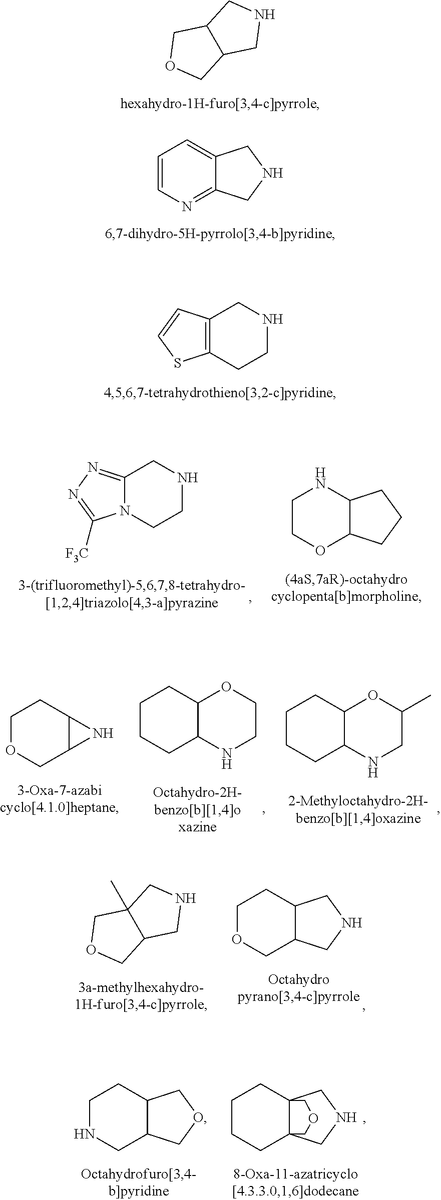

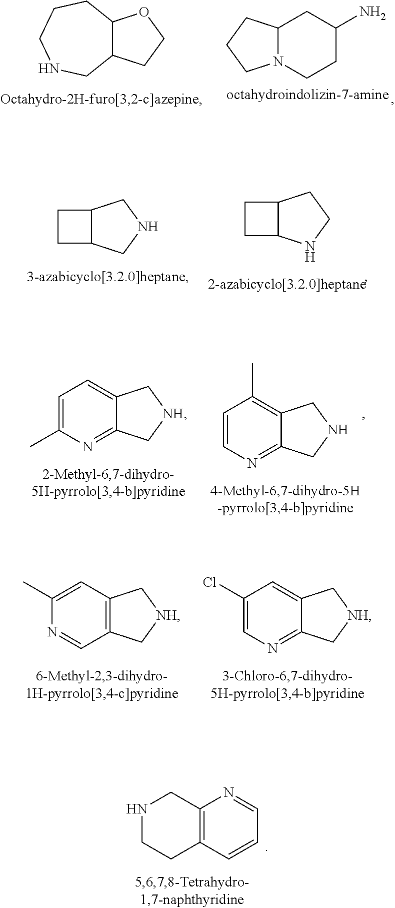

In one embodiment, the bicyclic compounds are optionally substituted with radicals selected from alkyl, alkenyl, alkynyl, cycloalkyl, heterocycloalkyl, aryl, heteroaryl, amino, alkylamino, acyl, acyloxy, acylamino, aminocarbonyl, alkoxycarbonyl, alkoxyalkyloxy, alkoxycarbonyloxy, carbamate, sulfinyl, sulfonyl, alkoxy, sulfanyl, halogen, carboxy, haloalkyl, cyano, hydroxy, mercapto, nitro, phosphate, oxo, alkylphosphate. In one embodiment, the bicyclic compounds can be attached through carbon or nitrogen atom.

In one embodiment of the present invention, spirocyclic compounds can be selected from group consisting of,

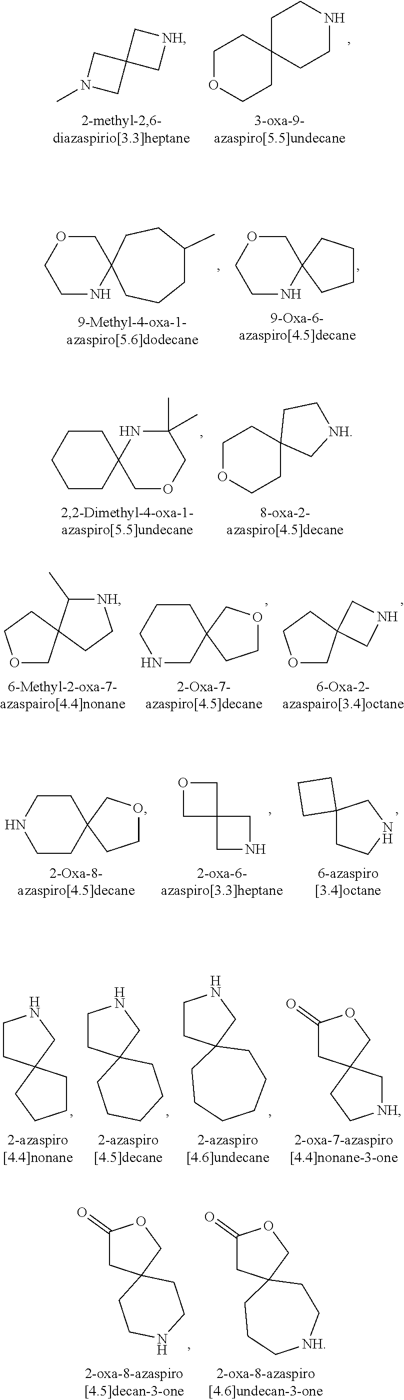

In one embodiment, the spirocyclic compounds is optionally substituted with radicals selected from alkyl, alkenyl, alkynyl, cycloalkyl, heterocycloalkyl, aryl, heteroaryl, amino, alkylamino, acyl, acyloxy, acylamino, aminocarbonyl, alkoxycarbonyl, alkoxyalkyloxy, alkoxycarbonyloxy, carbamate, sulfinyl, sulfonyl, alkoxy, sulfanyl, halogen, carboxy, haloalkyl, cyano, hydroxy, mercapto, nitro, phosphate, oxo, alkylphosphate. In one embodiment, the spirocyclic compounds can be attached through carbon or nitrogen atom.

In one embodiment of the present invention, bridged bicyclic compounds can be selected from group consisting of,

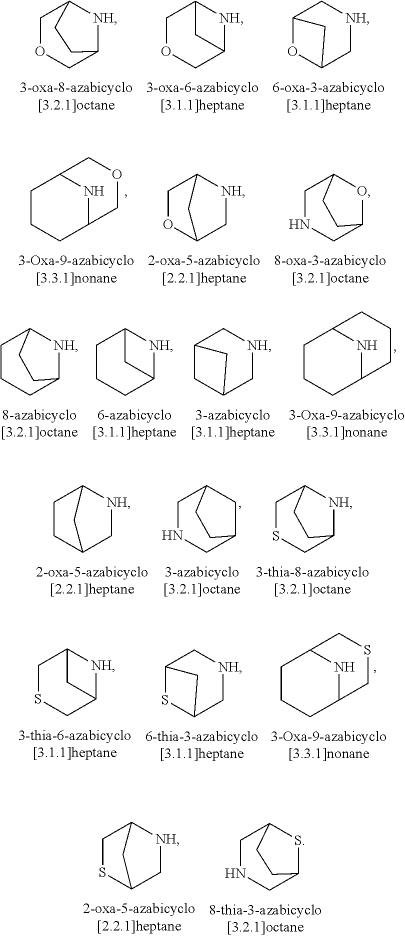

In one embodiment, the bridged bicyclic compounds is optionally substituted with radicals selected from alkyl, alkenyl, alkynyl, cycloalkyl, heterocycloalkyl, aryl, heteroaryl, amino, alkylamino, acyl, acyloxy, acylamino, aminocarbonyl, alkoxycarbonyl, alkoxyalkyloxy, alkoxycarbonyloxy, carbamate, sulfinyl, sulfonyl, alkoxy, sulfanyl, halogen, carboxy, trihalomethyl, cyano, hydroxy, mercapto, nitro, phosphate, oxo, alkylphosphate. In one embodiment, the bridged bicyclic compounds can be attached through carbon or nitrogen atom.

In one embodiment, the present invention relates to compound of formula II:

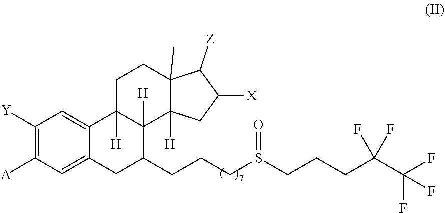

wherein;
X is halogen or hydrogen;
Y is alkyl, —O-alkyl or hydrogen;
A is selected from

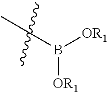

or —O—R₂, wherein R₁ is selected from hydrogen or alkyl; R₂ is selected independently from group consisting of
i)

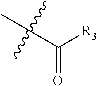

wherein R₃ is selected from NH₂, NHR₄, or NR₅R₆; R₄ is selected from group comprising optionally substituted alkyl, alkenyl, alkynyl, cycloalkyl, heterocycloalkyl, aryl, or heteroaryl; R₅ and R₆ are selected independently from group comprising optionally substituted alkyl, aryl, heteroaryl, heterocycloalkyl, cycloalkyl; or R₅ and R₆ are taken together along with the nitrogen to which they are attached to form a three to seven membered heterocyclic ring and may optionally be substituted at a substitutable position with one or more R₇ radicals, wherein the one or more R₇ radicals are independently selected at each occurrence from the group consisting of optionally substituted alkyl, alkylcarbonyl, formyl, halo, haloalkyl, alkylphosphate, phosphate, oxo, cyano, nitro, amino, hydroxyalkylamino, alkoxy, alkoxycarbonyl, carboxyalkyl, hydroxyalkyl, alkenyl, alkynyl, alkylthio, cycloalkyl, aryl, heteroaryl, aralkyl, heterocycloalkyl, alkylthioalkyl, arylcarbonyl, aralkylcarbonyl, alkoxyalkyl;
ii)

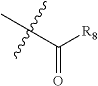

wherein R₈ is selected from group comprising optionally substituted alkyl, alkenyl, alkynyl, cycloalkyl, aryl, heteroaryl; or —CR₉R₁₀; where R₉ and R₁₀ are taken together along with the carbon to which they are attached to form a three to seven membered saturated, partially unsaturated or unsaturated heterocyclic ring in which up to 4 carbon atoms are replaced by heteroatoms chosen from the group consisting of O, S or N and may optionally be substituted at a substitutable position with one or more R₁₁ radicals, wherein one or more R₁₁ radicals are independently selected at each occurrence from the group consisting of optionally substituted alkyl, cycloalkyl, heterocycloalkyl, aryl, heteroaryl, alkylcarbonyl, formyl, halo, alkylphosphate, phosphate, cyano, nitro, alkoxy, amino, alkoxycarbonyl, alkenyl, alkynyl, alkylthio, arylcarbonyl;
iii)

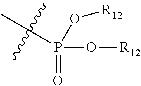

wherein each R₁₂ is selected independently from hydrogen; optionally substituted optionally substituted alkyl, aryl, acyl, heterocycloalkyl or heteroaryl;
iv)

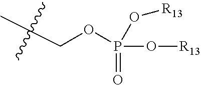

wherein each R₁₃ is selected independently from hydrogen; optionally substituted alkyl, cycloalkyl, heterocycloalkyl, aryl, or heteroaryl;
v) amino acids, wherein the amino acid is linked via ester linkage at the point of attachment;
vi)

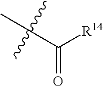

wherein R¹⁴ is

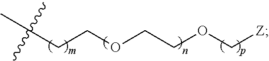

wherein m and p are independently selected from 0 to 5, n refers to degree of polymerization and is selected from 1 to 250 and Z is optionally substituted alkyl or cycloalkyl;
vii)

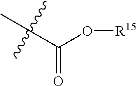

wherein R¹⁵ is selected from group comprising of optionally substituted alkyl, alkenyl, alkynyl, cycloalkyl, heterocycloalkyl, aryl and heteroaryl;
viii)

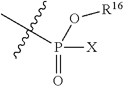

wherein R¹⁶ is selected from group comprising of hydrogen; optionally substituted alkyl, aryl, acyl, heteroaryl; and X is optionally substituted heterocycloalkyl;
ix)

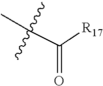

wherein R₁₇ is selected from optionally substituted bicyclic compounds, spirocyclic compounds or bridged bicyclic compounds; pharmaceutically acceptable salts, enantiomers, diastereomers, racemates, or solvates thereof.

In one embodiment, the present invention relates to compound of formula III:

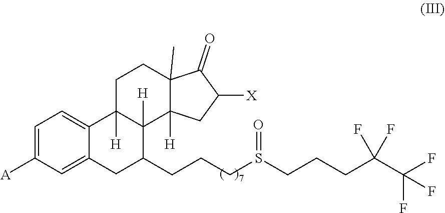

wherein X and A has the same meaning as given in compound of formula (II); pharmaceutically acceptable salts, enantiomers, diastereomers, racemates, or solvates thereof.

In one embodiment, the present invention relates to compound of formula IV:

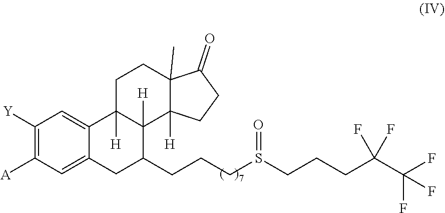

wherein Y and A has the same meaning as given in compound of formula (II); pharmaceutically acceptable salts, enantiomers, diastereomers, racemates, or solvates thereof.

In one embodiment, the present invention relates to compound of formula V:

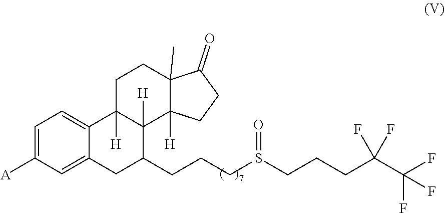

wherein;
A is selected from

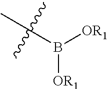

or —O—R₂, wherein R₁ is selected from hydrogen or alkyl; R₂ is selected independently from group consisting of
i)

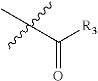

wherein R₃ is selected from NH₂, NHR₄, or NR₅R₆; R₄ is selected from group comprising optionally substituted alkyl, alkenyl, alkynyl, cycloalkyl, heterocycloalkyl, aryl, or heteroaryl; R₅ and R₆ are selected independently from group comprising optionally substituted alkyl, aryl, heteroaryl, heterocycloalkyl, cycloalkyl; or R₅ and R₆ are taken together along with the nitrogen to which they are attached to form a three to seven membered heterocyclic ring and may optionally be substituted at a substitutable position with one or more R₇ radicals, wherein the one or more R₇ radicals are independently selected at each occurrence from the group consisting of optionally substituted alkyl, alkylcarbonyl, formyl, halo, haloalkyl, alkylphosphate, phosphate, oxo, cyano, nitro, amino, hydroxyalkylamino, alkoxy, alkoxycarbonyl, carboxyalkyl, hydroxyalkyl, alkenyl, alkynyl, alkylthio, cycloalkyl, aryl, heteroaryl, aralkyl, heterocycloalkyl, alkylthioalkyl, arylcarbonyl, aralkylcarbonyl, alkoxyalkyl;
ii)

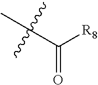

wherein R₈ is selected from group comprising optionally substituted alkyl, alkenyl, alkynyl, cycloalkyl, aryl, heteroaryl; or —CR₉R₁₀; where R₉ and R₁₀ are taken together along with the carbon to which they are attached to form a three to seven membered saturated, partially unsaturated or unsaturated heterocyclic ring in which up to 4 carbon atoms are replaced by heteroatoms chosen from the group consisting of O, S or N and may optionally be substituted at a substitutable position with one or more R₁₁ radicals, wherein one or more R₁₁ radicals are independently selected at each occurrence from the group consisting of optionally substituted alkyl, cycloalkyl, heterocycloalkyl, aryl, heteroaryl, alkylcarbonyl, formyl, halo, alkylphosphate, phosphate, cyano, nitro, alkoxy, amino, alkoxycarbonyl, alkenyl, alkynyl, alkylthio, arylcarbonyl;
iii)

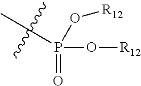

wherein each R₁₂ is selected independently from hydrogen; optionally substituted optionally substituted alkyl, aryl, acyl, heterocycloalkyl or heteroaryl;
iv)

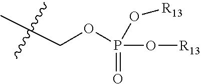

wherein each R₁₃ is selected independently from hydrogen; optionally substituted alkyl, cycloalkyl, heterocycloalkyl, aryl, or heteroaryl;
v) amino acids, wherein the amino acid is linked via ester linkage at the point of attachment;
vi)

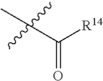

wherein R¹⁴ is

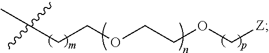

wherein m and p are independently selected from 0 to 5, n refers to degree of polymerization and is selected from 1 to 250 and Z is optionally substituted alkyl or cycloalkyl;
vii)

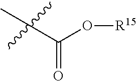

wherein R¹⁵ is selected from group comprising of optionally substituted alkyl, alkenyl, alkynyl, cycloalkyl, heterocycloalkyl, aryl and heteroaryl;
viii)

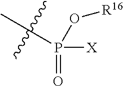

wherein R¹⁶ is selected from group comprising of hydrogen; optionally substituted alkyl, aryl, acyl, heteroaryl; and X is optionally substituted heterocycloalkyl;
ix)

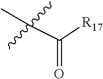

wherein R₁₇ is selected from optionally substituted bicyclic compounds, spirocyclic compounds or bridged bicyclic compounds; pharmaceutically acceptable salts, enantiomers, diastereomers, racemates, or solvates thereof.

In one embodiment, the present invention relates to compound of formula V:

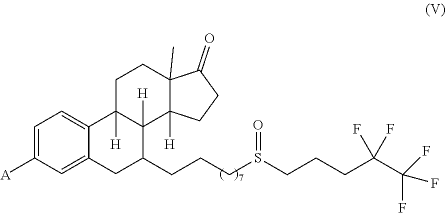

wherein;
A is selected as —O—R₂, wherein R₂ is selected independently from group consisting of
i)

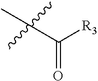

R₃ is selected from NH₂, NHR₄, or NR₅R₆; R₄ is selected from group comprising optionally substituted alkyl, alkenyl, alkynyl, cycloalkyl, heterocycloalkyl, aryl, or heteroaryl; R₅ and R₆ are selected independently from group comprising optionally substituted alkyl, aryl, heteroaryl, heterocycloalkyl, cycloalkyl; or R₅ and R₆ are taken together along with the nitrogen to which they are attached to form a three to seven membered heterocyclic ring and may optionally be substituted at a substitutable position with one or more R₇ radicals, wherein the one or more R₇ radicals are independently selected at each occurrence from the group consisting of optionally substituted alkyl, alkylcarbonyl, formyl, halo, haloalkyl, alkylphosphate, phosphate, oxo, cyano, nitro, amino, hydroxyalkylamino, alkoxy, alkoxycarbonyl, carboxyalkyl, hydroxyalkyl, alkenyl, alkynyl, alkylthio, cycloalkyl, aryl, heteroaryl, aralkyl, heterocycloalkyl, alkylthioalkyl, arylcarbonyl, aralkylcarbonyl, alkoxyalkyl;
ii)

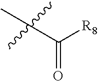

wherein R₈ is selected from group comprising optionally substituted alkyl, alkenyl, alkynyl, cycloalkyl, aryl, heteroaryl; or —CR₉R₁₀; where R₉ and R₁₀ are taken together along with the carbon to which they are attached to form a three to seven membered saturated, partially unsaturated or unsaturated heterocyclic ring in which up to 4 carbon atoms are replaced by heteroatoms chosen from the group consisting of O, S or N and may optionally be substituted at a substitutable position with one or more R₁₁ radicals, wherein one or more R₁₁ radicals are independently selected at each occurrence from the group consisting of optionally substituted alkyl, cycloalkyl, heterocycloalkyl, aryl, heteroaryl, alkylcarbonyl, formyl, halo, alkylphosphate, phosphate, cyano, nitro, alkoxy, amino, alkoxycarbonyl, alkenyl, alkynyl, alkylthio, arylcarbonyl;
iii)

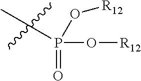

wherein each R₁₂ is selected independently from hydrogen; optionally substituted optionally substituted alkyl, aryl, acyl, heterocycloalkyl or heteroaryl;
iv)

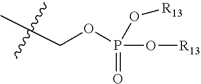

wherein each R₁₃ is selected independently from hydrogen; optionally substituted alkyl, cycloalkyl, heterocycloalkyl, aryl, or heteroaryl;
v) amino acids, wherein the amino acid is linked via ester linkage at the point of attachment;
vi)

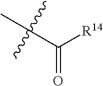

wherein R¹⁴ is

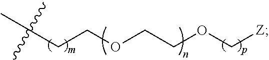

wherein m and p are independently selected from 0 to 5, n refers to degree of polymerization and is selected from 1 to 250 and Z is optionally substituted alkyl or cycloalkyl;
vii)

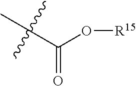

wherein R¹⁵ is selected from group comprising of optionally substituted alkyl, alkenyl, alkynyl, cycloalkyl, heterocycloalkyl, aryl and heteroaryl;
viii)

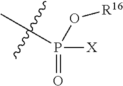

wherein R¹⁶ is selected from group comprising of hydrogen; optionally substituted alkyl, aryl, acyl, heteroaryl; and X is optionally substituted heterocycloalkyl;
ix)

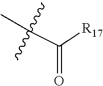

wherein R₁₇ is selected from optionally substituted bicyclic compounds, spirocyclic compounds or bridged bicyclic compounds; pharmaceutically acceptable salts, enantiomers, diastereomers, racemates, or solvates thereof.

In one embodiment, the present invention relates to compound of formula VI:

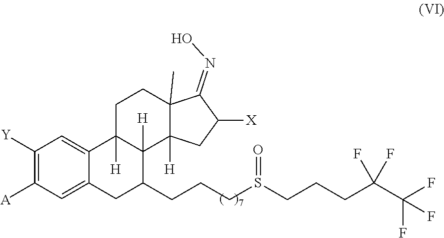

wherein;
X is halogen or hydrogen;
Y is alkyl, —O-alkyl or hydrogen;
A is selected from

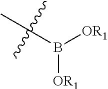

or —O—R₂, wherein R₁ is selected from hydrogen or alkyl; R₂ is selected independently from group consisting of
i)

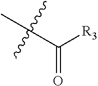

wherein R₃ is selected from NH₂, NHR₄, or NR₅R₆; R₄ is selected from group comprising optionally substituted alkyl, alkenyl, alkynyl, cycloalkyl, heterocycloalkyl, aryl, or heteroaryl; R₅ and R₆ are selected independently from group comprising optionally substituted alkyl, aryl, heteroaryl, heterocycloalkyl, cycloalkyl; or R₅ and R₆ are taken together along with the nitrogen to which they are attached to form a three to seven membered heterocyclic ring and may optionally be substituted at a substitutable position with one or more R₇ radicals, wherein the one or more R₇ radicals are independently selected at each occurrence from the group consisting of optionally substituted alkyl, alkylcarbonyl, formyl, halo, haloalkyl, alkylphosphate, phosphate, oxo, cyano, nitro, amino, hydroxyalkylamino, alkoxy, alkoxycarbonyl, carboxyalkyl, hydroxyalkyl, alkenyl, alkynyl, alkylthio, cycloalkyl, aryl, heteroaryl, aralkyl, heterocycloalkyl, alkylthioalkyl, arylcarbonyl, aralkylcarbonyl, alkoxyalkyl;
ii)

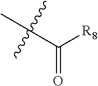

wherein R₈ is selected from group comprising optionally substituted alkyl, alkenyl, alkynyl, cycloalkyl, aryl, heteroaryl; or —CR₉R₁₀; where R₉ and R₁₀ are taken together along with the carbon to which they are attached to form a three to seven membered saturated, partially unsaturated or unsaturated heterocyclic ring in which up to 4 carbon atoms are replaced by heteroatoms chosen from the group consisting of O, S or N and may optionally be substituted at a substitutable position with one or more R₁₁ radicals, wherein one or more R₁₁ radicals are independently selected at each occurrence from the group consisting of optionally substituted alkyl, cycloalkyl, heterocycloalkyl, aryl, heteroaryl, alkylcarbonyl, formyl, halo, alkylphosphate, phosphate, cyano, nitro, alkoxy, amino, alkoxycarbonyl, alkenyl, alkynyl, alkylthio, arylcarbonyl;
iii)

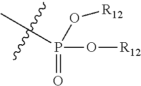

wherein each R₁₂ is selected independently from hydrogen; optionally substituted optionally substituted alkyl, aryl, acyl, heterocycloalkyl or heteroaryl;
iv)

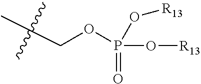

wherein each R₁₃ is selected independently from hydrogen; optionally substituted alkyl, cycloalkyl, heterocycloalkyl, aryl, or heteroaryl;
v) amino acids, wherein the amino acid is linked via ester linkage at the point of attachment;
vi)

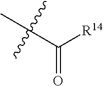

wherein R¹⁴ is

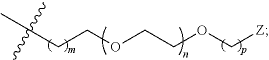

wherein m and p are independently selected from 0 to 5, n refers to degree of polymerization and is selected from 1 to 250 and Z is optionally substituted alkyl or cycloalkyl;
vii)

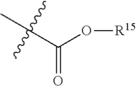

wherein R¹⁵ is selected from group comprising of optionally substituted alkyl, alkenyl, alkynyl, cycloalkyl, heterocycloalkyl, aryl and heteroaryl;
viii)

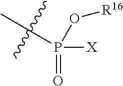

wherein R¹⁶ is selected from group comprising of hydrogen; optionally substituted alkyl, aryl, acyl, heteroaryl; and X is optionally substituted heterocycloalkyl;
ix)

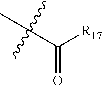

wherein R₁₇ is selected from optionally substituted bicyclic compounds, spirocyclic compounds or bridged bicyclic compounds; pharmaceutically acceptable salts, enantiomers, diastereomers, racemates, or solvates thereof.

In one embodiment, the present invention relates to compound of formula VII:

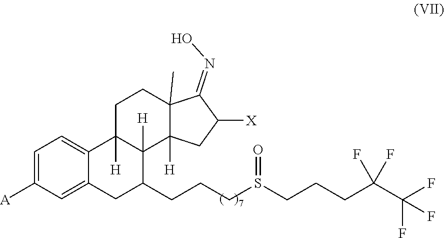

wherein X and A has the same meaning as given in compound of formula (VI); pharmaceutically acceptable salts, enantiomers, diastereomers, racemates, or solvates thereof.

In one embodiment, the present invention relates to compound of formula VIII:

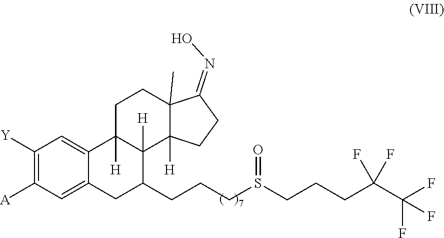

wherein Y and A has the same meaning as given in compound of formula (VI); pharmaceutically acceptable salts, enantiomers, diastereomers, racemates, or solvates thereof.

In one embodiment, the present invention relates to compound of formula IX:

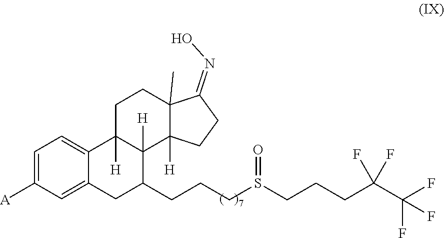

A is selected from

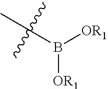

or —O—R2, wherein R1 is selected from hydrogen or alkyl; R₂ is selected independently from group consisting of
i)

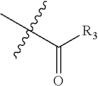

wherein R₃ is selected from NH₂, NHR₄, or NR₅R₆; R₄ is selected from group comprising optionally substituted alkyl, alkenyl, alkynyl, cycloalkyl, heterocycloalkyl, aryl, or heteroaryl; R₅ and R₆ are selected independently from group comprising optionally substituted alkyl, aryl, heteroaryl, heterocycloalkyl, cycloalkyl; or R₅ and R₆ are taken together along with the nitrogen to which they are attached to form a three to seven membered heterocyclic ring and may optionally be substituted at a substitutable position with one or more R₇ radicals, wherein the one or more R₇ radicals are independently selected at each occurrence from the group consisting of optionally substituted alkyl, alkylcarbonyl, formyl, halo, haloalkyl, alkylphosphate, phosphate, oxo, cyano, nitro, amino, hydroxyalkylamino, alkoxy, alkoxycarbonyl, carboxyalkyl, hydroxyalkyl, alkenyl, alkynyl, alkylthio, cycloalkyl, aryl, heteroaryl, aralkyl, heterocycloalkyl, alkylthioalkyl, arylcarbonyl, aralkylcarbonyl, alkoxyalkyl;
ii)

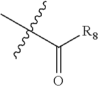

wherein R₈ is selected from group comprising optionally substituted alkyl, alkenyl, alkynyl, cycloalkyl, aryl, heteroaryl; or —CR₉R₁₀; where R₉ and R₁₀ are taken together along with the carbon to which they are attached to form a three to seven membered saturated, partially unsaturated or unsaturated heterocyclic ring in which up to 4 carbon atoms are replaced by heteroatoms chosen from the group consisting of O, S or N and may optionally be substituted at a substitutable position with one or more R₁₁ radicals, wherein one or more R₁₁ radicals are independently selected at each occurrence from the group consisting of optionally substituted alkyl, cycloalkyl, heterocycloalkyl, aryl, heteroaryl, alkylcarbonyl, formyl, halo, alkylphosphate, phosphate, cyano, nitro, alkoxy, amino, alkoxycarbonyl, alkenyl, alkynyl, alkylthio, arylcarbonyl;
iii)

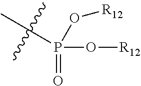

wherein each R₁₂ is selected independently from hydrogen; optionally substituted optionally substituted alkyl, aryl, acyl, heterocycloalkyl or heteroaryl;
iv)

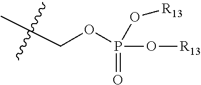

wherein each R₁₃ is selected independently from hydrogen; optionally substituted alkyl, cycloalkyl, heterocycloalkyl, aryl, or heteroaryl;
v) amino acids, wherein the amino acid is linked via ester linkage at the point of attachment;
vi)

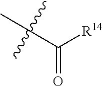

wherein R¹⁴ is

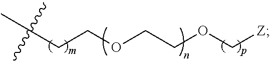

wherein m and p are independently selected from 0 to 5, n refers to degree of polymerization and is selected from 1 to 250 and Z is optionally substituted alkyl or cycloalkyl;
vii)

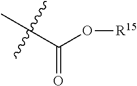

wherein R¹⁵ is selected from group comprising of optionally substituted alkyl, alkenyl, alkynyl, cycloalkyl, heterocycloalkyl, aryl and heteroaryl;
viii)

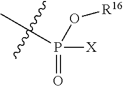

wherein R¹⁶ is selected from group comprising of hydrogen; optionally substituted alkyl, aryl, acyl, heteroaryl; and X is optionally substituted heterocycloalkyl;
ix)

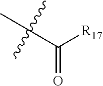

wherein R₁₇ is selected from optionally substituted bicyclic compounds, spirocyclic compounds or bridged bicyclic compounds; pharmaceutically acceptable salts, enantiomers, diastereomers, racemates, or solvates thereof.

In one embodiment, the present invention relates to compound of formula X:

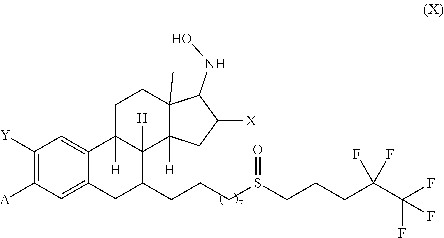

X is halogen or hydrogen;
Y is alkyl, —O-alkyl or hydrogen;
A is selected from

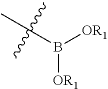

or —O—R₂, wherein R₁ is selected from hydrogen or alkyl; R₂ is selected independently from group consisting of
i)

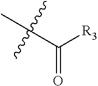

wherein R₃ is selected from NH₂, NHR₄, or NR₅R₆; R₄ is selected from group comprising optionally substituted alkyl, alkenyl, alkynyl, cycloalkyl, heterocycloalkyl, aryl, or heteroaryl; R₅ and R₆ are selected independently from group comprising optionally substituted alkyl, aryl, heteroaryl, heterocycloalkyl, cycloalkyl; or R₅ and R₆ are taken together along with the nitrogen to which they are attached to form a three to seven membered heterocyclic ring and may optionally be substituted at a substitutable position with one or more R₇ radicals, wherein the one or more R₇ radicals are independently selected at each occurrence from the group consisting of optionally substituted alkyl, alkylcarbonyl, formyl, halo, haloalkyl, alkylphosphate, phosphate, oxo, cyano, nitro, amino, hydroxyalkylamino, alkoxy, alkoxycarbonyl, carboxyalkyl, hydroxyalkyl, alkenyl, alkynyl, alkylthio, cycloalkyl, aryl, heteroaryl, aralkyl, heterocycloalkyl, alkylthioalkyl, arylcarbonyl, aralkylcarbonyl, alkoxyalkyl;
ii)

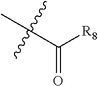

wherein R₈ is selected from group comprising optionally substituted alkyl, alkenyl, alkynyl, cycloalkyl, aryl, heteroaryl; or —CR₉R₁₀; where R₉ and R₁₀ are taken together along with the carbon to which they are attached to form a three to seven membered saturated, partially unsaturated or unsaturated heterocyclic ring in which up to 4 carbon atoms are replaced by heteroatoms chosen from the group consisting of O, S or N and may optionally be substituted at a substitutable position with one or more R₁₁ radicals, wherein one or more R₁₁ radicals are independently selected at each occurrence from the group consisting of optionally substituted alkyl, cycloalkyl, heterocycloalkyl, aryl, heteroaryl, alkylcarbonyl, formyl, halo, alkylphosphate, phosphate, cyano, nitro, alkoxy, amino, alkoxycarbonyl, alkenyl, alkynyl, alkylthio, arylcarbonyl;
iii)

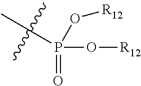

wherein each R₁₂ is selected independently from hydrogen; optionally substituted optionally substituted alkyl, aryl, acyl, heterocycloalkyl or heteroaryl;
iv)

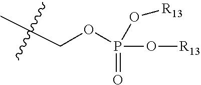

wherein each R₁₃ is selected independently from hydrogen; optionally substituted alkyl, cycloalkyl, heterocycloalkyl, aryl, or heteroaryl;
v) amino acids, wherein the amino acid is linked via ester linkage at the point of attachment;
vi)

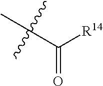

wherein R¹⁴ is

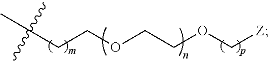

wherein m and p are independently selected from 0 to 5, n refers to degree of polymerization and is selected from 1 to 250 and Z is optionally substituted alkyl or cycloalkyl;
vii)

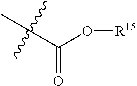

wherein R¹⁵ is selected from group comprising of optionally substituted alkyl, alkenyl, alkynyl, cycloalkyl, heterocycloalkyl, aryl and heteroaryl;
viii)

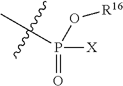

wherein R¹⁶ is selected from group comprising of hydrogen; optionally substituted alkyl, aryl, acyl, heteroaryl; and X is optionally substituted heterocycloalkyl;
ix)

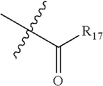

wherein R₁₇ is selected from optionally substituted bicyclic compounds, spirocyclic compounds or bridged bicyclic compounds; pharmaceutically acceptable salts, enantiomers, diastereomers, racemates, or solvates thereof.

In one embodiment, the present invention relates to compound of formula XI:

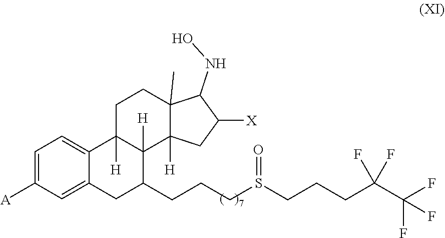

wherein X and A has the same meaning as given in compound of formula (X); pharmaceutically acceptable salts, enantiomers, diastereomers, racemates, or solvates thereof.

In one embodiment, the present invention relates to compound of formula XII:

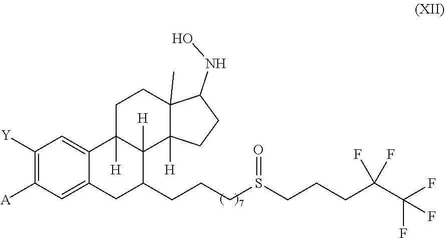

wherein Y and A has the same meaning as given in compound of formula (X); pharmaceutically acceptable salts, enantiomers, diastereomers, racemates, or solvates thereof.

In one embodiment, the present invention relates to compound of formula XIII:

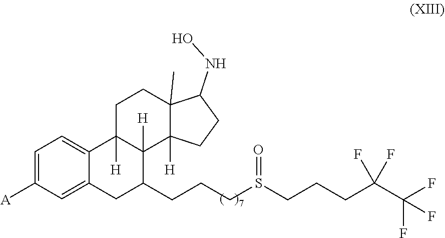

wherein;
A is selected from

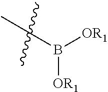

or —O—R2, wherein R1 is selected from hydrogen or alkyl; R₂ is selected independently from group consisting of
i)

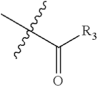

wherein R₃ is selected from NH₂, NHR₄, or NR₅R₆; R₄ is selected from group comprising optionally substituted alkyl, alkenyl, alkynyl, cycloalkyl, heterocycloalkyl, aryl, or heteroaryl; R₅ and R₆ are selected independently from group comprising optionally substituted alkyl, aryl, heteroaryl, heterocycloalkyl, cycloalkyl; or R₅ and R₆ are taken together along with the nitrogen to which they are attached to form a three to seven membered heterocyclic ring and may optionally be substituted at a substitutable position with one or more R₇ radicals, wherein the one or more R₇ radicals are independently selected at each occurrence from the group consisting of optionally substituted alkyl, alkylcarbonyl, formyl, halo, haloalkyl, alkylphosphate, phosphate, oxo, cyano, nitro, amino, hydroxyalkylamino, alkoxy, alkoxycarbonyl, carboxyalkyl, hydroxyalkyl, alkenyl, alkynyl, alkylthio, cycloalkyl, aryl, heteroaryl, aralkyl, heterocycloalkyl, alkylthioalkyl, arylcarbonyl, aralkylcarbonyl, alkoxyalkyl;
ii)

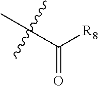

wherein R₈ is selected from group comprising optionally substituted alkyl, alkenyl, alkynyl, cycloalkyl, aryl, heteroaryl; or —CR₉R₁₀; where R₉ and R₁₀ are taken together along with the carbon to which they are attached to form a three to seven membered saturated, partially unsaturated or unsaturated heterocyclic ring in which up to 4 carbon atoms are replaced by heteroatoms chosen from the group consisting of O, S or N and may optionally be substituted at a substitutable position with one or more R₁₁ radicals, wherein one or more R₁₁ radicals are independently selected at each occurrence from the group consisting of optionally substituted alkyl, cycloalkyl, heterocycloalkyl, aryl, heteroaryl, alkylcarbonyl, formyl, halo, alkylphosphate, phosphate, cyano, nitro, alkoxy, amino, alkoxycarbonyl, alkenyl, alkynyl, alkylthio, arylcarbonyl;
iii)

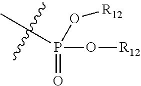

wherein each R₁₂ is selected independently from hydrogen; optionally substituted optionally substituted alkyl, aryl, acyl, heterocycloalkyl or heteroaryl;
iv)

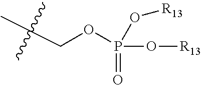

wherein each R₁₃ is selected independently from hydrogen; optionally substituted alkyl, cycloalkyl, heterocycloalkyl, aryl, or heteroaryl;
v) amino acids, wherein the amino acid is linked via ester linkage at the point of attachment;
vi)

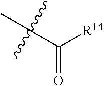

wherein R¹⁴ is

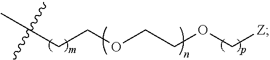

wherein m and p are independently selected from 0 to 5, n refers to degree of polymerization and is selected from 1 to 250 and Z is optionally substituted alkyl or cycloalkyl;
vii)

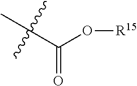

wherein R¹⁵ is selected from group comprising of optionally substituted alkyl, alkenyl, alkynyl, cycloalkyl, heterocycloalkyl, aryl and heteroaryl;
viii)

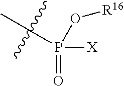

wherein R¹⁶ is selected from group comprising of hydrogen; optionally substituted alkyl, aryl, acyl, heteroaryl; and X is optionally substituted heterocycloalkyl;
ix)

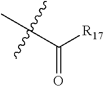

wherein R₁₇ is selected from optionally substituted bicyclic compounds, spirocyclic compounds or bridged bicyclic compounds; pharmaceutically acceptable salts, enantiomers, diastereomers, racemates, or solvates thereof.

In one embodiment, the present invention relates to compound of formula XIV:

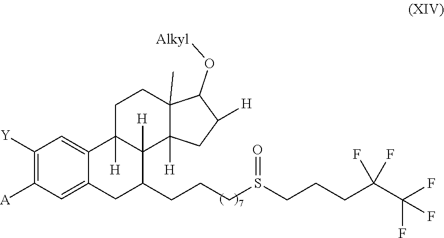

wherein;
X is halogen or hydrogen;
Y is alkyl, —O-alkyl or hydrogen;
A is selected from

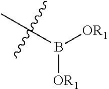

or —O—R2, wherein R1 is selected from hydrogen or alkyl; R₂ is selected independently from group consisting of
i)

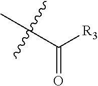

wherein R₃ is selected from NH₂, NHR₄, or NR₅R₆; R₄ is selected from group comprising optionally substituted alkyl, alkenyl, alkynyl, cycloalkyl, heterocycloalkyl, aryl, or heteroaryl; R₅ and R₆ are selected independently from group comprising optionally substituted alkyl, aryl, heteroaryl, heterocycloalkyl, cycloalkyl; or R₅ and R₆ are taken together along with the nitrogen to which they are attached to form a three to seven membered heterocyclic ring and may optionally be substituted at a substitutable position with one or more R₇ radicals, wherein the one or more R₇ radicals are independently selected at each occurrence from the group consisting of optionally substituted alkyl, alkylcarbonyl, formyl, halo, haloalkyl, alkylphosphate, phosphate, oxo, cyano, nitro, amino, hydroxyalkylamino, alkoxy, alkoxycarbonyl, carboxyalkyl, hydroxyalkyl, alkenyl, alkynyl, alkylthio, cycloalkyl, aryl, heteroaryl, aralkyl, heterocycloalkyl, alkylthioalkyl, arylcarbonyl, aralkylcarbonyl, alkoxyalkyl;
ii)

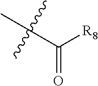

wherein R₈ is selected from group comprising optionally substituted alkyl, alkenyl, alkynyl, cycloalkyl, aryl, heteroaryl; or —CR₉R₁₀; where R₉ and R₁₀ are taken together along with the carbon to which they are attached to form a three to seven membered saturated, partially unsaturated or unsaturated heterocyclic ring in which up to 4 carbon atoms are replaced by heteroatoms chosen from the group consisting of O, S or N and may optionally be substituted at a substitutable position with one or more R₁₁ radicals, wherein one or more R₁₁ radicals are independently selected at each occurrence from the group consisting of optionally substituted alkyl, cycloalkyl, heterocycloalkyl, aryl, heteroaryl, alkylcarbonyl, formyl, halo, alkylphosphate, phosphate, cyano, nitro, alkoxy, amino, alkoxycarbonyl, alkenyl, alkynyl, alkylthio, arylcarbonyl;
iii)

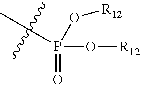

wherein each R₁₂ is selected independently from hydrogen; optionally substituted optionally substituted alkyl, aryl, acyl, heterocycloalkyl or heteroaryl;
iv)

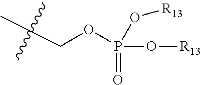

wherein each R₁₃ is selected independently from hydrogen; optionally substituted alkyl, cycloalkyl, heterocycloalkyl, aryl, or heteroaryl;
v) amino acids, wherein the amino acid is linked via ester linkage at the point of attachment;
vi)

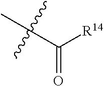

wherein R¹⁴ is

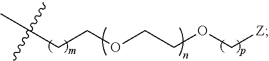

wherein m and p are independently selected from 0 to 5, n refers to degree of polymerization and is selected from 1 to 250 and Z is optionally substituted alkyl or cycloalkyl;
vii)

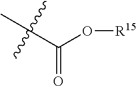

wherein R¹⁵ is selected from group comprising of optionally substituted alkyl, alkenyl, alkynyl, cycloalkyl, heterocycloalkyl, aryl and heteroaryl;
viii)

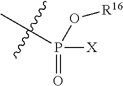

wherein R¹⁶ is selected from group comprising of hydrogen; optionally substituted alkyl, aryl, acyl, heteroaryl; and X is optionally substituted heterocycloalkyl;
ix)

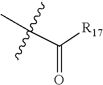

wherein R₁₇ is selected from optionally substituted bicyclic compounds, spirocyclic compounds or bridged bicyclic compounds; pharmaceutically acceptable salts, enantiomers, diastereomers, racemates, or solvates thereof.

In one embodiment, the present invention relates to compound of formula XV:

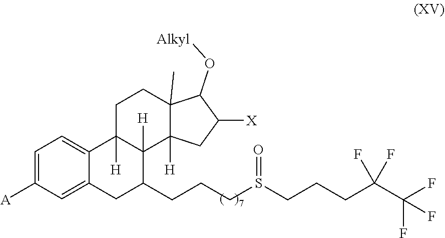

wherein X and A has the same meaning as given in compound of formula (XIV); pharmaceutically acceptable salts, enantiomers, diastereomers, racemates, or solvates thereof.

In one embodiment, the present invention relates to compound of formula XVI:

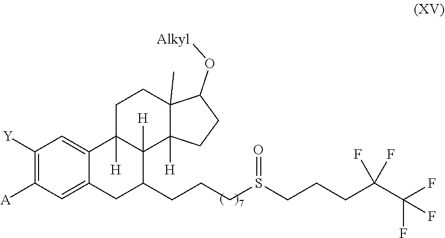

wherein Y and A has the same meaning as given in compound of formula (XIV); pharmaceutically acceptable salts, enantiomers, diastereomers, racemates, or solvates thereof.

In one embodiment, the present invention relates to compound of formula XVII:

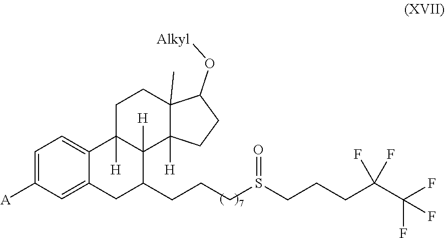

wherein;
A is selected from

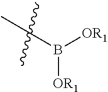

or —O—R2, wherein R1 is selected from hydrogen or alkyl; R₂ is selected independently from group consisting of
i)

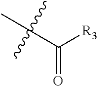

wherein R₃ is selected from NH₂, NHR₄, or NR₅R₆; R₄ is selected from group comprising optionally substituted alkyl, alkenyl, alkynyl, cycloalkyl, heterocycloalkyl, aryl, or heteroaryl; R₅ and R₆ are selected independently from group comprising optionally substituted alkyl, aryl, heteroaryl, heterocycloalkyl, cycloalkyl; or R₅ and R₆ are taken together along with the nitrogen to which they are attached to form a three to seven membered heterocyclic ring and may optionally be substituted at a substitutable position with one or more R₇ radicals, wherein the one or more R₇ radicals are independently selected at each occurrence from the group consisting of optionally substituted alkyl, alkylcarbonyl, formyl, halo, haloalkyl, alkylphosphate, phosphate, oxo, cyano, nitro, amino, hydroxyalkylamino, alkoxy, alkoxycarbonyl, carboxyalkyl, hydroxyalkyl, alkenyl, alkynyl, alkylthio, cycloalkyl, aryl, heteroaryl, aralkyl, heterocycloalkyl, alkylthioalkyl, arylcarbonyl, aralkylcarbonyl, alkoxyalkyl;
ii)

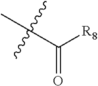

wherein R₈ is selected from group comprising optionally substituted alkyl, alkenyl, alkynyl, cycloalkyl, aryl, heteroaryl; or —CR₉R₁₀; where R₉ and R₁₀ are taken together along with the carbon to which they are attached to form a three to seven membered saturated, partially unsaturated or unsaturated heterocyclic ring in which up to 4 carbon atoms are replaced by heteroatoms chosen from the group consisting of O, S or N and may optionally be substituted at a substitutable position with one or more R₁₁ radicals, wherein one or more R₁₁ radicals are independently selected at each occurrence from the group consisting of optionally substituted alkyl, cycloalkyl, heterocycloalkyl, aryl, heteroaryl, alkylcarbonyl, formyl, halo, alkylphosphate, phosphate, cyano, nitro, alkoxy, amino, alkoxycarbonyl, alkenyl, alkynyl, alkylthio, arylcarbonyl;
iii)

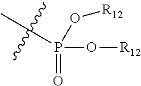

wherein each R₁₂ is selected independently from hydrogen; optionally substituted optionally substituted alkyl, aryl, acyl, heterocycloalkyl or heteroaryl;
iv)

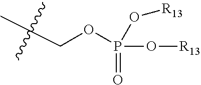

wherein each R₁₃ is selected independently from hydrogen; optionally substituted alkyl, cycloalkyl, heterocycloalkyl, aryl, or heteroaryl;
v) amino acids, wherein the amino acid is linked via ester linkage at the point of attachment;
vi)

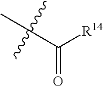

wherein R¹⁴ is

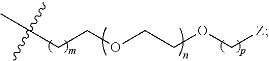

wherein m and p are independently selected from 0 to 5, n refers to degree of polymerization and is selected from 1 to 250 and Z is optionally substituted alkyl or cycloalkyl;
vii)

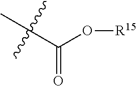

wherein R¹⁵ is selected from group comprising of optionally substituted alkyl, alkenyl, alkynyl, cycloalkyl, heterocycloalkyl, aryl and heteroaryl;
viii)

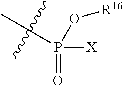

wherein R¹⁶ is selected from group comprising of hydrogen; optionally substituted alkyl, aryl, acyl, heteroaryl; and X is optionally substituted heterocycloalkyl;
ix)

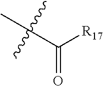

wherein R₁₇ is selected from optionally substituted bicyclic compounds, spirocyclic compounds or bridged bicyclic compounds; pharmaceutically acceptable salts, enantiomers, diastereomers, racemates, or solvates thereof.

In one embodiment, the present invention relates to compound of formula XVIII:

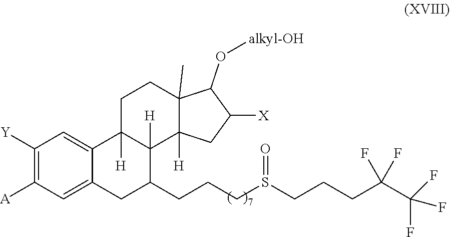

wherein;
X is halogen or hydrogen;
Y is alkyl, —O-alkyl or hydrogen;
A is selected from

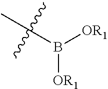

or —O—R2, wherein R1 is selected from hydrogen or alkyl; R₂ is selected independently from group consisting of
i)

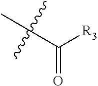

wherein R₃ is selected from NH₂, NHR₄, or NR₅R₆; R₄ is selected from group comprising optionally substituted alkyl, alkenyl, alkynyl, cycloalkyl, heterocycloalkyl, aryl, or heteroaryl; R₅ and R₆ are selected independently from group comprising optionally substituted alkyl, aryl, heteroaryl, heterocycloalkyl, cycloalkyl; or R₅ and R₆ are taken together along with the nitrogen to which they are attached to form a three to seven membered heterocyclic ring and may optionally be substituted at a substitutable position with one or more R₇ radicals, wherein the one or more R₇ radicals are independently selected at each occurrence from the group consisting of optionally substituted alkyl, alkylcarbonyl, formyl, halo, haloalkyl, alkylphosphate, phosphate, oxo, cyano, nitro, amino, hydroxyalkylamino, alkoxy, alkoxycarbonyl, carboxyalkyl, hydroxyalkyl, alkenyl, alkynyl, alkylthio, cycloalkyl, aryl, heteroaryl, aralkyl, heterocycloalkyl, alkylthioalkyl, arylcarbonyl, aralkylcarbonyl, alkoxyalkyl;
ii)

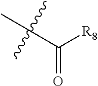

wherein R₈ is selected from group comprising optionally substituted alkyl, alkenyl, alkynyl, cycloalkyl, aryl, heteroaryl; or —CR₉R₁₀; where R₉ and R₁₀ are taken together along with the carbon to which they are attached to form a three to seven membered saturated, partially unsaturated or unsaturated heterocyclic ring in which up to 4 carbon atoms are replaced by heteroatoms chosen from the group consisting of O, S or N and may optionally be substituted at a substitutable position with one or more R₁₁ radicals, wherein one or more R₁₁ radicals are independently selected at each occurrence from the group consisting of optionally substituted alkyl, cycloalkyl, heterocycloalkyl, aryl, heteroaryl, alkylcarbonyl, formyl, halo, alkylphosphate, phosphate, cyano, nitro, alkoxy, amino, alkoxycarbonyl, alkenyl, alkynyl, alkylthio, arylcarbonyl;
iii)

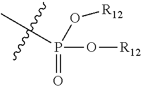

wherein each R₁₂ is selected independently from hydrogen; optionally substituted optionally substituted alkyl, aryl, acyl, heterocycloalkyl or heteroaryl;
iv)

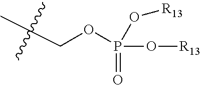

wherein each R₁₃ is selected independently from hydrogen; optionally substituted alkyl, cycloalkyl, heterocycloalkyl, aryl, or heteroaryl;
v) amino acids, wherein the amino acid is linked via ester linkage at the point of attachment;
vi)

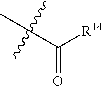

wherein R₁₄ is

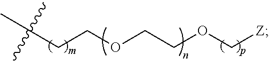

wherein m and p are independently selected from 0 to 5, n refers to degree of polymerization and is selected from 1 to 250 and Z is optionally substituted alkyl or cycloalkyl;
vii)

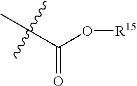

wherein R¹⁵ is selected from group comprising of optionally substituted alkyl, alkenyl, alkynyl, cycloalkyl, heterocycloalkyl, aryl and heteroaryl;
viii)

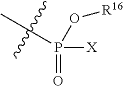

wherein R¹⁶ is selected from group comprising of hydrogen; optionally substituted alkyl, aryl, acyl, heteroaryl; and X is optionally substituted heterocycloalkyl;
ix)

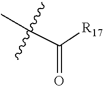

wherein R₁₇ is selected from optionally substituted bicyclic compounds, spirocyclic compounds or bridged bicyclic compounds; pharmaceutically acceptable salts, enantiomers, diastereomers, racemates, or solvates thereof.

In one embodiment, the present invention relates to compound of formula XIX:

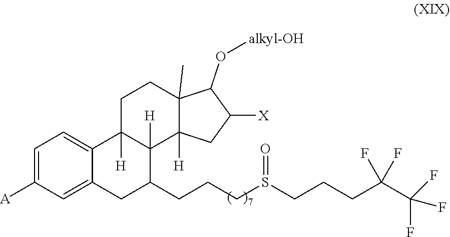

wherein X and A has the same meaning as given in compound of formula (XVIII); pharmaceutically acceptable salts, enantiomers, diastereomers, racemates, or solvates thereof.

In one embodiment, the present invention relates to compound of formula XX:

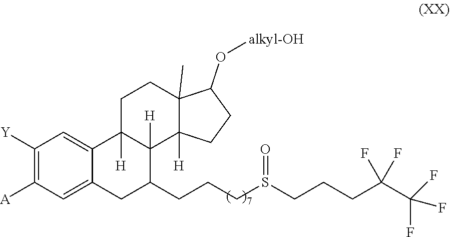

wherein Y and A has the same meaning as given in compound of formula XVIII; pharmaceutically acceptable salts, enantiomers, diastereomers, racemates, or solvates thereof.

In one embodiment, the present invention relates to compound of formula XXI:

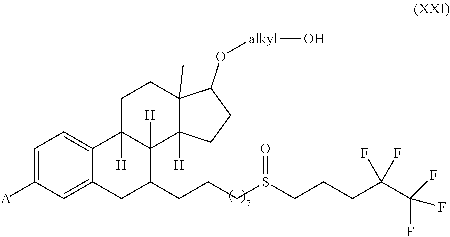

wherein;
A is selected from

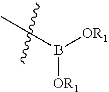

or —O—R2, wherein R1 is selected from hydrogen or alkyl; R₂ is selected independently from group consisting of
i)

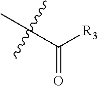

wherein R₃ is selected from NH₂, NHR₄, or NR₅R₆; R₄ is selected from group comprising optionally substituted alkyl, alkenyl, alkynyl, cycloalkyl, heterocycloalkyl, aryl, or heteroaryl; R₅ and R₆ are selected independently from group comprising optionally substituted alkyl, aryl, heteroaryl, heterocycloalkyl, cycloalkyl; or R₅ and R₆ are taken together along with the nitrogen to which they are attached to form a three to seven membered heterocyclic ring and may optionally be substituted at a substitutable position with one or more R₇ radicals, wherein the one or more R₇ radicals are independently selected at each occurrence from the group consisting of optionally substituted alkyl, alkylcarbonyl, formyl, halo, haloalkyl, alkylphosphate, phosphate, oxo, cyano, nitro, amino, hydroxyalkylamino, alkoxy, alkoxycarbonyl, carboxyalkyl, hydroxyalkyl, alkenyl, alkynyl, alkylthio, cycloalkyl, aryl, heteroaryl, aralkyl, heterocycloalkyl, alkylthioalkyl, arylcarbonyl, aralkylcarbonyl, alkoxyalkyl;
ii)

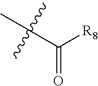

wherein R₈ is selected from group comprising optionally substituted alkyl, alkenyl, alkynyl, cycloalkyl, aryl, heteroaryl; or —CR₉R₁₀; where R₉ and R₁₀ are taken together along with the carbon to which they are attached to form a three to seven membered saturated, partially unsaturated or unsaturated heterocyclic ring in which up to 4 carbon atoms are replaced by heteroatoms chosen from the group consisting of O, S or N and may optionally be substituted at a substitutable position with one or more R₁₁ radicals, wherein one or more R₁₁ radicals are independently selected at each occurrence from the group consisting of optionally substituted alkyl, cycloalkyl, heterocycloalkyl, aryl, heteroaryl, alkylcarbonyl, formyl, halo, alkylphosphate, phosphate, cyano, nitro, alkoxy, amino, alkoxycarbonyl, alkenyl, alkynyl, alkylthio, arylcarbonyl;
iii)

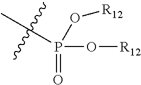

wherein each R₁₂ is selected independently from hydrogen; optionally substituted optionally substituted alkyl, aryl, acyl, heterocycloalkyl or heteroaryl;
iv)

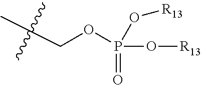

wherein each R₁₃ is selected independently from hydrogen; optionally substituted alkyl, cycloalkyl, heterocycloalkyl, aryl, or heteroaryl;
v) amino acids, wherein the amino acid is linked via ester linkage at the point of attachment;
vi)

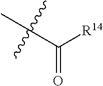

wherein R¹⁴ is

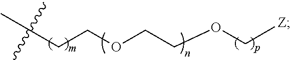

wherein m and p are independently selected from 0 to 5, n refers to degree of polymerization and is selected from 1 to 250 and Z is optionally substituted alkyl or cycloalkyl;
vii)

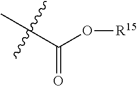

wherein R¹⁵ is selected from group comprising of optionally substituted alkyl, alkenyl, alkynyl, cycloalkyl, heterocycloalkyl, aryl and heteroaryl;
viii)

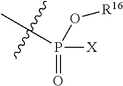

wherein R¹⁶ is selected from group comprising of hydrogen; optionally substituted alkyl, aryl, acyl, heteroaryl; and X is optionally substituted heterocycloalkyl;
ix)

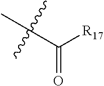

wherein R₁₇ is selected from optionally substituted bicyclic compounds, spirocyclic compounds or bridged bicyclic compounds; pharmaceutically acceptable salts, enantiomers, diastereomers, racemates, or solvates thereof.

In one embodiment, the present invention relates to compound of formula XXII:

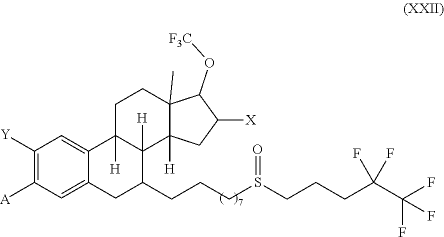

wherein;
X is halogen or hydrogen;
Y is alkyl, —O-alkyl or hydrogen;
A is selected from

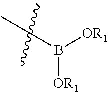

or —O—R2, wherein R1 is selected from hydrogen or alkyl; R₂ is selected independently from group consisting of
i)

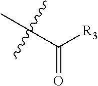

wherein R₃ is selected from NH₂, NHR₄, or NR₅R₆; R₄ is selected from group comprising optionally substituted alkyl, alkenyl, alkynyl, cycloalkyl, heterocycloalkyl, aryl, or heteroaryl; R₅ and R₆ are selected independently from group comprising optionally substituted alkyl, aryl, heteroaryl, heterocycloalkyl, cycloalkyl; or R₅ and R₆ are taken together along with the nitrogen to which they are attached to form a three to seven membered heterocyclic ring and may optionally be substituted at a substitutable position with one or more R₇ radicals, wherein the one or more R₇ radicals are independently selected at each occurrence from the group consisting of optionally substituted alkyl, alkylcarbonyl, formyl, halo, haloalkyl, alkylphosphate, phosphate, oxo, cyano, nitro, amino, hydroxyalkylamino, alkoxy, alkoxycarbonyl, carboxyalkyl, hydroxyalkyl, alkenyl, alkynyl, alkylthio, cycloalkyl, aryl, heteroaryl, aralkyl, heterocycloalkyl, alkylthioalkyl, arylcarbonyl, aralkylcarbonyl, alkoxyalkyl;
ii)

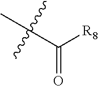

wherein R₈ is selected from group comprising optionally substituted alkyl, alkenyl, alkynyl, cycloalkyl, aryl, heteroaryl; or —CR₉R₁₀; where R₉ and R₁₀ are taken together along with the carbon to which they are attached to form a three to seven membered saturated, partially unsaturated or unsaturated heterocyclic ring in which up to 4 carbon atoms are replaced by heteroatoms chosen from the group consisting of O, S or N and may optionally be substituted at a substitutable position with one or more R₁₁ radicals, wherein one or more R₁₁ radicals are independently selected at each occurrence from the group consisting of optionally substituted alkyl, cycloalkyl, heterocycloalkyl, aryl, heteroaryl, alkylcarbonyl, formyl, halo, alkylphosphate, phosphate, cyano, nitro, alkoxy, amino, alkoxycarbonyl, alkenyl, alkynyl, alkylthio, arylcarbonyl;
iii)

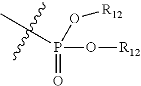

wherein each R₁₂ is selected independently from hydrogen; optionally substituted optionally substituted alkyl, aryl, acyl, heterocycloalkyl or heteroaryl;
iv)

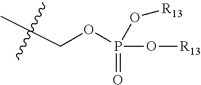

wherein each R₁₃ is selected independently from hydrogen; optionally substituted alkyl, cycloalkyl, heterocycloalkyl, aryl, or heteroaryl;
v) amino acids, wherein the amino acid is linked via ester linkage at the point of attachment;
vi)

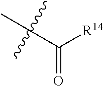

wherein R¹⁴ is

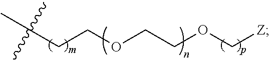

wherein m and p are independently selected from 0 to 5, n refers to degree of polymerization and is selected from 1 to 250 and Z is optionally substituted alkyl or cycloalkyl;
vii)

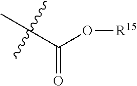

wherein R¹⁵ is selected from group comprising of optionally substituted alkyl, alkenyl, alkynyl, cycloalkyl, heterocycloalkyl, aryl and heteroaryl;
viii)

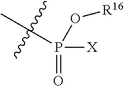

wherein R¹⁶ is selected from group comprising of hydrogen; optionally substituted alkyl, aryl, acyl, heteroaryl; and X is optionally substituted heterocycloalkyl;
ix)

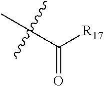

wherein R₁₇ is selected from optionally substituted bicyclic compounds, spirocyclic compounds or bridged bicyclic compounds; pharmaceutically acceptable salts, enantiomers, diastereomers, racemates, or solvates thereof.

In one embodiment, the present invention relates to compound of formula XXIII:

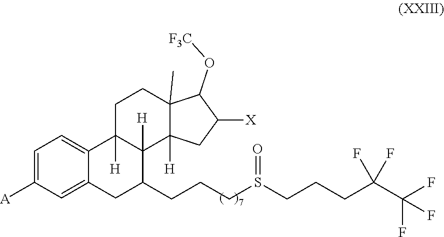

wherein X and A has the same meaning as given in compound of formula XXII; pharmaceutically acceptable salts, enantiomers, diastereomers, racemates, or solvates thereof.

In one embodiment, the present invention relates to compound of formula XXIV:

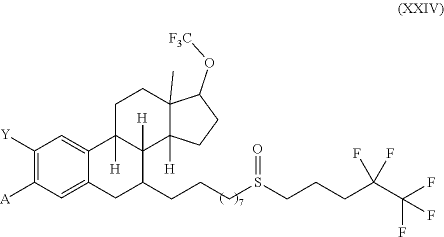

wherein Y and A has the same meaning as given in compound of formula XXII; pharmaceutically acceptable salts, enantiomers, diastereomers, racemates, or solvates thereof.

In one embodiment, the present invention relates to compound of formula XXV:

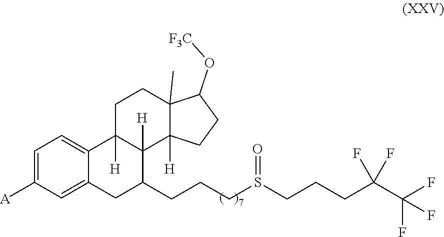

wherein;
A is selected from

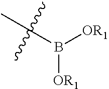

or —O—R2, wherein R1 is selected from hydrogen or alkyl; R₂ is selected independently from group consisting of
i)

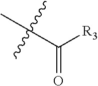

wherein R₃ is selected from NH₂, NHR₄, or NR₅R₆; R₄ is selected from group comprising optionally substituted alkyl, alkenyl, alkynyl, cycloalkyl, heterocycloalkyl, aryl, or heteroaryl; R₅ and R₆ are selected independently from group comprising optionally substituted alkyl, aryl, heteroaryl, heterocycloalkyl, cycloalkyl; or R₅ and R₆ are taken together along with the nitrogen to which they are attached to form a three to seven membered heterocyclic ring and may optionally be substituted at a substitutable position with one or more R₇ radicals, wherein the one or more R₇ radicals are independently selected at each occurrence from the group consisting of optionally substituted alkyl, alkylcarbonyl, formyl, halo, haloalkyl, alkylphosphate, phosphate, oxo, cyano, nitro, amino, hydroxyalkylamino, alkoxy, alkoxycarbonyl, carboxyalkyl, hydroxyalkyl, alkenyl, alkynyl, alkylthio, cycloalkyl, aryl, heteroaryl, aralkyl, heterocycloalkyl, alkylthioalkyl, arylcarbonyl, aralkylcarbonyl, alkoxyalkyl;
ii)

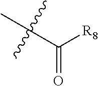

wherein R₈ is selected from group comprising optionally substituted alkyl, alkenyl, alkynyl, cycloalkyl, aryl, heteroaryl; or —CR₉R₁₀; where R₉ and R₁₀ are taken together along with the carbon to which they are attached to form a three to seven membered saturated, partially unsaturated or unsaturated heterocyclic ring in which up to 4 carbon atoms are replaced by heteroatoms chosen from the group consisting of O, S or N and may optionally be substituted at a substitutable position with one or more R₁₁ radicals, wherein one or more R₁₁ radicals are independently selected at each occurrence from the group consisting of optionally substituted alkyl, cycloalkyl, heterocycloalkyl, aryl, heteroaryl, alkylcarbonyl, formyl, halo, alkylphosphate, phosphate, cyano, nitro, alkoxy, amino, alkoxycarbonyl, alkenyl, alkynyl, alkylthio, arylcarbonyl;
iii)

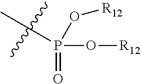

wherein each R₁₂ is selected independently from hydrogen; optionally substituted optionally substituted alkyl, aryl, acyl, heterocycloalkyl or heteroaryl;
iv)

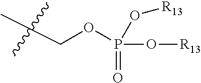

wherein each R₁₃ is selected independently from hydrogen; optionally substituted alkyl, cycloalkyl, heterocycloalkyl, aryl, or heteroaryl;
v) amino acids, wherein the amino acid is linked via ester linkage at the point of attachment;
vi)

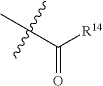

wherein R¹⁴ is

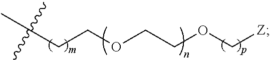

wherein m and p are independently selected from 0 to 5, n refers to degree of polymerization and is selected from 1 to 250 and Z is optionally substituted alkyl or cycloalkyl;
vii)

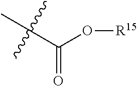

wherein R¹⁵ is selected from group comprising of optionally substituted alkyl, alkenyl, alkynyl, cycloalkyl, heterocycloalkyl, aryl and heteroaryl;
viii)

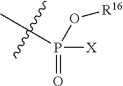

wherein R¹⁶ is selected from group comprising of hydrogen; optionally substituted alkyl, aryl, acyl, heteroaryl; and X is optionally substituted heterocycloalkyl;
ix)

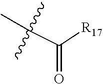

wherein R₁₇ is selected from optionally substituted bicyclic compounds, spirocyclic compounds or bridged bicyclic compounds; pharmaceutically acceptable salts, enantiomers, diastereomers, racemates, or solvates thereof.

In another embodiment, the present invention relates to compound of formula XXVI:

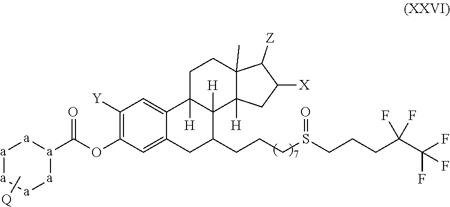

X is halogen or hydrogen;
Y is alkyl, —O-alkyl or hydrogen;
Z is selected from ═O, ═N—OH, —O-alkyl, O-haloalkyl, —NH—OH or —O-alkyl-OH wherein the bond between the Z substituent attached to carbon can be single or double bond depend on the substituent selected,
a is selected from carbon or nitrogen;
Q is selected from group consisting of optionally substituted alkyl, alkylcarbonyl, formyl, halo, haloalkyl, alkylphosphate, phosphate, oxo, cyano, nitro, amino, alkoxy, alkoxycarbonyl, carboxyalkyl, hydroxyalkyl, alkenyl, alkynyl, alkylthio, cycloalkyl, aryl, heteroaryl, aralkyl, heterocycloalkyl, alkylthioalkyl, arylcarbonyl, aralkylcarbonyl, alkoxyalkyl; pharmaceutically acceptable salts, enantiomers, diastereomers, racemates, or solvates thereof.

In another embodiment, the present invention relates to compound of formula XXVII:

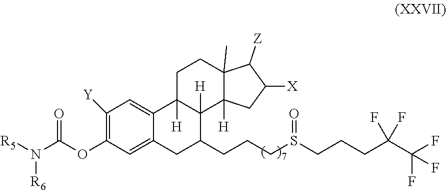

X is halogen or hydrogen;
Y is alkyl, —O-alkyl or hydrogen;
Z is selected from ═O, ═N—OH, —O-alkyl, O-haloalkyl, —NH—OH or —O-alkyl-OH wherein the bond between the Z substituent attached to carbon can be single or double bond depend on the substituent selected;
wherein R₅ and R₆ are taken together along with the nitrogen to which they are attached to form a three to seven membered saturated, partially unsaturated or unsaturated heterocyclic ring and substituted at a substitutable position with one or more R₇ radicals, wherein the one or more R₇ radicals are independently selected at each occurrence from the group consisting of substituted alkyl, alkylcarbonyl, formyl, halo, haloalkyl, alkylphosphate, phosphate, phosphonyl, oxo, cyano, nitro, amino, alkoxy, alkoxycarbonyl, carboxyalkyl, hydroxyalkyl, alkenyl, alkynyl, alkylthio, cycloalkyl, aryl, heteroaryl, aralkyl, heterocycloalkyl, alkylthioalkyl, arylcarbonyl, aralkylcarbonyl, alkoxyalkyl and; pharmaceutically acceptable salts, enantiomers, diastereomers, racemates, or solvates thereof. In one embodiment, one or more substitution on radical R₇ is further substituted with one or more substitution selected from group consisting of alkyl, alkenyl, alkynyl, cycloalkyl, heterocycloalkyl, aryl, heteroaryl, amino, alkylamino, acyl, acyloxy, acylamino, aminocarbonyl, alkoxycarbonyl, alkoxyalkyloxy, alkoxycarbonyloxy, carbamate, sulfinyl, sulfonyl, alkoxy, sulfanyl, halogen, carboxy, trihalomethyl, cyano, hydroxy, mercapto, nitro, phosphate, alkylphosphate; pharmaceutically acceptable salts, enantiomers, diastereomers, racemates, or solvates thereof.

In another embodiment, the present invention relates to compounds formula XXVIII:

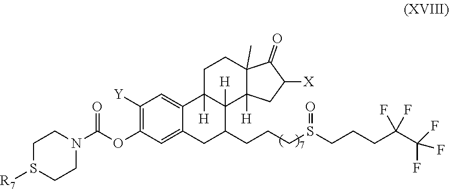

X is halogen or hydrogen;
Y is alkyl, —O-alkyl or hydrogen;
S is selected from O, C, or N; and
wherein R₇ is selected from group consisting of optionally substituted alkyl, alkylcarbonyl, formyl, halo, haloalkyl, alkylphosphate, phosphate, oxo, cyano, nitro, amino, alkoxy, alkoxycarbonyl, carboxyalkyl, hydroxyalkyl, alkenyl, alkynyl, alkylthio, cycloalkyl, aryl, heteroaryl, aralkyl, heterocycloalkyl, alkylthioalkyl, arylcarbonyl, aralkylcarbonyl, alkoxyalkyl; pharmaceutically acceptable salts, enantiomers, diastereomers, racemates, or solvates thereof.

In another embodiment, the present invention relates to compound of formula XXIX:

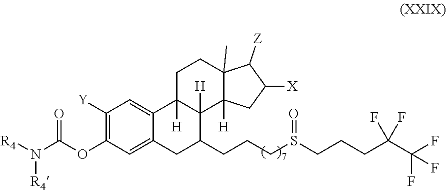

X is halogen or hydrogen;
Y is alkyl, —O-alkyl or hydrogen;
Z is selected from ═O, ═N—OH, —O-alkyl, O-haloalkyl, —NH—OH or —O-alkyl-OH wherein the bond between the Z substituent attached to carbon can be single or double bond depend on the substituent selected;
R₄′ is selected from group Hydrogen and optionally substituted alkyl.

R₄ is selected from group comprising optionally substituted alkyl, alkenyl, alkynyl, cycloalkyl, heterocycloalkyl, aryl, or heteroaryl; pharmaceutically acceptable salts, enantiomers, diastereomers, racemates, or solvates thereof.

In one embodiment, optionally substituted substituents on R4 and R4′ is selected from the group consisting of radicals such as C1-C8-alkyl, C2-C8-alkenyl, C2-C8-alkynyl, cycloalkyl, heterocycloalkyl, aryl, heteroaryl, amino, alkylamino, dialkylamino acyl, acyloxy, acylamino, aminocarbonyl, alkoxycarbonyl, alkoxyalkyloxy, alkoxycarbonyloxy, carbamate, sulfinyl, sulfonyl, alkoxy, sulfanyl, halogen, carboxy, haloalkyl, cyano, hydroxy, mercapto, nitro, phosphate, oxo, alkylphosphate.

In another embodiment, the present invention relates to compound of formula XXX:

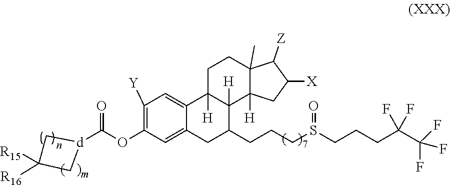

X is halogen or hydrogen;
Y is alkyl, —O-alkyl or hydrogen;
Z is selected from ═O, ═N—OH, —O-alkyl, O-haloalkyl, —NH—OH or —O-alkyl-OH wherein the bond between the Z substituent attached to carbon can be single or double bond depend on the substituent selected;
d is selected from carbon or nitrogen; wherein R₁₅ and R₁₆ together forms a optionally substituted carbocyclic or heterocyclic structure;
m and n can be independently 1 to 3;
pharmaceutically acceptable salts, enantiomers, diastereomers, racemates, or solvates thereof.

In one embodiment, spirocyclic compound of formula XXX is optionally substituted with substituents selected from group consisting of alkyl, alkylcarbonyl, formyl, halo, haloalkyl, alkylphosphate, phosphate, oxo, cyano, nitro, amino, alkoxy, alkoxycarbonyl, carboxyalkyl, hydroxyalkyl, alkenyl, alkynyl, alkylthio, cycloalkyl, aryl, heteroaryl, aralkyl, heterocycloalkyl, alkylthioalkyl, arylcarbonyl, aralkylcarbonyl, alkoxyalkyl.

In an embodiment, the present invention relates to compound of formula XXXI

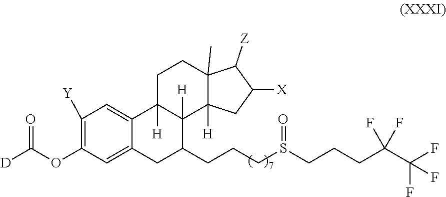

X is halogen or hydrogen;
Y is alkyl, —O-alkyl or hydrogen;
Z is selected from ═O, ═N—OH, —O-alkyl, O-haloalkyl, —NH—OH or —O-alkyl-OH wherein the bond between the Z substituent attached to carbon can be single or double bond depend on the substituent selected;
D is selected from optionally substituted bicyclic compound, spirocyclic compound or bridged bicyclic compounds, wherein the attachment of the bicyclic compound, spirocyclic compound or bridged bicyclic compounds is through either carbon or nitrogen atom; pharmaceutically acceptable salts, enantiomers, diastereomers, racemates, or solvates thereof.

In one embodiment, optionally substituents on D can be selected from group consisting of alkyl, alkylcarbonyl, formyl, halo, haloalkyl, alkylphosphate, phosphate, oxo, cyano, nitro, amino, alkoxy, alkoxycarbonyl, carboxyalkyl, hydroxyalkyl, alkenyl, alkynyl, alkylthio, cycloalkyl, aryl, heteroaryl, aralkyl, heterocycloalkyl, alkylthioalkyl, arylcarbonyl, aralkylcarbonyl, alkoxyalkyl.

In one embodiment, the present invention provides synthesised and isolated novel compounds in pure form. In one embodiment, the purity of the isolated novel compounds is from about 80%. In one embodiment, the purity of the isolated novel compound is selected from group consisting of about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 99.5%.

In another embodiment, the compounds of the present invention further can be delivered in a form of pharmaceutical composition comprising compound of invention; pharmaceutically acceptable salts, enantiomers, diastereomers, racemates, or solvates thereof; and a pharmaceutically acceptable excipient.

In another embodiment, the present invention relates to pharmaceutical composition comprising at least one formula of compound selected from formula I to XXXI; pharmaceutically acceptable salts, enantiomers, diastereomers, racemates, or solvates thereof; and at least one pharmaceutically acceptable excipient. In one embodiment, the novel formula of compound for the said pharmaceutical composition is having at least one formula of compound selected from formula I, formula I-A, formula II, formula III, formula IV, formula V, formula VI, formula VII, formula VIII, formula IX, formula X, formula XI, formula XII, formula XIII, formula XIV, formula XV, formula XVI, formula XVII, formula XVIII, formula XIX, formula XX, formula XXI, formula XXII, formula XXIII, formula XXIV, formula XXV, formula XXVI, formula XXVII, formula XXVIII, formula XXIX, formula XXX and formula XXXI; pharmaceutically acceptable salts, enantiomers, diastereomers, racemates, or solvates thereof. In one embodiment, the present invention relates to pharmaceutical composition comprising at least one compound of invention; pharmaceutically acceptable salts, enantiomers, diastereomers, racemates or solvates thereof; and at least one pharmaceutically acceptable excipient.

The pharmaceutical compositions of the present disclosure can be in any form known to those of skill in the art. The pharmaceutical compositions of this invention may be administered orally, parenterally, by inhalation spray, topically, rectally, nasally, buccally or via an implanted reservoir, preferably oral administration or administration by injection.

For instance, in some embodiments the pharmaceutical composition comprising desired product is formulated for oral delivery. In one embodiment the pharmaceutical composition comprising at least one compound is delivered through dosage form selected from a group consisting of a concentrate, dried powder, tablet, liquid, capsule, pellet, emulsion or pill.

In one embodiment, the present invention provides a pharmaceutical compositions comprises pharmaceutically acceptable excipients selected from carriers, binders, diluents, bulking agents, preservatives, disintegrants, flow regulators, plasticizers, wetting agents, dispersants, emulsifiers, solvents, antioxidants.

The compound of the present invention possesses anti-cancer activity. In one embodiment, the present invention provides a compound useful for the treatment of cancer.

In another embodiment, the present invention provides compound for the treatment of a benign or malignant disease of the breast or reproductive tract, prostate cancer, or endometrial cancer. In one embodiment benign or malignant disease of the breast is breast cancer.

In another embodiment, the invention provides use of compound of the present invention in the preparation of a pharmaceutical formulation as describe hereinabove, for the treatment of a benign or malignant disease of the breast or reproductive tract, prostate cancer or endometrial cancer.

In another embodiment, the invention provides novel compound useful in the treatment of oestrogen-dependent indications such as breast cancer and gynaecological conditions, such as endometriosis.

In one embodiment, the present invention provides a method of treating cancer comprising administering at least one formula of compound selected from formula I to XXXI to the subject. In one embodiment, the present invention provides a method of treating cancer comprising administering at least one formula of compound selected from formula I, formula I-A, formula II, formula III, formula IV, formula V, formula VI, formula VII, formula VIII, formula IX, formula X, formula XI, formula XII, formula XIII, formula XIV, formula XV, formula XVI, formula XVII, formula XVIII, formula XIX, formula XX, formula XXI, formula XXII, formula XXIII, formula XXIV, formula XXV, formula XXVI, formula XXVII, formula XXVIII, formula XXIX, formula XXX and formula XXXI; pharmaceutically acceptable salts, enantiomers, diastereomers, racemates, or solvates thereof. In one embodiment, the present invention provides a method of treating cancer comprising administering at least one compound of invention to the subject.

In one embodiment, the present invention provides a method of treating benign or malignant disease of the breast or reproductive tract comprising administering at least one formula of compound selected from formula I to XXXI to the subject. In one embodiment, the present invention provides a method of treating benign or malignant disease of the breast or reproductive tract comprising administering at least one formula of compound selected from formula I, formula I-A, formula II, formula III, formula IV, formula V, formula VI, formula VII, formula VIII, formula IX, formula X, formula XI, formula XII, formula XIII, formula XIV, formula XV, formula XVI, formula XVII, formula XVIII, formula XIX, formula XX, formula XXI, formula XXII, formula XXIII, formula XXIV, formula XXV, formula XXVI, formula XXVII, formula XXVIII, formula XXIX, formula XXX and formula XXXI; pharmaceutically acceptable salts, enantiomers, diastereomers, racemates, or solvates thereof.

In one embodiment, the present invention provides at least one compound selected from table 1; pharmaceutically acceptable salts, enantiomers, diastereomers, racemates, or solvates thereof.

In one embodiment, the present invention provides at least one compound selected from compound 1 to compound 250 in table 1; pharmaceutically acceptable salts, enantiomers, diastereomers, racemates, or solvates thereof.

In one embodiment, the present invention provides a method of treating benign or malignant disease of the breast or reproductive tract comprising administering at least one compound selected from compound 1 to compound 250 in table 1 to the subject.

In one embodiment, the present invention provides a method of treating cancer comprising administering at least one compound selected from compound 1 to compound 250 in table 1 to the subject.

In one embodiment, the present invention provides at least one compound selected from compound 1 to compound 250 in table 1 for treating benign or malignant disease of the breast or reproductive tract.

In one embodiment, the present invention relates to pharmaceutical composition comprising at least one compound selected from compound 1 to compound 250 in table 1; pharmaceutically acceptable salts, enantiomers, diastereomers, racemates, or solvates thereof; and at least one pharmaceutically acceptable excipient.

In one embodiment, the present invention relates to pharmaceutical composition for use in treating benign or malignant disease of the breast or reproductive tract comprising at least one compound selected from compound 1 to compound 250 in table 1; pharmaceutically acceptable salts, enantiomers, diastereomers, racemates, or solvates thereof; and at least one pharmaceutically acceptable excipient.

TABLE 1
Novel compound of invention

| Compound | |
|---|---|
| No. | Structures |
| 1 | 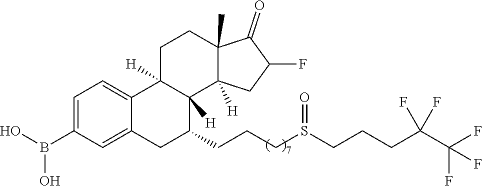 |
| 2 | 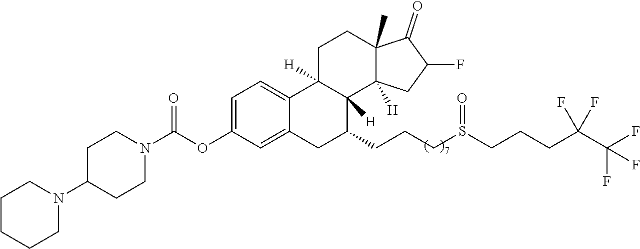 |
| 3 | 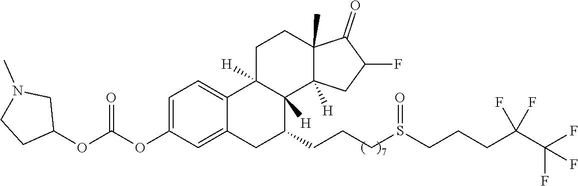 |
| 4 | 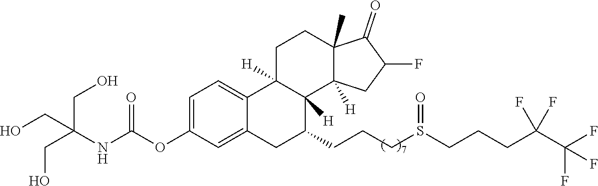 |
| 5 | 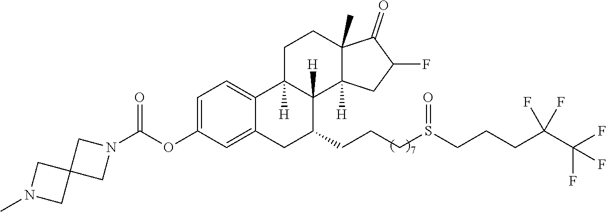 |
| 6 | 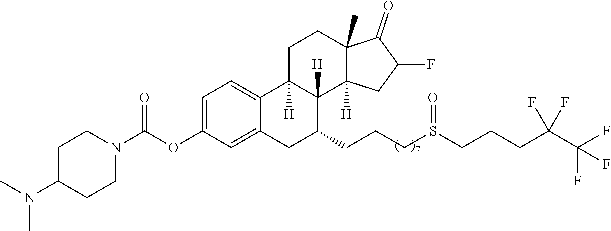 |
| 7 | 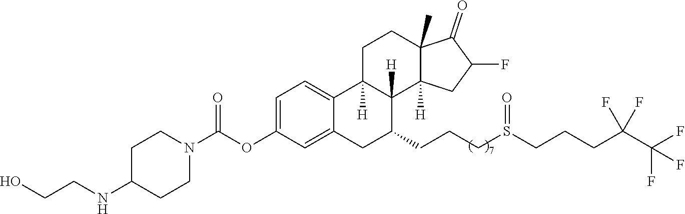 |
| 8 | 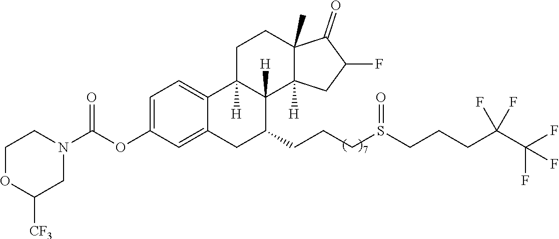 |
| 9 | 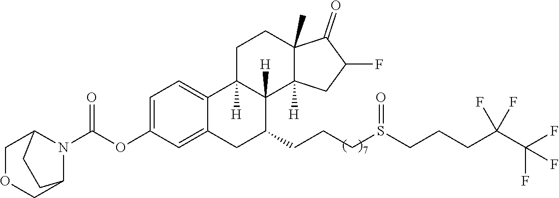 |
| 10 | 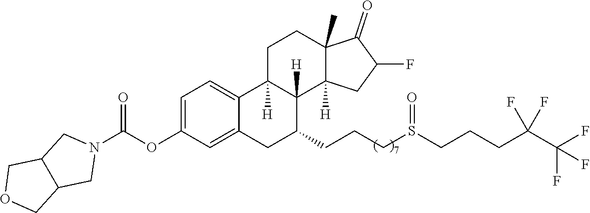 |
| 11 | 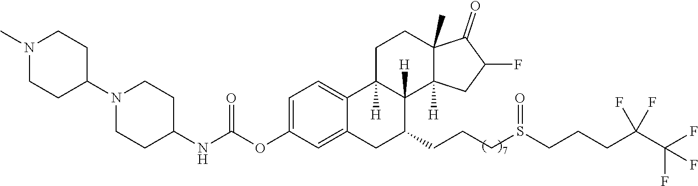 |
| 12 | 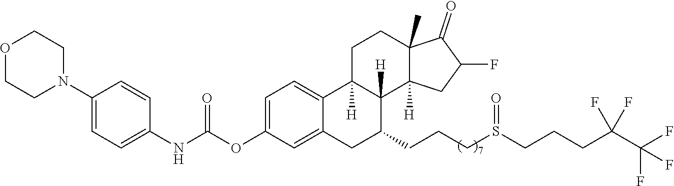 |
| 13 | 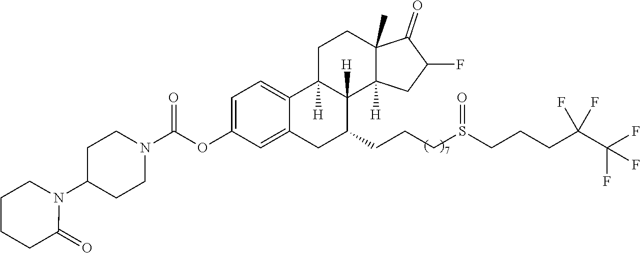 |
| 14 | 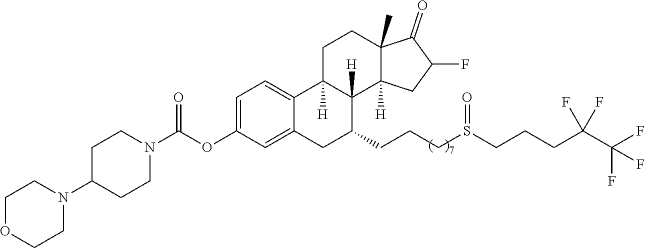 |
| 15 | 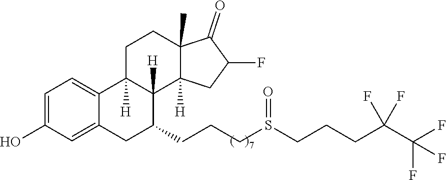 |
| 16 | 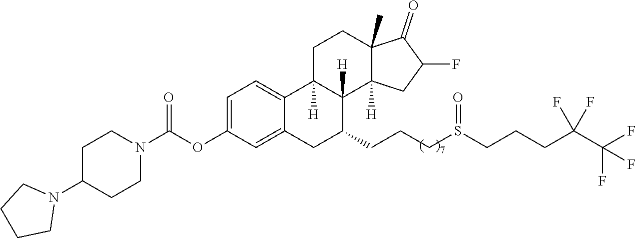 |
| 17 | 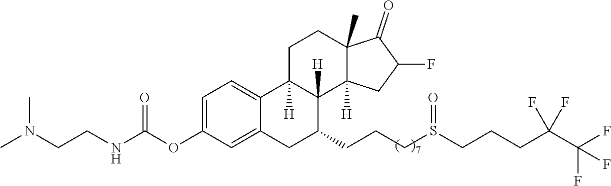 |
| 18 | 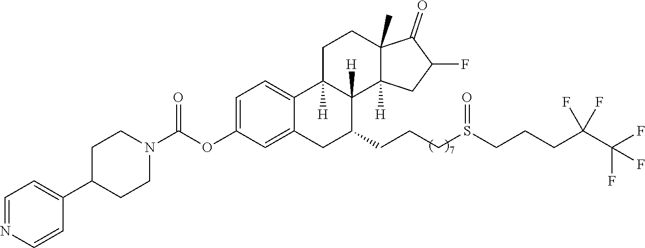 |
| 19 | 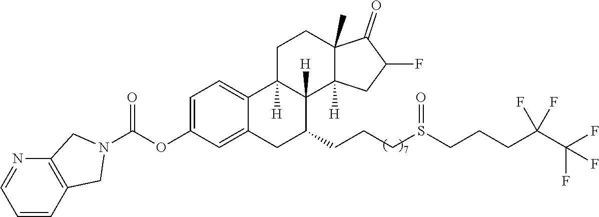 |
| 20 | 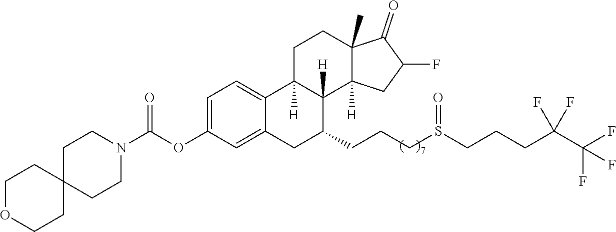 |
| 21 | 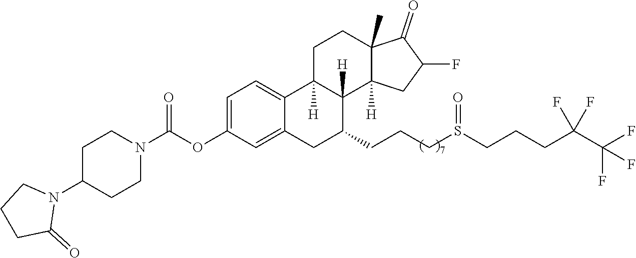 |
| 22 | 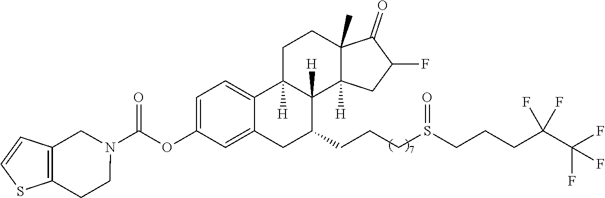 |
| 23 | 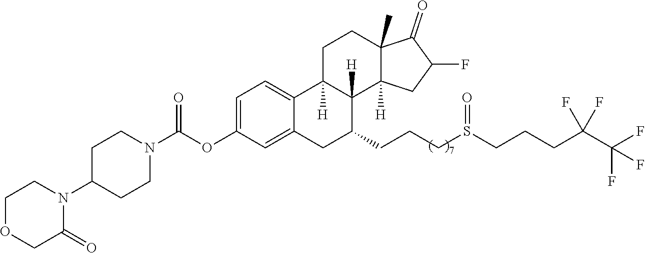 |
| 24 | 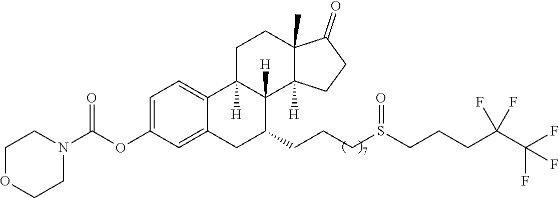 |
| 25 | 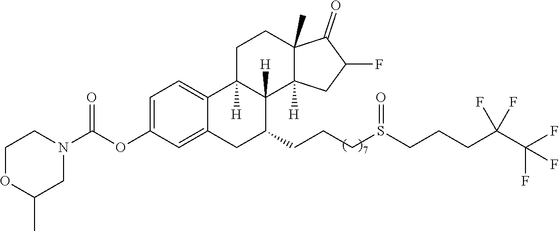 |
| 26 | 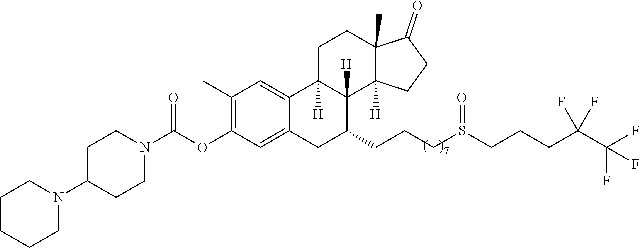 |
| 27 | 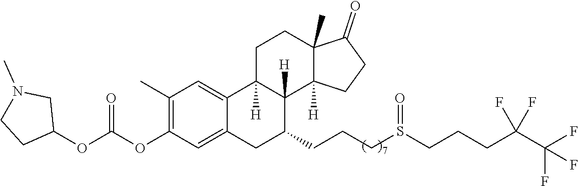 |
| 28 | 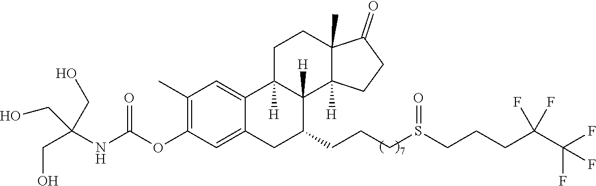 |
| 29 | 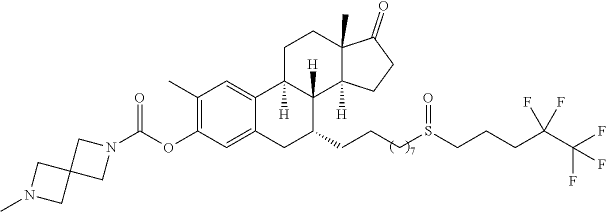 |
| 30 | 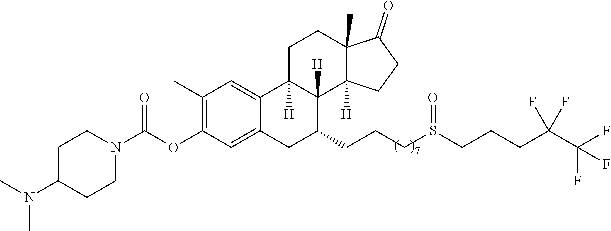 |
| 31 | 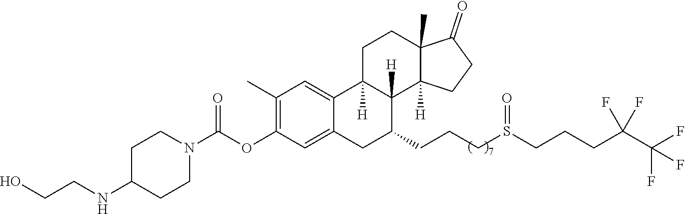 |
| 32 | 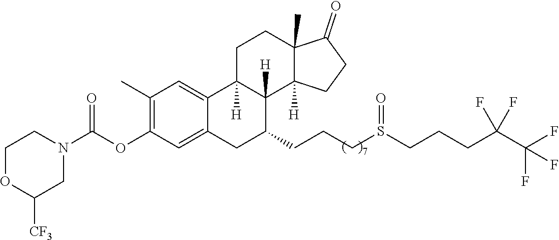 |
| 33 | 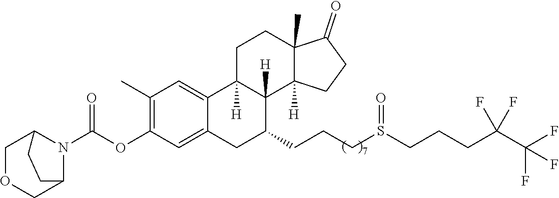 |
| 34 | 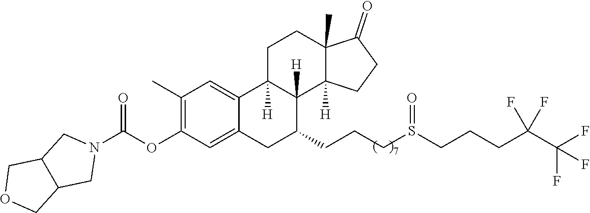 |
| 35 | 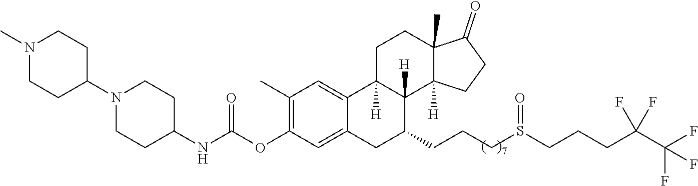 |
| 36 | 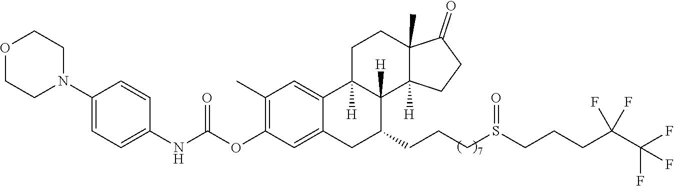 |
| 37 | 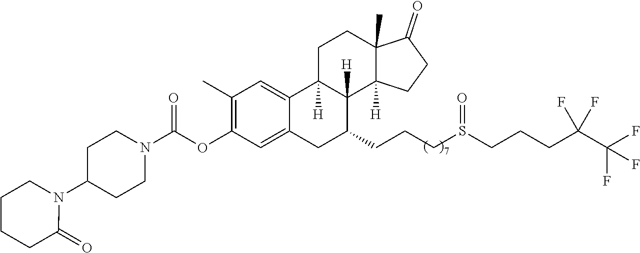 |
| 38 | 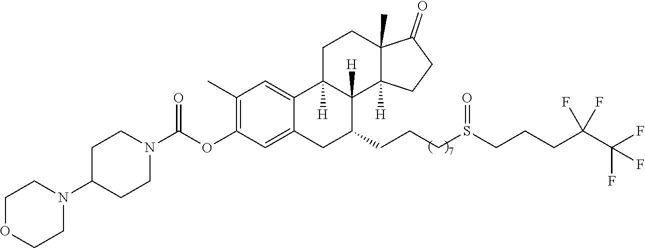 |
| 39 | 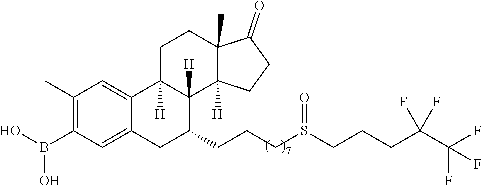 |
| 40 | 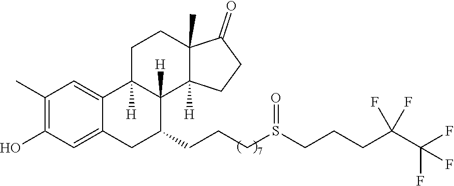 |
| 41 | 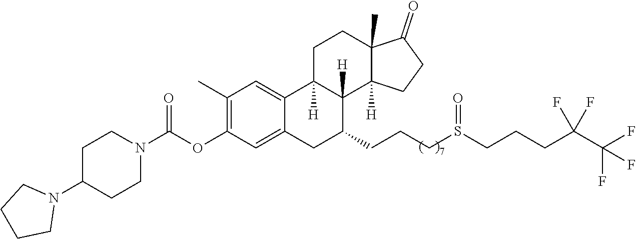 |
| 42 | 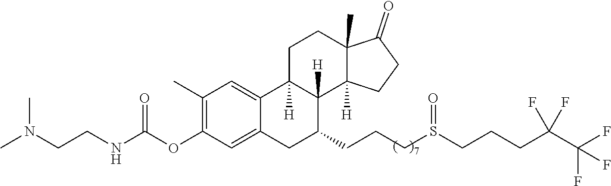 |
| 43 | 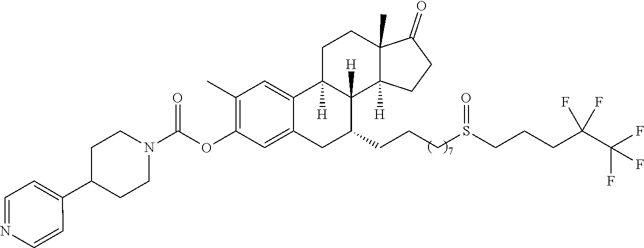 |
| 44 | 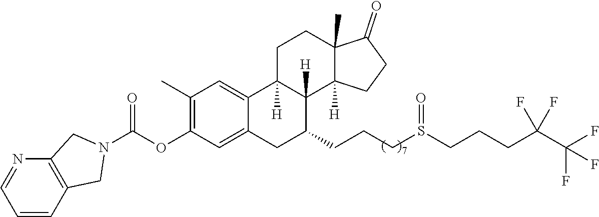 |
| 45 | 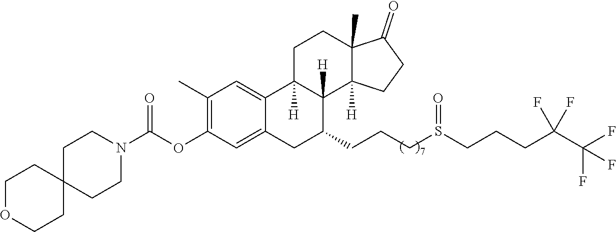 |
| 46 | 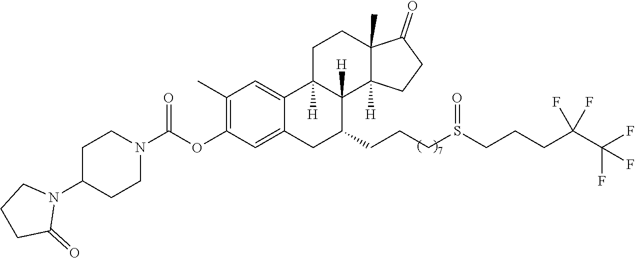 |
| 47 | 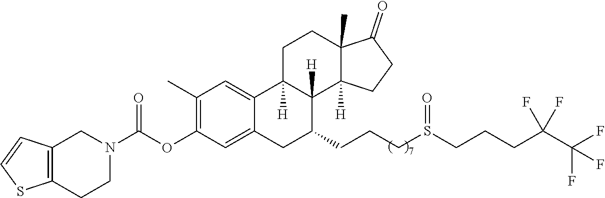 |
| 48 | 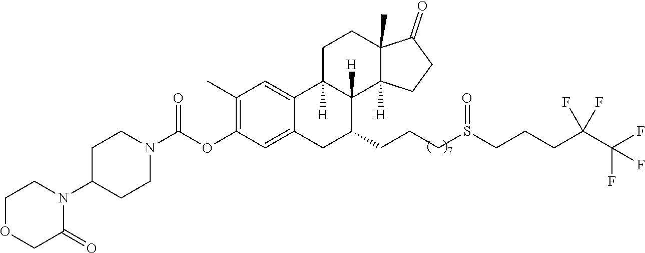 |
| 49 | 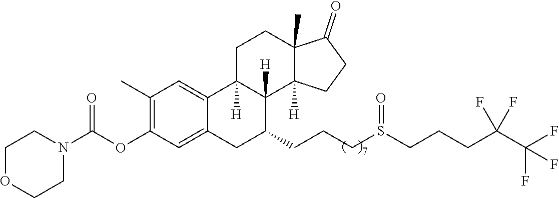 |
| 50 | 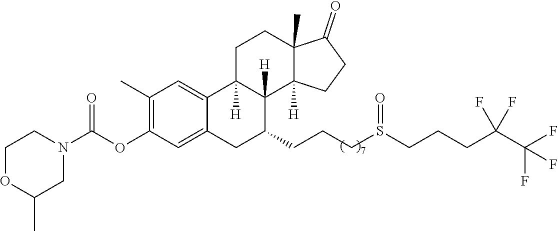 |
| 51 | 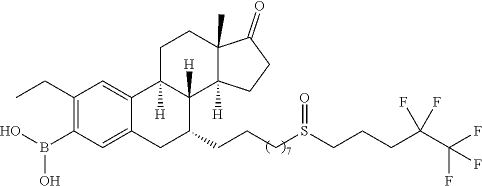 |
| 52 | 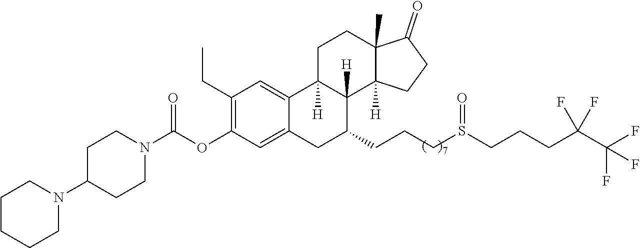 |
| 53 | 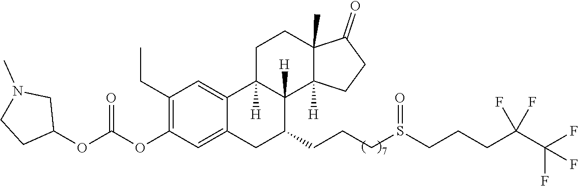 |
| 54 | 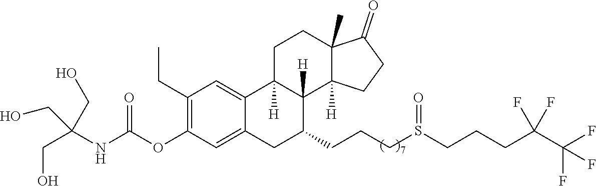 |
| 55 | 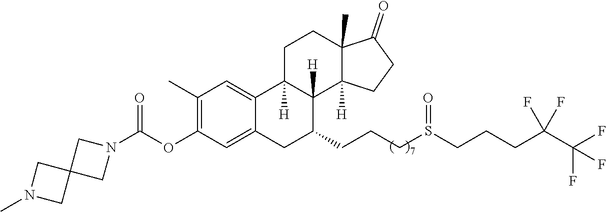 |
| 56 | 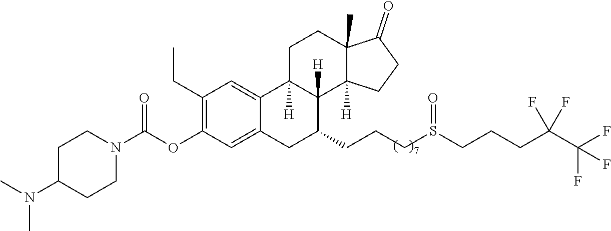 |
| 57 | 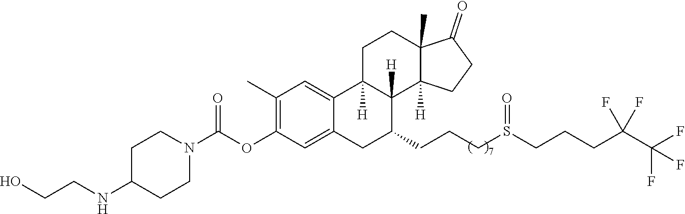 |
| 58 | 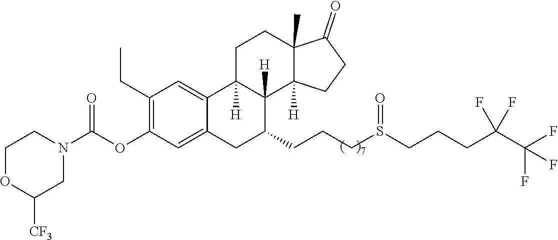 |
| 59 | 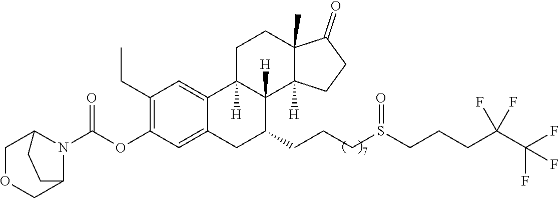 |
| 60 | 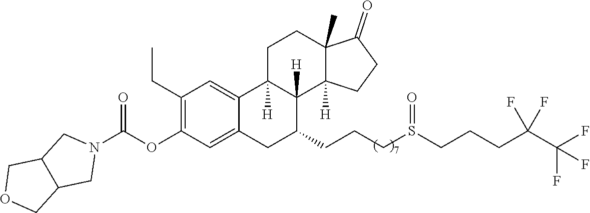 |
| 61 | 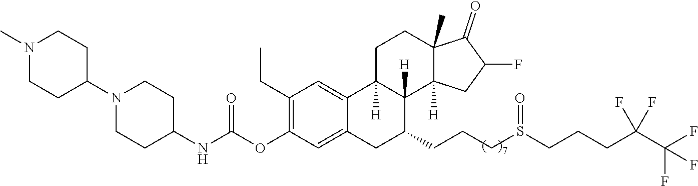 |
| 62 | 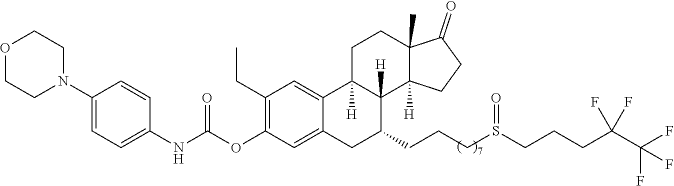 |
| 63 | 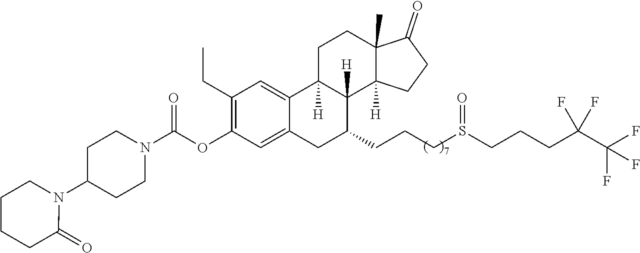 |
| 64 | 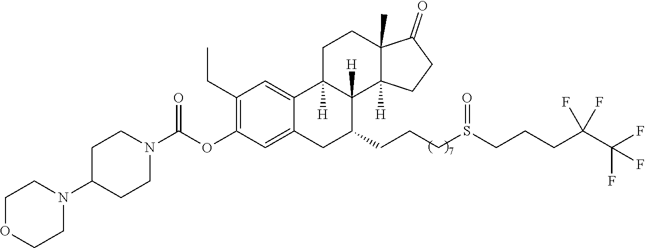 |
| 65 | 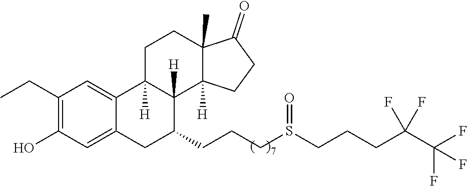 |
| 66 | 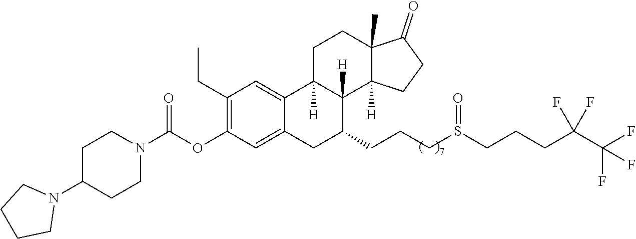 |
| 67 | 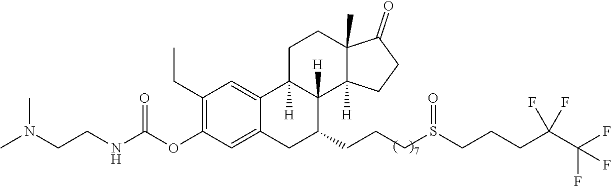 |
| 68 | 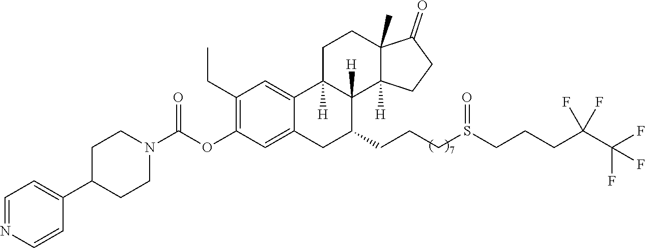 |
| 69 | 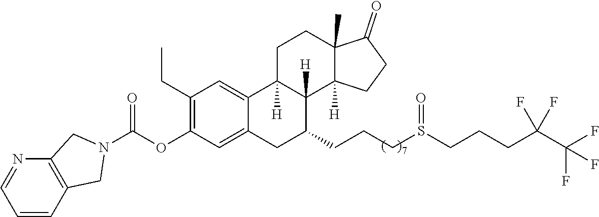 |
| 70 | 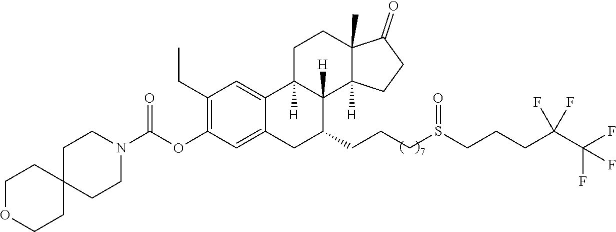 |
| 71 | 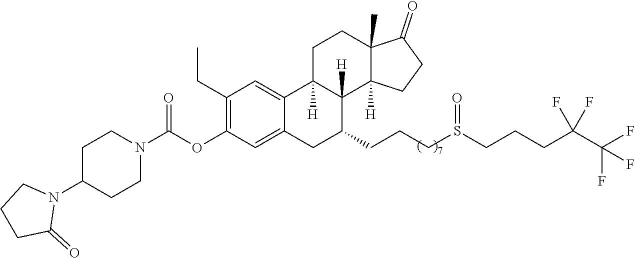 |
| 72 | 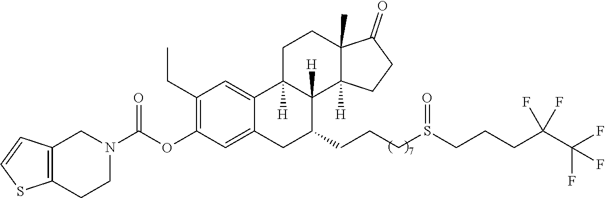 |
| 73 | 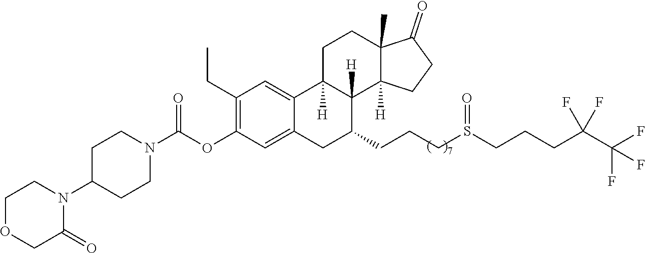 |
| 74 | 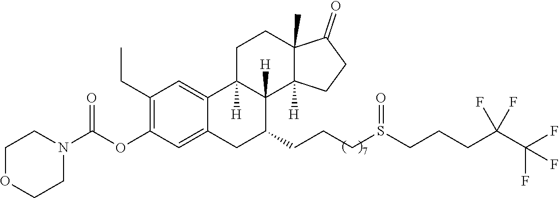 |
| 75 | 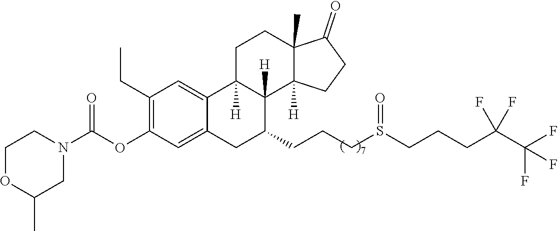 |
| 76 | 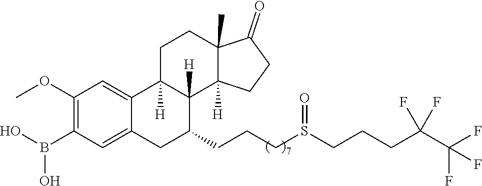 |
| 77 | 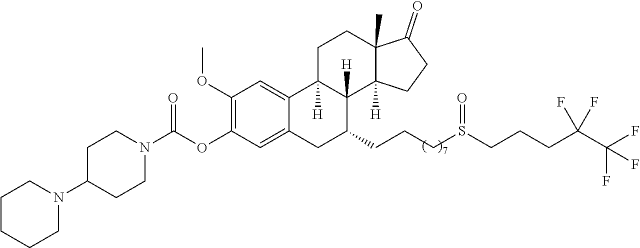 |
| 78 | 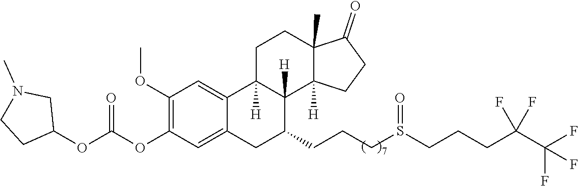 |
| 79 | 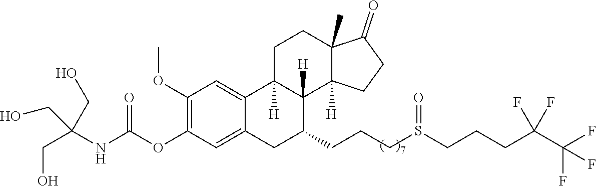 |
| 80 | 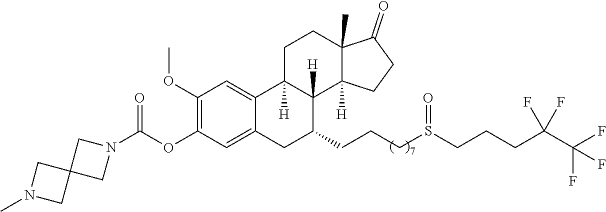 |
| 81 | 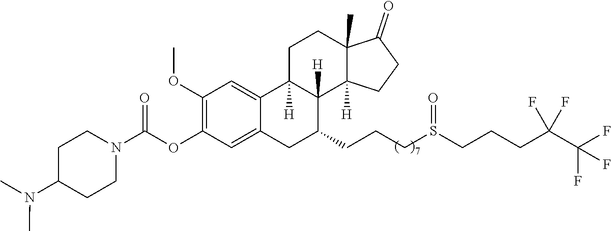 |
| 82 | 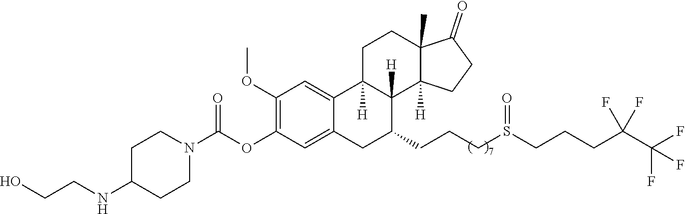 |
| 83 | 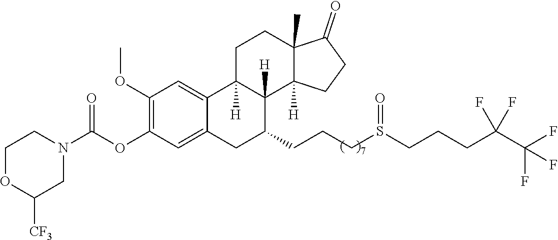 |
| 84 | 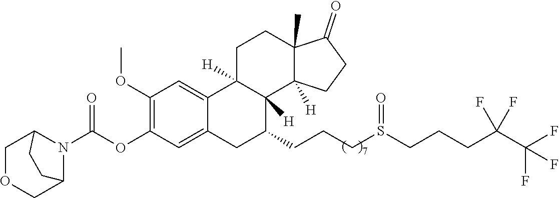 |
| 85 | 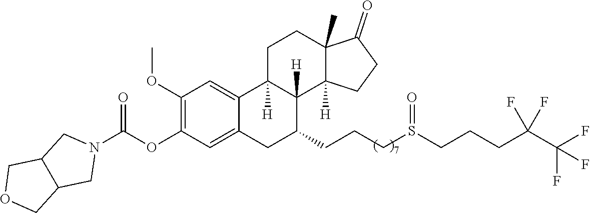 |
| 86 | 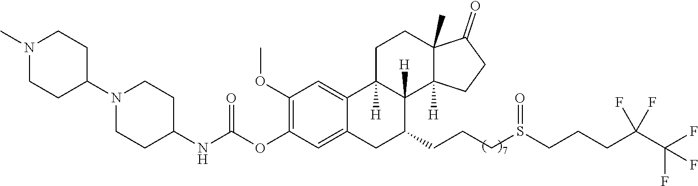 |
| 87 | 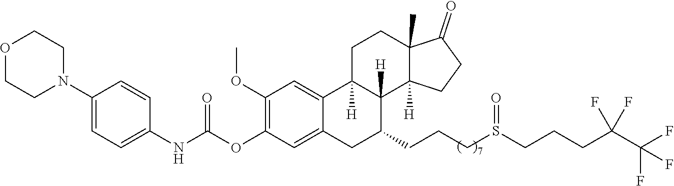 |
| 88 | 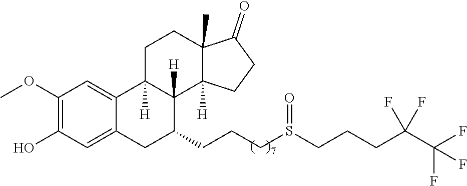 |
| 89 | 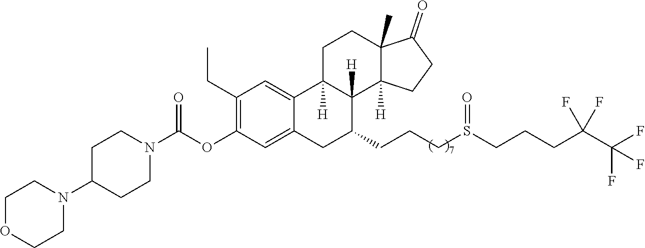 |
| 90 | 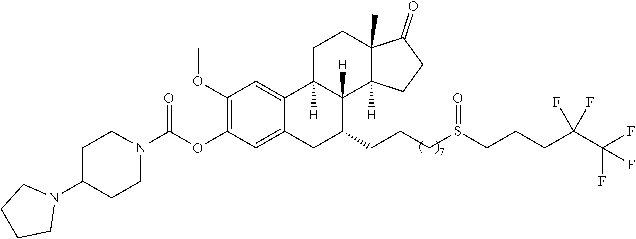 |
| 91 | 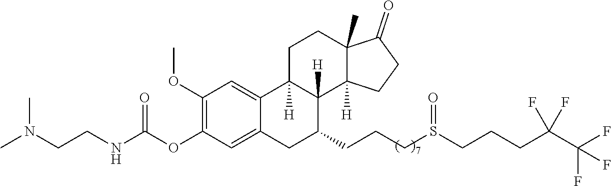 |
| 92 | 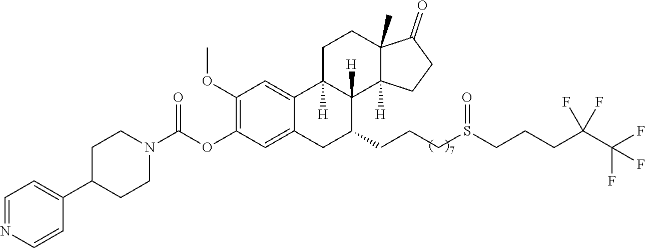 |
| 93 | 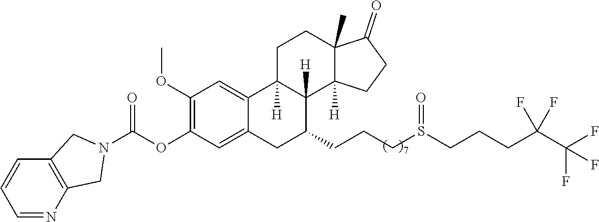 |
| 94 | 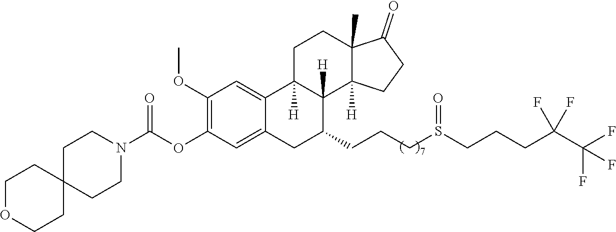 |
| 95 | 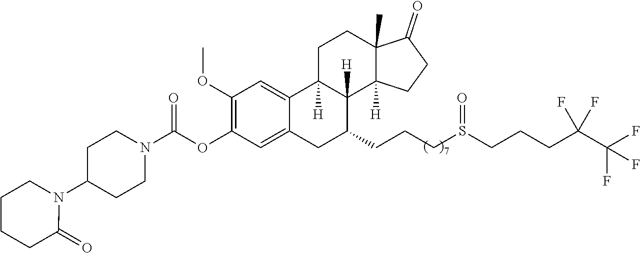 |
| 96 | 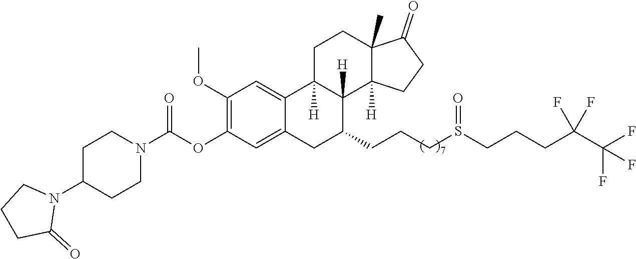 |
| 97 | 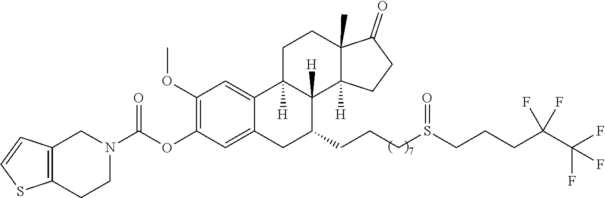 |
| 98 | 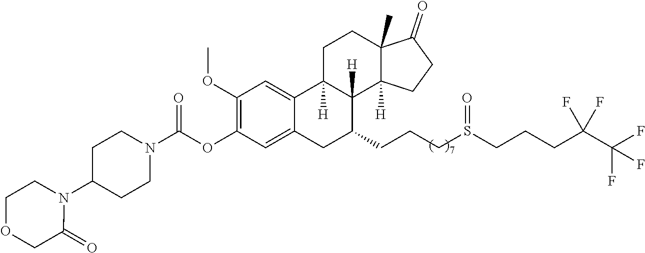 |
| 99 | 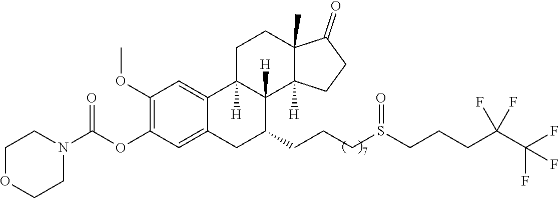 |
| 100 | 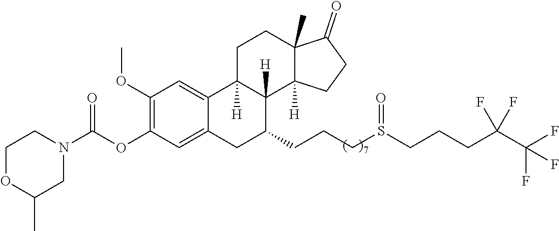 |
| 101 | 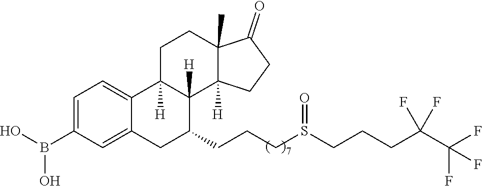 |
| 102 | 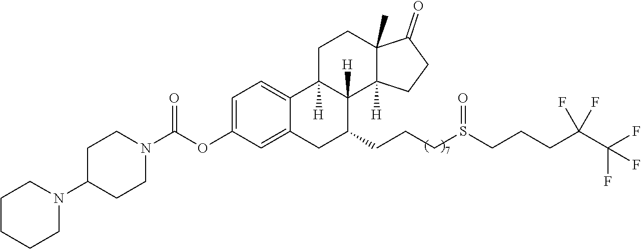 |
| 103 | 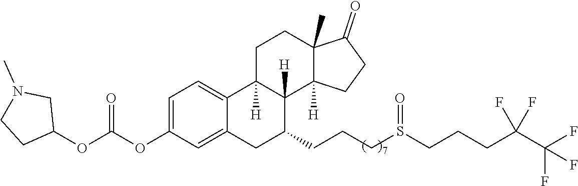 |
| 104 | 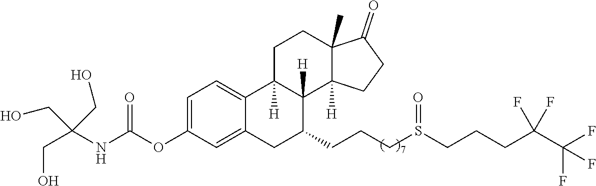 |
| 105 | 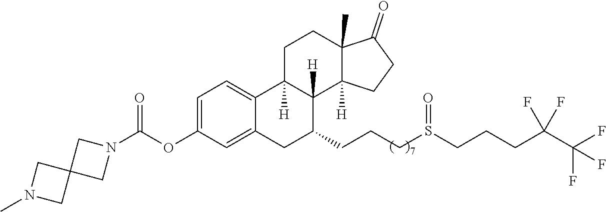 |
| 106 | 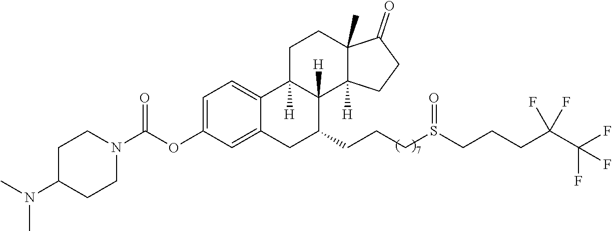 |
| 107 | 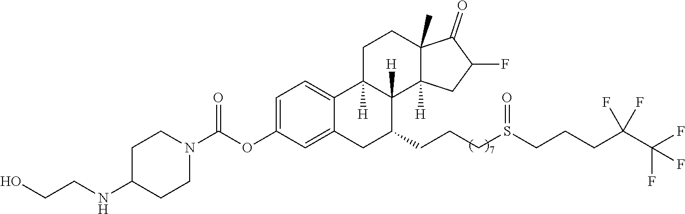 |
| 108 | 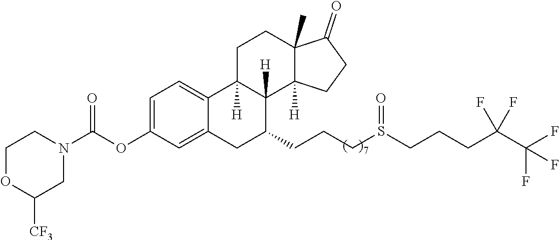 |
| 109 | 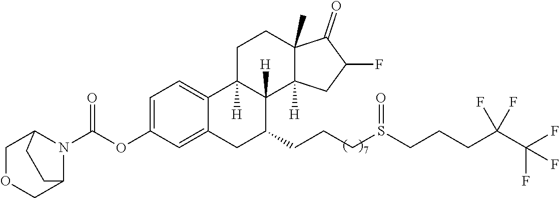 |
| 110 | 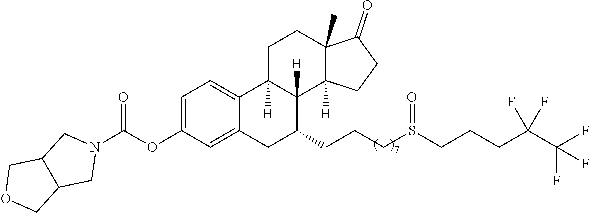 |
| 111 | 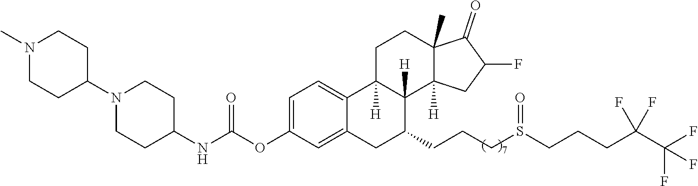 |
| 112 | 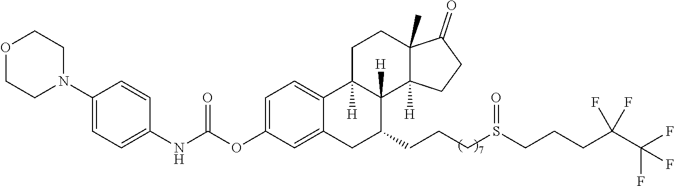 |
| 113 | 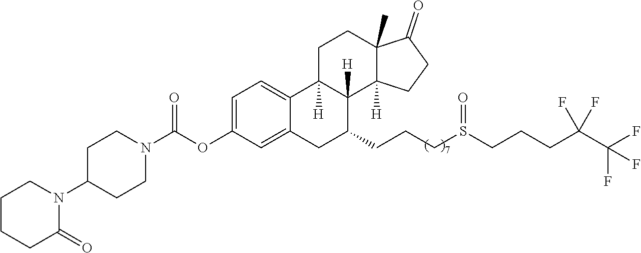 |
| 114 | 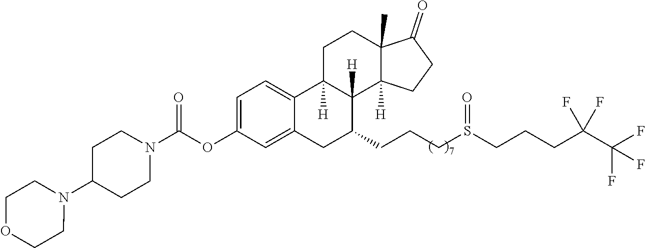 |
| 115 | 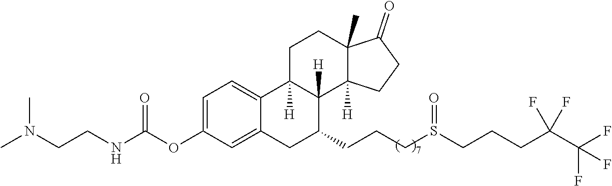 |
| 116 | 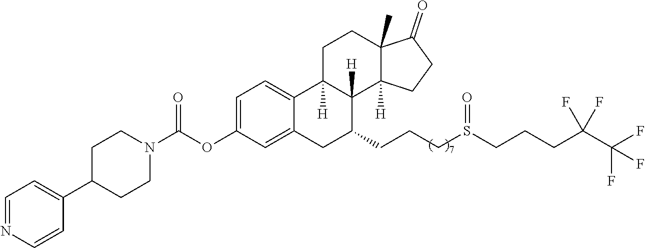 |
| 117 | 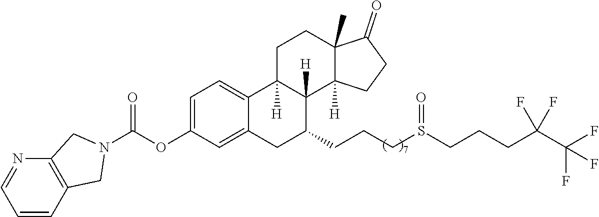 |
| 118 | 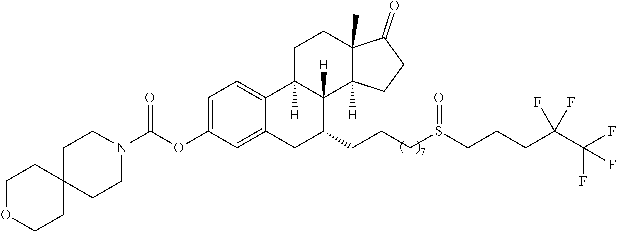 |
| 119 | 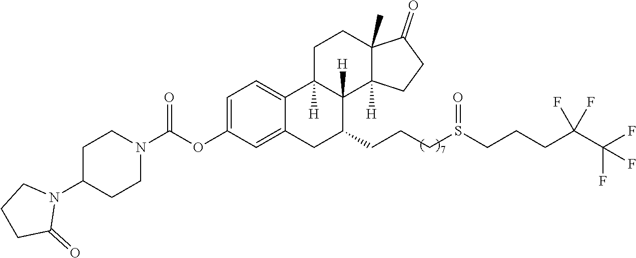 |
| 120 | 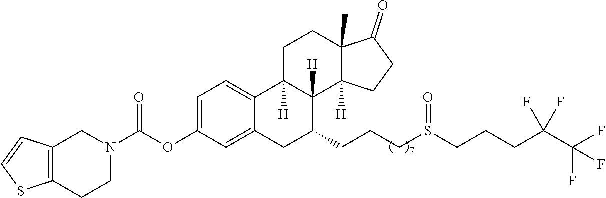 |
| 121 | 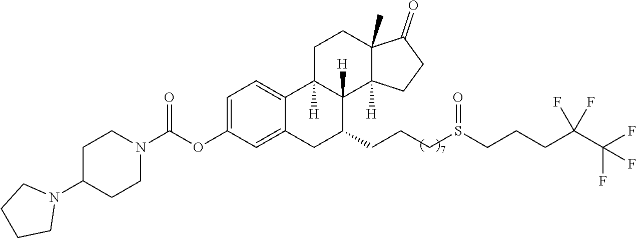 |
| 122 | 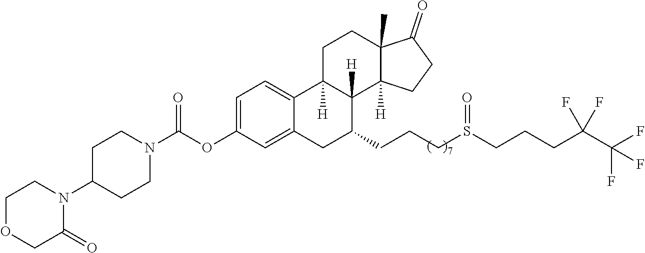 |
| 123 | 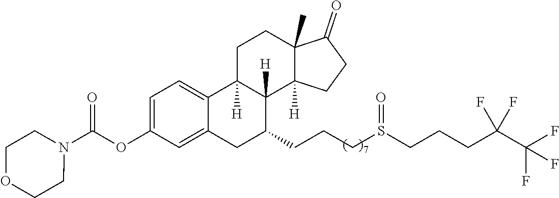 |
| 124 | 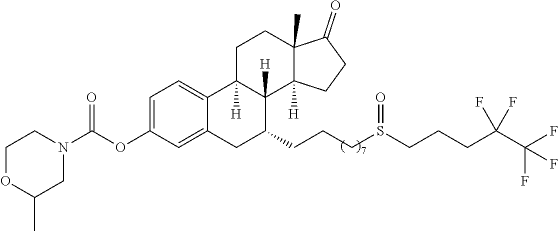 |
| 125 | 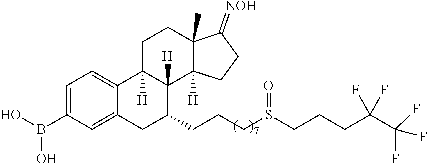 |
| 126 | 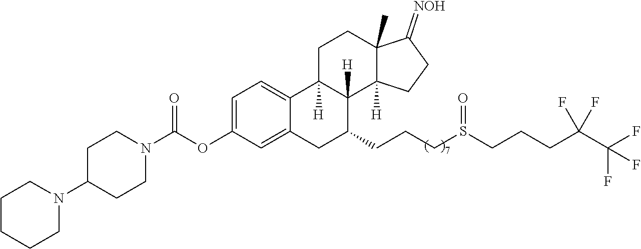 |
| 127 | 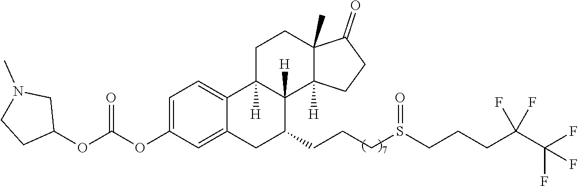 |
| 128 | 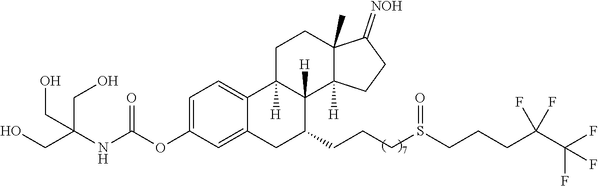 |
| 129 | 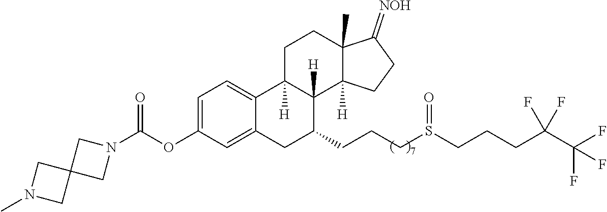 |
| 130 | 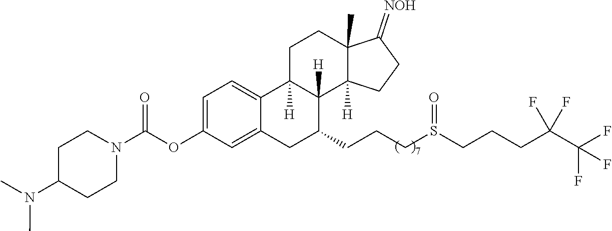 |
| 131 | 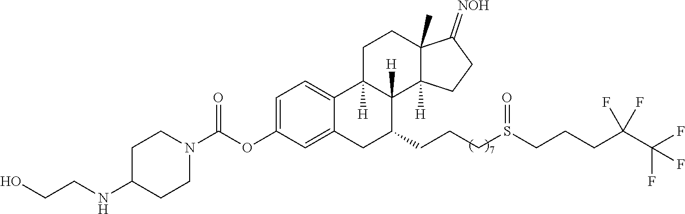 |
| 132 | 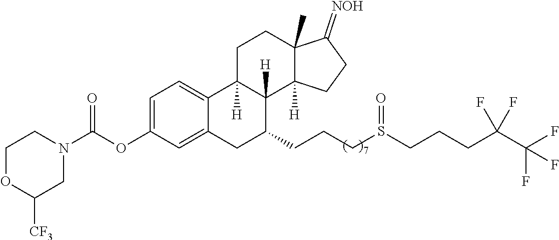 |
| 133 | 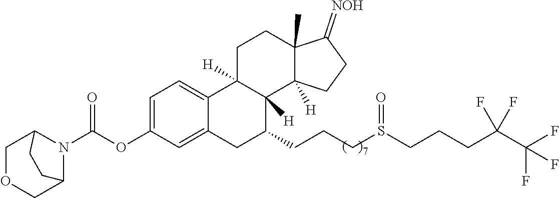 |
| 134 | 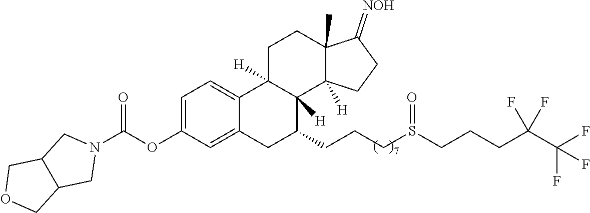 |
| 135 | 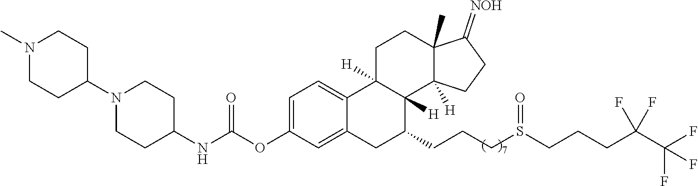 |
| 136 | 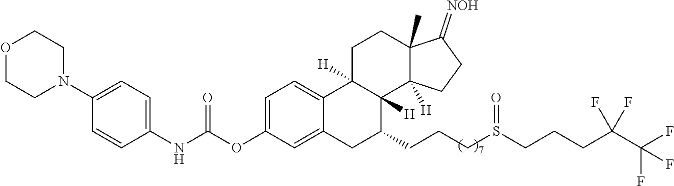 |
| 137 | 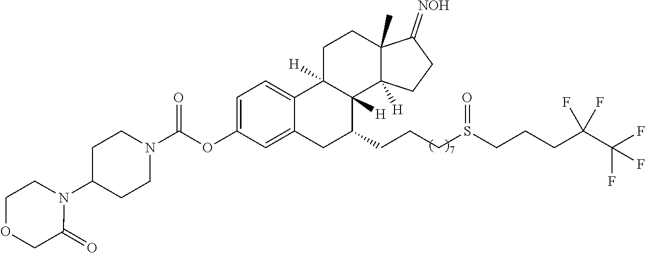 |
| 138 | 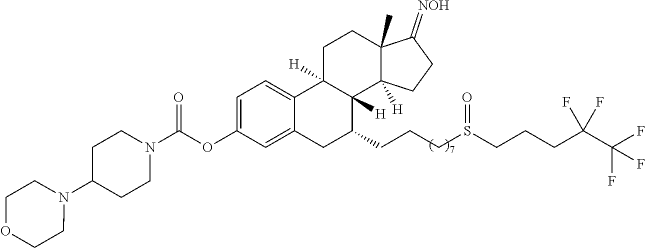 |
| 139 | 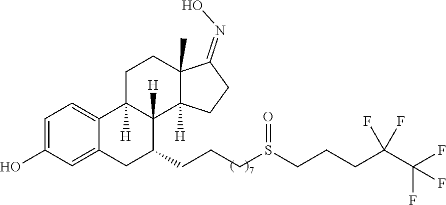 |
| 140 | 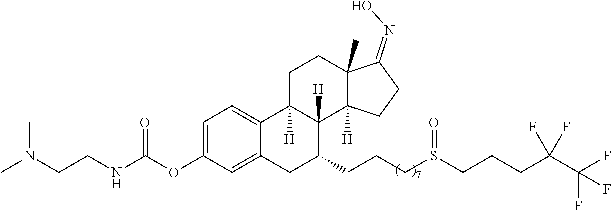 |
| 141 | 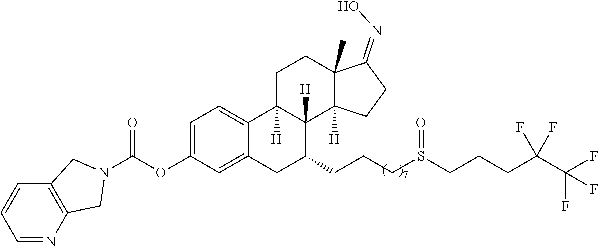 |
| 142 | 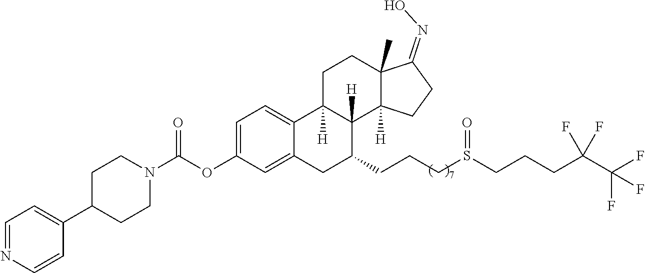 |
| 143 | 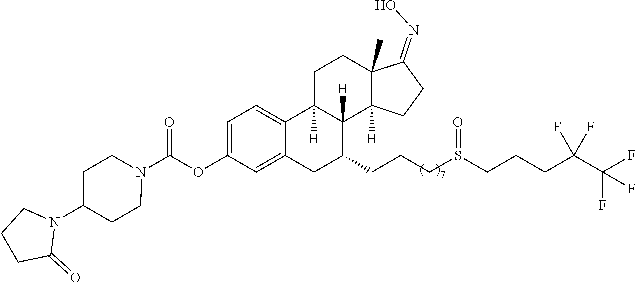 |
| 144 | 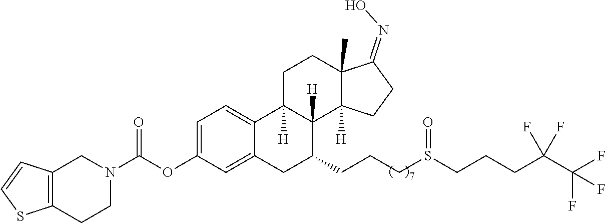 |
| 145 | 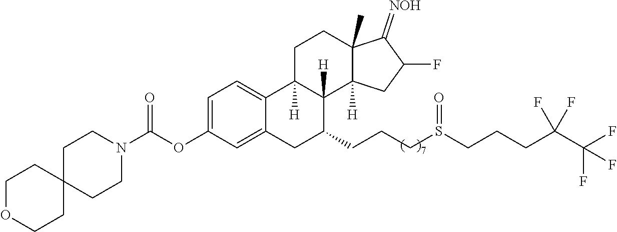 |
| 146 | 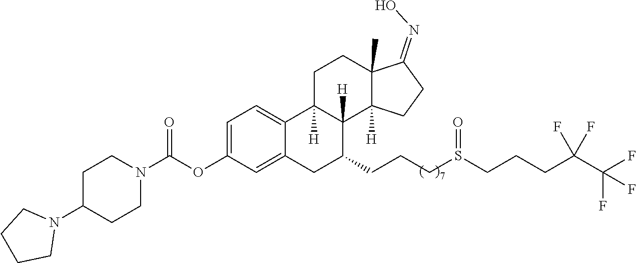 |
| 147 | 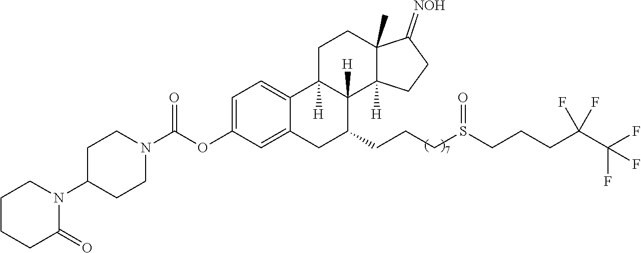 |
| 148 | 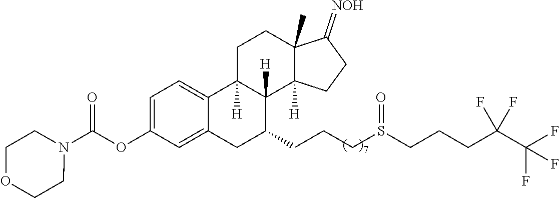 |
| 149 | 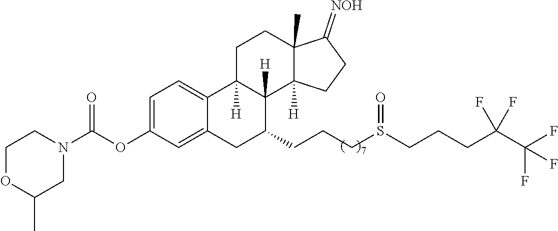 |
| 150 | 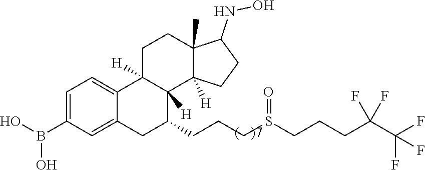 |
| 151 | 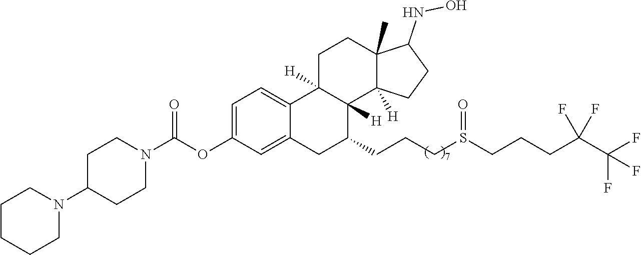 |
| 152 | 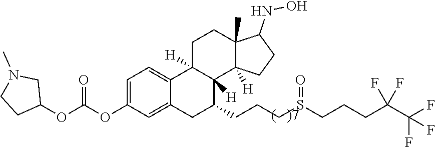 |
| 153 | 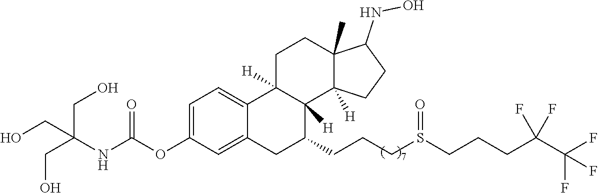 |
| 154 | 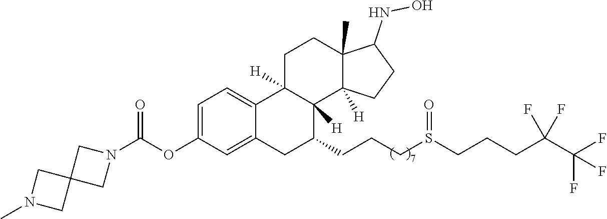 |
| 155 | 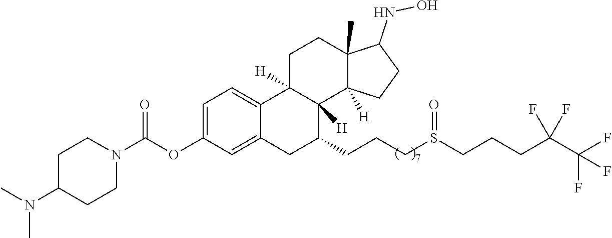 |
| 156 | 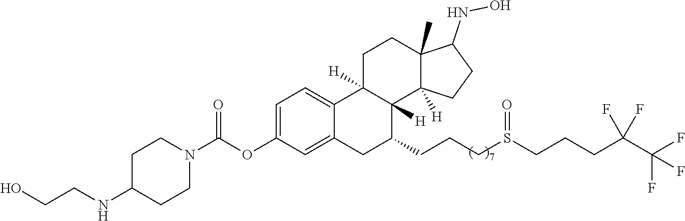 |
| 157 | 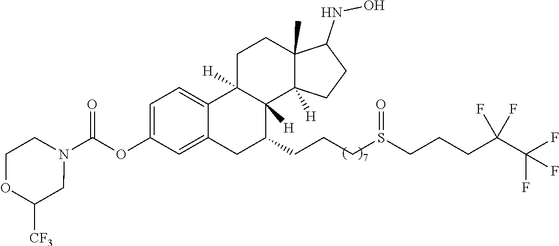 |
| 158 | 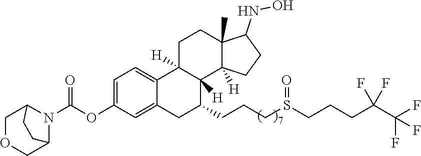 |
| 159 | 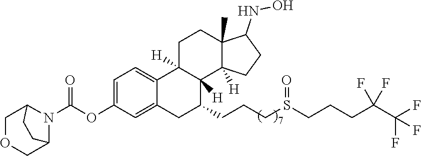 |
| 160 | 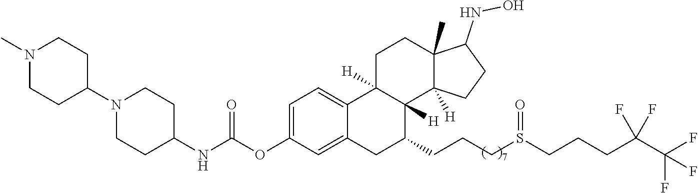 |
| 161 | 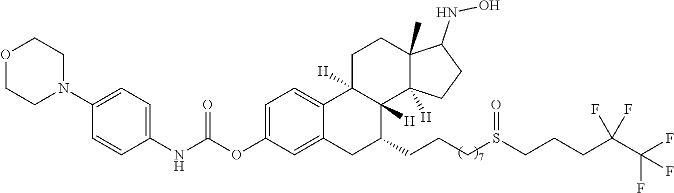 |
| 162 | 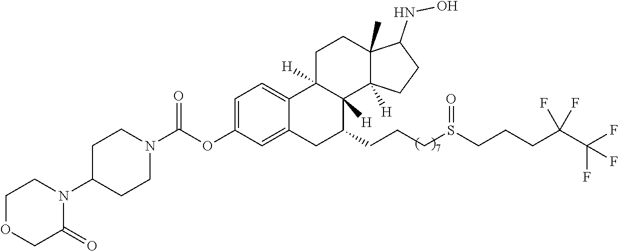 |
| 163 | 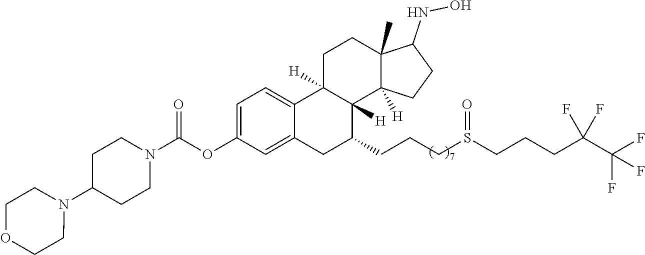 |
| 164 | 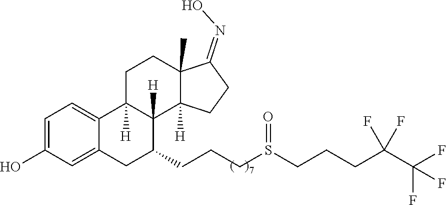 |
| 165 | 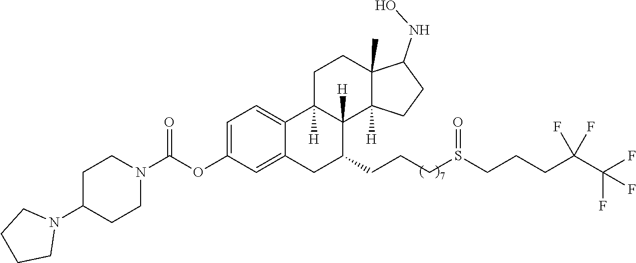 |
| 166 | 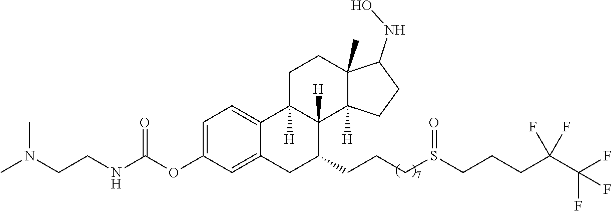 |
| 167 | 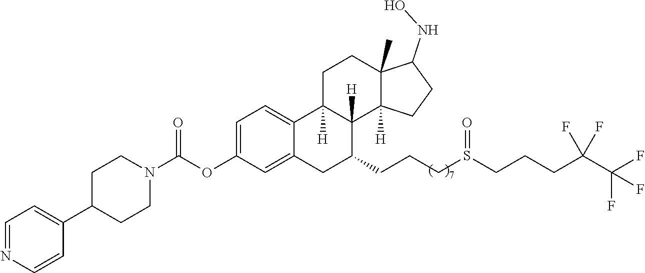 |
| 168 | 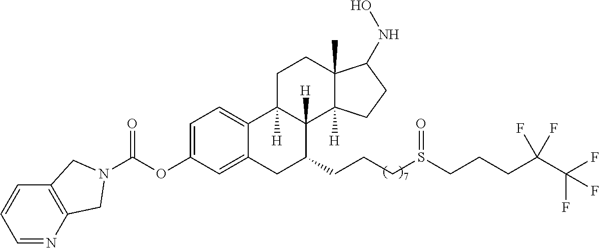 |
| 169 | 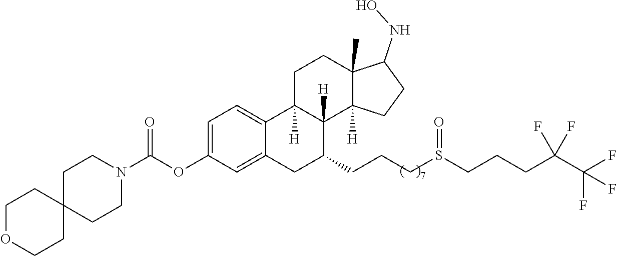 |
| 170 | 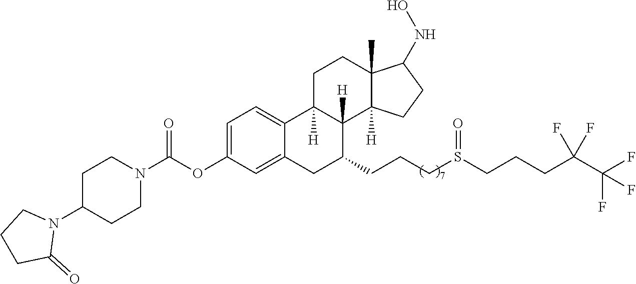 |
| 171 | 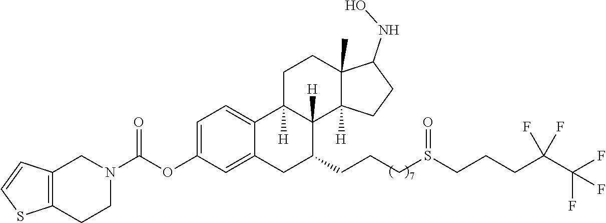 |
| 172 | 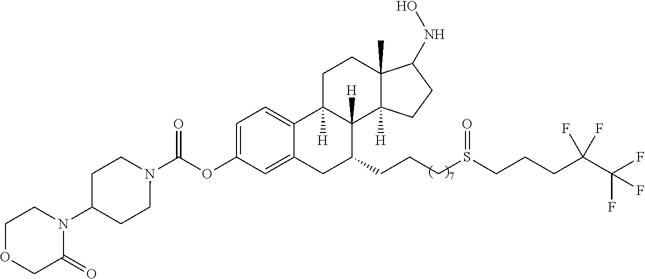 |
| 173 | 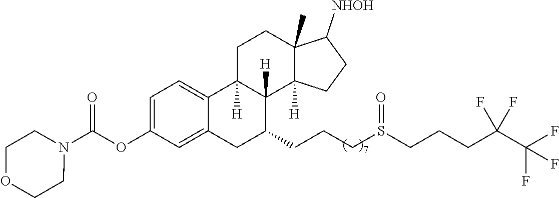 |
| 174 | 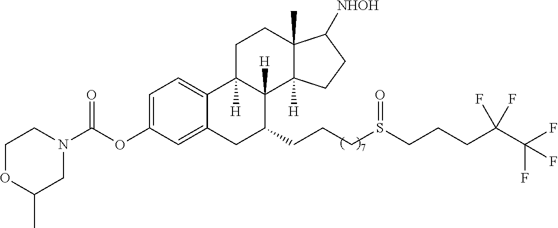 |
| 175 | 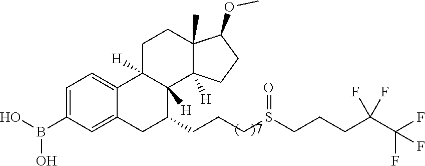 |
| 176 | 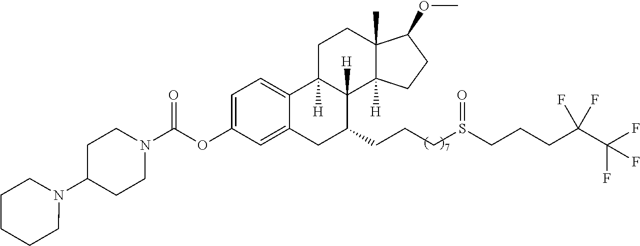 |
| 177 | 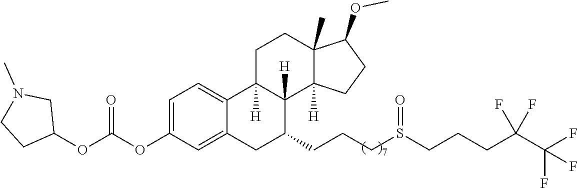 |
| 178 | 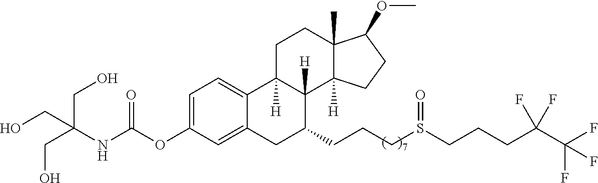 |
| 179 | 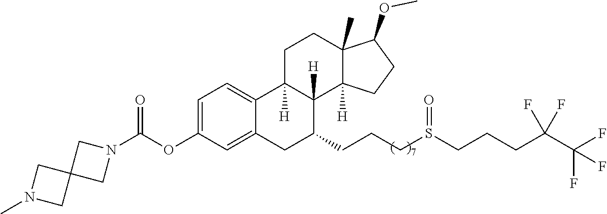 |
| 180 | 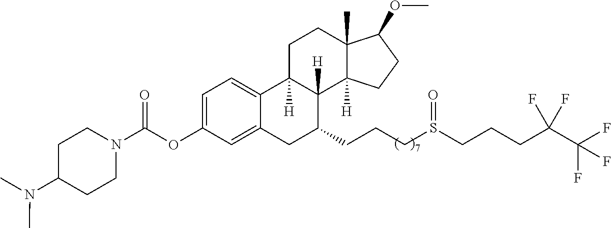 |
| 181 | 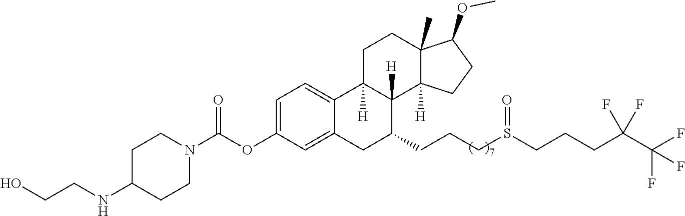 |
| 182 | 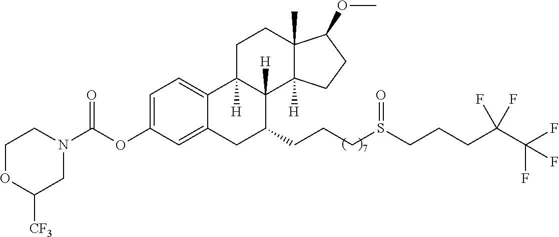 |
| 183 | 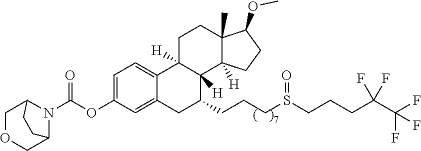 |
| 184 | 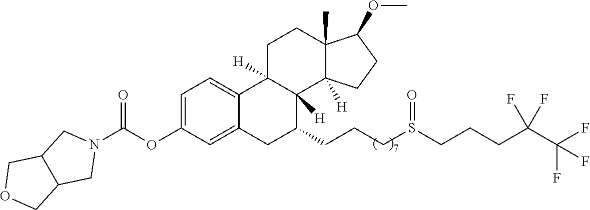 |
| 185 | 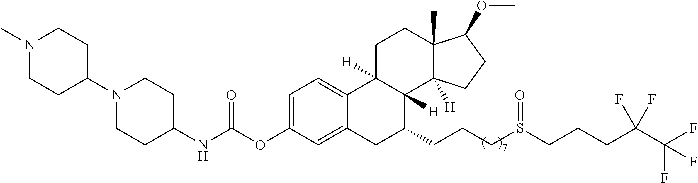 |
| 186 | 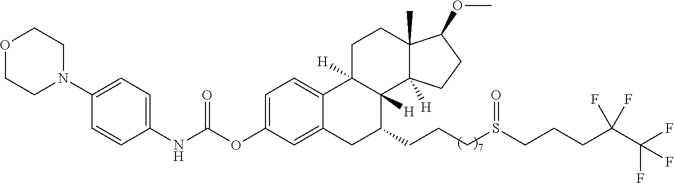 |
| 187 | 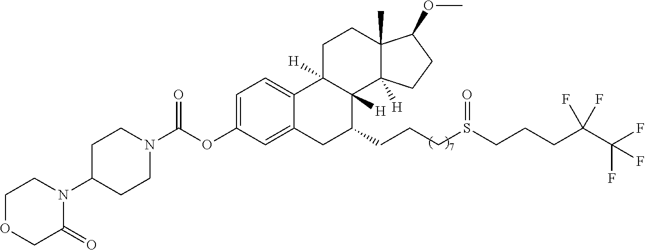 |
| 188 | 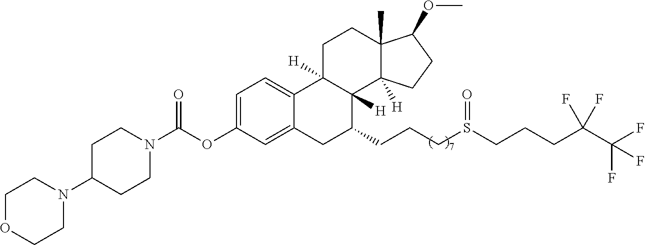 |
| 189 | 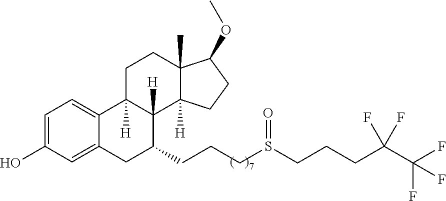 |
| 190 | 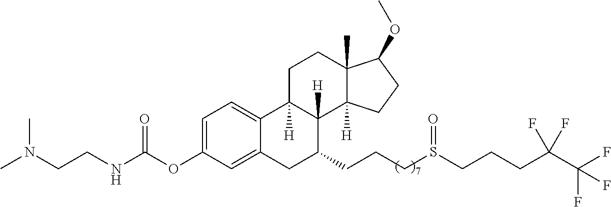 |
| 191 | 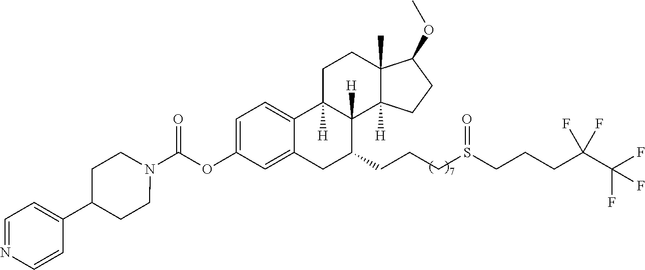 |
| 192 | 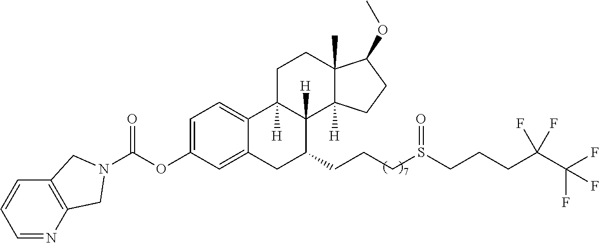 |
| 193 | 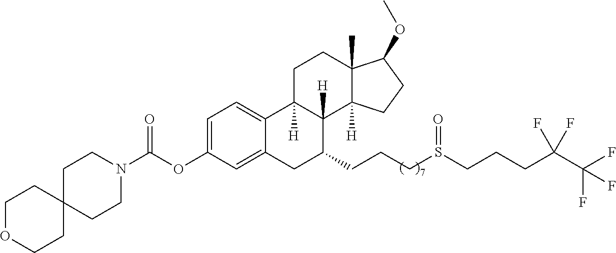 |
| 194 | 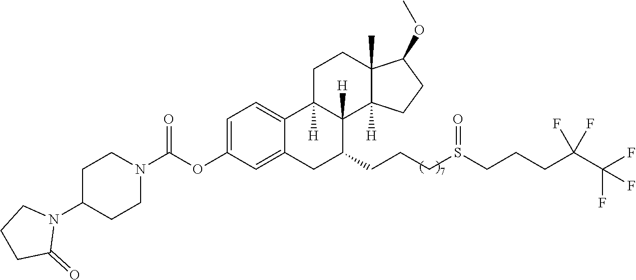 |
| 195 | 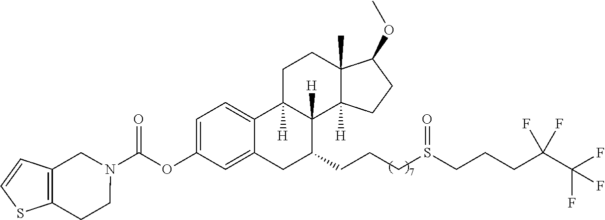 |
| 196 | 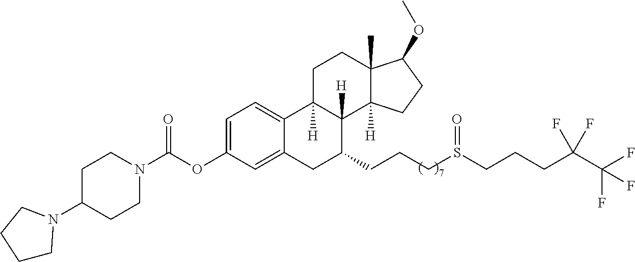 |
| 197 | 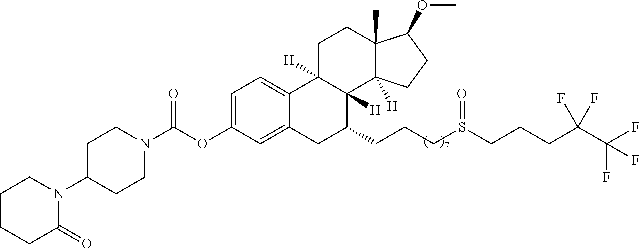 |
| 198 | 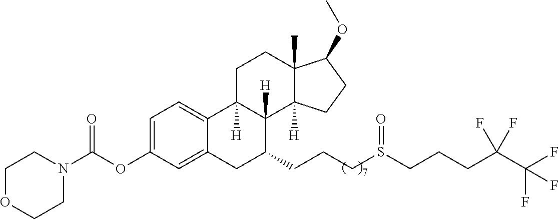 |
| 199 | 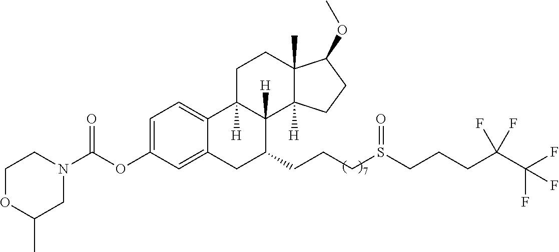 |
| 200 | 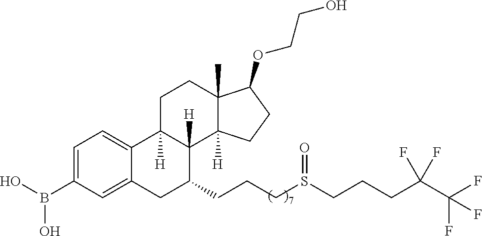 |
| 201 | 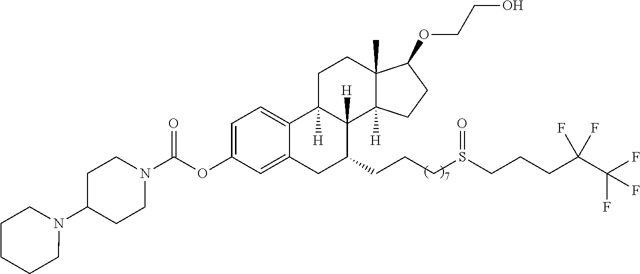 |
| 202 | 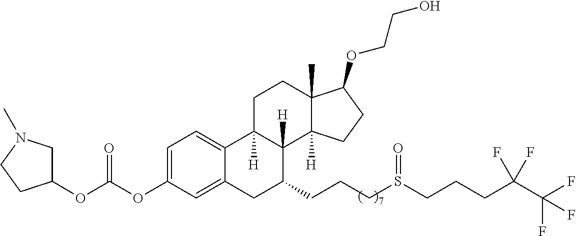 |
| 203 | 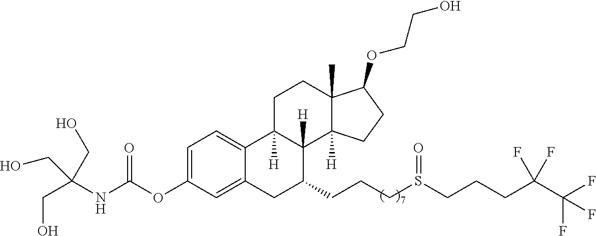 |
| 204 | 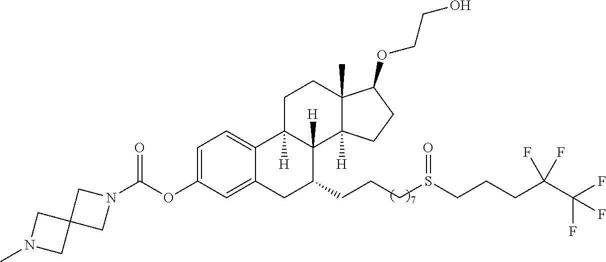 |
| 205 | 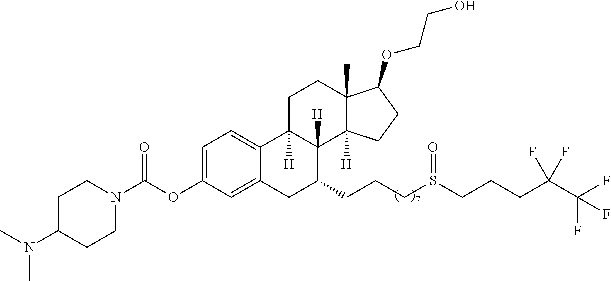 |
| 206 | 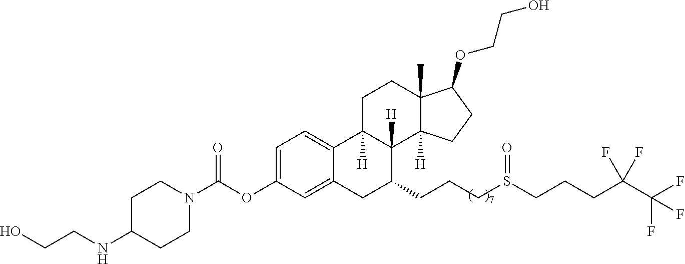 |
| 207 | 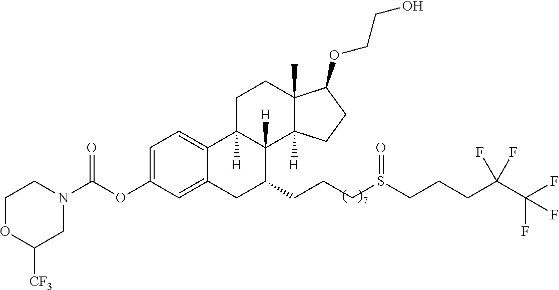 |
| 208 | 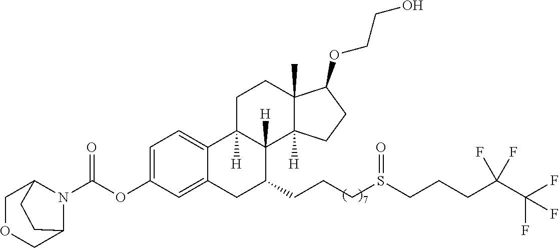 |
| 209 | 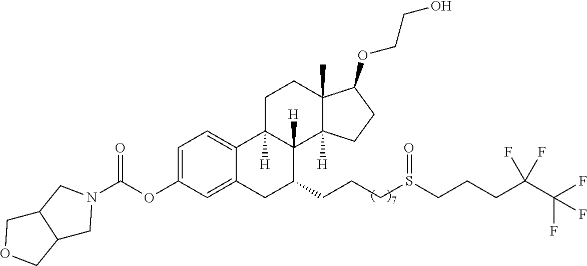 |
| 210 | 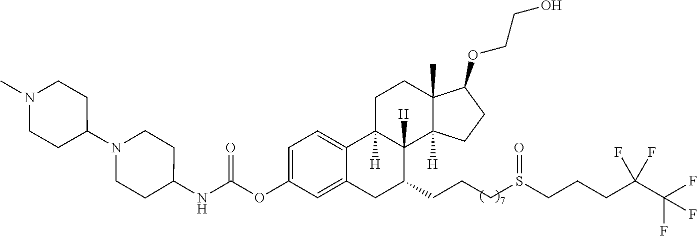 |
| 211 | 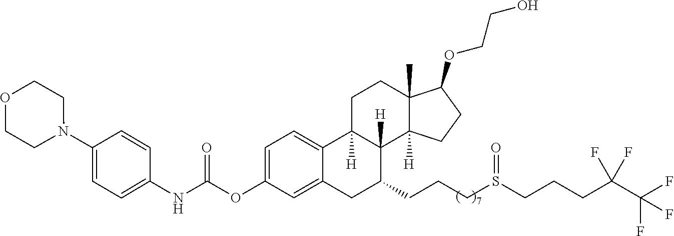 |
| 212 | 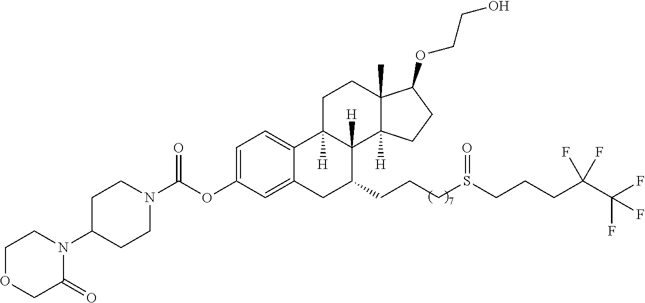 |
| 213 | 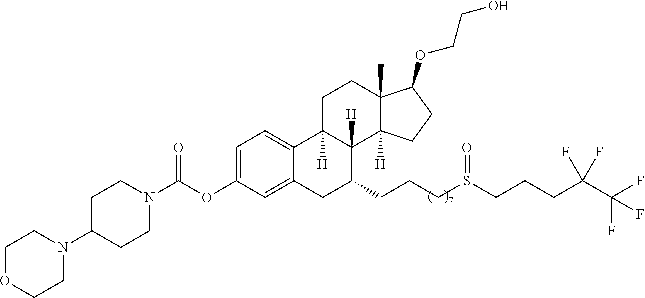 |
| 214 | 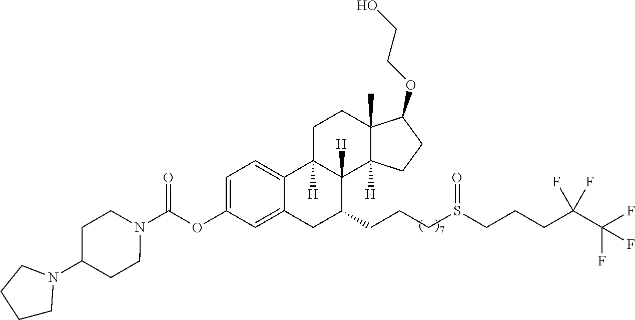 |
| 215 | 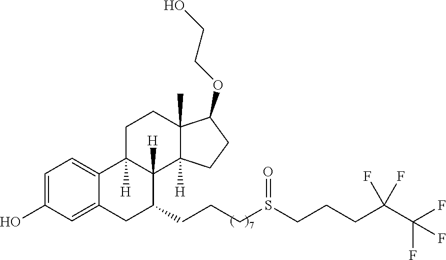 |
| 216 | 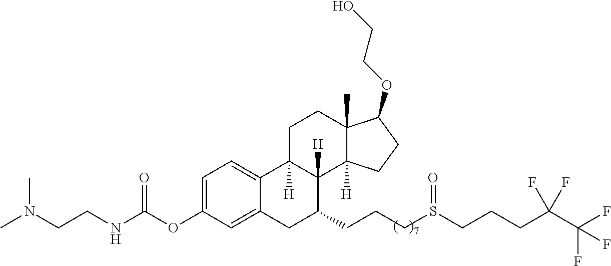 |
| 217 | 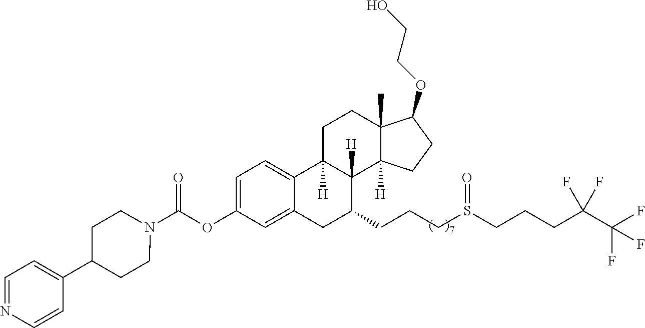 |
| 218 | 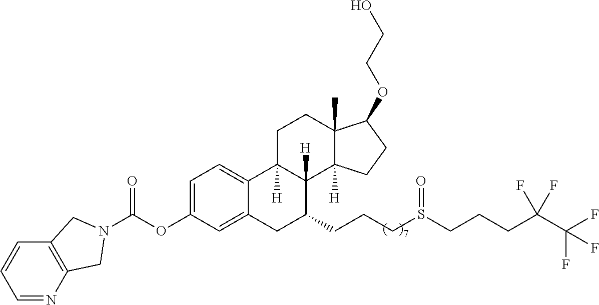 |
| 219 | 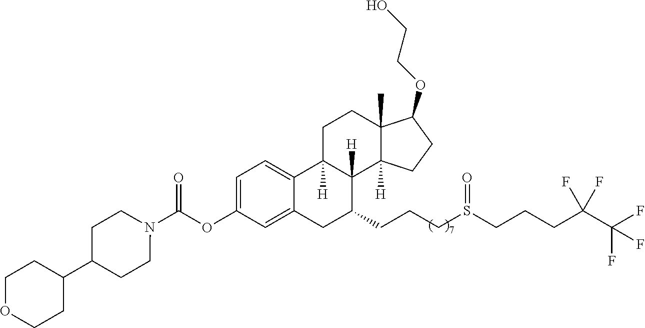 |
| 220 | 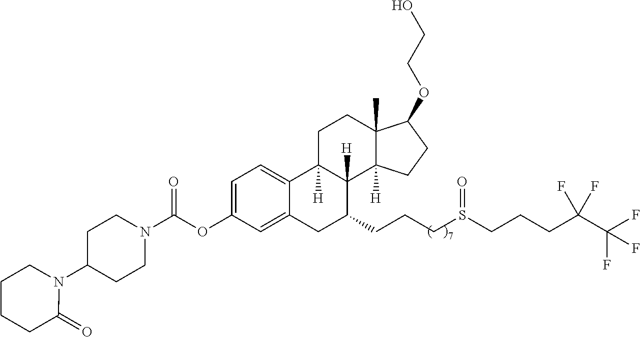 |
| 221 | 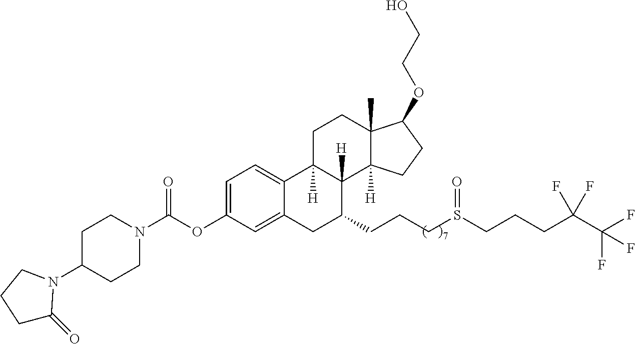 |
| 222 | 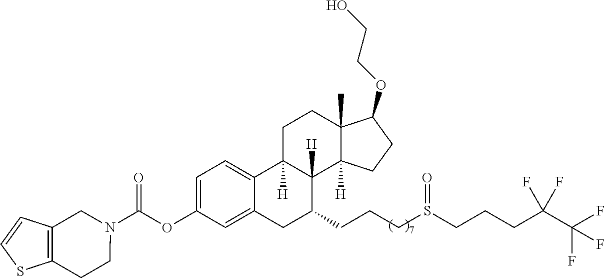 |
| 223 | 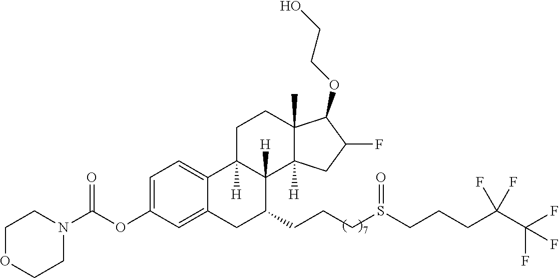 |
| 224 | 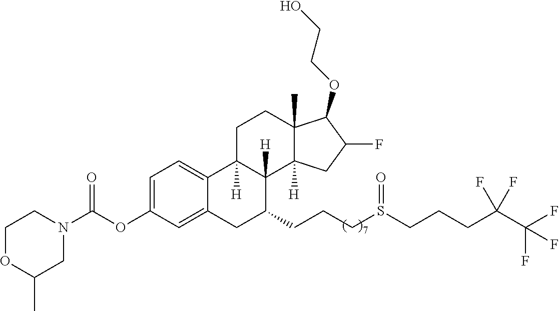 |
| 225 | 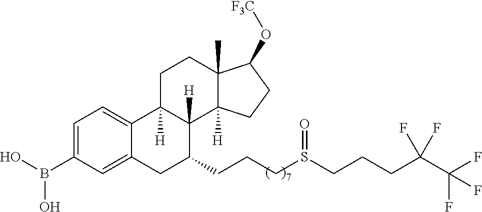 |
| 226 | 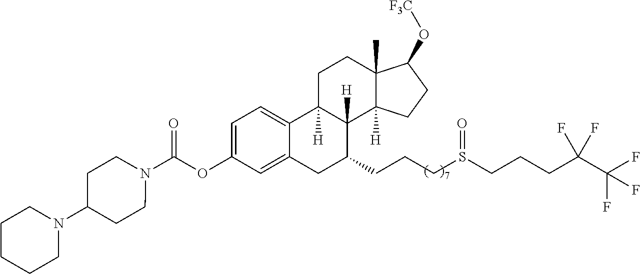 |
| 227 | 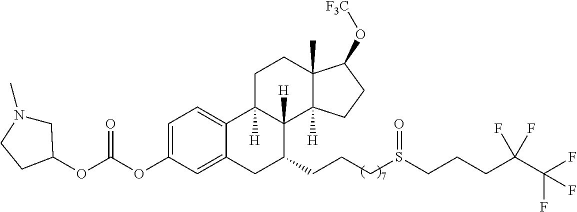 |
| 228 | 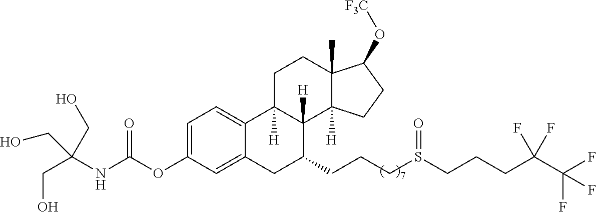 |
| 229 | 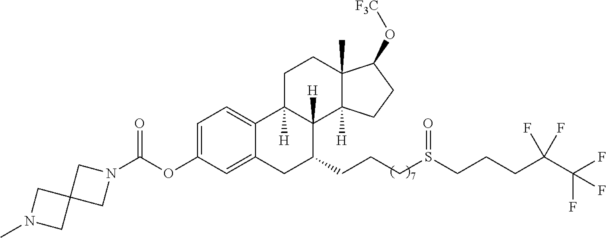 |
| 230 | 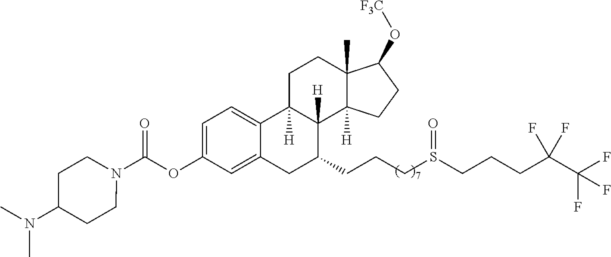 |
| 231 | 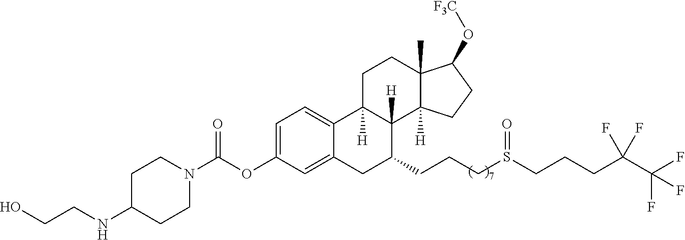 |
| 232 | 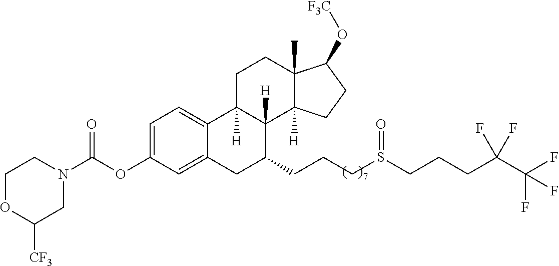 |
| 233 | 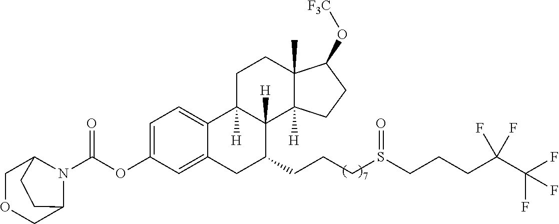 |
| 234 | 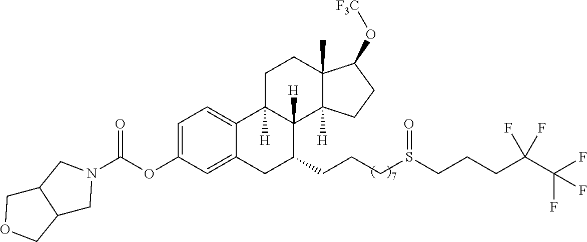 |
| 235 | 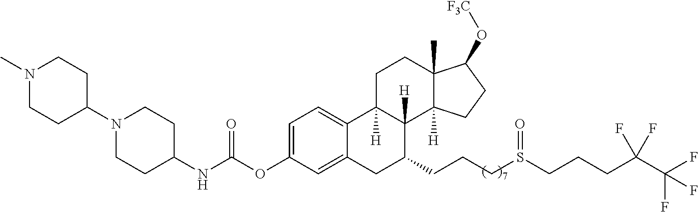 |
| 236 | 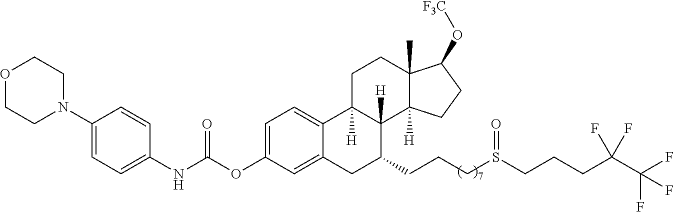 |
| 237 | 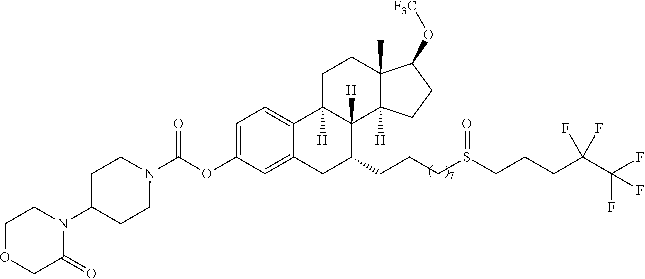 |
| 238 | 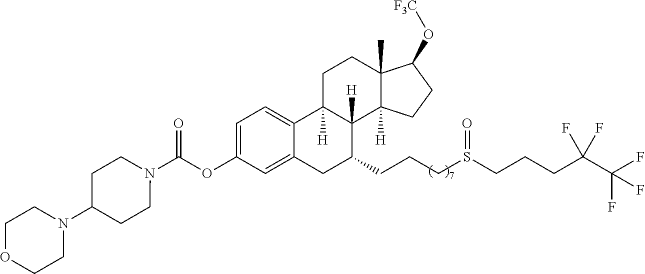 |
| 239 | 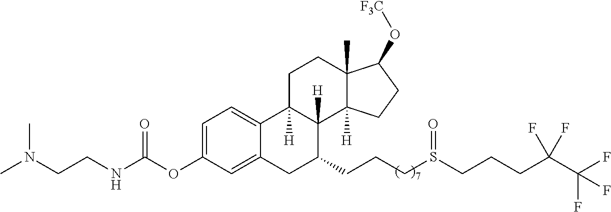 |
| 240 | 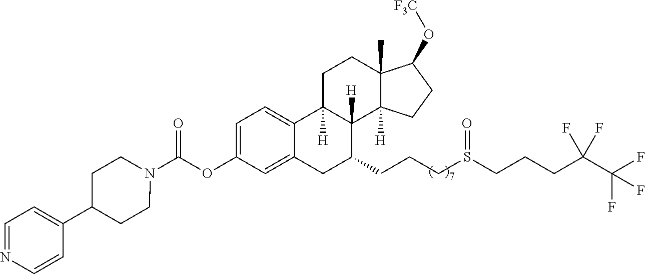 |
| 241 | 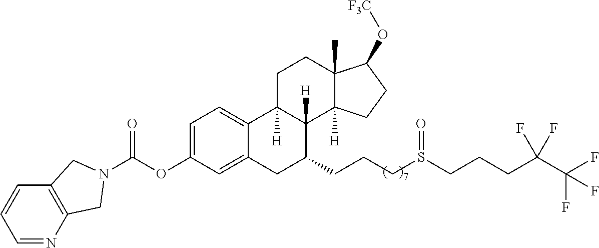 |
| 242 | 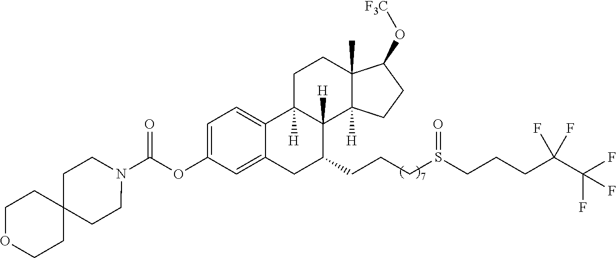 |
| 243 | 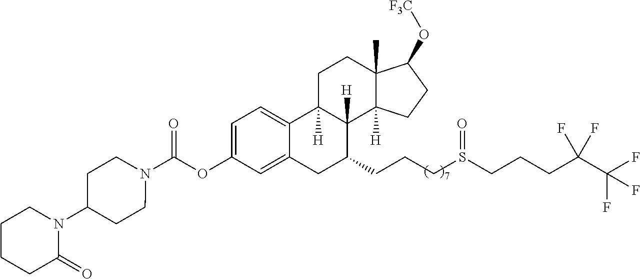 |
| 244 | 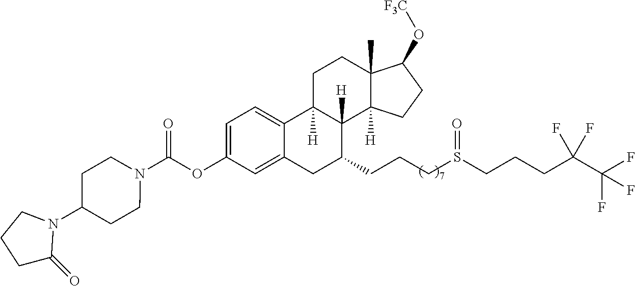 |
| 245 | 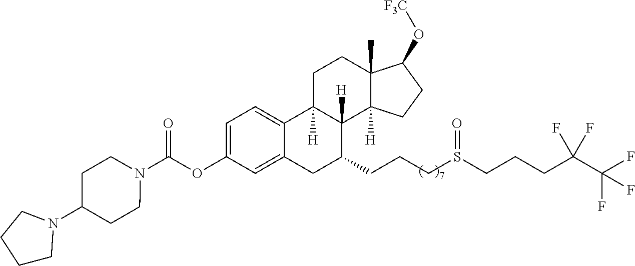 |
| 246 | 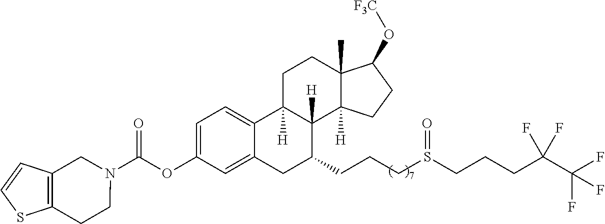 |
| 247 | 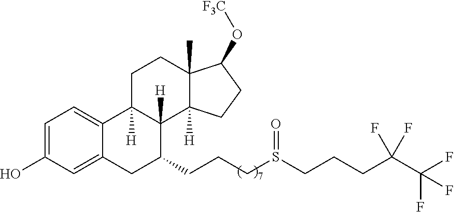 |
| 248 | 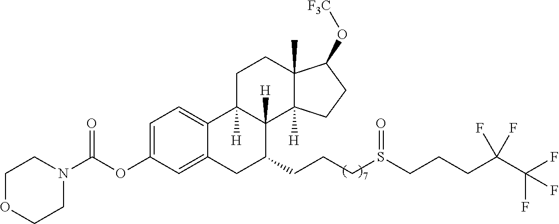 |
| 249 | 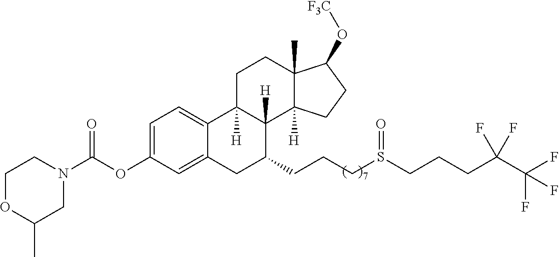 |
| 250 | 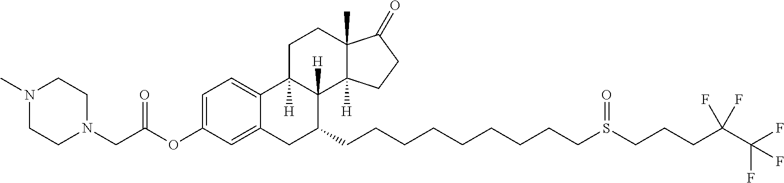 |

In one embodiment, the present invention relates to compound of formula A:

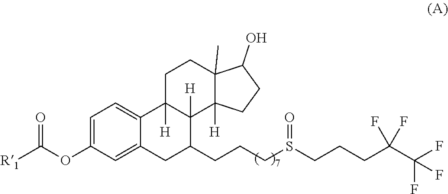

wherein,
R′₁ is selected from optionally substituted heterocycloalkyl, heteroaryl, and

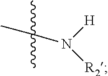

wherein one or more substitution on optionally substituted heterocycloalkyl, heteroaryl is selected from alkyl, haloalkyl, hydroxy, amino, alkylamino, dialkylamino, halo, and oxo group;
R′₂ is selected from group comprising optionally substituted heterocycloalkyl, heteroaryl and aryl group, wherein one or more substitution on heterocycloalkyl, heteroaryl and aryl is selected from alkyl, haloalkyl, hydroxy, amino, alkylamino, dialkylamino, unsubstituted heterocycloalkyl, substituted heterocycloalkyl, halo, and oxo group, wherein one or more substitution on substituted heterocycloalkyl is selected from alkyl, haloalkyl, hydroxy, amino, alkylamino, dialkylamino, halo, and oxo group;
enantiomers, diastereomers, racemates, pharmaceutically acceptable salts or solvates thereof.

In another embodiment, the present invention relates to compound of formula B:

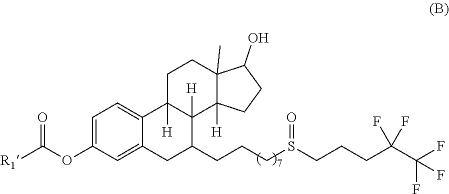

wherein; R′ᵢ is selected from

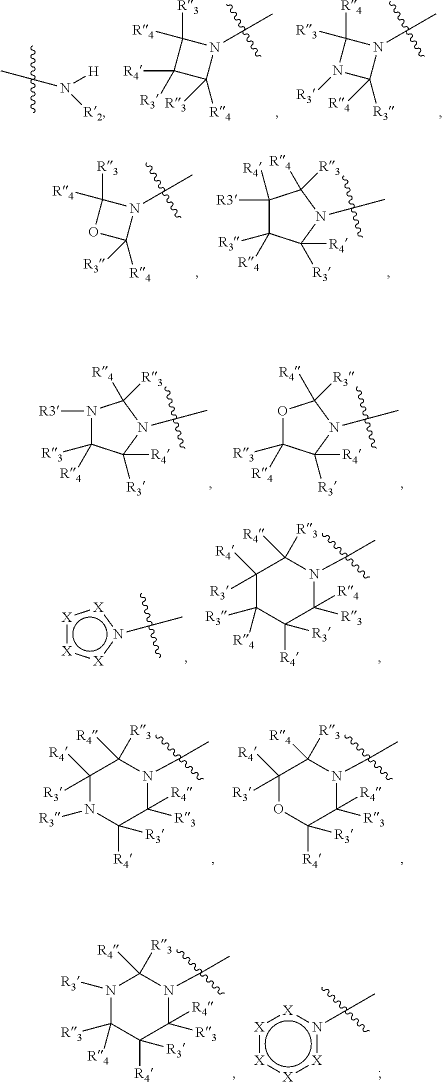

wherein R′₂ is selected from group comprising optionally substituted heterocycloalkyl, heteroaryl and aryl group,
wherein one or more substitution on heterocycloalkyl, heteroaryl and aryl is selected from alkyl, haloalkyl, hydroxy, amino, alkylamino, dialkylamino, unsubstituted heterocycloalkyl, substituted heterocycloalkyl, halo, and oxo group,
wherein one or more substitution on the substituted heterocycloalkyl is selected from alkyl, haloalkyl, hydroxy, amino, alkylamino, dialkylamino, halo, and oxo group;
[symbol] represents the aromatic ring;
X is each independently selected from N, O, S,

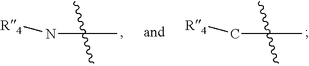

wherein R″₃, R″₄, R₃′, R₄′ is each independently selected from hydrogen, alkyl, haloalkyl, hydroxy, amino, alkylamino, dialkylamino, heterocycloalkyl, substituted heterocycloalkyl, and halo group, wherein the substituted heterocycloalkyl is substituted by one or more substitution is selected from alkyl, haloalkyl, hydroxy, amino, alkylamino, dialkylamino, halo, and oxo group; or each R″₃ and R₃′ or R″₄, and R₄′ or R″₃ and R″₄, or R₃′ and R₄′ are taken together along with the atom to which they are attached to form a three to seven membered saturated, partially unsaturated or unsaturated ring which may contain one or more heteroatoms selected from N, O and S, and the ring may optionally be substituted at a substitutable position with one or more R′₅ radicals, wherein the one or more R′₅ radicals are independently selected from hydrogen, alkyl, haloalkyl, hydroxy, amino, alkylamino, dialkylamino, halo, and oxo group; or each R″₃ and R₃′ or R″₄, and R₄′ are taken together along with the atom to which they are attached to form double bond; or each R″₃ and R″₄, or R₃′ and R₄′ are taken together form oxo( [symbol] ) bond;
enantiomers, diastereomers, racemates, pharmaceutically acceptable salts or solvates thereof.

In another embodiment, the present invention relates to compound of formula (C):

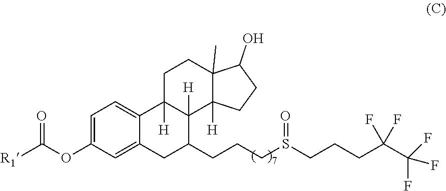

wherein;
R′₁ is selected from

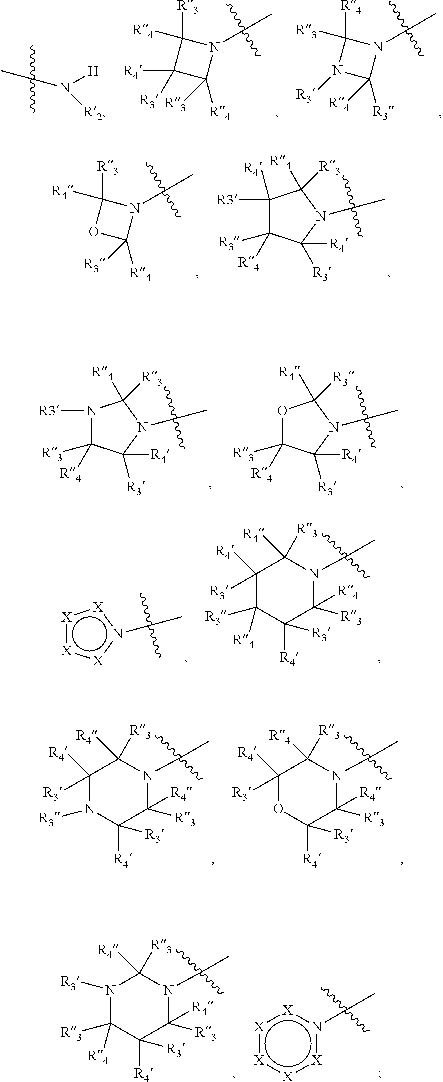

R′₂ is selected from,

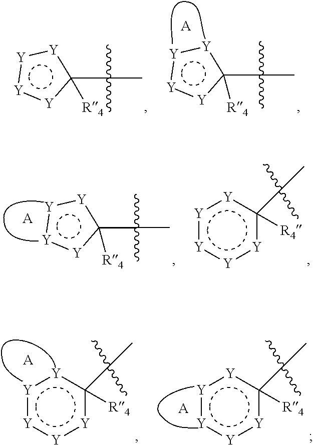

[symbol] represents the aromatic ring;
[symbol] represents the saturation, partial unsaturation or the complete unsaturation in the ring;
X is each independently selected from N, O, S,

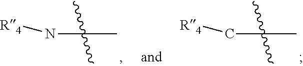

Y is selected from C,

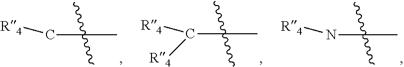

N, O, and S;
fused ring A represents an optionally substituted saturated, unsaturated, or aromatic four, five, six, or seven member ring having zero or more heteroatoms in the ring, the remaining atoms of the ring being carbon with each heteroatom being independently selected from nitrogen, oxygen and sulfur, wherein ring A is optionally substituted with one or more R″₆ radicals independently selected from hydrogen, alkyl, haloalkyl, hydroxy, amino, alkylamino, dialkylamino, unsubstituted heterocycloalkyl, substituted heterocycloalkyl, halo, and oxo group,
wherein the one or more substitution on substituted heterocycloalkyl is selected from alkyl, haloalkyl, hydroxy, amino, alkylamino, dialkylamino, halo, and oxo group;
R″₃, R″₄, R₃′, R₄′ each independently selected from H, alkyl, haloalkyl, hydroxy, amino, alkylamino, dialkylamino, heterocycloalkyl, substituted heterocycloalkyl, and halo group, wherein the substituted heterocycloalkyl is substituted by one or more substitution is selected from alkyl, haloalkyl, hydroxy, amino, alkylamino, dialkylamino, halo, and oxo group; or each R″₃ and R₃′ or R″₄, and R₄′ or R″₃ and R″₄, or R₃′ and R₄′ are taken together along with the atom to which they are attached to form a three to seven membered saturated, partially unsaturated or unsaturated ring which may contain one or more heteroatoms selected from N, O and S, and the ring may optionally be substituted at a substitutable position with one or more R′₅ radicals, wherein the one or more R′₅ radicals are independently selected from hydrogen, alkyl, haloalkyl, hydroxy, amino, alkylamino, dialkylamino, halo, and oxo group; or each R″₃ and R₃′ or R″₄, and R₄′ are taken together along with the atom to which they are attached to form double bond; or each R″₃ and R″₄, O [symbol] or R₃′ and R₄′ are taken together form oxo( [symbol] ) bond;
enantiomers, diastereomers, racemates, pharmaceutically acceptable salts or solvates thereof.

In another embodiment, the present invention relates to compound of formula (A) wherein, R′₂ is selected from group consisting of

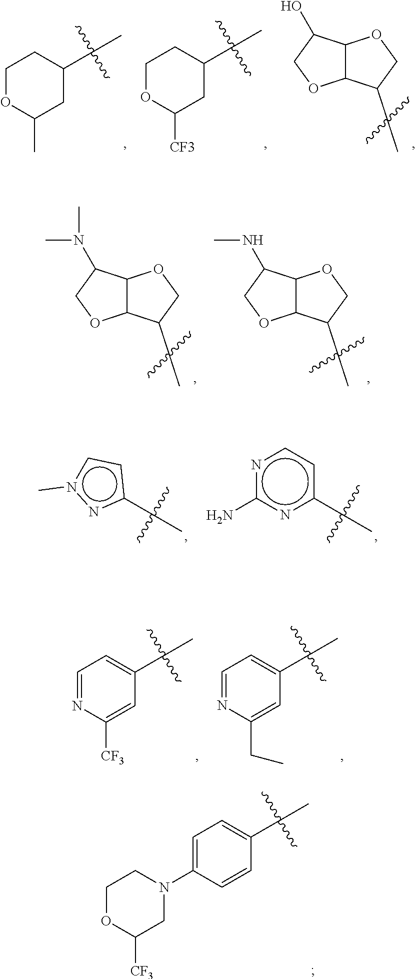

enantiomers, diastereomers, racemates, pharmaceutically acceptable salts or solvates thereof.

In another embodiment, the present invention relates to compound of formula (A) wherein, R′₁ is selected from optionally substituted heterocycloalkyl and heteroaryl is selected from group consisting of,

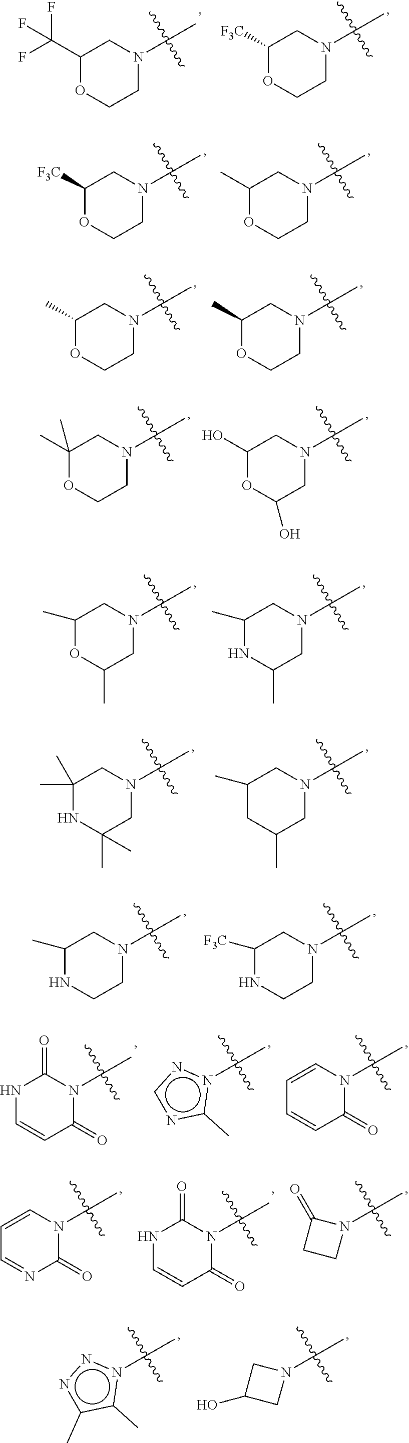

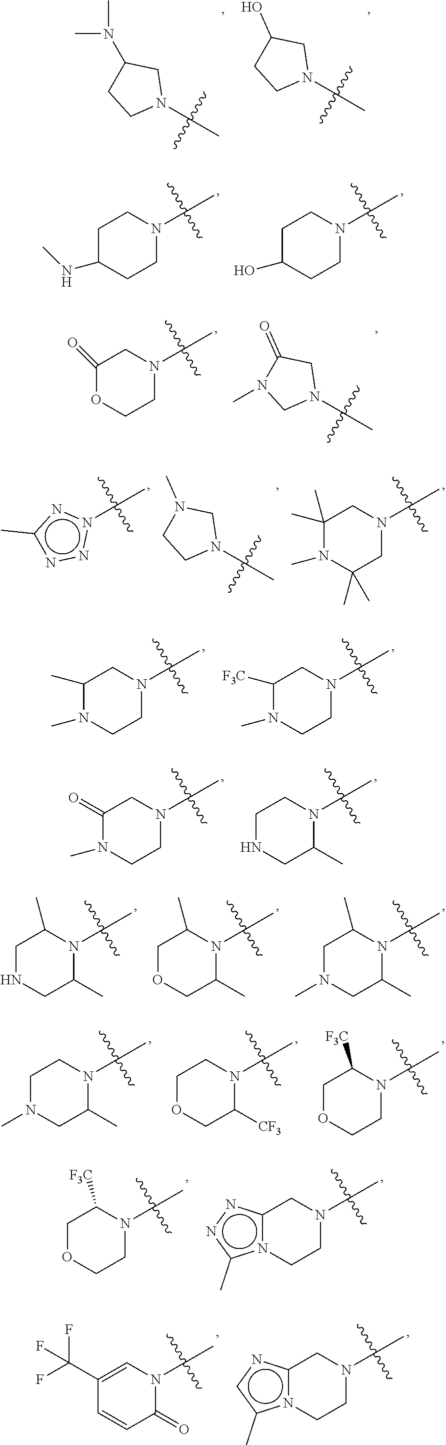

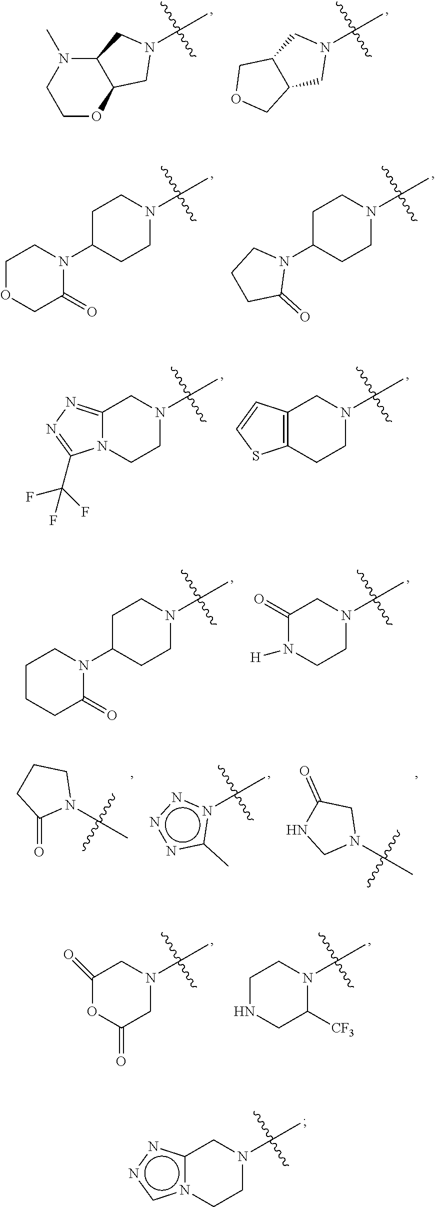

enantiomers, diastereomers, racemates, pharmaceutically acceptable salts or solvates thereof.

In another embodiment, the present invention relates to compound of formula (B):

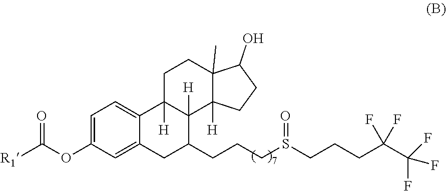

wherein;
R′₁ is selected from

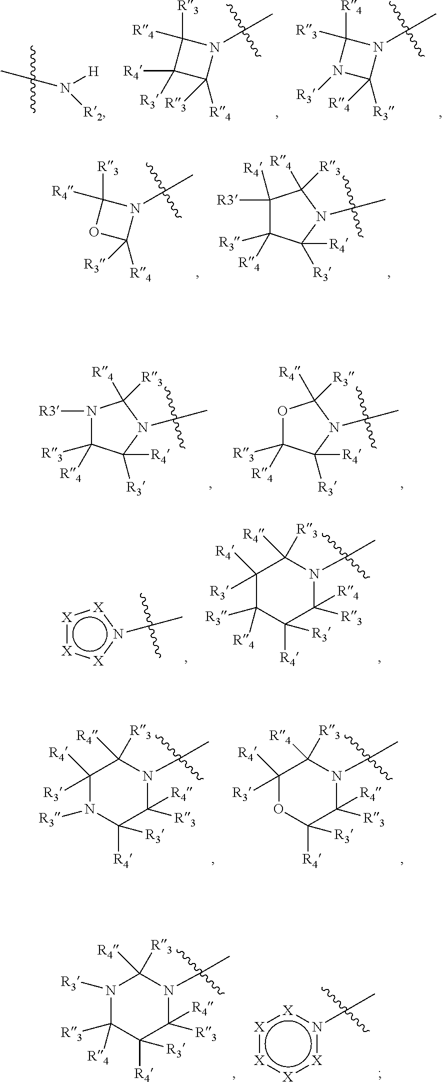

wherein R′₂ is selected from group comprising optionally substituted heterocycloalkyl, heteroaryl and aryl group,
wherein one or more substitution on heterocycloalkyl, heteroaryl and aryl is selected from alkyl, haloalkyl, hydroxy, amino, alkylamino, dialkylamino, unsubstituted heterocycloalkyl, substituted heterocycloalkyl, halo, and oxo group,
wherein one or more substitution on the substituted heterocycloalkyl is selected from alkyl, haloalkyl, hydroxy, amino, alkylamino, dialkylamino, halo, and oxo group;
[symbol] represents the aromatic ring;
X is each independently selected from N, O, S,

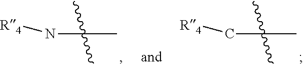

wherein R″₃, R″₄, R₃′, R₄′ is each independently selected from hydrogen, alkyl, haloalkyl, hydroxy, amino, alkylamino, dialkylamino, heterocycloalkyl, substituted heterocycloalkyl, and halo group, wherein the substituted heterocycloalkyl is substituted by one or more substitution is selected from alkyl, haloalkyl, hydroxy, amino, alkylamino, dialkylamino, halo, and oxo group; or each R″₃ and R₃′ or R″₄, and R₄′ or R″₃ and R″₄, or R₃′ and R₄′ are taken together along with the atom to which they are attached to form a three to seven membered saturated, partially unsaturated or unsaturated ring which may contain one or more heteroatoms selected from N, O and S, and the ring may optionally be substituted at a substitutable position with one or more R′₅ radicals, wherein the one or more R′₅ radicals are independently selected from hydrogen, alkyl, haloalkyl, hydroxy, amino, alkylamino, dialkylamino, halo, and oxo group; or each R″₃ and R₃′ or R″₄, and R₄′ are taken together along with the atom to which they are attached to form double bond; or each R″₃ and R″₄, or R₃′ and R₄′ are taken together form oxo( [symbol] ) bond;
enantiomers, diastereomers, racemates, pharmaceutically acceptable salts or solvates thereof.

In another embodiment, the present invention relates to compound of formula (C):

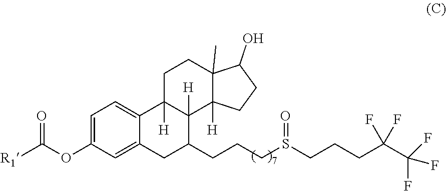

wherein;
R′₁ is selected from

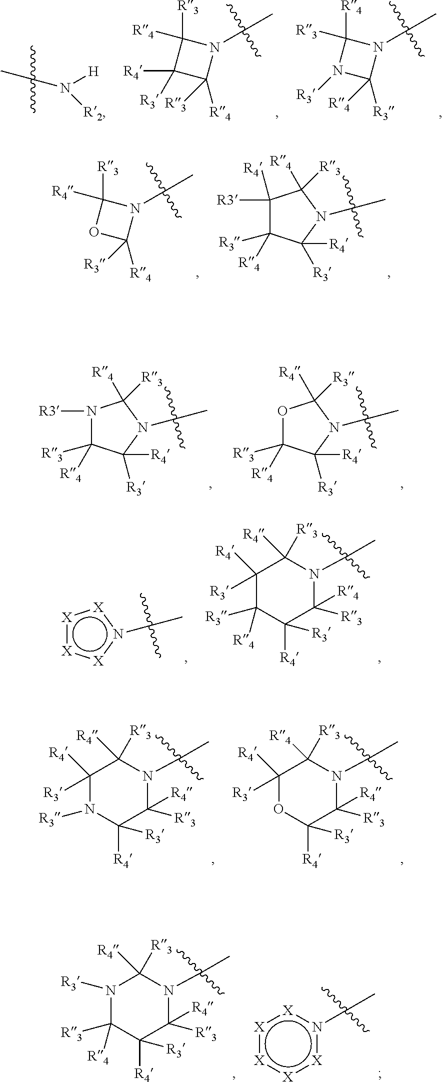

R′₂ is selected from,

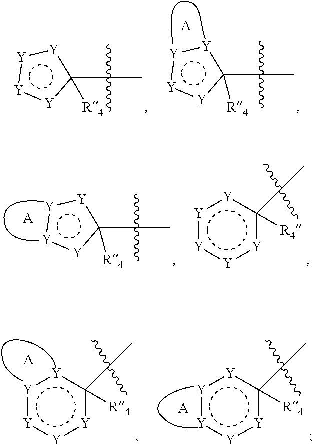

[symbol]

represents the aromatic ring;
[symbol] represents the saturation, partial unsaturation or the complete unsaturation in the ring;
X is each independently selected from N, O, S,

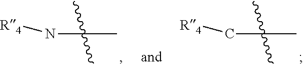

Y is selected from C,

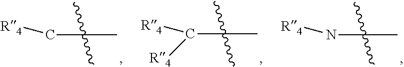

N, O, and S;
fused ring A represents an optionally substituted saturated, unsaturated, or aromatic four, five, six, or seven member ring having zero or more heteroatoms in the ring, the remaining atoms of the ring being carbon with each heteroatom being independently selected from nitrogen, oxygen and sulfur, wherein ring A is optionally substituted with one or more R″₆ radicals independently selected from hydrogen, alkyl, haloalkyl, hydroxy, amino, alkylamino, dialkylamino, unsubstituted heterocycloalkyl, substituted heterocycloalkyl, halo, and oxo group, wherein the one or more substitution on substituted heterocycloalkyl is selected from alkyl, haloalkyl, hydroxy, amino, alkylamino, dialkylamino, halo, and oxo group;
R″₃, R″₄, R₃′, R₄′ each independently selected from H, alkyl, haloalkyl, hydroxy, amino, alkylamino, dialkylamino, heterocycloalkyl, substituted heterocycloalkyl, and halo group, wherein the substituted heterocycloalkyl is substituted by one or more substitution is selected from alkyl, haloalkyl, hydroxy, amino, alkylamino, dialkylamino, halo, and oxo group; or each R″₃ and R₃′ or R″₄, and R₄′ or R″₃ and R″₄, or R₃′ and R₄′ are taken together along with the atom to which they are attached to form a three to seven membered saturated, partially unsaturated or unsaturated ring which may contain one or more heteroatoms selected from N, O and S, and the ring may optionally be substituted at a substitutable position with one or more R′₅ radicals, wherein the one or more R′₅ radicals are independently selected from hydrogen, alkyl, haloalkyl, hydroxy, amino, alkylamino, dialkylamino, halo, and oxo group; or each R″₃ and R₃′ or R″₄, and R₄′ are taken together along with the atom to which they are attached to form double bond; or each R″₃ and R″₄, or R₃′ and R₄′ are taken together form oxo( [symbol] ) bond;
enantiomers, diastereomers, racemates, pharmaceutically acceptable salts or solvates thereof.

In another embodiment, the present invention relates to a compound of formula (D):

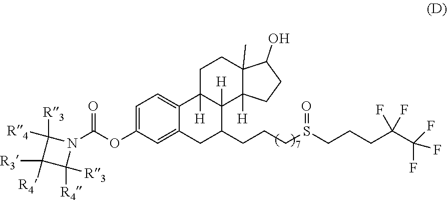

wherein,
R″₃, R″₄, R₃′, R₄′ is each independently selected from hydrogen, alkyl, haloalkyl, hydroxy, amino, alkylamino, dialkylamino, heterocycloalkyl, substituted heterocycloalkyl, and halo group, wherein the substituted heterocycloalkyl is substituted by one or more substitution is selected from alkyl, haloalkyl, hydroxy, amino, alkylamino, dialkylamino, halo, and oxo group; or
each R″₃ and R₃′ or R″₄, and R₄′ or R″₃ and R″₄, or R₃′ and R₄′ are taken together along with the atom to which they are attached to form a three to seven membered saturated, partially unsaturated or unsaturated ring which may contain one or more heteroatoms selected from N, O and S, and the ring may optionally be substituted at a substitutable position with one or more R′₅ radicals, wherein the one or more R′₅ radicals are independently selected from hydrogen, alkyl, haloalkyl, hydroxy, amino, alkylamino, dialkylamino, halo, and oxo group; or
each R″₃ and R₃′ or R″₄, and R₄′ are taken together along with the atom to which they are attached to form double bond; or
each R″₃ and R″₄, or R₃′ and R₄′ are taken together form oxo( [symbol] ) bond;
enantiomers, diastereomers, racemates, pharmaceutically acceptable salts or solvates thereof.

In another embodiment, the present invention provides the compound selected from group consisting of,

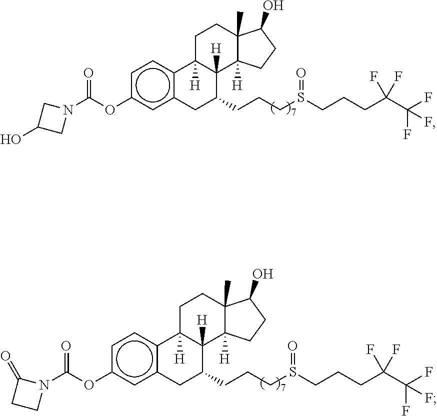

enantiomers, diastereomers, racemates, pharmaceutically acceptable salts or solvates thereof.

In another embodiment, the present invention relates to a compound of formula (E):

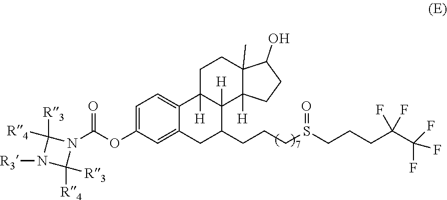

wherein,
R″₃, R″₄, R₃′, R₄′ is each independently selected from hydrogen, alkyl, haloalkyl, hydroxy, amino, alkylamino, dialkylamino, heterocycloalkyl, substituted heterocycloalkyl, and halo group, wherein the substituted heterocycloalkyl is substituted by one or more substitution is selected from alkyl, haloalkyl, hydroxy, amino, alkylamino, dialkylamino, halo, and oxo group; or
each R″₃ and R₃′ or R″₄, and R₄′ or R″₃ and R″₄, or R₃′ and R₄′ are taken together along with the atom to which they are attached to form a three to seven membered saturated, partially unsaturated or unsaturated ring which may contain one or more heteroatoms selected from N, O and S, and the ring may optionally be substituted at a substitutable position with one or more R′₅ radicals, wherein the one or more R′₅ radicals are independently selected from hydrogen, alkyl, haloalkyl, hydroxy, amino, alkylamino, dialkylamino, halo, and oxo group; or
each R″₃ and R₃′ or R″₄, and R₄′ are taken together along with the atom to which they are attached to form double bond; or
each R″₃ and R″₄, or R₃′ and R₄′ are taken together form oxo( [symbol] ) bond;
enantiomers, diastereomers, racemates, pharmaceutically acceptable salts or solvates thereof.

In another embodiment, the present invention relates to a compound of formula (F):

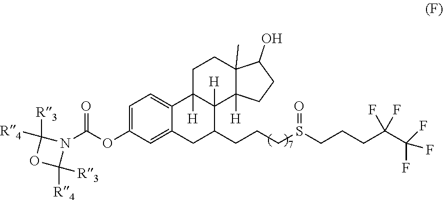

wherein,
R″₃, R″₄, R₃′, R₄′ is each independently selected from hydrogen, alkyl, haloalkyl, hydroxy, amino, alkylamino, dialkylamino, heterocycloalkyl, substituted heterocycloalkyl, and halo group, wherein the substituted heterocycloalkyl is substituted by one or more substitution is selected from alkyl, haloalkyl, hydroxy, amino, alkylamino, dialkylamino, halo, and oxo group; or
each R″₃ and R₃′ or R″₄, and R₄′ or R″₃ and R″₄, or R₃′ and R₄′ are taken together along with the atom to which they are attached to form a three to seven membered saturated, partially unsaturated or unsaturated ring which may contain one or more heteroatoms selected from N, O and S, and the ring may optionally be substituted at a substitutable position with one or more R′₅ radicals, wherein the one or more R′₅ radicals are independently selected from hydrogen, alkyl, haloalkyl, hydroxy, amino, alkylamino, dialkylamino, halo, and oxo group; or
each R″₃ and R₃′ or R″₄, and R₄′ are taken together along with the atom to which they are attached to form double bond; or
each R″₃ and R″₄, or R₃′ and R₄′ are taken together form oxo( [symbol] ) bond;
enantiomers, diastereomers, racemates, pharmaceutically acceptable salts or solvates thereof.

In another embodiment, the present invention relates to a compound of formula (G):

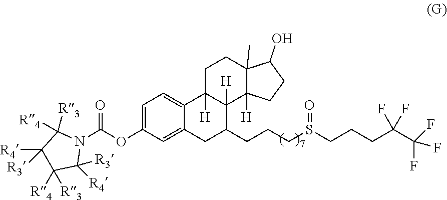

wherein,
R″₃, R″₄, R₃′, R₄′ is each independently selected from hydrogen, alkyl, haloalkyl, hydroxy, amino, alkylamino, dialkylamino, heterocycloalkyl, substituted heterocycloalkyl, and halo group, wherein the substituted heterocycloalkyl is substituted by one or more substitution is selected from alkyl, haloalkyl, hydroxy, amino, alkylamino, dialkylamino, halo, and oxo group; or
each R″₃ and R₃′ or R″₄, and R₄′ or R″₃ and R″₄, or R₃′ and R₄′ are taken together along with the atom to which they are attached to form a three to seven membered saturated, partially unsaturated or unsaturated ring which may contain one or more heteroatoms selected from N, O and S, and the ring may optionally be substituted at a substitutable position with one or more R′₅ radicals, wherein the one or more R′₅ radicals are independently selected from hydrogen, alkyl, haloalkyl, hydroxy, amino, alkylamino, dialkylamino, halo, and oxo group; or
each R″₃ and R₃′ or R″₄, and R₄′ are taken together along with the atom to which they are attached to form double bond; or
each R″₃ and R″₄, or R₃′ and R₄′ are taken together form oxo( [symbol] ) bond;
enantiomers, diastereomers, racemates, pharmaceutically acceptable salts or solvates thereof.

In another embodiment, the present invention relates to the compound selected from the group consisting of:

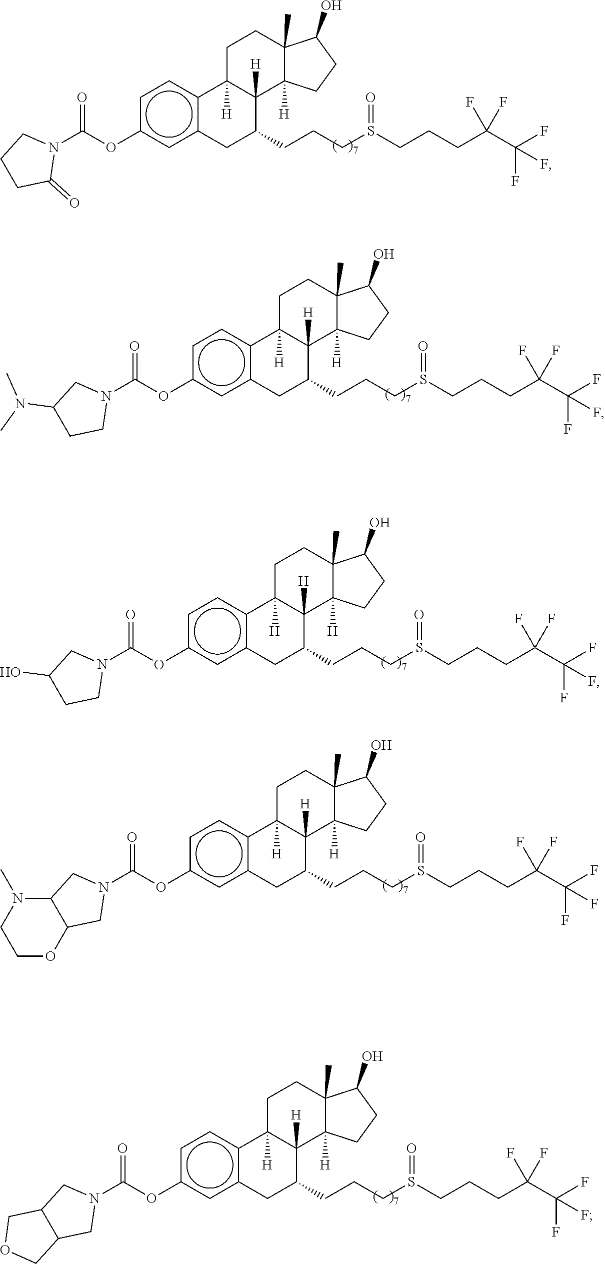

enantiomers, diastereomers, racemates, pharmaceutically acceptable salts or solvates thereof.

In another embodiment, the present invention relates to compound of formula H:

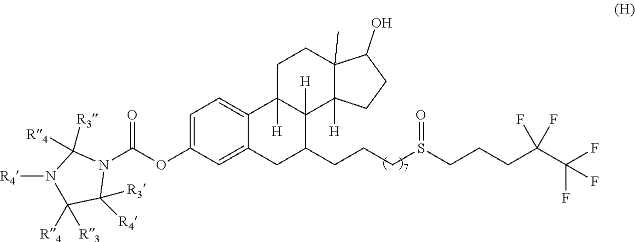

wherein,
R″₃, R″₄, R₃′, R₄′ is each independently selected from hydrogen, alkyl, haloalkyl, hydroxy, amino, alkylamino, dialkylamino, heterocycloalkyl, substituted heterocycloalkyl, and halo group, wherein the substituted heterocycloalkyl is substituted by one or more substitution is selected from alkyl, haloalkyl, hydroxy, amino, alkylamino, dialkylamino, halo, and oxo group; or
each R″₃ and R₃′ or R″₄, and R₄′ or R″₃ and R″₄, or R₃′ and R₄′ are taken together along with the atom to which they are attached to form a three to seven membered saturated, partially unsaturated or unsaturated ring which may contain one or more heteroatoms selected from N, O and S, and the ring may optionally be substituted at a substitutable position with one or more R′₅ radicals, wherein the one or more R′₅ radicals are independently selected from hydrogen, alkyl, haloalkyl, hydroxy, amino, alkylamino, dialkylamino, halo, and oxo group; or
each R″₃ and R₃′ or R″₄, and R₄′ are taken together along with the atom to which they are attached to form double bond; or
each R″₃ and R″₄, or R₃′ and R₄′ are taken together form oxo( [symbol] ) bond;
enantiomers, diastereomers, racemates, pharmaceutically acceptable salts or solvates thereof.

In another embodiment, the present invention relates to the compound selected from the group consisting of,

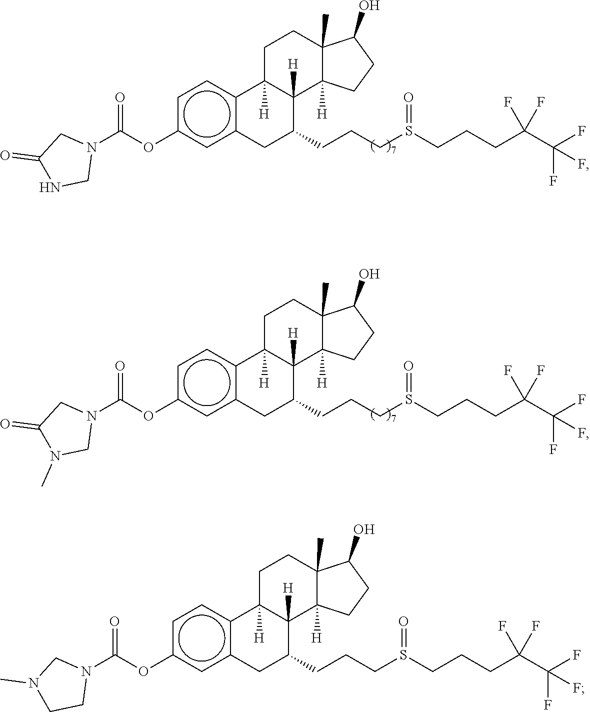

enantiomers, diastereomers, racemates, pharmaceutically acceptable salts or solvates thereof.

In another embodiment, the present invention relates to compound of formula (W):

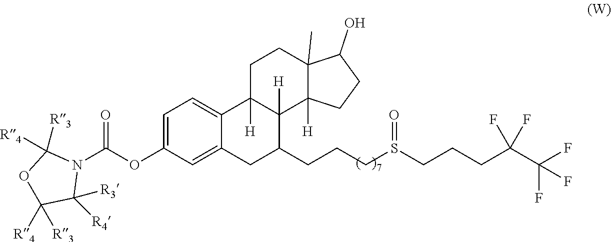

wherein,
R″₃, R″₄, R₃′, R₄′ is each independently selected from hydrogen, alkyl, haloalkyl, hydroxy, amino, alkylamino, dialkylamino, heterocycloalkyl, substituted heterocycloalkyl, and halo group, wherein the substituted heterocycloalkyl is substituted by one or more substitution is selected from alkyl, haloalkyl, hydroxy, amino, alkylamino, dialkylamino, halo, and oxo group; or
each R″₃ and R₃′ or R″₄, and R₄′ or R″₃ and R″₄, or R₃′ and R₄′ are taken together along with the atom to which they are attached to form a three to seven membered saturated, partially unsaturated or unsaturated ring which may contain one or more heteroatoms selected from N, O and S, and the ring may optionally be substituted at a substitutable position with one or more R′₅ radicals, wherein the one or more R′₅ radicals are independently selected from hydrogen, alkyl, haloalkyl, hydroxy, amino, alkylamino, dialkylamino, halo, and oxo group; or
each R″₃ and R₃′ or R″₄, and R₄′ are taken together along with the atom to which they are attached to form double bond; or
each R″₃ and R″₄, orR3′ and R₄′ are taken together form oxo( [symbol] ) bond;
enantiomers, diastereomers, racemates, pharmaceutically acceptable salts or solvates thereof.

In another embodiment, the present invention relates to compound of formula (J):

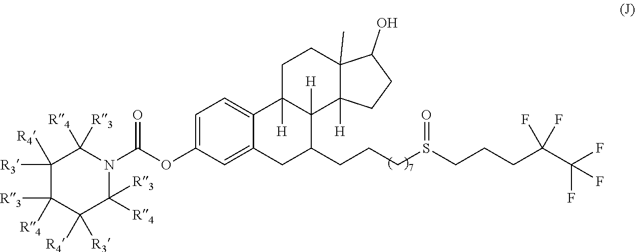

wherein,
R″₃, R″₄, R₃′, R₄′ is each independently selected from hydrogen, alkyl, haloalkyl, hydroxy, amino, alkylamino, dialkylamino, heterocycloalkyl, substituted heterocycloalkyl, and halo group, wherein the substituted heterocycloalkyl is substituted by one or more substitution is selected from alkyl, haloalkyl, hydroxy, amino, alkylamino, dialkylamino, halo, and oxo group; or
each R″₃ and R₃′ or R″₄, and R₄′ or R″₃ and R″₄, or R₃′ and R₄′ are taken together along with the atom to which they are attached to form a three to seven membered saturated, partially unsaturated or unsaturated ring which may contain one or more heteroatoms selected from N, O and S, and the ring may optionally be substituted at a substitutable position with one or more R′₅ radicals, wherein the one or more R′₅ radicals are independently selected from hydrogen, alkyl, haloalkyl, hydroxy, amino, alkylamino, dialkylamino, halo, and oxo group; or
each R″₃ and R₃′ or R″₄, and R₄′ are taken together along with the atom to which they are attached to form double bond; or each R″₃ and R″₄, or R₃′ and R₄′ are taken together form oxo( [symbol] ) bond;
enantiomers, diastereomers, racemates, pharmaceutically acceptable salts or solvates thereof.

In another embodiment, the present invention relates to the compound selected from group consisting of,

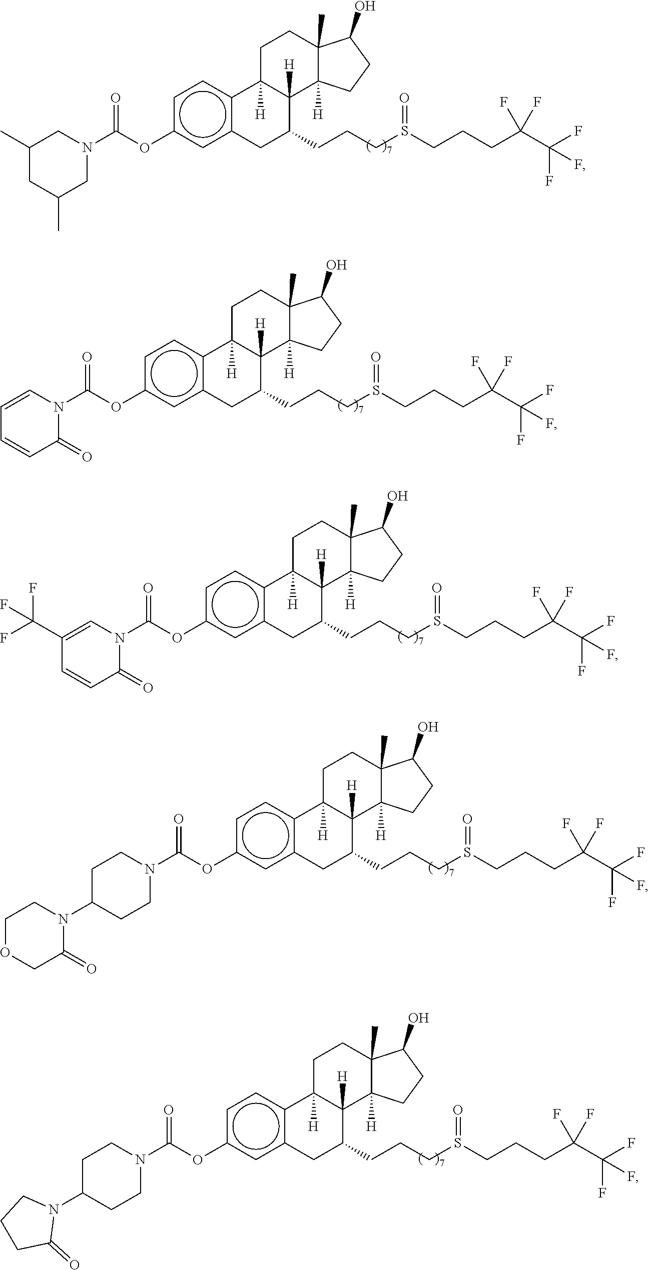

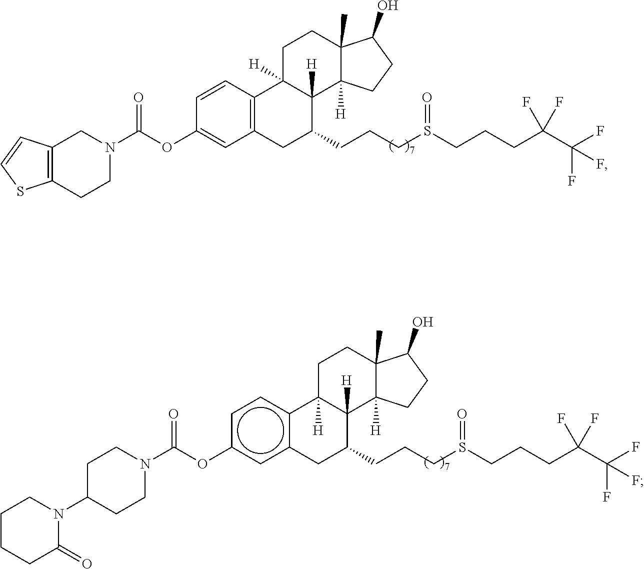

enantiomers, diastereomers, racemates, pharmaceutically acceptable salts or solvates thereof.

In another embodiment, the present invention relates to compound of formula (K):

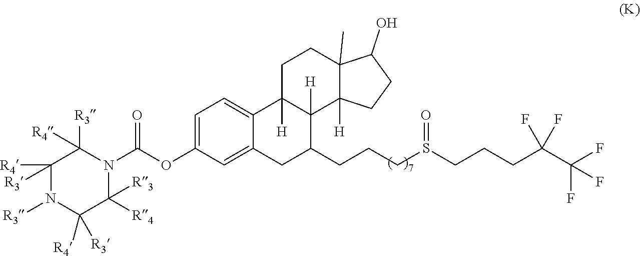

wherein,
R″₃, R″₄, R₃′, R₄′ is each independently selected from hydrogen, alkyl, haloalkyl, hydroxy, amino, alkylamino, dialkylamino, heterocycloalkyl, substituted heterocycloalkyl, and halo group, wherein the substituted heterocycloalkyl is substituted by one or more substitution is selected from alkyl, haloalkyl, hydroxy, amino, alkylamino, dialkylamino, halo, and oxo group; or
each R″₃ and R₃′ or R″₄, and R₄′ or R″₃ and R″₄, or R₃′ and R₄′ are taken together along with the atom to which they are attached to form a three to seven membered saturated, partially unsaturated or unsaturated ring which may contain one or more heteroatoms selected from N, O and S, and the ring may optionally be substituted at a substitutable position with one or more R′₅ radicals, wherein the one or more R′₅ radicals are independently selected from hydrogen, alkyl, haloalkyl, hydroxy, amino, alkylamino, dialkylamino, halo, and oxo group; or
each R″₃ and R₃′ or R″₄, and R₄′ are taken together along with the atom to which they are attached to form double bond; or
each R″₃ and R″₄, or R₃′ and R₄′ are taken together form oxo( [symbol] ) bond;
enantiomers, diastereomers, racemates, pharmaceutically acceptable salts or solvates thereof.

In another embodiment, the present invention relates to the compound selected from group consisting,

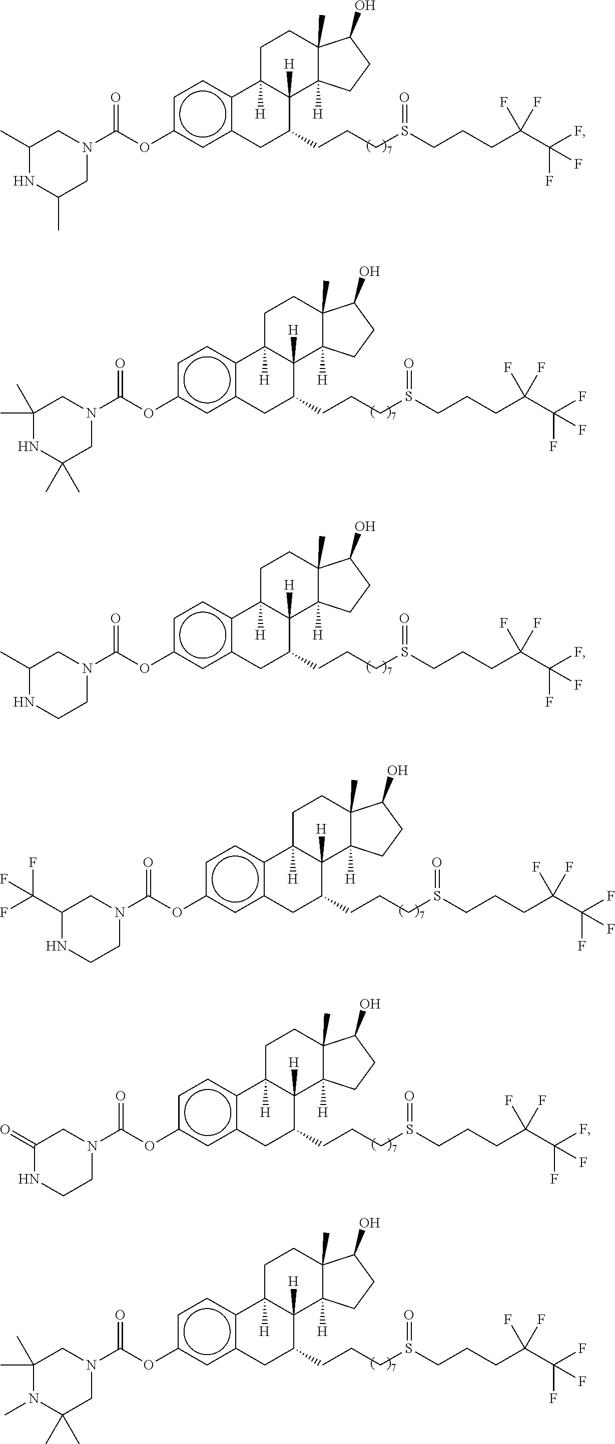

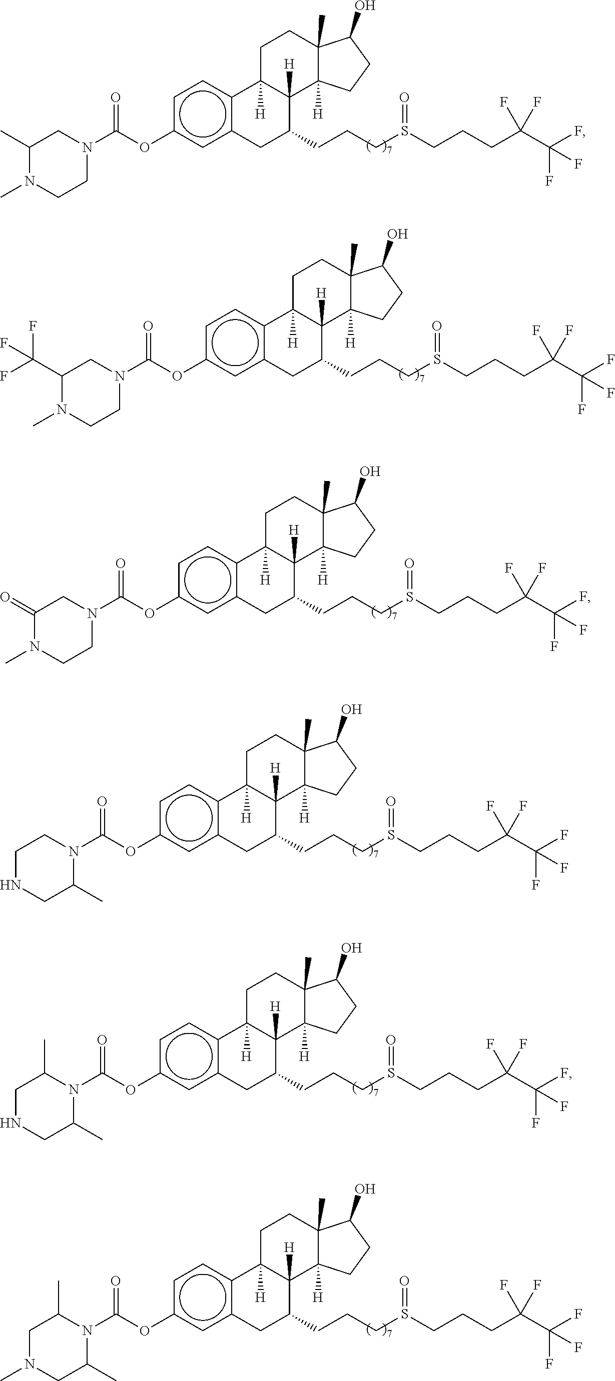

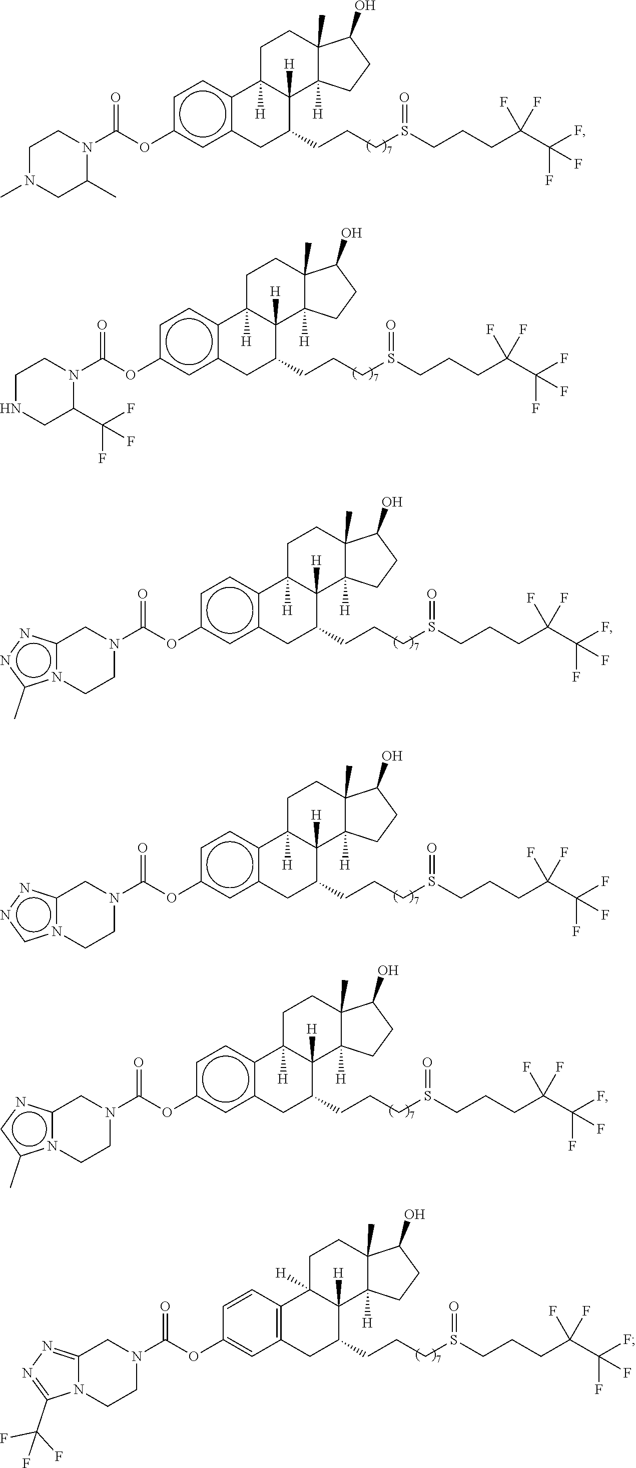

enantiomers, diastereomers, racemates, pharmaceutically acceptable salts or solvates thereof.

In another embodiment, the present invention relates to compound of formula (L):

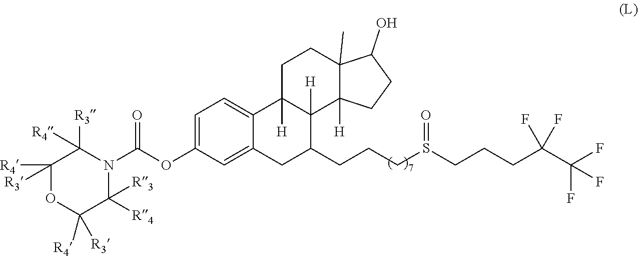

wherein,
R″₃, R″₄, R₃′, R₄′ is each independently selected from hydrogen, alkyl, haloalkyl, hydroxy, amino, alkylamino, dialkylamino, heterocycloalkyl, substituted heterocycloalkyl, and halo group, wherein the substituted heterocycloalkyl is substituted by one or more substitution is selected from alkyl, haloalkyl, hydroxy, amino, alkylamino, dialkylamino, halo, and oxo group; or
each R″₃ and R₃′ or R″₄, and R₄′ or R″₃ and R″₄, or R₃′ and R₄′ are taken together along with the atom to which they are attached to form a three to seven membered saturated, partially unsaturated or unsaturated ring which may contain one or more heteroatoms selected from N, O and S, and the ring may optionally be substituted at a substitutable position with one or more R′₅ radicals, wherein the one or more R′₅ radicals are independently selected from hydrogen, alkyl, haloalkyl, hydroxy, amino, alkylamino, dialkylamino, halo, and oxo group; or
each R″₃ and R₃′ or R″₄, and R₄′ are taken together along with the atom to which they are attached to form double bond; or
each R″₃ and R″₄, or R₃′ and R₄′ are taken together form oxo( [symbol] ) bond;
enantiomers, diastereomers, racemates, pharmaceutically acceptable salts or solvates thereof.

In another embodiment, the present invention relates to the compound selected from group consisting of:

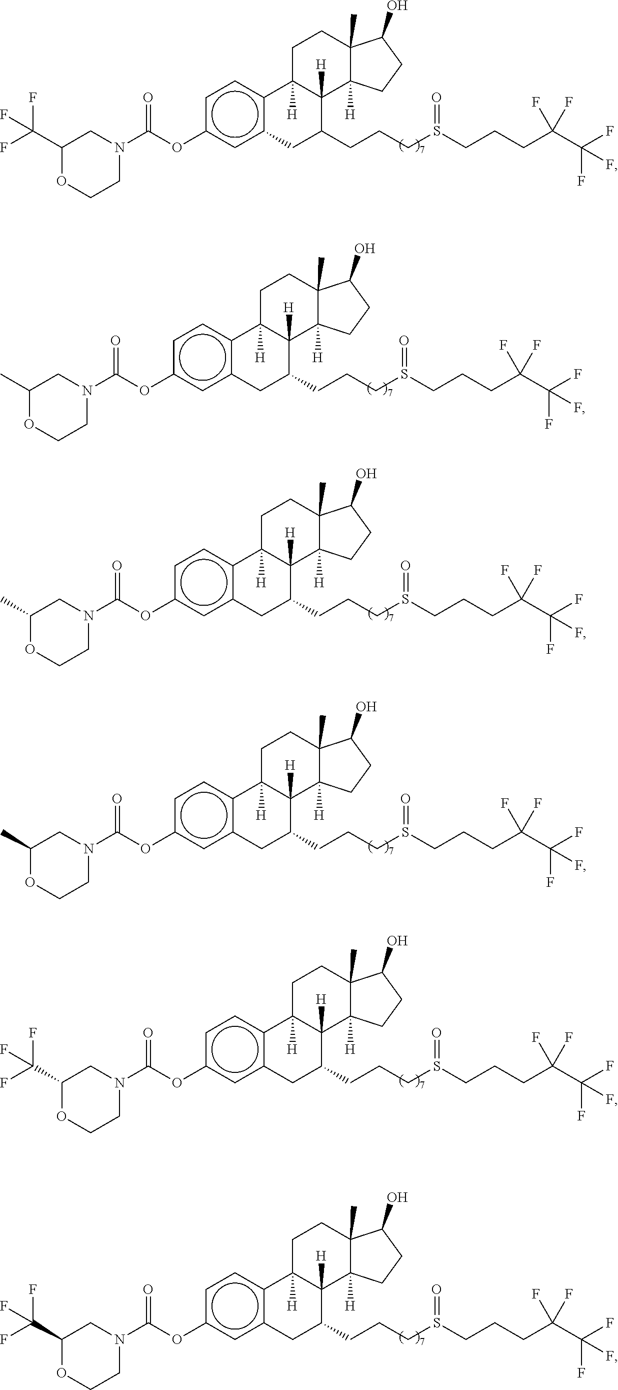

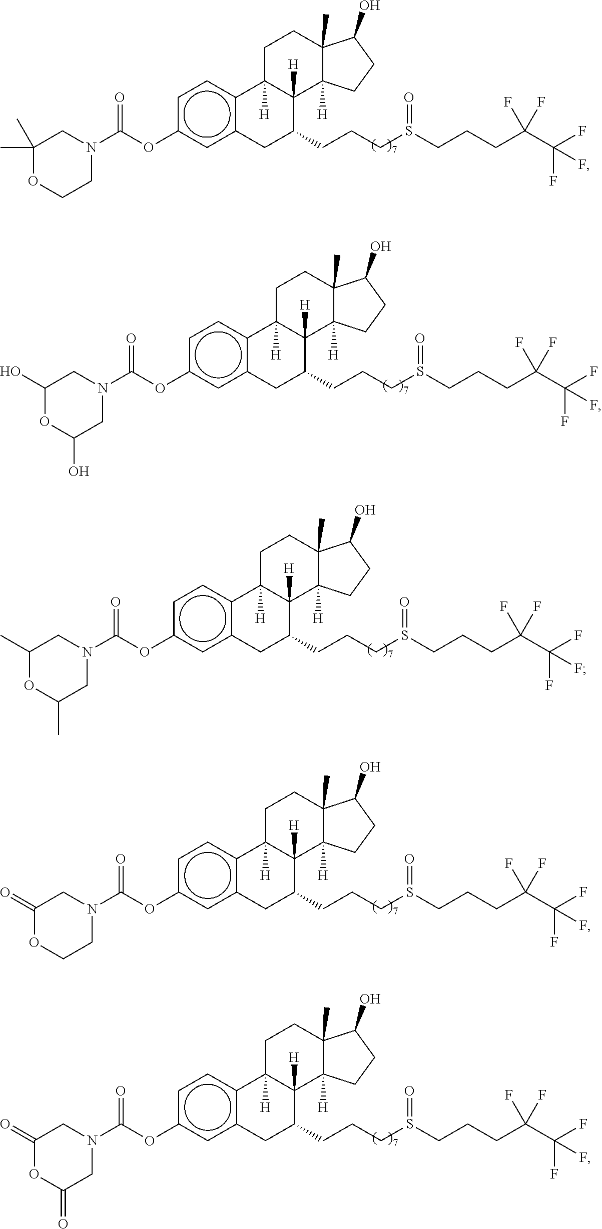

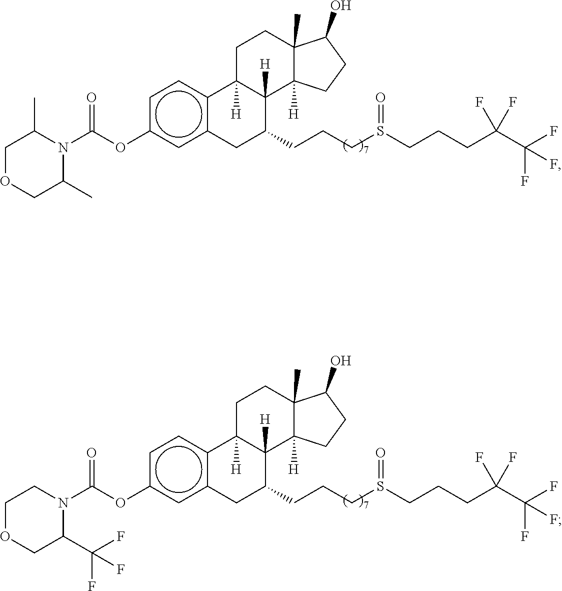

enantiomers, diastereomers, racemates, pharmaceutically acceptable salts or solvates thereof.

In another embodiment, the present invention relates to compound of formula (M):

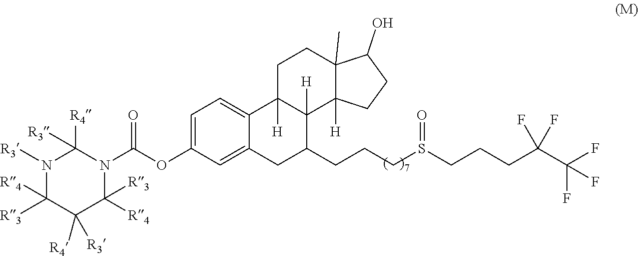

wherein,
R″₃, R″₄, R₃′, R₄′ is each independently selected from hydrogen, alkyl, haloalkyl, hydroxy, amino, alkylamino, dialkylamino, heterocycloalkyl, substituted heterocycloalkyl, and halo group, wherein the substituted heterocycloalkyl is substituted by one or more substitution is selected from alkyl, haloalkyl, hydroxy, amino, alkylamino, dialkylamino, halo, and oxo group; or
each R″₃ and R₃′ or R″₄, and R₄′ or R″₃ and R″₄, or R₃′ and R₄′ are taken together along with the atom to which they are attached to form a three to seven membered saturated, partially unsaturated or unsaturated ring which may contain one or more heteroatoms selected from N, O and S, and the ring may optionally be substituted at a substitutable position with one or more R′₅ radicals, wherein the one or more R′₅ radicals are independently selected from hydrogen, alkyl, haloalkyl, hydroxy, amino, alkylamino, dialkylamino, halo, and oxo group; or
each R″₃ and R₃′ or R″₄, and R₄′ are taken together along with the atom to which they are attached to form double bond; or
each R″₃ and R″₄, or R₃′ and R₄′ are taken together form oxo( [symbol] ) bond;
enantiomers, diastereomers, racemates, pharmaceutically acceptable salts or solvates thereof.

In another embodiment, the present invention relates to the compound selected from group consisting of,

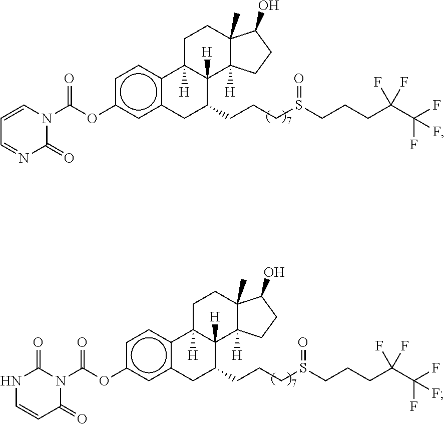

enantiomers, diastereomers, racemates, pharmaceutically acceptable salts or solvates thereof.

In another embodiment, the present invention relates to compound of formula (N):

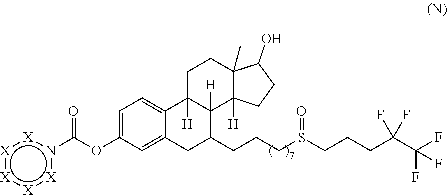

[symbol] represents the aromatic ring;
X is each independently selected from N, O, S,

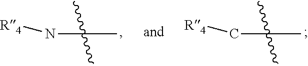

R″₄ is each independently selected from hydrogen, alkyl, haloalkyl, hydroxy, amino, alkylamino, dialkylamino, heterocycloalkyl, substituted heterocycloalkyl, and halo group, wherein the substituted heterocycloalkyl is substituted by one or more substitution is selected from alkyl, haloalkyl, hydroxy, amino, alkylamino, dialkylamino, halo, and oxo group;
enantiomers, diastereomers, racemates, pharmaceutically acceptable salts or solvates thereof.

In another embodiment, the present invention relates to compound of formula (O):

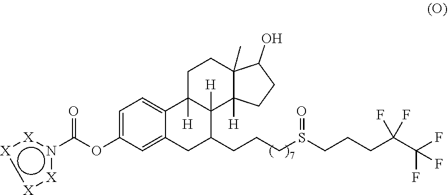

[symbol] represents the aromatic ring;
X is each independently selected from N, O, S,

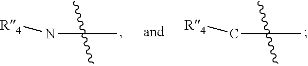

R″₄ is each independently selected from hydrogen, alkyl, haloalkyl, hydroxy, amino, alkylamino, dialkylamino, heterocycloalkyl, substituted heterocycloalkyl, and halo group, wherein the substituted heterocycloalkyl is substituted by one or more substitution is selected from alkyl, haloalkyl, hydroxy, amino, alkylamino, dialkylamino, halo, and oxo group; enantiomers, diastereomers, racemates, pharmaceutically acceptable salts or solvates thereof.

In another embodiment, the present invention relates to the compound selected from group consisting of,

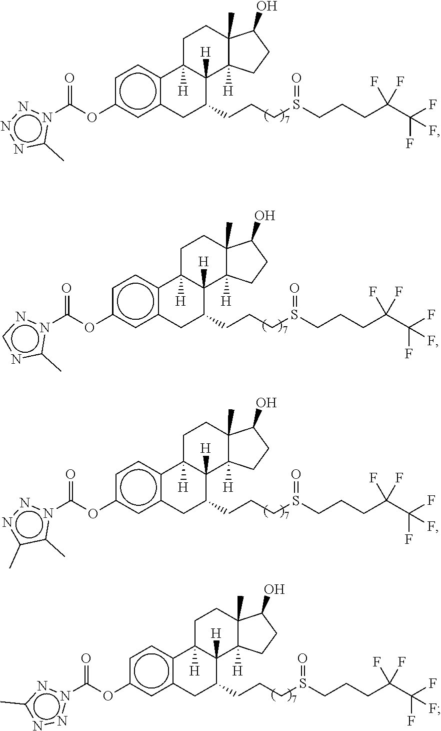

enantiomers, diastereomers, racemates, pharmaceutically acceptable salts or solvates thereof.

In another embodiment, the present invention relates to compound of formula (P):

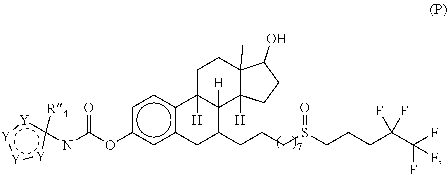

[symbol] represents the saturation, partial unsaturation or the complete unsaturation in the ring;
Y is selected from C,

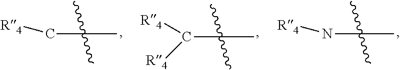

N, O, and S;
R″₄ is each independently selected from hydrogen, alkyl, haloalkyl, hydroxy, amino, alkylamino, dialkylamino, heterocycloalkyl, substituted heterocycloalkyl, and halo group, wherein the substituted heterocycloalkyl is substituted by one or more substitution is selected from alkyl, haloalkyl, hydroxy, amino, alkylamino, dialkylamino, halo, and oxo group;
enantiomers, diastereomers, racemates, pharmaceutically acceptable salts or solvates thereof.

In another embodiment, the present invention relates to the compound,

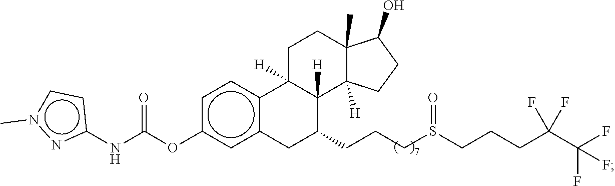

enantiomers, diastereomers, racemates, pharmaceutically acceptable salts or solvates thereof.

In another embodiment, the present invention relates to compound of formula Q:

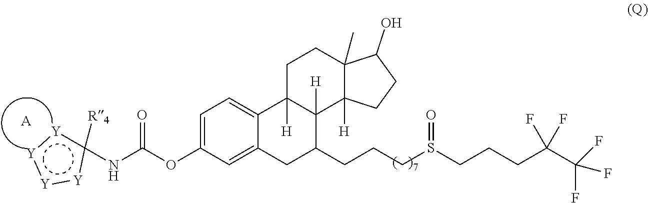

wherein;
[symbol] represents the saturation, partial unsaturation or the complete unsaturation in the ring;
Y is selected from C,

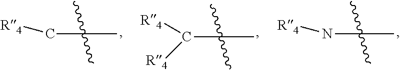

N, O, and S;
fused ring A represents an optionally substituted saturated, unsaturated, or aromatic four, five, six, or seven member ring having zero or more heteroatoms in the ring, the remaining atoms of the ring being carbon with each heteroatom being independently selected from nitrogen, oxygen and sulfur, wherein ring A is optionally substituted with one or more R″₆ radicals independently selected from hydrogen, alkyl, haloalkyl, hydroxy, amino, alkylamino, dialkylamino, unsubstituted heterocycloalkyl, substituted heterocycloalkyl, halo, and oxo group,
wherein the one or more substitution on substituted heterocycloalkyl is selected from alkyl, haloalkyl, hydroxy, amino, alkylamino, dialkylamino, halo, and oxo group;
R″₄, is each independently selected from hydrogen, alkyl, haloalkyl, hydroxy, amino, alkylamino, dialkylamino, heterocycloalkyl, substituted heterocycloalkyl, and halo group, wherein the substituted heterocycloalkyl is substituted by one or more substitution is selected from alkyl, haloalkyl, hydroxy, amino, alkylamino, dialkylamino, halo, and oxo group;
enantiomers, diastereomers, racemates, pharmaceutically acceptable salts or solvates thereof.

In another embodiment, the present invention relates to the compound selected from the group consisting of,

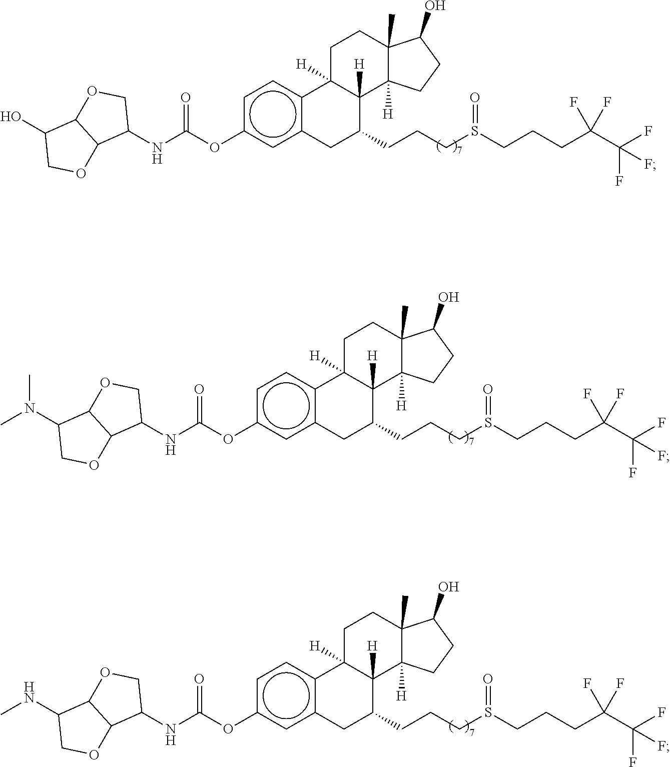

enantiomers, diastereomers, racemates, pharmaceutically acceptable salts or solvates thereof.

In another embodiment, the present invention relates to compound of formula (R):

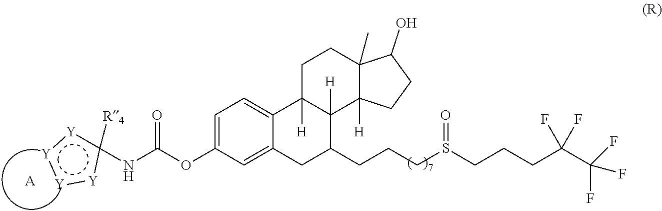

[symbol]

represents the saturation, partial unsaturation or the complete unsaturation in the ring;
Y is selected from C,

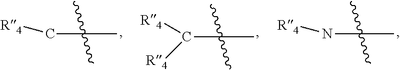

N, O, and S;
fused ring A represents an optionally substituted saturated, unsaturated, or aromatic four, five, six, or seven member ring having zero or more heteroatoms in the ring, the remaining atoms of the ring being carbon with each heteroatom being independently selected from nitrogen, oxygen and sulfur, wherein ring A is optionally substituted with one or more R″₆ radicals independently selected from hydrogen, alkyl, haloalkyl, hydroxy, amino, alkylamino, dialkylamino, unsubstituted heterocycloalkyl, substituted heterocycloalkyl, halo, and oxo group, wherein the one or more substitution on substituted heterocycloalkyl is selected from alkyl, haloalkyl, hydroxy, amino, alkylamino, dialkylamino, halo, and oxo group;
R″₄, is each independently selected from hydrogen, alkyl, haloalkyl, hydroxy, amino, alkylamino, dialkylamino, heterocycloalkyl, substituted heterocycloalkyl, and halo group, wherein the substituted heterocycloalkyl is substituted by one or more substitution is selected from alkyl, haloalkyl, hydroxy, amino, alkylamino, dialkylamino, halo, and oxo group;
enantiomers, diastereomers, racemates, pharmaceutically acceptable salts or solvates thereof.

In another embodiment, the present invention relates to compound of formula (S):

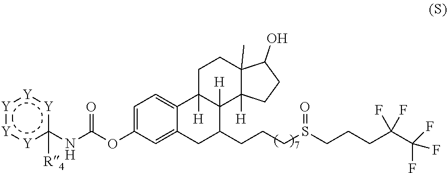

wherein
[symbol] represents the saturation, partial unsaturation or the complete unsaturation in the ring;
Y is selected from C,

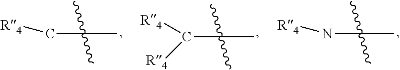

N, O, and S;
R″₄, is each independently selected from hydrogen, alkyl, haloalkyl, hydroxy, amino, alkylamino, dialkylamino, heterocycloalkyl, substituted heterocycloalkyl, and halo group, wherein the substituted heterocycloalkyl is substituted by one or more substitution is selected from alkyl, haloalkyl, hydroxy, amino, alkylamino, dialkylamino, halo, and oxo group;
enantiomers, diastereomers, racemates, pharmaceutically acceptable salts or solvates thereof.

In another embodiment, the present invention relates to the compound selected from group consisting of,

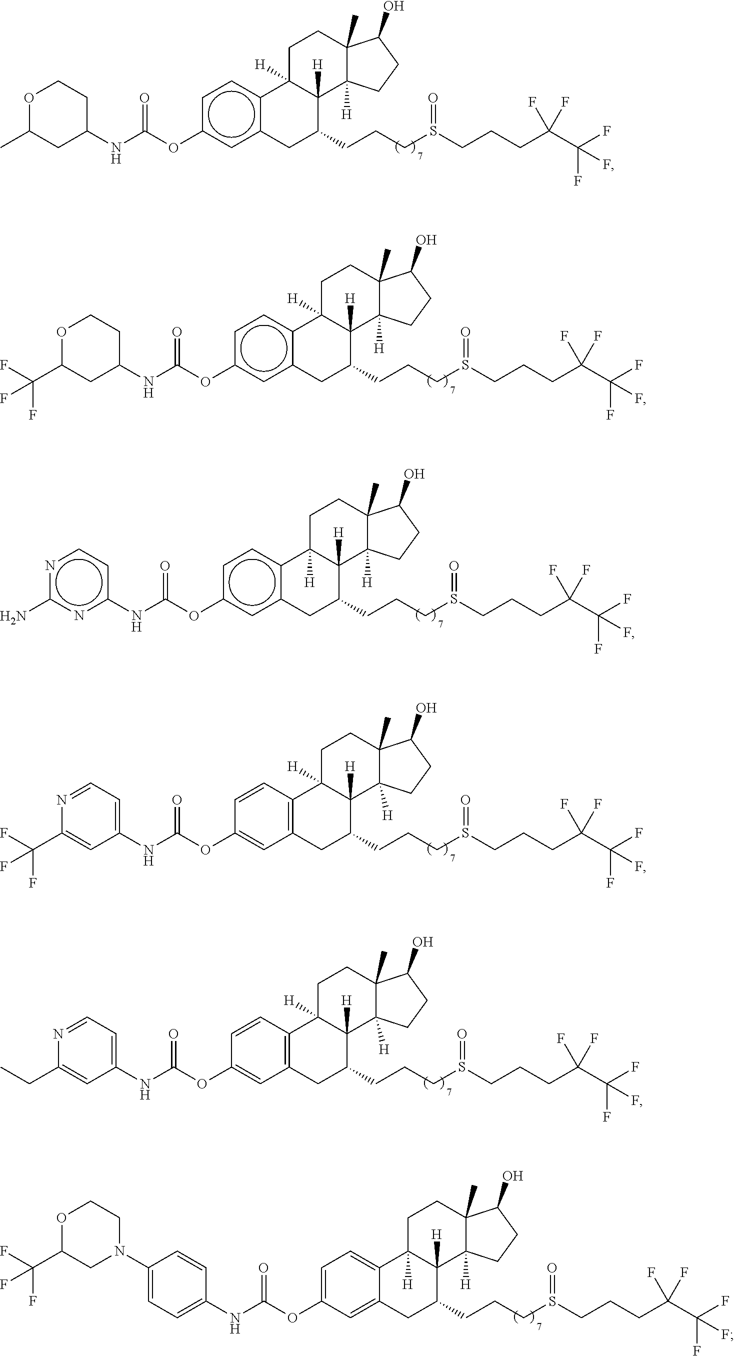

enantiomers, diastereomers, racemates, pharmaceutically acceptable salts or solvates thereof.

In another embodiment, the present invention relates to compound of formula T:

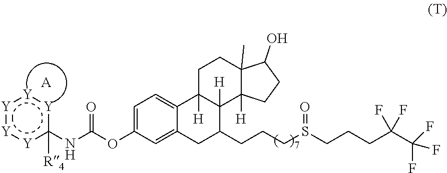

wherein
[symbol] represents the saturation, partial unsaturation or the complete unsaturation in the ring;
Y is selected from C,

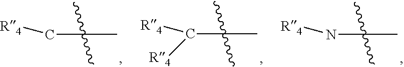

N, O, and S;
fused ring A represents an optionally substituted saturated, unsaturated, or aromatic four, five, six, or seven member ring having zero or more heteroatoms in the ring, the remaining atoms of the ring being carbon with each heteroatom being independently selected from nitrogen, oxygen and sulfur, wherein ring A is optionally substituted with one or more R″₆ radicals independently selected from hydrogen, alkyl, haloalkyl, hydroxy, amino, alkylamino, dialkylamino, unsubstituted heterocycloalkyl, substituted heterocycloalkyl, halo, and oxo group,
wherein the one or more substitution on substituted heterocycloalkyl is selected from alkyl, haloalkyl, hydroxy, amino, alkylamino, dialkylamino, halo, and oxo group;
R″₄, is each independently selected from hydrogen, alkyl, haloalkyl, hydroxy, amino, alkylamino, dialkylamino, heterocycloalkyl, substituted heterocycloalkyl, and halo group, wherein the substituted heterocycloalkyl is substituted by one or more substitution is selected from alkyl, haloalkyl, hydroxy, amino, alkylamino, dialkylamino, halo, and oxo group;
enantiomers, diastereomers, racemates, pharmaceutically acceptable salts or solvates thereof.

In another embodiment, the present invention relates to compound of formula U:

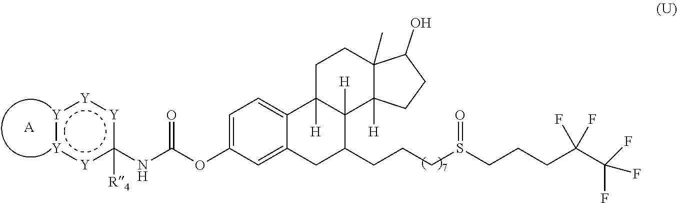

wherein;
[symbol] represents the saturation, partial unsaturation or the complete unsaturation in the ring;
Y is selected from C,

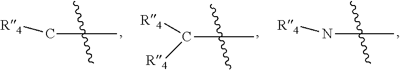

N, O, and S;
fused ring A represents an optionally substituted saturated, unsaturated, or aromatic four, five, six, or seven member ring having zero or more heteroatoms in the ring, the remaining atoms of the ring being carbon with each heteroatom being independently selected from nitrogen, oxygen and sulfur, wherein ring A is optionally substituted with one or more R″₆ radicals independently selected from hydrogen, alkyl, haloalkyl, hydroxy, amino, alkylamino, dialkylamino, unsubstituted heterocycloalkyl, substituted heterocycloalkyl, halo, and oxo group,
wherein the one or more substitution on substituted heterocycloalkyl is selected from alkyl, haloalkyl, hydroxy, amino, alkylamino, dialkylamino, halo, and oxo group;
R″₄, is each independently selected from hydrogen, alkyl, haloalkyl, hydroxy, amino, alkylamino, dialkylamino, heterocycloalkyl, substituted heterocycloalkyl, and halo group, wherein the substituted heterocycloalkyl is substituted by one or more substitution is selected from alkyl, haloalkyl, hydroxy, amino, alkylamino, dialkylamino, halo, and oxo group;
enantiomers, diastereomers, racemates, pharmaceutically acceptable salts or solvates thereof.

In one embodiment, the present invention relates to the compound selected from group consisting of,

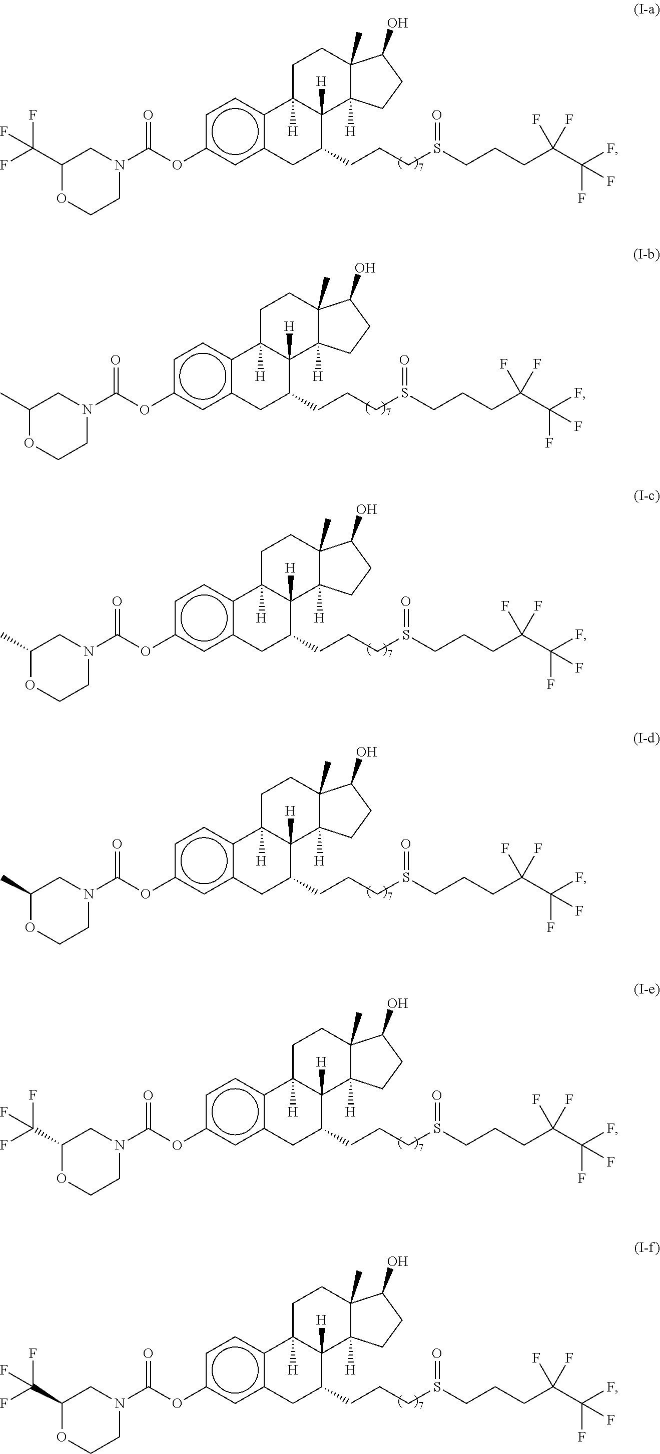

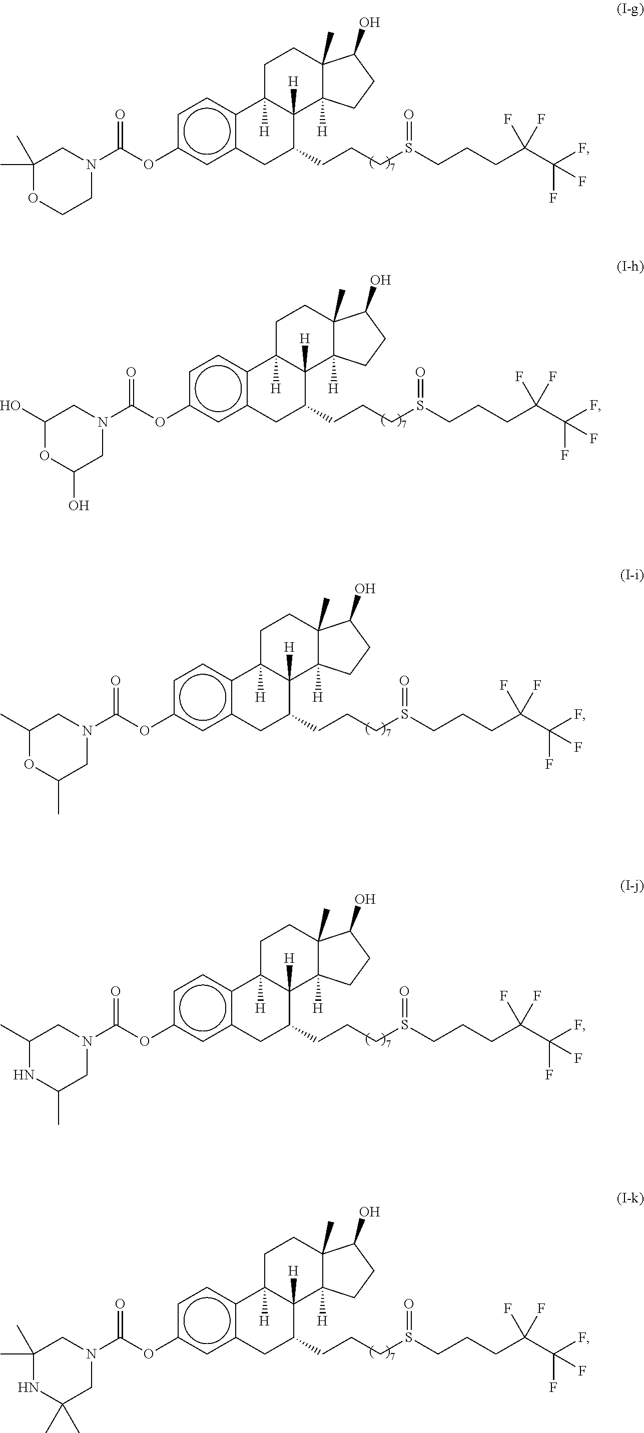

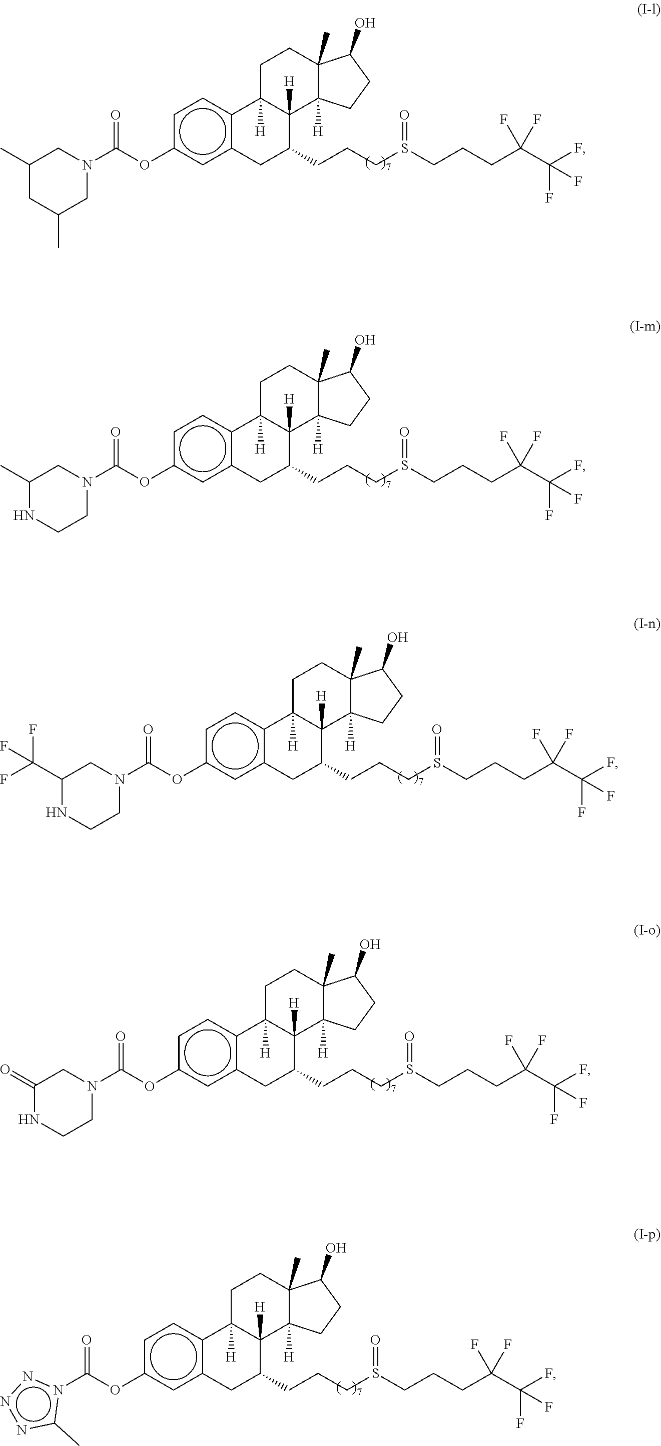

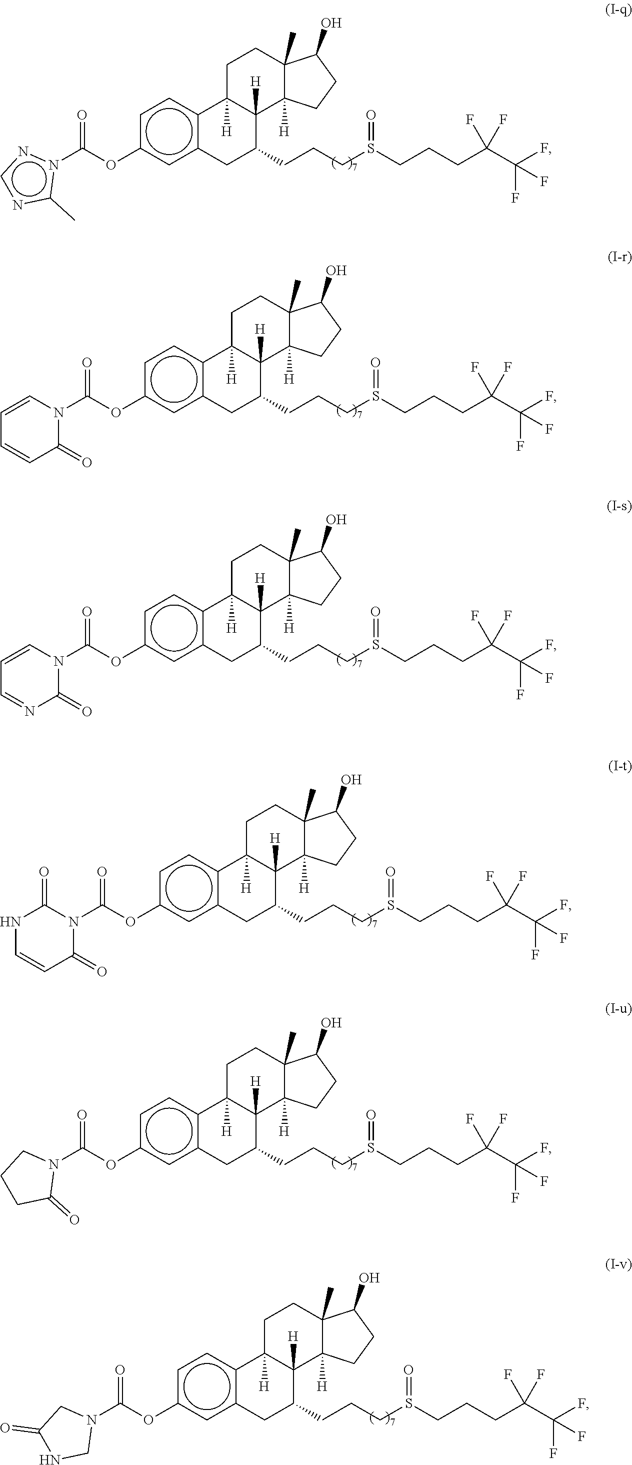

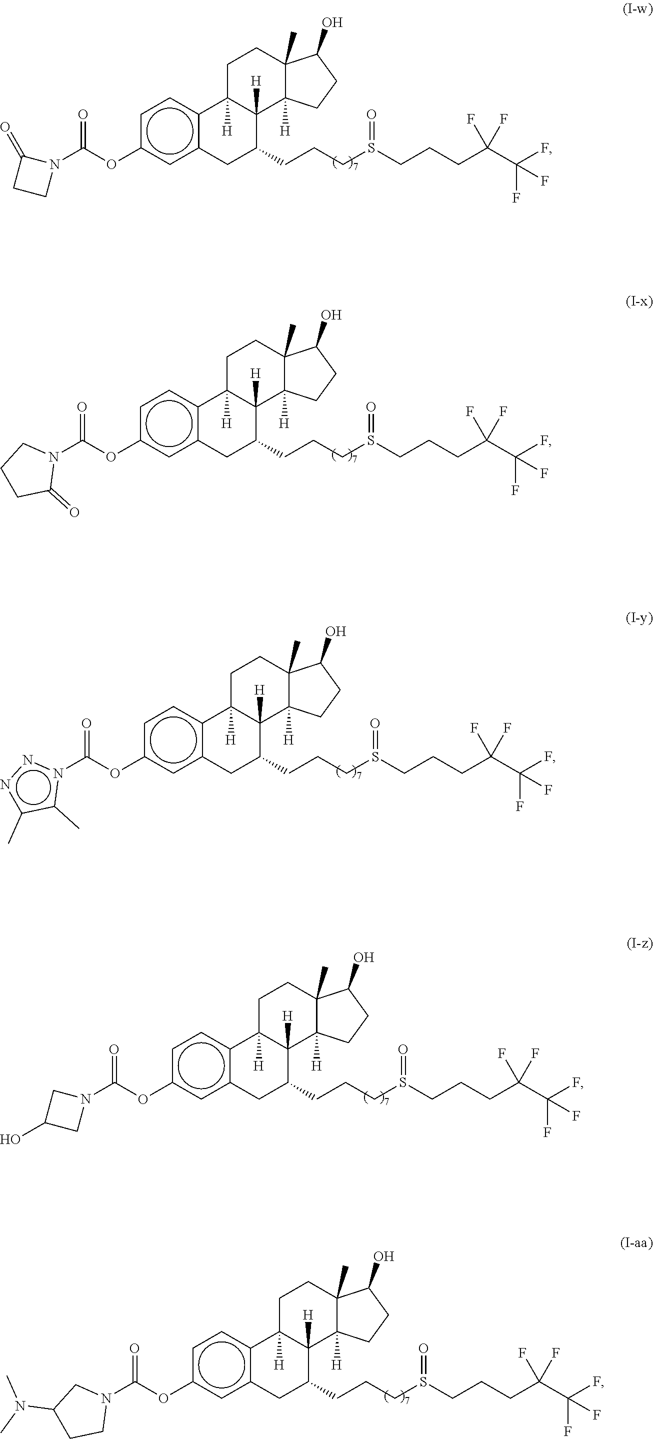

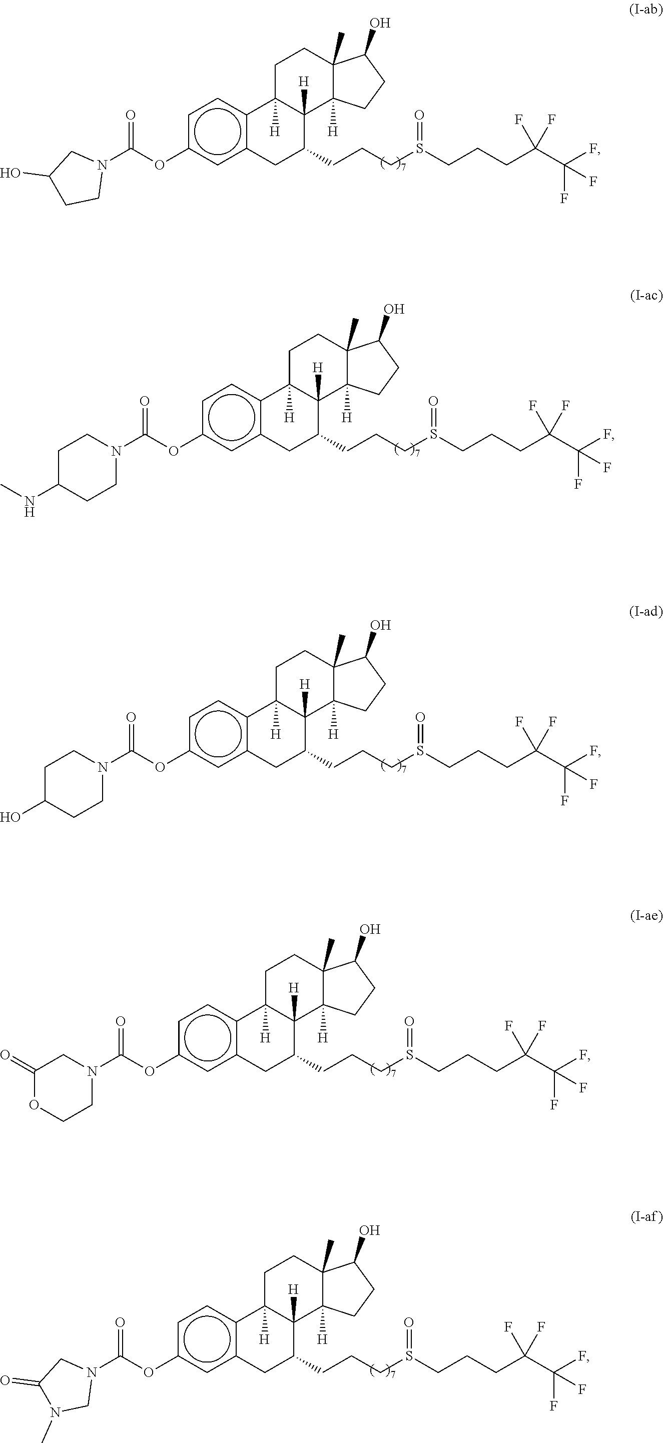

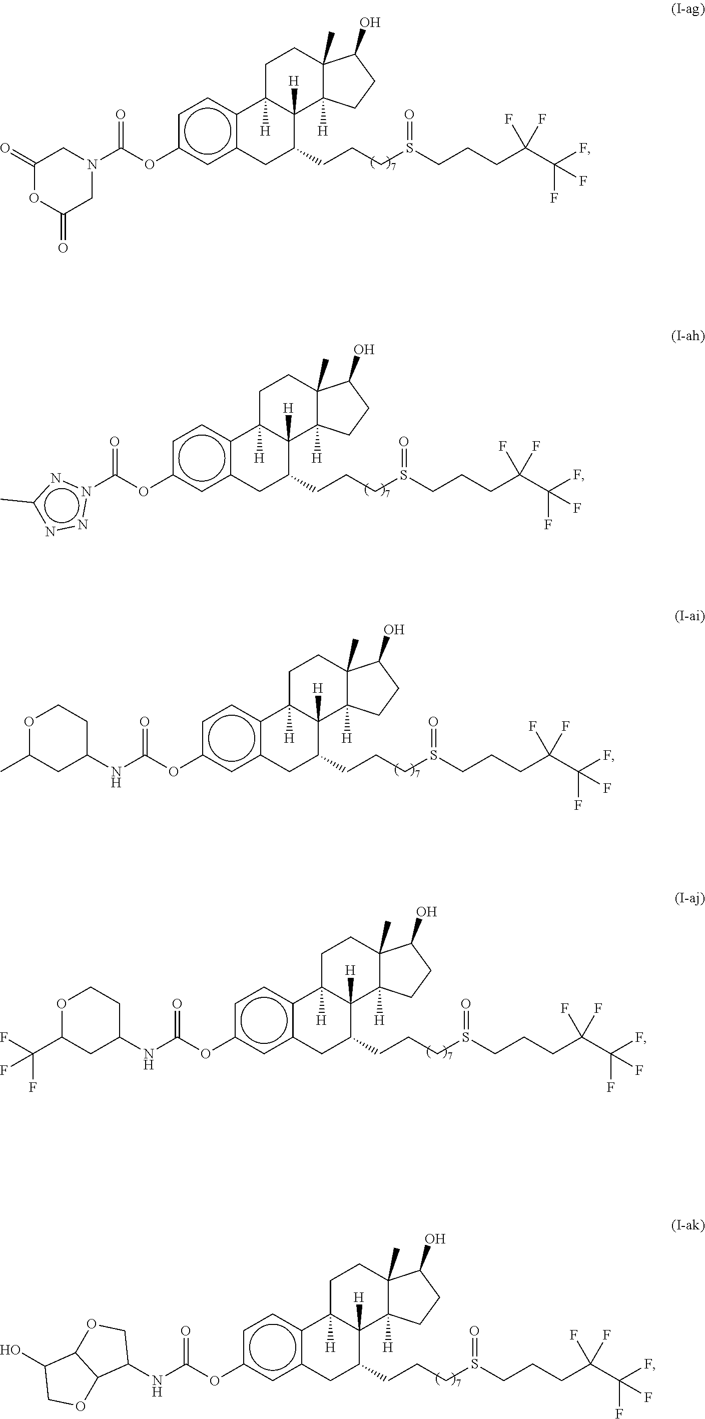

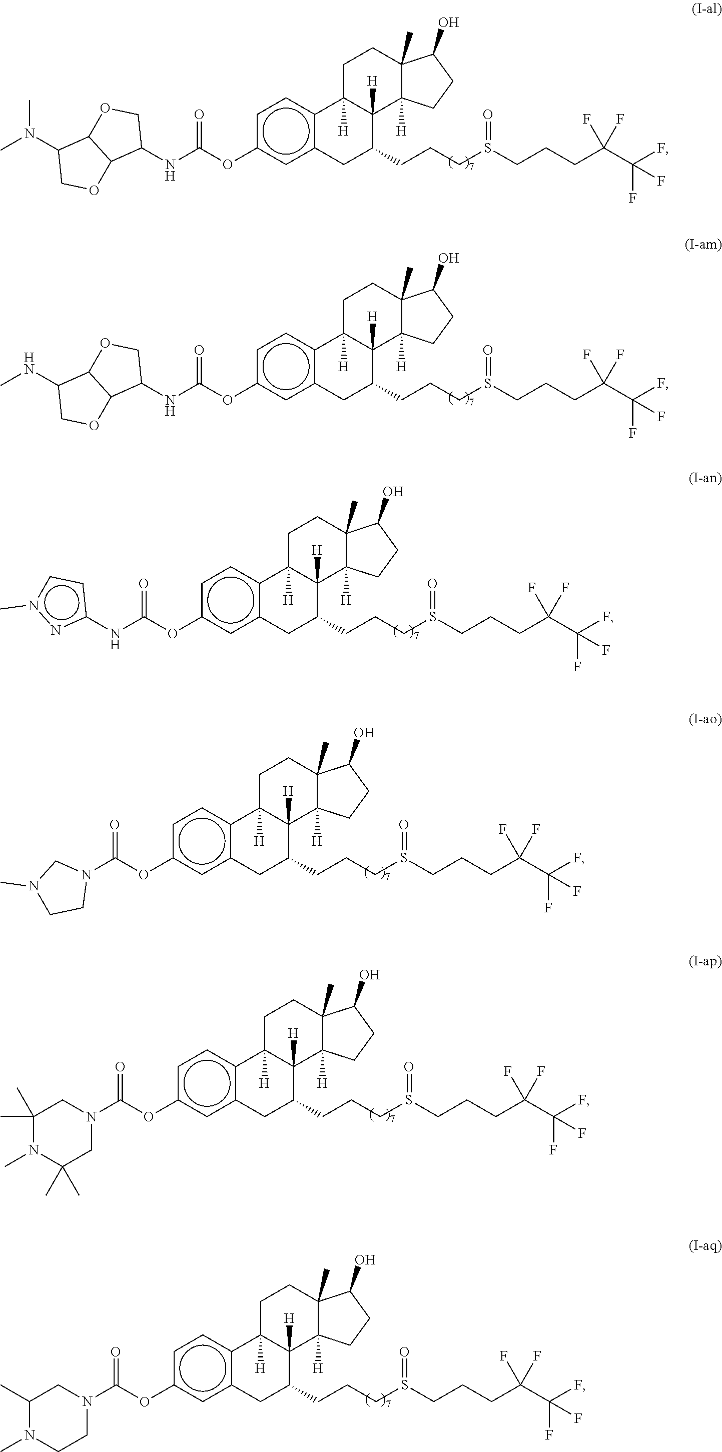

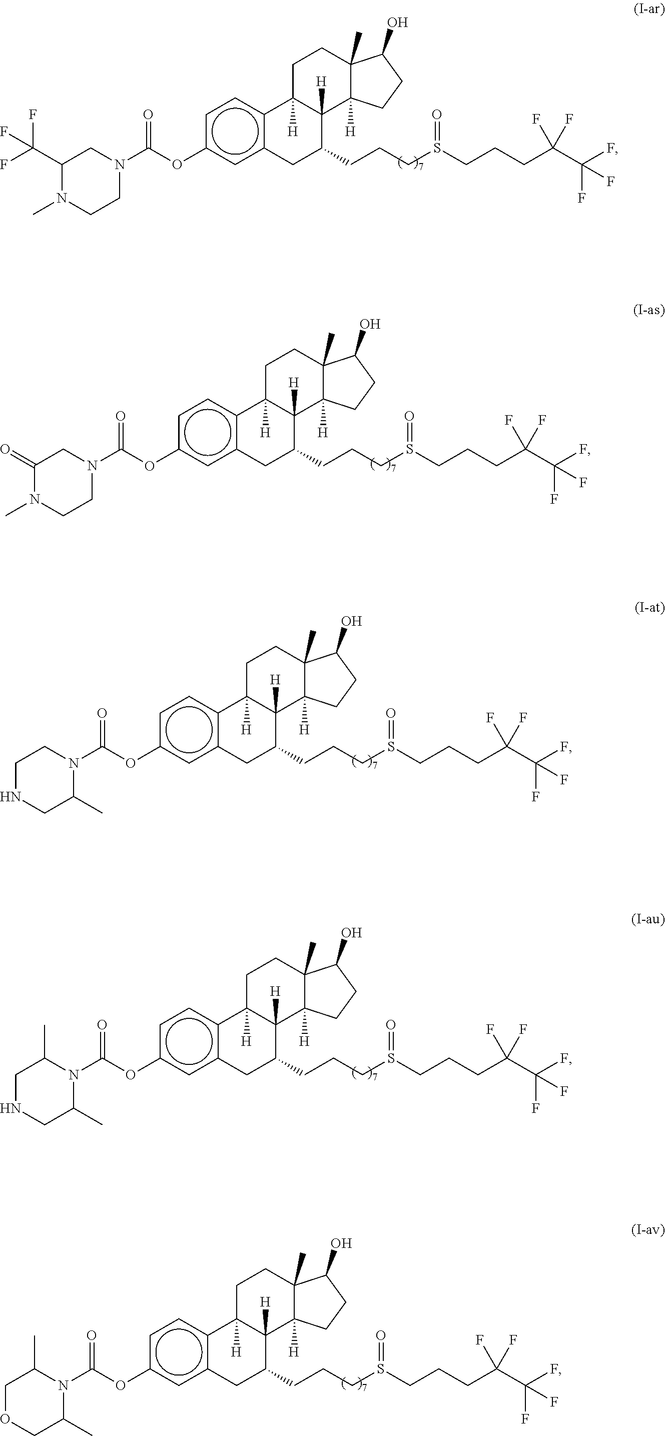

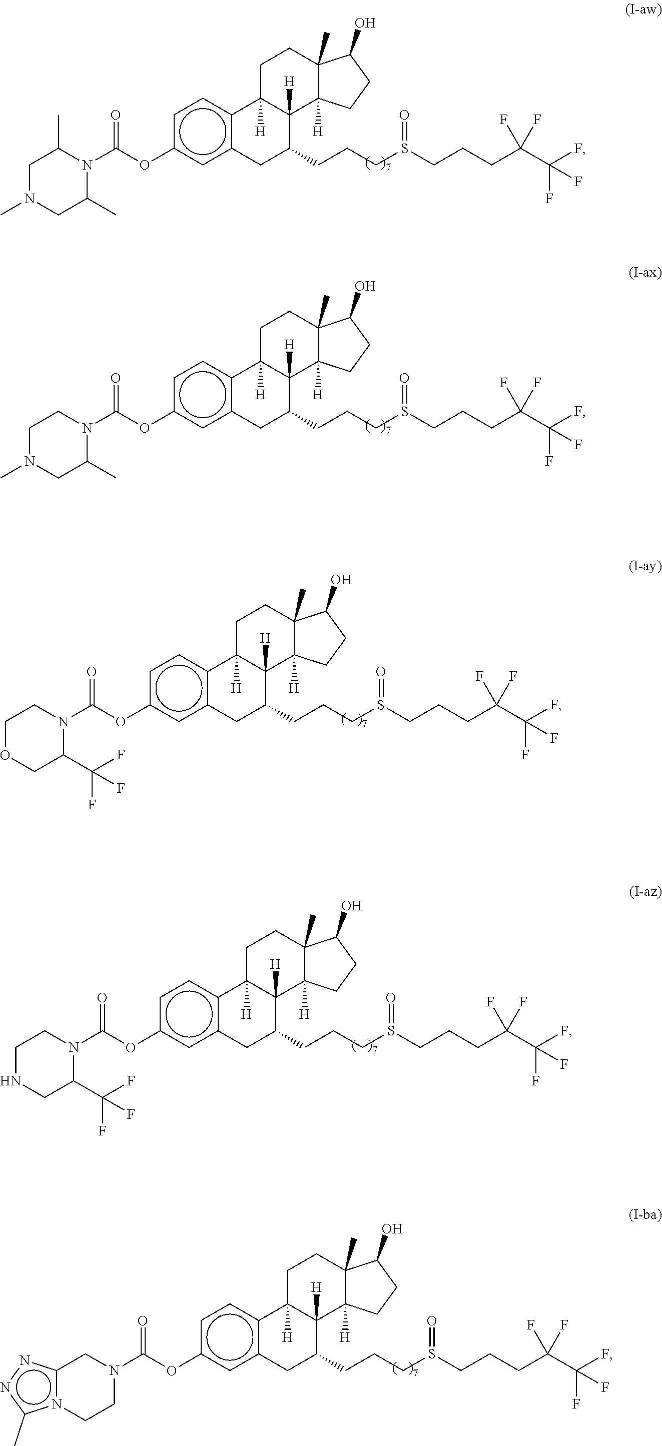

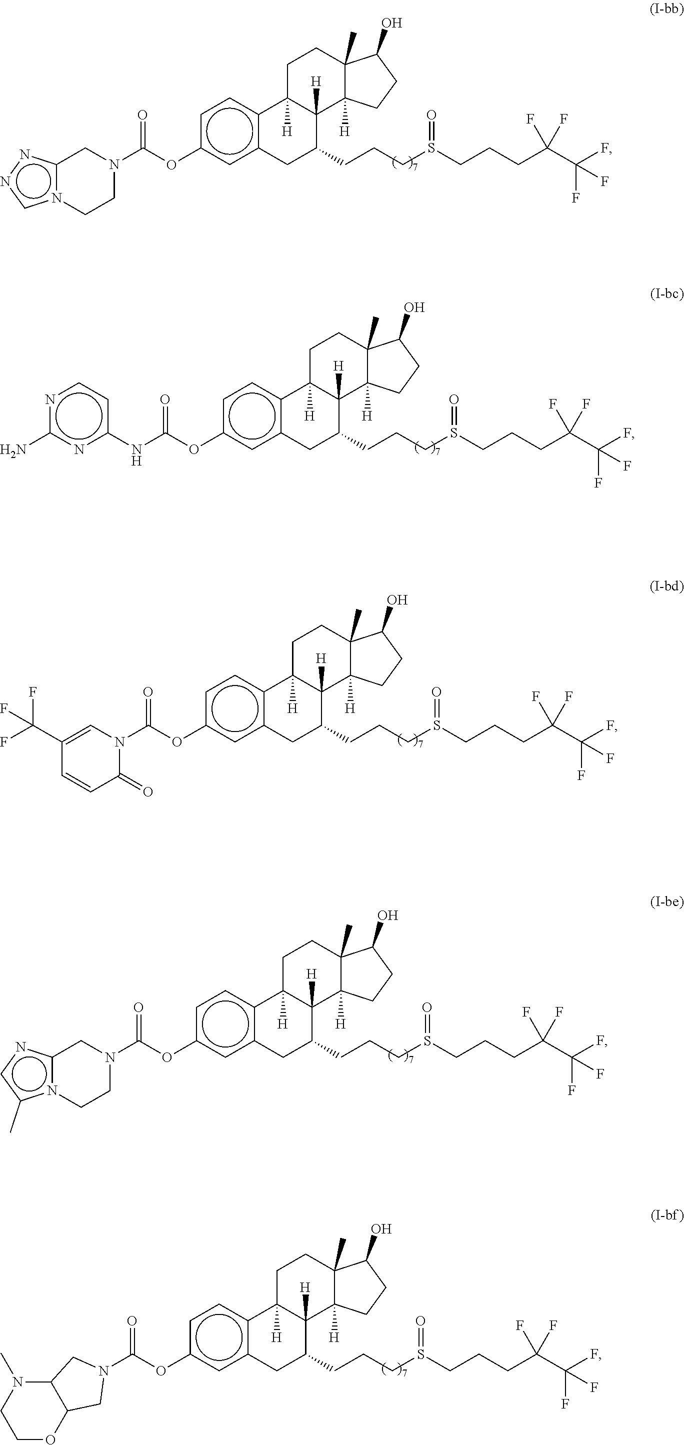

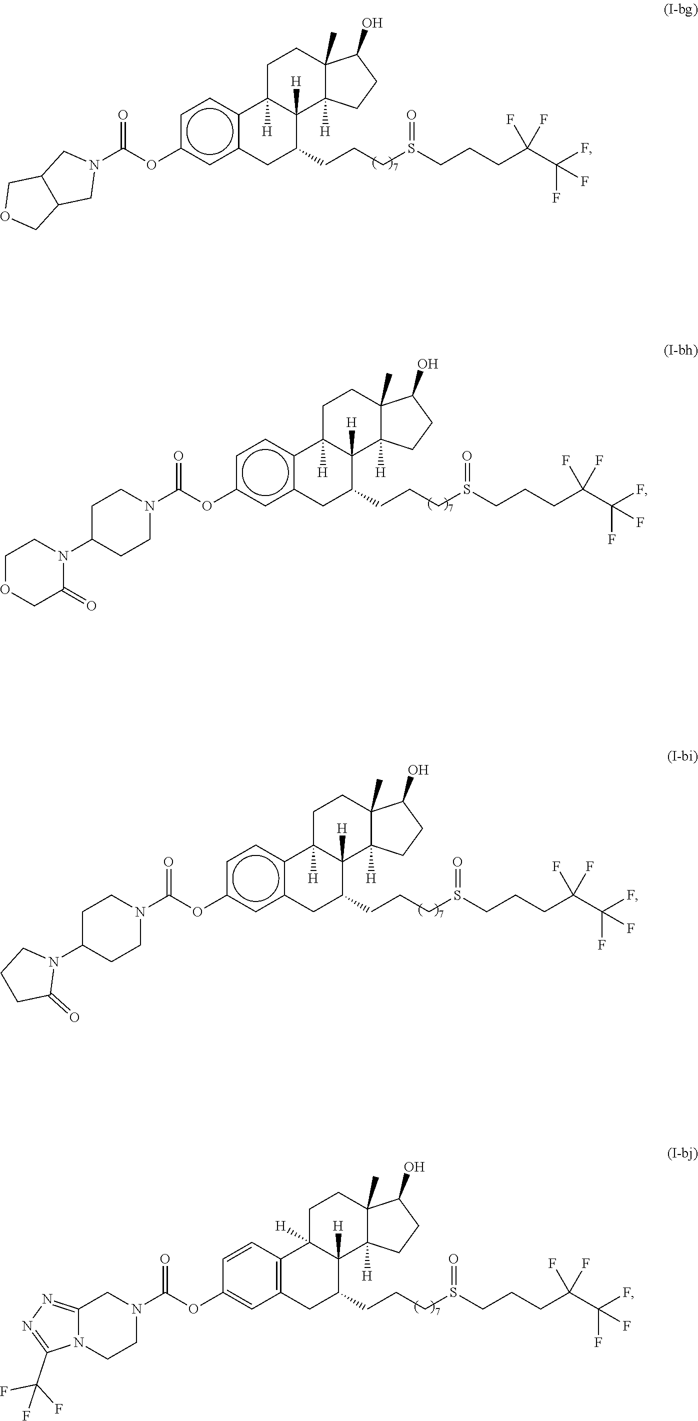

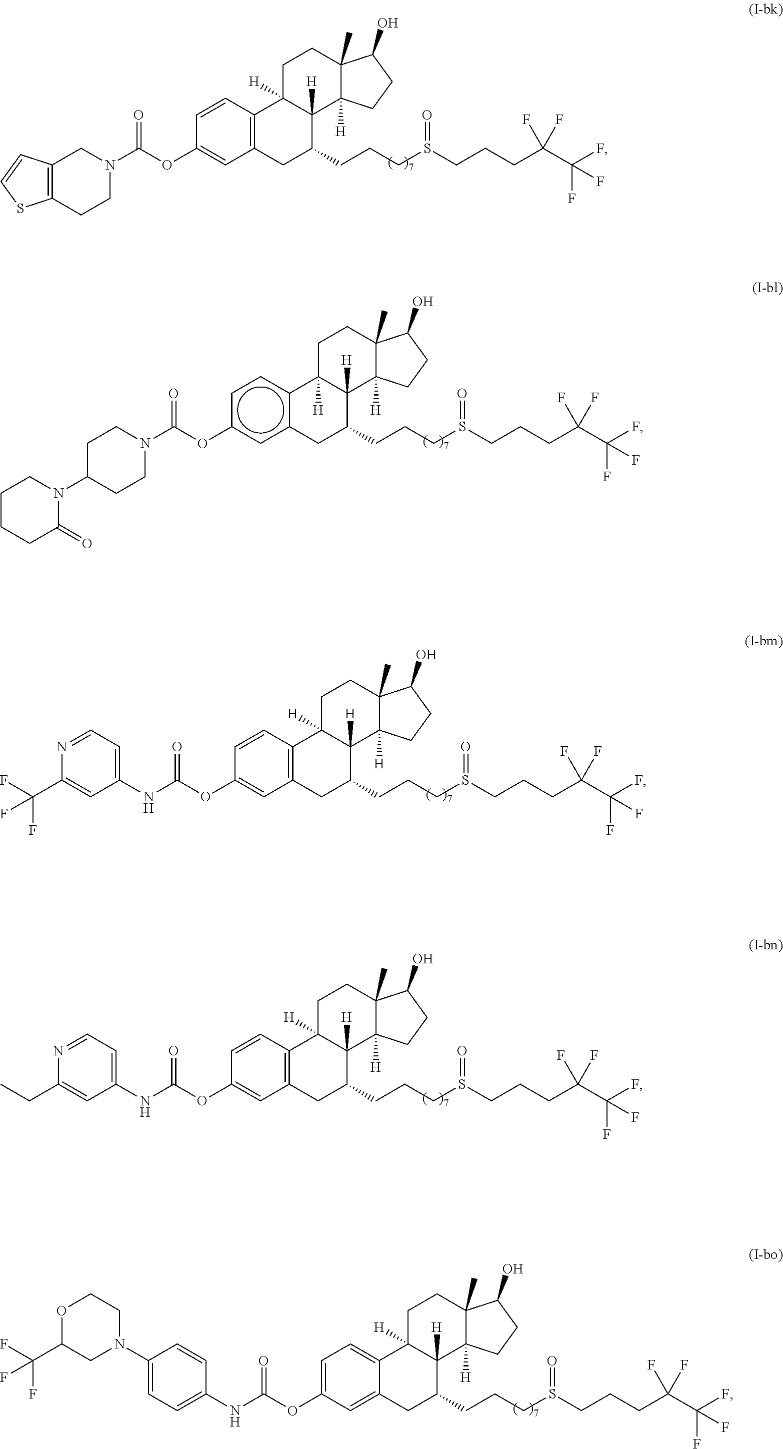

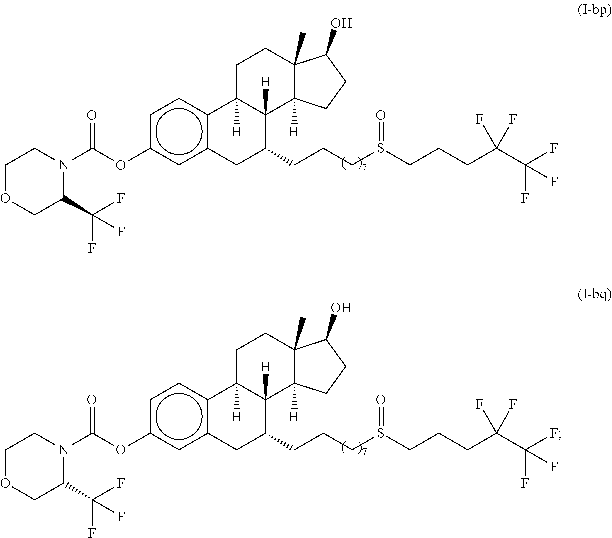

enantiomers, diastereomers, racemates, pharmaceutically acceptable salts or solvates thereof.

In an embodiment, a pharmaceutical composition comprising compound of formula (A); enantiomers, diastereomers, racemates, pharmaceutically acceptable salts and solvates thereof; and a pharmaceutically acceptable excipients.

In an embodiment, a pharmaceutical composition comprising compound of formula (B); enantiomers, diastereomers, racemates, pharmaceutically acceptable salts and solvates thereof; and a pharmaceutically acceptable excipients.

In an embodiment, a pharmaceutical composition comprising compound of formula (C); enantiomers, diastereomers, racemates, pharmaceutically acceptable salts and solvates thereof; and a pharmaceutically acceptable excipients.

In an embodiment, a method of treating a benign or malignant diseases of the breast or reproductive tract, preferably treating breast cancer, comprising compound of formula (A):

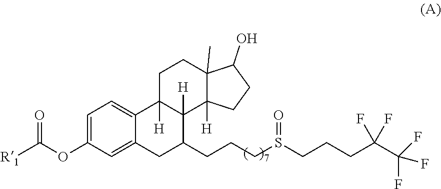

R′₁ is selected from optionally substituted heterocycloalkyl, heteroaryl, and

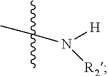

wherein one or more substitution on optionally substituted heterocycloalkyl, heteroaryl is selected from alkyl, haloalkyl, hydroxy, amino, alkylamino, dialkylamino, halo, and oxo group;
R′₂ is selected from group comprising optionally substituted heterocycloalkyl, heteroaryl and aryl group, wherein one or more substitution on heterocycloalkyl, heteroaryl and aryl is selected from alkyl, haloalkyl, hydroxy, amino, alkylamino, dialkylamino, unsubstituted heterocycloalkyl, substituted heterocycloalkyl, halo, and oxo group, wherein one or more substitution on substituted heterocycloalkyl is selected from alkyl, haloalkyl, hydroxy, amino, alkylamino, dialkylamino, halo, and oxo group;
enantiomers, diastereomers, racemates, pharmaceutically acceptable salts or solvates thereof.

In an embodiment, a method of treating a benign or malignant diseases of the breast or reproductive tract, preferably treating breast cancer, comprising compound of formula (B):

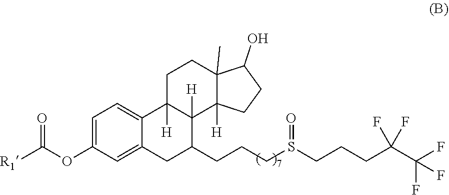

wherein;
R′₁ is selected from

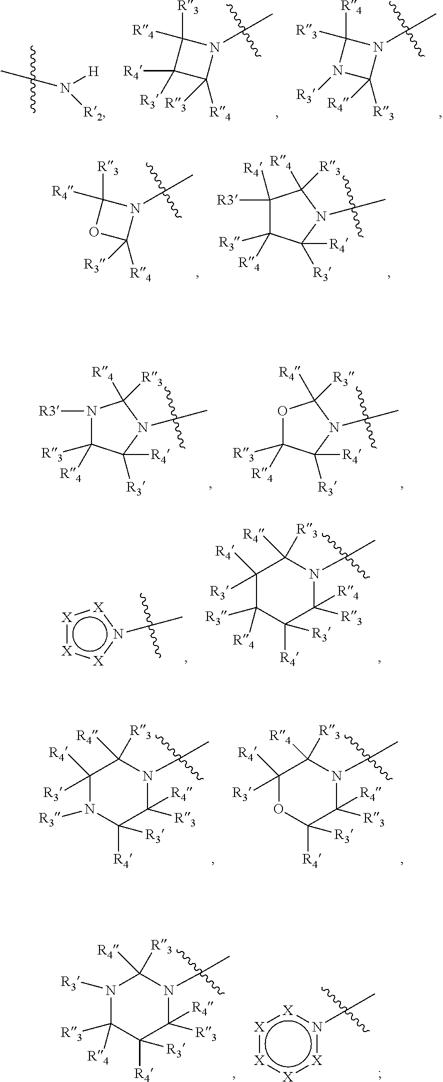

wherein R′₂ is selected from group comprising optionally substituted heterocycloalkyl, heteroaryl and aryl group,
wherein one or more substitution on heterocycloalkyl, heteroaryl and aryl is selected from alkyl, haloalkyl, hydroxy, amino, alkylamino, dialkylamino, unsubstituted heterocycloalkyl, substituted heterocycloalkyl, halo, and oxo group,
wherein one or more substitution on the substituted heterocycloalkyl is selected from alkyl, haloalkyl, hydroxy, amino, alkylamino, dialkylamino, halo, and oxo group;
[symbol] represents the aromatic ring;
X is each independently selected from N, O, S,

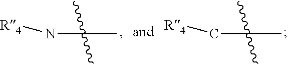

wherein R″₃, R″₄, R₃, R₄′ is each independently selected from hydrogen, alkyl, haloalkyl, hydroxy, amino, alkylamino, dialkylamino, heterocycloalkyl, substituted heterocycloalkyl, and halo group, wherein the substituted heterocycloalkyl is substituted by one or more substitution is selected from alkyl, haloalkyl, hydroxy, amino, alkylamino, dialkylamino, halo, and oxo group; or
each R″₃ and R₃′ or R″₄, and R₄′ or R″₃ and R″₄, or R₃′ and R₄′ are taken together along with the atom to which they are attached to form a three to seven membered saturated, partially unsaturated or unsaturated ring which may contain one or more heteroatoms selected from N, O and S, and the ring may optionally be substituted at a substitutable position with one or more R′₅ radicals, wherein the one or more R′₅ radicals are independently selected from hydrogen, alkyl, haloalkyl, hydroxy, amino, alkylamino, dialkylamino, halo, and oxo group; or each R′₃ and R₃′ or R″₄, and R₄′ are taken together along with the atom to which they are attached to form double bond; or each R″₃ and R″₄, or R₃′ and R₄′ are taken together form oxo( [symbol] ) bond;
enantiomers, diastereomers, racemates, pharmaceutically acceptable salts or solvates thereof.

In an embodiment, a method of treating a benign or malignant diseases of the breast or reproductive tract, preferably treating breast cancer, comprising compound of formula (C):

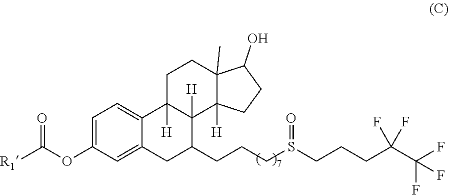

wherein;
R′₁ is selected from

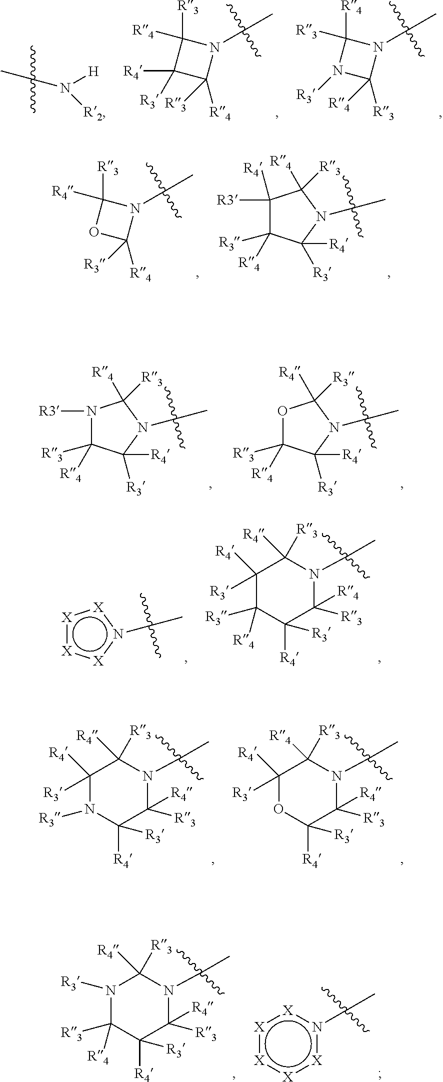

R′₂ is selected from,

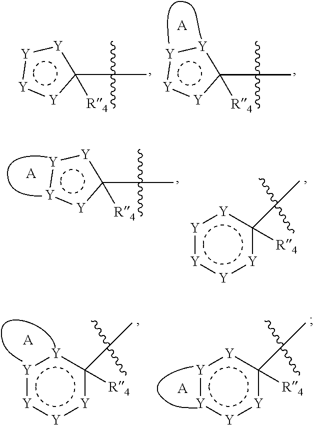

[symbol] represents the aromatic ring;
[symbol] represents the saturation, partial unsaturation or the complete unsaturation in the ring;
X is each independently selected from N, O, S,

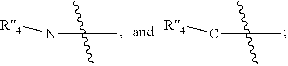

Y is selected from C,

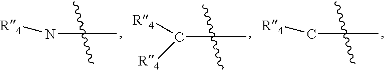

N, O, and S;
fused ring A represents an optionally substituted saturated, unsaturated, or aromatic four, five, six, or seven member ring having zero or more heteroatoms in the ring, the remaining atoms of the ring being carbon with each heteroatom being independently selected from nitrogen, oxygen and sulfur, wherein ring A is optionally substituted with one or more R″₆ radicals independently selected from hydrogen, alkyl, haloalkyl, hydroxy, amino, alkylamino, dialkylamino, unsubstituted heterocycloalkyl, substituted heterocycloalkyl, halo, and oxo group,
wherein the one or more substitution on substituted heterocycloalkyl is selected from alkyl, haloalkyl, hydroxy, amino, alkylamino, dialkylamino, halo, and oxo group;
R″₃, R″₄, R₃′, R₄′ each independently selected from H, alkyl, haloalkyl, hydroxy, amino, alkylamino, dialkylamino, heterocycloalkyl, substituted heterocycloalkyl, and halo group, wherein the substituted heterocycloalkyl is substituted by one or more substitution is selected from alkyl, haloalkyl, hydroxy, amino, alkylamino, dialkylamino, halo, and oxo group; or each R″₃ and R₃′ or R″₄, and R₄′ or R″₃ and R″₄, or R₃′ and R₄′ are taken together along with the atom to which they are attached to form a three to seven membered saturated, partially unsaturated or unsaturated ring which may contain one or more heteroatoms selected from N, O and S, and the ring may optionally be substituted at a substitutable position with one or more R′₅ radicals, wherein the one or more R′₅ radicals are independently selected from hydrogen, alkyl, haloalkyl, hydroxy, amino, alkylamino, dialkylamino, halo, and oxo group; or each R″₃ and R₃′ or R″₄, and R₄′ are taken together along with the atom to which they are attached to form double bond; or each R″₃ and R″₄, or R₃′ and R₄′ are taken together form oxo( [symbol] ) bond;
enantiomers, diastereomers, racemates, pharmaceutically acceptable salts or solvates thereof.

In another embodiment, the present invention relates to pharmaceutical composition comprising at least one compound selected from compound of formula (I-a) to (I-bq); enantiomers, diastereomers, racemates, pharmaceutically acceptable salts or solvates thereof and a pharmaceutically acceptable excipients. The pharmaceutical compositions of the present disclosure can be in any form known to those of skill in the art. The pharmaceutical compositions of this invention may be administered orally, parenterally, by inhalation spray, topically, rectally, nasally, buccally or via an implanted reservoir, preferably oral administration or administration by injection.

For instance, in some embodiments the pharmaceutical composition comprising desired product is formulated for oral delivery. In embodiment the pharmaceutical composition comprising desired product is selected from a group consisting of a concentrate, dried powder, liquid, capsule, pellet, and pill.

For instance, in some embodiments the pharmaceutical composition comprising desired product is for parenteral. In embodiment the pharmaceutical composition comprising desired product is selected from a group consisting of a intravenous injection, intramuscular injection, subcutaneous injection, powder for solution for injection, powder for suspension for injection, liposome, oily injection, sustained release particles.

In one embodiment, the compound for the said pharmaceutical composition is selected from the compound of formula A, formula B, formula C, formula D, formula E, formula F, formula G, formula H, formula W, formula J, formula K, formula L, formula M, formula N, formula O, formula P, formula Q, formula R, formula S, Formula T, formula U; and enantiomers, diastereomers, racemates, pharmaceutically acceptable salts and solvates thereof.

In one embodiment, the present invention provides a pharmaceutical compositions comprises pharmaceutically acceptable excipients selected from carriers, binders, diluents, bulking agents, preservatives, disintegrants, flow regulators, plasticizers, wetting agents, dispersants, emulsifiers, solvents, antioxidants.

In another embodiment, the invention provides use of compounds of the present invention in the preparation of a pharmaceutical formulation as describe hereinabove, for the treatment of a benign or malignant disease of the breast or reproductive tract, preferably treating breast cancer. In another embodiment, the invention provides compounds for the treatment of a benign or malignant disease of the breast or reproductive tract, preferably treating breast cancer.

In another embodiment, the compound of the present invention provides use in the treatment of oestrogen-dependent indications such as breast cancer and gynaecological conditions, such as endometriosis.

In another embodiment, the pharmaceutical composition of the present invention comprising at least one compound selected from compound (I-a) to (I-bq) and a pharmaceutically acceptable excipient in the treatment of oestrogen-dependent indications such as breast cancer and gynaecological conditions, such as endometriosis.

In another embodiment, the invention provides a method of treating a benign or malignant diseases of the breast or reproductive tract, preferably treating breast cancer, comprising administration of at least one formula of compound selected from formula A, formula B, formula C, formula D, formula E, formula F, formula G, formula H, formula W, formula J, formula K, formula L, formula M, formula N, formula O, formula P, formula Q, formula R, formula S, Formula T, formula U; enantiomers, diastereomers, racemates, pharmaceutically acceptable salts or solvates thereof.

In another embodiment, the invention provides a method of treating a benign or malignant diseases of the breast or reproductive tract, preferably treating breast cancer, comprising administration of a pharmaceutical composition comprising at least one formula of compound selected from formula A, formula B, formula C, formula D, formula E, formula F, formula G, formula H, formula W, formula J, formula K, formula L, formula M, formula N, formula O, formula P, formula Q, formula R, formula S, Formula T, formula U; and enantiomers, diastereomers, racemates, pharmaceutically acceptable salts and solvates thereof and a pharmaceutical acceptable excipient.

In another embodiment, the invention provides a method of treating a benign or malignant diseases of the breast or reproductive tract, preferably treating breast cancer, comprising administration of at least one compound selected from compound (I-a) to (I-bq).

In another embodiment, the invention provides a method of treating a benign or malignant diseases of the breast or reproductive tract, preferably treating breast cancer, comprising administration of a pharmaceutical composition comprising at least one compound selected from compound (I-a) to (I-bq) and a pharmaceutically acceptable excipient..

In another embodiment, the present invention provides a pharmaceutical composition for treating a benign or malignant diseases of the breast or reproductive tract comprising at least one compound selected from compound (I-a) to (I-bq) and a pharmaceutically acceptable excipient.

In another embodiment, the invention provides at least one compound selected from compound (I-a) to (I-bq) for treating a benign or malignant diseases of the breast or reproductive tract.

Also, in other embodiment, the present disclosure relates to new novel compounds and any stereochemically isomeric form, hydrate, solvate or pharmaceutically acceptable salt thereof; either alone or in combination with at least one additional therapeutic agent, in the treatment of diseases and/or symptoms meant to be treated by the original drugs. The combination with an additional therapeutic agent may take the form of combining the new novel compounds compounds with any known therapeutic agent.

The following examples are given for the purpose of illustrating the present invention and should not be considered as limiting the scope of the invention.

### EXAMPLES

### List of Abbreviations

PCC—Pyridinium chlorochromate
DMP—Dess-Martin Periodinane
DCM—Dichloromethane
DIPEA—Diisopropylethylamine
NaBH(OAc)₃—Sodium triacetoxyborohydride
BnBr—Benzyl bromide
MeI—Methyl iodide

### Example-1: Preparation of (7R,8R,9S,13S,14S)-3-hydroxy-13-methyl-7-(9-((4,4,5,5,5-pentafluoropentyl)sulfinyl)nonyl)-6,7,8,9,11,12,13,14,15,16-decahydro-17H-cyclopenta[a]phenanthren-17-one

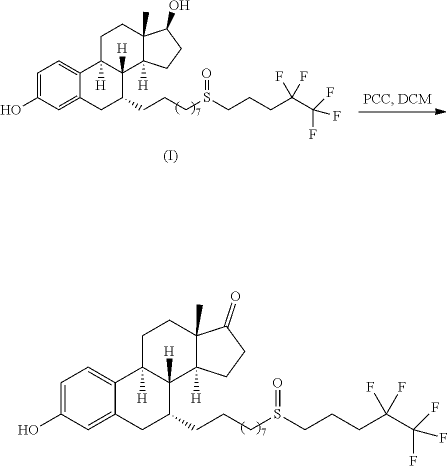

To a stirred solution of (7R,8R,9S,13S,14S,17S)-13-methyl-7-(9-((4,4,5,5,5-pentafluoropentyl)sulfinyl)nonyl)-7,8,9,11,12,13,14,15,16,17-decahydro-6H-cyclopenta[a]phenanthrene-3,17-diol (0.9 g) in dichloromethane (10 mL) was added pyridinium chlorochromate (0.323 g) under nitrogen atmosphere and resulting suspension was stirred for 10 min at room temperature. After confirmation of reaction completion by TLC, the reaction mixture was diluted with water (10 mL). The organic compound was extracted with dichloromethane (3×200 mL), washed with water (2×200 mL), dried the organic layer over anhydrous sodium sulfate and concentrated under reduced pressure to obtain the crude material. The crude was purified by preparative HPLC using trifluoroacetic acid as a modifier to afford 0.061 g of (7R,8R,9S,13S,14S)-3-hydroxy-13-methyl-7-(9-((4,4,5,5,5-pentafluoropentyl)sulfinyl)nonyl)-6,7,8,9,11,12,13,14,15,16-decahydro-17H-cyclopenta[a]phenanthren-17-one as a white solid.

LCMS purity: 98.74%

¹H-NMR (DMSO-d6): δ 9.00 (br s, 1H), 7.05-7.03 (d, 1H), 6.50-6.51 (dd, 1H), 6.43-6.42 (d, 1H), 2.85-2.61 (m, 6H), 2.41-2.23 (m, 5H), 2.11-2.01 (m, 1H), 1.93-1.81 (m, 4H), 1.69-1.51 (m, 6H), 1.36-1.14 (m, 15H), 0.89-0.92 (m, 1H), 0.80 (s, 3H).

### Example-2: Preparation of (7R,8R,9S,13S,14S)-13-methyl-17-oxo-7-(9-((4,4,5,5,5-pentafluoropentyl)sulfinyl)nonyl)-7,8,9,11,12,13,14,15,16,17-decahydro-6H-cyclopenta[a]phenanthren-3-yl-4-(pyrrolidin-1-yl)piperidine-1-carboxylate

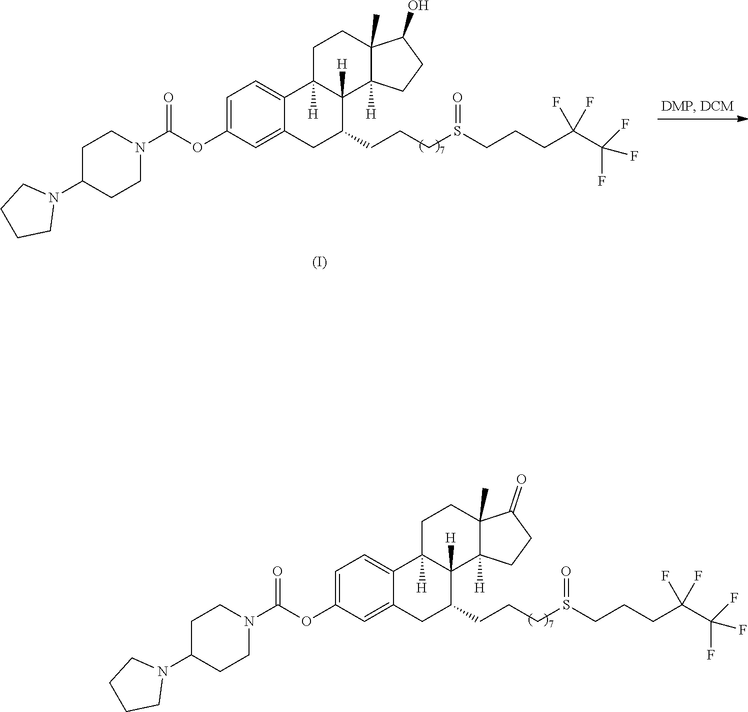

To a stirred solution of (7R,8R,9S,13S,14S,17S)-17-hydroxy-13-methyl-7-(9-((4,4,5,5,5-pentafluoropentyl)sulfinyl)nonyl)-7,8,9,11,12,13,14,15,16,17-decahydro-6H-cyclopenta[a]phenanthren-3-yl-4-(pyrrolidin-1-yl)piperidine-1-carboxylate (0.5 g) in dichloromethane (10 mL) was added Dess-Martin periodinane (DMP) (0.323 g) at 0° C. temperature under nitrogen atmosphere. The resulting suspension was stirred for 4 h at room temperature. After confirmation of reaction completion by TLC, the reaction mixture was diluted with water (10 mL). The organic compound was extracted with dichloromethane (3×50 mL), washed with water (2×200 mL) and dried over anhydrous sodium sulfate. Dried organic layer was concentrated under reduced pressure to obtain the crude material. This crude was purified by preparative HPLC using trifluoroacetic acid as a modifier to afford 0.156 g of (7R,8R,9S,13S,14S)-13-methyl-17-oxo-7-(9-((4,4,5,5,5-pentafluoropentyl)sulfinyl)nonyl)-7,8,9,11,12,13,14,15,16,17-decahydro-6H-cyclopenta[a]phenanthren-3-yl-4-(pyrrolidin-1-yl)piperidine-1-carboxylate as a white solid.

LCMS purity: 95.67%

¹H-NMR (DMSO-d6): δ 7.29-7.26 (d, 1H), 6.83-6.81 (d, 1H), 6.78 (s, 1H), 4.22-4.08 (m, 2H), 3.61-3.41 (m, 2H), 3.34-3.32 (t, 1H), 3.07-3.03 (m, 3H), 2.93-2.81 (m, 3H), 2.74-2.63 (m, 3H), 2.40-2.29 (m, 3H), 2.07-2.02 (m, 6H), 1.93-1.83 (m, 7H), 1.75-1.53 (m, 9H), 1.43-1.13 (m, 15H), 0.89-0.87 (m, 1H), 0.80 (s, 3H).

### Example-3: Preparation of (7R,8R,9S,13S,14S)-13-methyl-17-oxo-7-(9-((4,4,5,5,5-pentafluoropentyl)sulfinyl)nonyl)-7,8,9,11,12,13,14,15,16,17-decahydro-6H-cyclopenta[a]phenanthren-3-yl 4-morpholinopiperidine-1-carboxylate

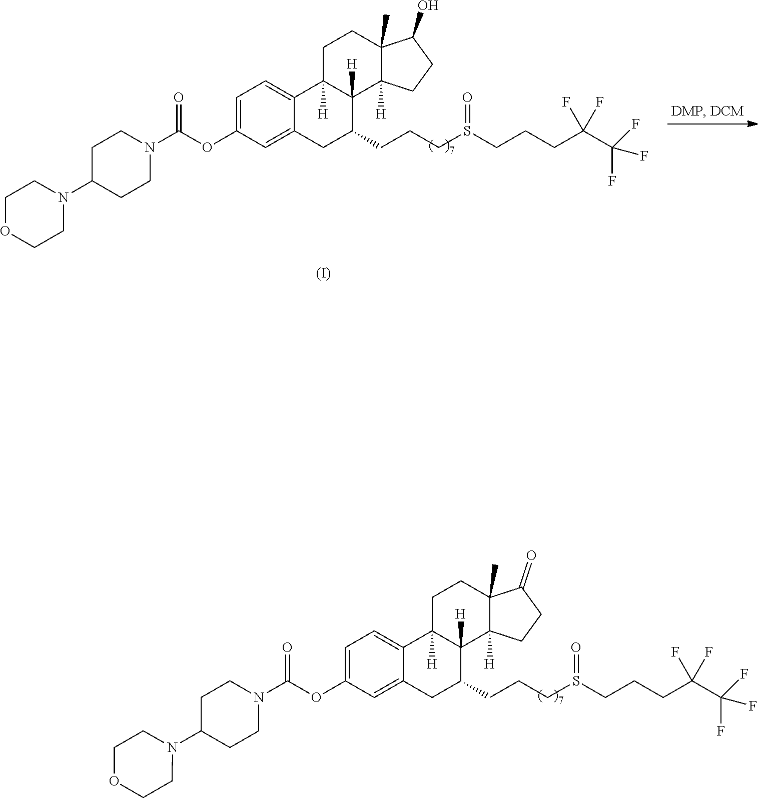

To a stirred solution of (7R,8R,9S,13S,14S,17S)-17-hydroxy-13-methyl-7-(9-((4,4,5,5,5-pentafluoropentyl)sulfinyl)nonyl)-7,8,9,11,12,13,14,15,16,17-decahydro-6H-cyclopenta[a]phenanthren-3-yl 4-morpholinopiperidine-1-carboxylate (0.5 g) in dichloromethane (10 mL) was added Dess-Martin periodinane (DMP) (0.323 g) at 0° C. temperature under nitrogen atmosphere. The resulting suspension was stirred for 6 h at room temperature. After confirmation of reaction completion by TLC, the reaction mixture was diluted with water (10 mL). The organic compound was extracted with dichloromethane (3×50 mL), washed with water (2×200 mL), dried the organic layer over anhydrous sodium sulfate and concentrated under reduced pressure to obtain the crude material. The crude was purified by preparative HPLC using trifluoroacetic acid as a modifier to afford 0.13 g of (7R,8R,9S,13S,14S)-13-methyl-17-oxo-7-(9-((4,4,5,5,5-pentafluoropentyl) sulfinyl)nonyl)-7,8,9,11,12,13,14,15,16,17-decahydro-6H-cyclopenta[a]phenanthrene-3-yl 4-morpholinopiperidine-1-carboxylate as a white sticky solid.

LCMS purity: 99.70%

¹H-NMR (DMSO-d6): δ 7.26-7.24 (d, 1H), 6.81-6.79 (d, 1H), 6.75 (s, 1H), 3.99-4.09 (m, 2H), 3.55-3.58 (m, 4H), 2.95-2.61 (m, 8H), 2.45-2.28 (m, 10H), 2.10-2.01 (m, 1H), 1.93-1.81 (m, 6H), 1.74-1.52 (m, 6H), 1.33-1.14 (m, 17H), 0.87-0.89 (m, 1H), 0.80 (s, 3H).

### Example-4: Preparation of (7R,8R,9S,13S,14S)-13-methyl-17-oxo-7-(9-((4,4,5,5,5-pentafluoropentyl)sulfinyl)nonyl)-7,8,9,11,12,13,14,15,16,17-decahydro-6H-cyclopenta[a]phenanthren-3-yl[1,4′-bipiperidine]-1′-carboxylate

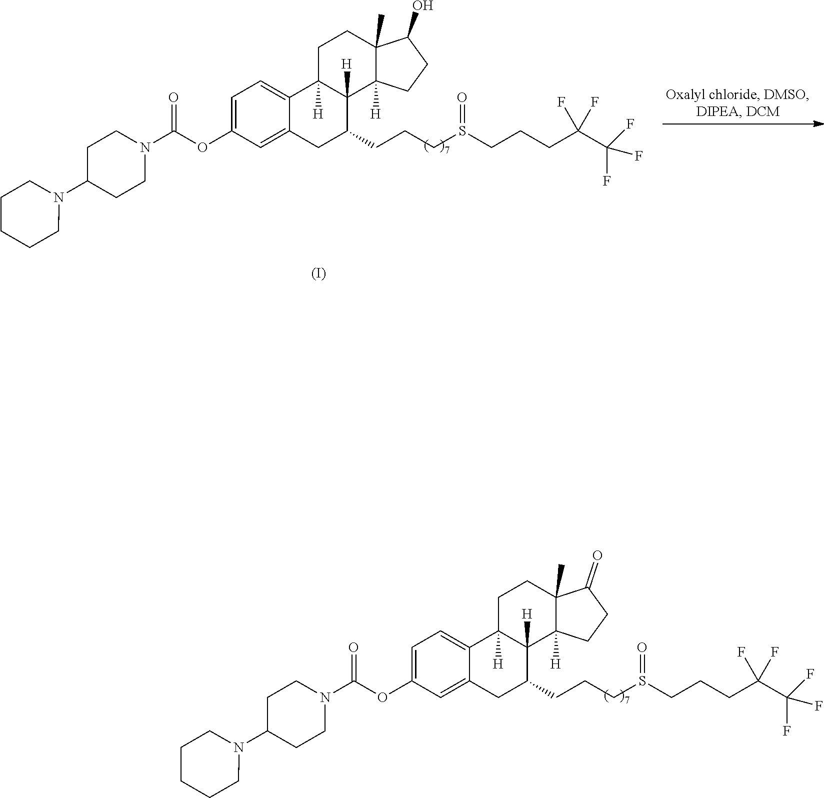

To a stirred solution of oxalyl chloride (0.310 mL) in dichloromethane (113 mL) was added dimethyl sulfoxide (0.625 mL) in a drop wise manner cooled at −75° C. temperature under nitrogen atmosphere. After that, (7R,8R,9S,13S,14S,17S)-17-hydroxy-13-methyl-7-(9-((4,4,5,5,5-pentafluoropentyl)sulfinyl)nonyl)-7,8,9,11,12,13,14,15,16,17-decahydro-6H-cyclopenta[a]phenanthren-3-yl[1,4′-bipiperidine]-1′-carboxylate (2.5 g) in dichloromethane (25 mL) was added to above mixture in a drop wise over a period of 30 min. The temperature of the reaction mixture was maintained at −75° C. for additional 2.5 h followed by the addition of N,N′-diisopropylamine (4.12 mL). Resultant reaction mixture was continued for stirring at room temperature for 30 min. After confirmation of reaction completion by TLC, the mixture was diluted with water (25 mL). The reaction mass was extracted with dichloromethane (3×20 mL), washed with water (2×10 mL), dried over anhydrous sodium sulfate and concentrated under reduced pressure to obtain the crude material. The crude was purified flash chromatography to obtain 0.75 g of (7R,8R,9S,13S,14S)-13-methyl-17-oxo-7-(9-((4,4,5,5,5-pentafluoropentyl)sulfinyl)nonyl)-7,8,9,11,12,13,14,15,16,17-decahydro-6H-cyclopenta[a]phenanthren-3-yl[1,4′-bipiperidine]-1′-carboxylate as a white sticky solid.

LCMS purity: 96.79%

¹H-NMR (DMSO-d6): δ 7.28-7.26 (d, 1H), 6.84-6.82 (d, 1H), 6.80 (s, 1H), 4.15-4.01 (m, 2H), 2.96-2.99 (m, 1H), 2.90-2.69 (m, 7H), 2.67-2.61 (m, 1H), 2.71-2.35 (m, 9H), 2.10-2.05 (m, 1H), 1.94-1.85 (m, 4H), 1.76-1.56 (m, 8H), 1.54-1.47 (m, 4H), 1.38-1.33 (m, 9H), 1.25-1.23 (m, 10H), 0.92-0.89 (m, 1H), 0.83 (s, 3H).

### Example-5: Preparation of (7R,8R,9S,13S,14S)-13-methyl-17-oxo-7-(9-((4,4,5,5,5-pentafluoropentyl)sulfinyl)nonyl)-7,8,9,11,12,13,14,15,16,17-decahydro-6H-cyclopenta[a]phenanthren-3-yl (1-methylpyrrolidin-3-yl) carbonate

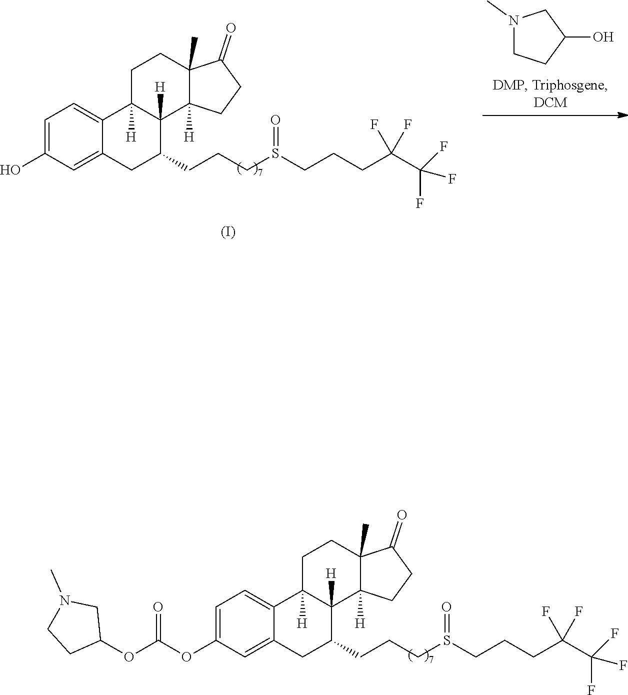

To a stirred solution of (7R,8R,9S,13S,14S)-3-hydroxy-13-methyl-7-(9-((4,4,5,5,5-pentafluoropentyl)sulfinyl)nonyl)-6,7,8,9,11,12,13,14,15,16-decahydro-17H-cyclopenta[a]phenanthren-17-one (0.5 g) in dichloromethane (6 mL) were added triphosgene (0.122 g) and DIPEA (0.160 g) at 0° C. temperature under nitrogen atmosphere. The resultant reaction mixture was stirred at 0° C. for 15 min. After that, 1-methylpyrrolidin-3-ol (0.125 g) was added to the above reaction mixture and reaction continued stirring at room temperature for 2 h. After confirmation of reaction completion by TLC, the mixture was diluted with water (10 mL). The organic compound was extracted with dichloromethane (3×20 mL) followed by washing with water (2×10 mL). Collected organics were dried the organic layer over anhydrous sodium sulfate and concentrated under reduced pressure to obtain the crude material. The crude was purified by preparative HPLC using ammonium bicarbonate as a modifier to afford 0.07 g of (7R,8R,9S,13S,14S)-13-methyl-17-oxo-7-(9-((4,4,5,5,5-pentafluoropentyl)sulfinyl)nonyl)-7,8,9,11,12,13,14,15,16,17-decahydro-6H-cyclopenta[a]phenanthren-3-yl(1-methylpyrrolidin-3-yl) carbonate as a white sticky solid.

LCMS purity: 99.28%

¹H-NMR (DMSO-d6): δ 7.33-7.31 (d, 1H), 6.97-6.94 (d, 1H), 6.93 (s, 1H), 2.96-2.80 (m, 3H), 2.76-2.69 (m, 4H), 2.64-2.59 (m, 2H), 2.46-2.34 (m, 8H), 2.28-2.18 (m, 5H), 2.15-2.05 (m, 1H), 1.94-1.77 (m, 6H), 1.73-1.54 (m, 6H), 1.43-1.34 (m, 6H), 1.29-1.17 (m, 6H), 0.94-0.89 (m, 1H), 0.84 (s, 3H).

### Example-6: Preparation of (7R,8R,9S,13S,14S)-13-methyl-17-oxo-7-(9-((4,4,5,5,5-pentafluoropentyl)sulfinyl)propyl)-7,8,9,11,12,13,14,15,16,17-decahydro-6H-cyclopenta[a]phenanthren-3-yl (1,3-dihydroxy-2-(hydroxymethyl)propan-2-yl)carbamate

### Step-1: Preparation of (7R,8R,9S,13S,14S)-13-methyl-17-oxo-7-(9-((4,4,5,5,5-pentafluoropentyl)sulfinyl)propyl)-7,8,9,11,12,13,14,15,16,17-decahydro-6H-cyclopenta[a]phenanthren-3-yl (4-nitrophenyl) carbonate

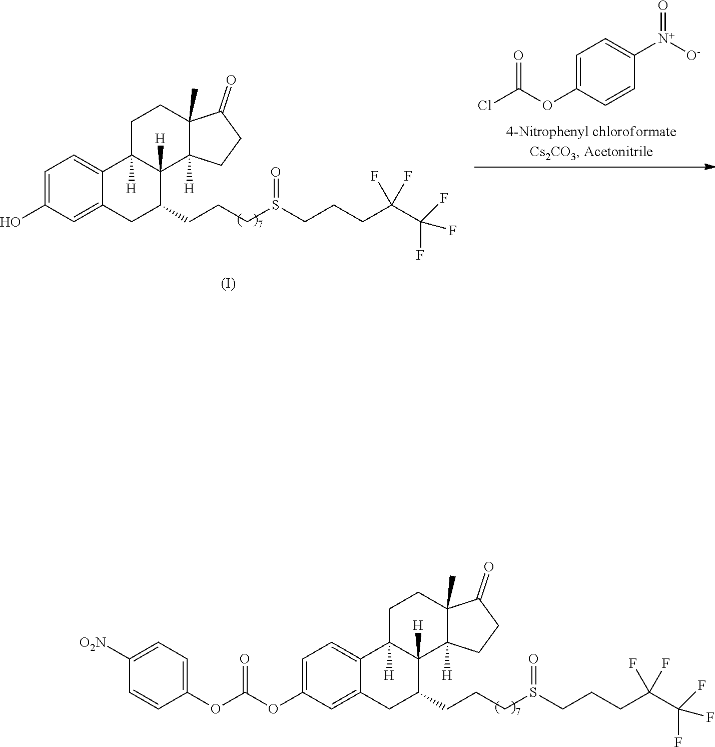

To a stirred solution of (7R,8R,9S,13S,14S)-3-hydroxy-13-methyl-7-(9-((4,4,5,5,5-pentafluoropentyl)sulfinyl)propyl)-6,7,8,9,11,12,13,14,15,16-decahydro-17H-cyclopenta[a]phenanthren-17-one (1 g) in acetonitrile (10 mL) were added caesium carbonate (1.62 g) and 4-nitrophenyl chloroformate (0.5 g) at room temperature and stirred the reaction mixture for 15 min at the same temperature. After confirmation of reaction completion by TLC, the reaction mass was diluted with water (15 mL) and extracted with ethyl acetate (2×50 mL), combined organic layer was dried over anhydrous sodium sulfate, and concentrated the organics under reduced pressure to give (7R,8R,9S,13S,14S)-13-methyl-17-oxo-7-(9-((4,4,5,5,5-pentafluoropentyl)sulfinyl)propyl)-7,8,9,11,12,13,14,15,16,17-decahydro-6H-cyclopenta[a]phenanthren-3-yl (4-nitrophenyl) carbonate (1.2 g crude), which was used for next step without further purification.

### Step-2: Preparation of (7R,8R,9S,13S,14S)-13-methyl-17-oxo-7-(9-((4,4,5,5,5-pentafluoropentyl)sulfinyl)propyl)-7,8,9,11,12,13,14,15,16,17-decahydro-6H-cyclopenta[a]phenanthren-3-yl (1,3-dihydroxy-2-(hydroxymethyl)propan-2-yl)carbamate

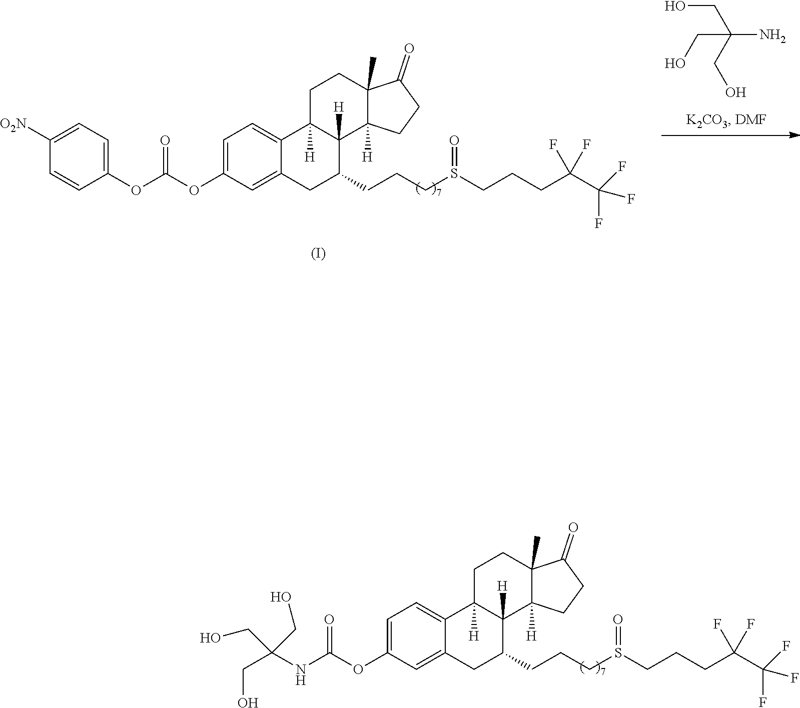

To a previously stirred suspension of (7R,8R,9S,13S,14S)-13-methyl-17-oxo-7-(9-((4,4,5,5,5-pentafluoropentyl)sulfinyl)propyl)-7,8,9,11,12,13,14,15,16,17-decahydro-6H-cyclopenta[a]phenanthren-3-yl (4-nitrophenyl) carbonate (1 g) and potassium carbonate (0.62 g) in dimethylformamide (12 mL) was added tromethamine (0.3 g) at room temperature under nitrogen atmosphere. The resulting reaction mixture was stirred at room temperature for additional 2 h. After confirmation of reaction completion by TLC, the mixture was diluted with water (20 mL), extracted with ethyl acetate (3×50 mL), washed with water (2×20 mL), dried the organic layer over anhydrous sodium sulfate and concentrated under reduced pressure to obtain the crude material. The crude was purified by preparative HPLC using trifluoroacetic acid as a modifier to afford 0.030 g of (7R,8R,9S,13S,14S)-13-methyl-17-oxo-7-(9-((4,4,5,5,5-pentafluoropentyl) sulfinyl)propyl)-7,8,9,11,12,13,14,15,16,17-decahydro-6H-cyclopenta[a]phenanthren-3-yl (1,3-dihydroxy-2-(hydroxymethyl)propan-2-yl)carbamate as an off-white semi-solid.

LCMS purity: 99.53%

¹H-NMR (DMSO-d6): δ 7.27 (d, 1H), 6.84 (d, 1H), 6.81 (s, 1H), 6.70 (br s, 1H), 4.54-4.51 (t, 3H), 3.57 (d, 6H), 2.88-2.71 (m, 5H), 2.64-2.62 (m, 1H), 2.44-2.37 (m, 4H), 2.12-2.08 (m, 1H), 1.92-1.56 (m, 11H), 1.40-1.14 (m, 15H), 0.91-0.88 (m, 1H), 0.84 (s, 3H).

### Example-7: Preparation of (7R,8R,9S,13S,14S)-13-methyl-17-oxo-7-(9-((4,4,5,5,5-pentafluoropentyl)sulfinyl)propyl)-7,8,9,11,12,13,14,15,16,17-decahydro-6H-cyclopenta[a]phenanthren-3-yl 6-methyl-2,6-diazaspiro[3.3]heptane-2-carboxylate

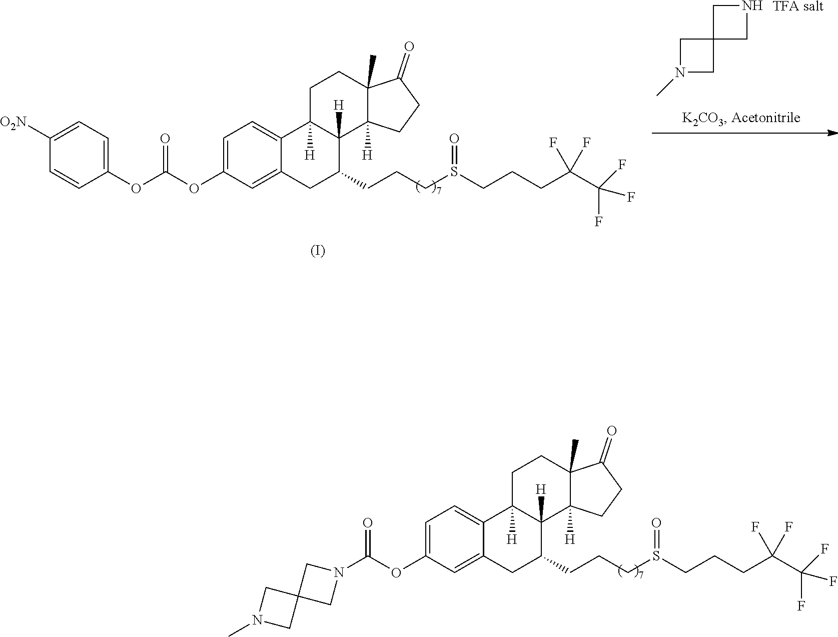

To a previously stirred suspension of (7R,8R,9S,13S,14S)-13-methyl-17-oxo-7-(9-((4,4,5,5,5-pentafluoropentyl)sulfinyl)propyl)-7,8,9,11,12,13,14,15,16,17-decahydro-6H-cyclopenta[a]phenanthren-3-yl (4-nitrophenyl) carbonate (1 g, as prepared in example-6, step-1) and potassium carbonate (1.8 g) in acetonitrile (40 mL) was added 2-Methyl-2,6-diazaspiro[3.3]heptane bis(trifluoroacetate) (0.66 g) at 0° C. temperature under nitrogen atmosphere. The resulting reaction mixture was stirred at room temperature for 16 h. After confirmation of reaction completion by TLC, the mixture was diluted with water (20 mL). The organic compound was extracted with ethyl acetate (3×50 mL), washed with water (2×20 mL), dried the organic layer over anhydrous sodium sulfate and concentrated under reduced pressure to obtain the crude material. The crude was purified by preparative-HPLC using trifluoroacetic acid as a modifier to afford 0.374 g of (7R,8R,9S,13S,14S)-13-methyl-17-oxo-7-(9-((4,4,5,5,5-pentafluoropentyl)sulfinyl)propyl)-7,8,9,11,12,13,14,15,16,17-decahydro-6H-cyclopenta[a]phenanthren-3-yl 6-methyl-2,6-diazaspiro[3.3]heptane-2-carboxylate mono-trifluoroacetate as a white semi-solid.

LCMS purity: 98.71%

¹H-NMR (DMSO-d6): δ 9.51 (br s, 1H), 7.28 (d, 1H), 6.82 (d, 1H), 6.78 (s, 1H), 4.40-4.36 (m, 2H), 4.32-4.27 (m, 2H), 4.15-4.11 (m, 4H), 2.96-2.71 (m, 9H), 2.44-2.39 (m, 4H), 2.14-2.05 (m, 1H), 1.92-1.53 (m, 11H), 1.44-1.15 (m, 15H), 0.93-0.88 (m, 1H), 0.83 (s, 3H).

### Example-8: Preparation of (7R,8R,9S,13S,14S)-13-methyl-17-oxo-7-(9-((4,4,5,5,5-pentafluoropentyl)sulfinyl)nonyl)-7,8,9,11,12,13,14,15,16,17-decahydro-6H-cyclopenta[a]phenanthren-3-yl 4-(dimethylamino)piperidine-1-carboxylate

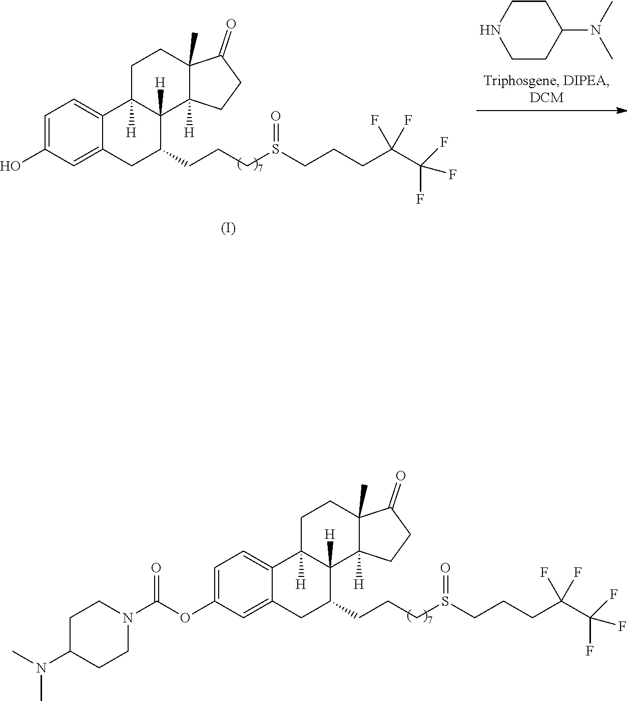

To a stirred solution of (7R,8R,9S,13S,14S)-3-hydroxy-13-methyl-7-(9-((4,4,5,5,5-pentafluoropentyl)sulfinyl)nonyl)-6,7,8,9,11,12,13,14,15,16-decahydro-17H-cyclopenta[a]phenanthren-17-one (0.5 g) in dichloromethane (10 mL) were added DIPEA (0.21 mL) and triphosgene (0.122 g) at 0° C. temperature under nitrogen atmosphere and resulting mixture was stirred for 10 min at the same temperature. After 10 min, N,N-dimethylpiperidin-4-amine (0.158 g) was added in the reaction mixture at 0° C. temperature, and the reaction mixture was stirred at room temperature for 1.5 h. After confirmation of reaction completion by TLC, the reaction mass was diluted with water (20 mL) and extracted with dichloromethane (3×20 mL). The combined organic layer was dried over anhydrous sodium sulfate and concentrated under reduced pressure. The crude was purified by flash chromatography to obtain 0.2 g of (7R,8R,9S,13S,14S)-13-methyl-17-oxo-7-(9-((4,4,5,5,5-pentafluoropentyl)sulfinyl)nonyl)-7,8,9,11,12,13,14,15,16,17-decahydro-6H-cyclopenta[a]phenanthren-3-yl 4-(dimethylamino)piperidine s-1-carboxylate as an off-white color solid.

LCMS purity: 95.06%

¹H-NMR (DMSO-d6): δ 7.28-7.26 (d, 1H), 6.86-6.83 (d, 1H), 6.81 (s, 1H), 4.12-4.01 (m, 2H), 3.01-2.89 (m, 1H), 2.90-2.61 (m, 7H), 2.43-2.33 (m, 11H), 2.14-2.05 (m, 1H), 1.92-1.84 (m, 6H), 1.79-1.68 (m, 3H), 1.65-1.54 (m, 3H), 1.39-1.33 (m, 8H), 1.23-1.18 (m, 10H), 0.94-0.91 (m, 1H), 0.84 (s, 3H).

### Example-9: Preparation of (7R,8R,9S,13S,14S)-13-methyl-17-oxo-7-(9-((4,4,5,5,5-pentafluoropentyl)sulfinyl)propyl)-7,8,9,11,12,13,14,15,16,17-decahydro-6H-cyclopenta[a]phenanthren-3-yl (1,3-dihydroxy-2-(hydroxymethyl)propan-2-yl)carbamate

### Step-1: Preparation of tert-butyl 4-((2-hydroxyethyl)amino)piperidine-1-carboxylate

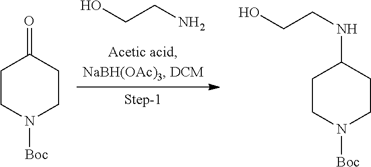

To a stirred solution of N-bocpiperidin-4-one (1 g) and 2-aminoethanol (0.31 g) in dichloromethane (20 mL) was added catalytic amount of glacial acetic acid (0.1 mL) at 0° C. temperature under nitrogen atmosphere and the resulting reaction mixture was stirred at 0° C. temperature for 1 h. After that, sodium triacetoxyborohydride (2.66 g) was added to the reaction mixture in portions and the resulting reaction mixture was allowed to stir at room temperature under nitrogen atmosphere for 4 h. After confirmation of reaction completion by TLC, the reaction mass was diluted with saturated sodium bicarbonate solution (50 mL) and extracted with dichloromethane (3×40 mL). The combined organic layer was dried over anhydrous sodium sulfate and concentrated under reduced pressure. The crude was purified by flash chromatography to obtain 0.75 g of tert-butyl 4-((2-hydroxyethyl)amino)piperidine-1-carboxylate as an off-white semi-solid.

### Step-2: Preparation of 2-(piperidin-4-ylamino)ethan-1-ol

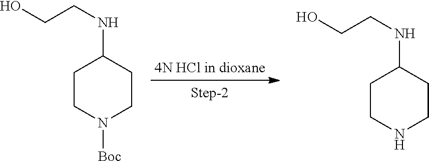 [SMILES: CC(C)(C)OC(=O)N1CCC(NCCO)CC1 ; OCCNC1CCNCC1]

To a stirred solution of tert-butyl 4-((2-hydroxyethyl)amino)piperidine-1-carboxylate (0.75 g) in dioxane (15 mL) was added 4 N hydrochloric acid in dioxane (8 mL) at 0° C. temperature under nitrogen atmosphere and the resulting reaction mixture was stirred at room temperature for 4 h. After confirmation of reaction completion by TLC, the reaction mixture was concentrated under reduced pressure to obtain the crude material. The crude material was further washed by diethyl ether (10 mL to obtain 2-(piperidin-4-ylamino)ethan-1-ol (0.7 g) as an off-white solid.

### Step-3: Preparation of (7R,8R,9S,13S,14S)-13-methyl-17-oxo-7-(9-((4,4,5,5,5-pentafluoropentyl)sulfinyl)propyl)-7,8,9,11,12,13,14,15,16,17-decahydro-6H-cyclopenta[a]phenanthren-3-yl (1,3-dihydroxy-2-(hydroxymethyl)propan-2-yl)carbamate

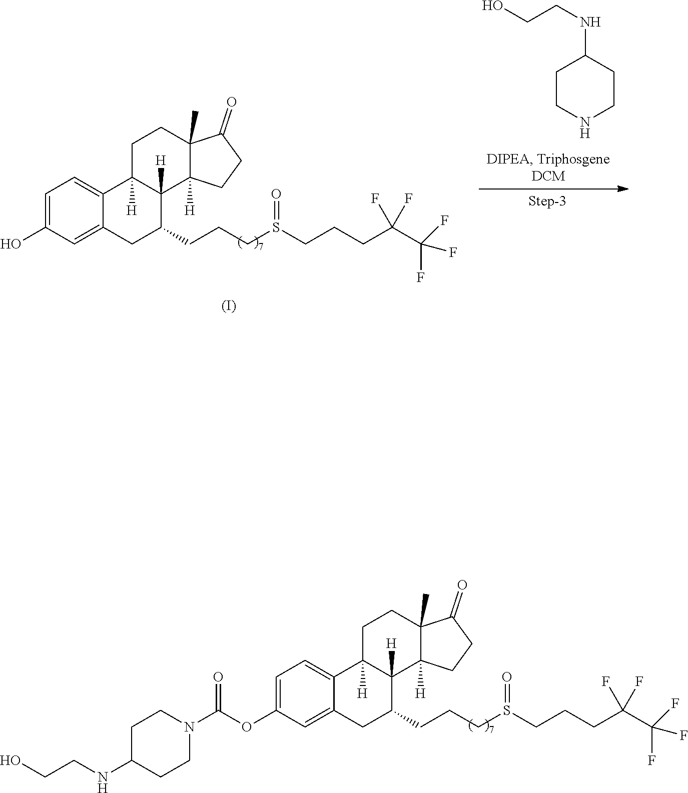

To a stirred solution of (7R,8R,9S,13S,14S)-3-hydroxy-13-methyl-7-(9-((4,4,5,5,5-pentafluoropentyl)sulfinyl)nonyl)-6,7,8,9,11,12,13,14,15,16-decahydro-17H-cyclopenta[a]phenanthren-17-one (0.6 g) in dichloromethane (12 mL) were added DIPEA (0.43 mL) and triphosgene (0.15 g) at 0° C. temperature and stirred the reaction mixture for 10 min at the same temperature. After 10 min, 2-(piperidin-4-ylamino)ethan-1-ol dihydrochloride (0.32 g) was added in the reaction mixture at 0° C. temperature, and the reaction mixture was stirred at room temperature for 1.5 h. After confirmation of reaction completion by TLC, the reaction mass was diluted with water (20 mL) and extracted with dichloromethane (3×25 mL). The combined organic layer was dried over anhydrous sodium sulfate and concentrated under reduced pressure.

The crude was purified by flash chromatography to afford 0.030 g of (7R,8R,9S,13S,14S)-13-methyl-17-oxo-7-(9-((4,4,5,5,5-pentafluoropentyl) sulfinyl)propyl)-7,8,9,11,12,13,14,15,16,17-decahydro-6H-cyclopenta[a]phenanthren-3-yl (1,3-dihydroxy-2-(hydroxymethyl) propan-2-yl)carbamate as an off-white semi solid.

LCMS purity: 95.22%
¹H-NMR (DMSO-d6): δ 7.27 (d, 1H), 6.85-6.81 (m, 2H), 4.62 (br s, 1H), 4.-01-3.92 (m, 2H), 3.49-3.48 (m, 2H), 3.09-3.06 (m, 1H), 2.96-2.71 (m, 9H), 2.64-2.61 (m, 2H), 2.44-2.39 (m, 4H), 2.14-2.06 (m, 1H), 1.94-1.54 (m, 13H), 1.39-1.24 (m, 17H), 0.94-0.89 (m, 1H), 0.84 (s, 3H).

### Example-10: Preparation of (7R,8R,9S,13S,14S)-13-methyl-17-oxo-7-(9-((4,4,5,5,5-pentafluoropentyl)sulfinyl)nonyl)-7,8,9,11,12,13,14,15,16,17-decahydro-6H-cyclopenta[a]phenanthren-3-yl 3-oxa-8-azabicyclo[3.2.1]octane-8-carboxylate

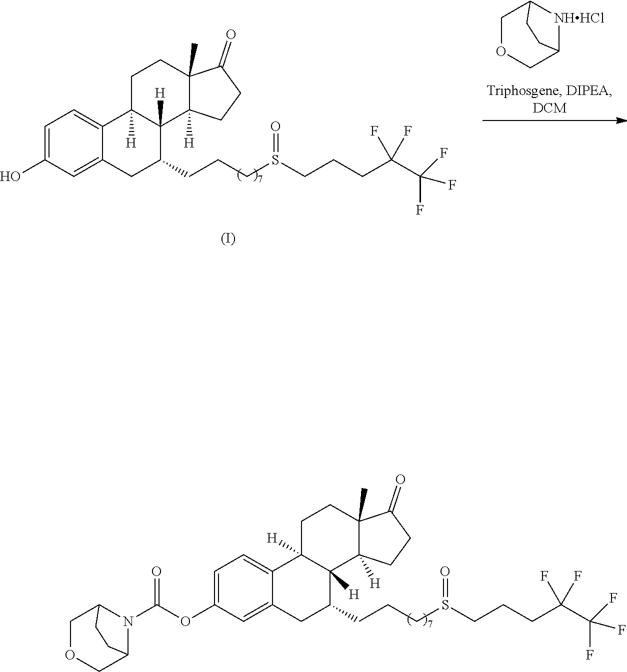

To a stirred solution of (7R,8R,9S,13S,14S)-3-hydroxy-13-methyl-7-(9-((4,4,5,5,5-pentafluoropentyl)sulfinyl)nonyl)-6,7,8,9,11,12,13,14,15,16-decahydro-17H-cyclopenta[a]phenanthren-17-one (0.5 g) in dichloromethane (10 mL) were added DIPEA (0.288 mL) and triphosgene (0.122 g) at 0° C. temperature and stirred the reaction mixture for 10 min at the same temperature. After 10 min, 3-oxa-8-azabicyclo[3.2.1]octane hydrochloride (0.185 g) was added in the reaction mixture at 0° C. temperature, and the reaction mixture was stirred at room temperature for 1.5 h. After confirmation of reaction completion by TLC, the reaction mass was diluted with water (20 mL) and extracted with dichloromethane (3×20 mL). The combined organic layer was dried over anhydrous sodium sulfate and concentrated under reduced pressure.

The crude was purified by preparative HPLC using ammonium bicarbonate as a modifier to afford 0.030 g of (7R,8R,9S,13S,14S)-13-methyl-17-oxo-7-(9-((4,4,5,5,5-pentafluoropentyl) sulfinyl)nonyl)-7,8,9,11,12,13,14,15,16,17-decahydro-6H-cyclopenta[a]phenanthren-3-yl 3-oxa-8-azabicyclo[3.2.1]octane-8-carboxylate as a white semi solid.

LCMS purity: 92.99%

¹H-NMR (DMSO-d6): δ 7.30-7.28 (d, 1H), 6.89-6.87 (d, 1H), 6.861 (s, 1H), 4.25-4.09 (m, 2H), 3.66-3.61 (m, 4H), 2.91-2.80 (m, 3H), 2.76-2.69 (m, 2H), 2.64-2.61 (m, 1H), 2.44-2.33 (m, 4H), 2.12-2.08 (m, 1H), 1.94-1.85 (m, 8H), 1.80-1.66 (m, 3H), 1.62-1.54 (m, 3H), 1.39-1.24 (m, 16H), 0.94-0.87 (m, 1H), 0.84 (s, 3H).

### Example-11: Preparation of (7R,8R,9S,13S,14S,17S)-17-methoxy-13-methyl-7-(9-((4,4,5,5,5-pentafluoropentyl)sulfinyl)nonyl)-7,8,9,11,12,13,14,15,16,17-decahydro-6H-cyclopenta[a]phenanthren-3-yl[1,4′-bipiperidine]-1′-carboxylate

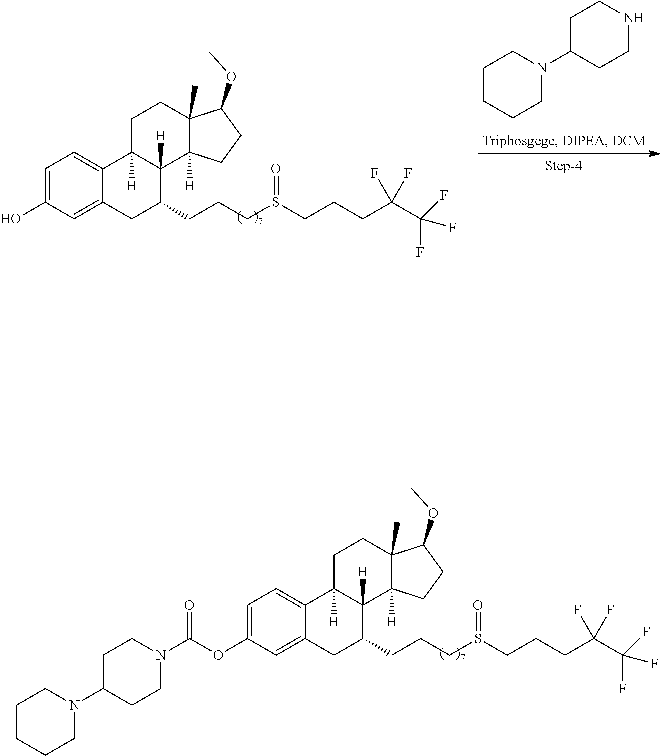

To a stirred solution of (7R,8R,9S,13S,14S,17S)-17-methoxy-13-methyl-7-(9-((4,4,5,5,5-pentafluoropentyl)sulfinyl)nonyl)-7,8,9,11,12,13,14,15,16,17-decahydro-6H-cyclopenta[a]phenanthren-3-ol (0.300 g) in dichloromethane (10 mL) were added DIPEA (0.092 g) and triphosgene (0.071 g) at 0° C. temperature under nitrogen atmosphere and the resulting mixture was stirred at 0° C. temperature for 10 min. After that, 1,4′-bipiperidine (0.121 g) was added to above mixture and reaction mass was stirred for 4 h at room temperature. After confirmation of reaction completion by TLC, the mixture was diluted with water (10 mL), extracted with dichloromethane (3×40 mL), washed with water (2×20 mL), dried over anhydrous sodium sulfate and concentrated under reduced pressure to obtain the crude material. The crude was purified by prep HPLC using trifluoracetic acid as a modifier to afford 0.118 g of (7R,8R,9S,13S,14S,17S)-17-methoxy-13-methyl-7-(9-((4,4,5,5,5-pentafluoropentyl)sulfinyl)nonyl)-7,8,9,11,12,13,14,15,16,17-decahydro-6H-cyclopenta[a]phenanthren-3-yl[1,4′-bipiperidine]-1′-carboxylate mono-trifluoroacetate as a white solid.

LCMS purity: 98.86%

¹H-NMR (DMSO-d6): δ 9.08 (br s, 1H), 7.28 (d, 1H), 6.84 (d, 1H), 6.79 (s, 1H), 4.24-4.16 (m, 2H), 3.43-3.29 (m, 5H), 3.26 (s, 3H), 2.98-2.61 (m, 10H), 2.39-2.33 (m, 3H), 2.07-1.84 (m, 8H), 1.72-1.52 (m, 9H), 1.39-1.15 (m, 19H), 0.90-0.85 (m, 1H), 0.72 (s, 3H).

### Example-12: Preparation of (7R,8R,9S,13S,14S,17S)-17-methoxy-13-methyl-7-(9-((4,4,5,5,5-pentafluoropentyl)sulfinyl)nonyl)-7,8,9,11,12,13,14,15,16,17-decahydro-6H-cyclopenta[a]phenanthren-3-yl 4-morpholinopiperidine-1-carboxylate

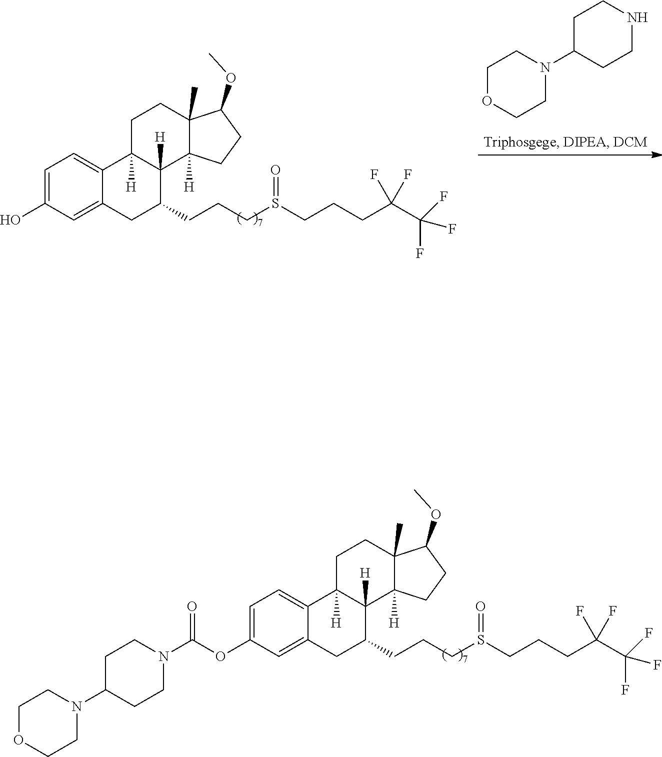

To a stirred solution of (7R,8R,9S,13S,14S,17S)-17-methoxy-13-methyl-7-(9-((4,4,5,5,5-pentafluoropentyl)sulfinyl)nonyl)-7,8,9,11,12,13,14,15,16,17-decahydro-6H-cyclopenta[a]phenanthren-3-ol (0.400 g) in dichloromethane (10 mL) were added DIPEA (0.092 g) and triphosgene (0.094 g) at 0° C. and resulting reaction mixture stirred at 0° C. for 10 min. After that, 4-(piperidin-4-yl)morpholine (0.164 g) was added and reaction mass was stirred for 4 h at room temperature. After completion of the reaction on TLC, the mixture was diluted with water (10 mL) followed by extraction with dichloromethane (3×40 mL). Collected organic layer was dried over anhydrous sodium sulfate and concentrated under reduced pressure to obtain the crude material. The crude was purified by preparative HPLC using trifluoroacetic acid as a modifier to afford 0.113 g of (7R,8R,9S,13S,14S,17S)-17-methoxy-13-methyl-7-(9-((4,4,5,5,5-pentafluoropentyl)sulfinyl)nonyl)-7,8,9,11,12,13,14,15,16,17-decahydro-6H-cyclopenta[a]phenanthren-3-yl 4-morpholinopiperidine-1-carboxylate mono-trifluoroacetate as a white solid.

LCMS purity: 97.76%

¹H-NMR (DMSO-d6): δ 9.72 (br s, 1H), 7.28 (d, 1H), 6.85-6.82 (dd, 1H), 6.78 (d, 1H), 4.23-3.90 (m, 4H), 3.70-3.33 (m, 3H), 3.28 (t, 1H), 3.23 (s, 3H), 3.14-2.87 (m, 3H), 2.85-2.63 (m, 8H), 2.24-2.27 (m, 3H), 2.13-1.86 (m, 6H), 1.72-1.52 (m, 7H), 1.42-1.14 (m, 19H), 0.90-0.87 (m, 1H), 0.72 (s, 3H).

### Example-13: Preparation of (7R,8R,9S,13S,14S,17S)-17-methoxy-13-methyl-7-(9-((4,4,5,5,5-pentafluoropentyl)sulfinyl)nonyl)-7,8,9,11,12,13,14,15,16,17-decahydro-6H-cyclopenta[a]phenanthren-3-yl 4-(pyrrolidin-1-yl)piperidine-1-carboxylate

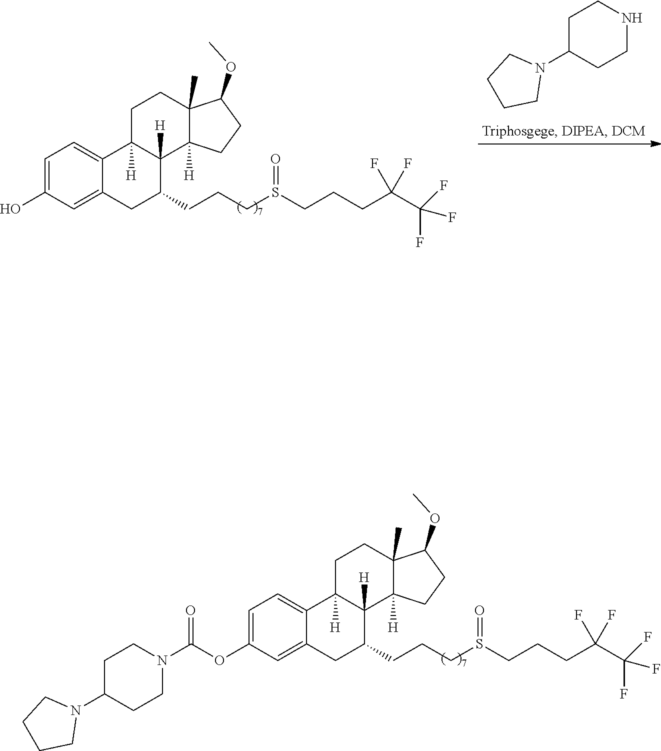

To a stirred solution of (7R,8R,9S,13S,14S,17S)-17-methoxy-13-methyl-7-(9-((4,4,5,5,5-pentafluoropentyl)sulfinyl)nonyl)-7,8,9,11,12,13,14,15,16,17-decahydro-6H-cyclopenta[a]phenanthren-3-ol (0.4 g) in dichloromethane (10 mL) were added DIPEA (0.092 g) and triphosgene (0.094 g) at 0° C. and resulting reaction mixture was stirred at 0° C. for 10 min. After that, 4-(pyrrolidin-1-yl)piperidine (0.164 g) was added to above solution and reaction continued stirred for 4 h at room temperature. After confirmation of reaction completion by TLC, the mixture was diluted with water (10 mL). The reaction mass was extracted with dichloromethane (3×40 mL) followed by washings with water (2×100 mL). Collected organic layer was dried anhydrous sodium sulfate and concentrated under reduced pressure to obtain the crude material. The crude was purified by preparative HPLC using trifluoroacetic acid as a modifier to afford 0.155 g of (7R,8R,9S,13S,14S,17S)-17-methoxy-13-methyl-7-(9-((4,4,5,5,5-pentafluoropentyl)sulfinyl)nonyl)-7,8,9,11,12,13,14,15,16,17-decahydro-6H-cyclopenta[a]phenanthren-3-yl 4-(pyrrolidin-1-yl)piperidine-1-carboxylate mono-trifluoroacetate as a white semi solid.

LCMS purity: 97.74%

¹H-NMR (DMSO-d6): δ 9.56 (br s, 1H), 7.27 (d, 1H), 6.82 (d, 1H), 6.76 (s, 1H), 4.19-4.09 (m, 2H), 3.51-3.27 (m, 3H), 3.24 (s, 3H), 3.08-3.01 (m, 3H), 2.93-2.70 (m, 6H), 2.29-2.09 (m, 4H), 2.01-1.83 (m, 10H), 1.69-1.43 (m, 7H), 1.37-1.14 (m, 19H), 0.85-0.87 (m, 1H), 0.72 (s, 3H).

### Example-14: Preparation of (7R,8R,9S,13S,14S,17S)-17-methoxy-13-methyl-7-(9-((4,4,5,5,5-pentafluoropentyl)sulfinyl)nonyl)-7,8,9,11,12,13,14,15,16,17-decahydro-6H-cyclopenta[a]phenanthren-3-ol

### Step-1: Preparation of (7R,8R,9S,13S,14S,17S)-3-(benzyloxy)-17-methoxy-13-methyl-7-(9-((4,4,5,5,5-pentafluoropentyl)sulfinyl)nonyl)-7,8,9,11,12,13,14,15,16,17-decahydro-6H-cyclopenta[a]phenanthrene

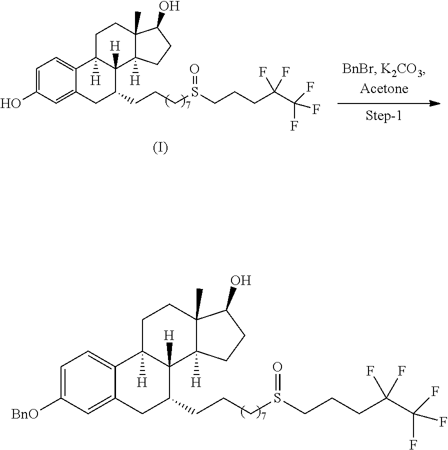

To a stirred solution of (7R,8R,9S,13S,14S,17S)-13-methyl-7-(9-((4,4,5,5,5-pentafluoropentyl)sulfinyl)nonyl)-7,8,9,11,12,13,14,15,16,17-decahydro-6H-cyclopenta[a]phenanthrene-3,17-diol (30 g) in acetone (300 mL) were added potassium carbonate (20.49 g) and benzyl bromide (15.23 g) at 0° C. The resulting solution was stirred at 60° C. for 16 h. After confirmation of reaction completion by TLC, the mixture was diluted with ethyl acetate (20 mL). The organic compound was extracted with ethyl acetate (3×70 mL), washed with water (2×200 mL), dried the organic layer over anhydrous sodium sulfate and concentrated under reduced pressure to obtain a crude material. The crude was purified by flash chromatography (23% ethyl acetate: n-heptane) afforded 17 g of (7R,8R,9S,13S,14S,17S)-3-(benzyloxy)-17-methoxy-13-methyl-7-(9-((4,4,5,5,5-pentafluoropentyl)sulfinyl)nonyl)-7,8,9,11,12,13,14,15,16,17-decahydro-6H-cyclopenta[a]phenanthrene as a colourless liquid.

### Step-2: Preparation of (7R,8R,9S,13S,14S,17S)-3-(benzyloxy)-17-methoxy-13-methyl-7-(9-((4,4,5,5,5-pentafluoropentyl)sulfinyl)nonyl)-7,8,9,11,12,13,14,15,16,17-decahydro-6H-cyclopenta[a]phenanthrene

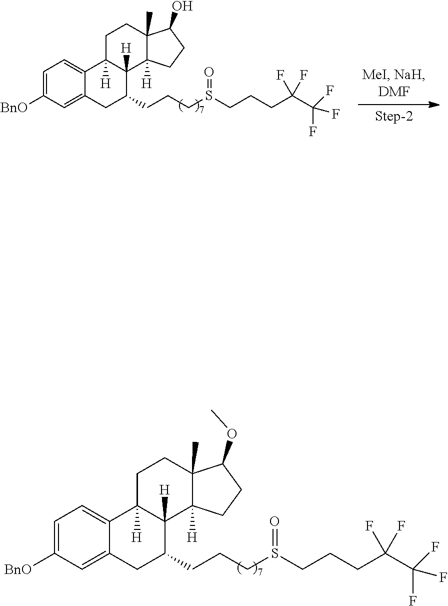

To a stirred solution of (7R,8R,9S,13S,14S,17S)-3-(benzyloxy)-17-methoxy-13-methyl-7-(9-((4,4,5,5,5-pentafluoropentyl)sulfinyl)nonyl)-7,8,9,11,12,13,14,15,16,17-decahydro-6H-cyclopenta[a]phenanthrene (8 g) in N,N-dimethylformamide (20 mL) was added sodium hydride (0.827 g) and resulting reaction mixture is stirred for 30 min. After that, methyl iodide (8.09 g) was added to above mixture and reaction mass was stirred at room temperature for 16 h. After confirmation of reaction completion by TLC, reaction mixture was quenched with cold water (300 mL). The organic compound was extracted with ethyl acetate (3×250 mL), washed with water (3×500 mL), dried the organic layer over anhydrous sodium sulfate and concentrated under reduced pressure to obtain a crude material. The crude was purified by flash chromatography (28% ethyl acetate in n-heptane) afforded 4.5 g of (7R,8R,9S,13S,14S,17S)-3-(benzyloxy)-17-methoxy-13-methyl-7-(9-((4,4,5,5,5-pentafluoropentyl)sulfinyl)nonyl)-7,8,9,11,12,13,14,15,16,17-decahydro-6H-cyclopenta[a]phenanthrene as colourless oil.

### Step-3: Preparation of (7R,8R,9S,13S,14S,17S)-17-methoxy-13-methyl-7-(9-((4,4,5,5,5-pentafluoropentyl)sulfinyl)nonyl)-7,8,9,11,12,13,14,15,16,17-decahydro-6H-cyclopenta[a]phenanthren-3-ol

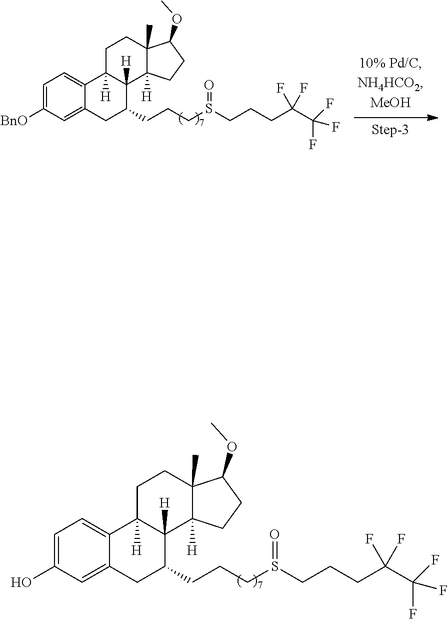

To a stirred solution of (7R,8R,9S,13S,14S,17S)-17-methoxy-13-methyl-7-(9-((4,4,5,5,5-pentafluoropentyl)sulfinyl)nonyl)-7,8,9,11,12,13,14,15,16,17-decahydro-6H-cyclopenta[a]phenanthren-3-ol (4.5 g) in methanol (50 mL) were added 10% Pd/C (1.5 g) and ammonium formate (3.1 g). Resulting mixture was stirred at room temperature for 5 h. After confirmation of reaction completion by TLC, reaction mixture was filtered through a celite pad and washed thoroughly with methanol. The filtrate obtained was concentrated under reduced pressure to obtain the crude material. The crude was enriched by flash chromatography (42% ethyl acetate in n-heptane, 78%, 1.8 g). Crude compound was purified by preparative HPLC (TFA method) afforded 0.086 g of (7R,8R,9S,13S,14S,17S)-17-methoxy-13-methyl-7-(9-((4,4,5,5,5-pentafluoropentyl)sulfinyl)nonyl)-7,8,9,11,12,13,14,15,16,17-decahydro-6H-cyclopenta[a]phenanthren-3-ol as a white solid.

LCMS purity: 98% (Peak-1, RT 6.16 min: 52.45% and Peak-2, RT: 6.24 min: 45.83%)

1H-NMR (DMSO-d6): δ 7.03 (d, 1H), 6.49 (dd, 1H), 6.40 (d, 1H), 3.28-3.26 (m, 2H), 3.25 (s, 3H), 2.88-2.71 (m, 4H), 2.64-2.58 (m, 2H), 2.42-2.16 (m, 4H), 1.93-1.83 (m, 4H), 1.64-1.56 (m, 3H), 1.49-1.44 (m, 2H), 1.33-1.13 (m, 17H), 0.90-0.81 (m, 1H), 0.67 (s, 3H).

### Example-15: Preparation of (7R,8R,9S,13S,14S,17S)-17-methoxy-13-methyl-7-(9-((4,4,5,5,5-pentafluoropentyl)sulfinyl)nonyl)-7,8,9,11,12,13,14,15,16,17-decahydro-6H-cyclopenta[a]phenanthren-3-yl (1,3-dihydroxy-2-(hydroxy methyl)propan-2-yl)carbamate

### Step-1: Preparation of (7R,8R,9S,13S,14S,17S)-17-methoxy-13-methyl-7-(9-((4,4,5,5,5-pentafluoropentyl)sulfinyl)nonyl)-7,8,9,11,12,13,14,15,16,17-decahydro-6H-cyclopenta[a]phenanthren-3-yl (4-nitrophenyl) carbonate

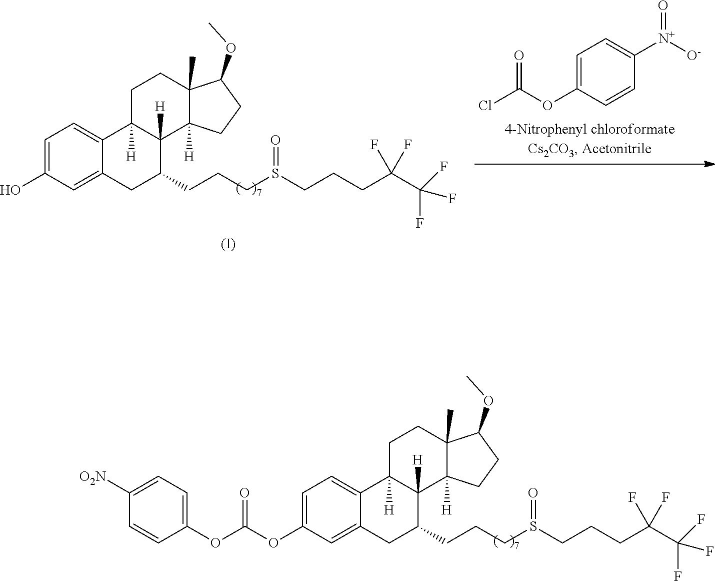

To a stirred solution of (7R,8R,9S,13S,14S,17S)-17-methoxy-13-methyl-7-(9-((4,4,5,5,5-pentafluoropentyl)sulfinyl)nonyl)-7,8,9,11,12,13,14,15,16,17-decahydro-6H-cyclopenta[a]phenanthren-3-ol (0.3 g) in acetonitrile (3 mL) were added caesium carbonate (0.47 g) and 4-nitrophenyl chloroformate (0.15 g) at room temperature and stirred the reaction mixture for 15 min at the same temperature. After confirmation of reaction completion by TLC, the reaction mass was diluted with water (15 mL) and extracted with ethyl acetate (2×25 mL). The combined organic layer was dried over anhydrous sodium sulfate and concentrated the organic layer under reduced pressure to give (7R,8R,9S,13S,14S,17S)-17-methoxy-13-methyl-7-(9-((4,4,5,5,5-pentafluoropentyl)sulfinyl)nonyl)-7,8,9,11,12,13,14,15,16,17-decahydro-6H-cyclopenta[a]phenanthren-3-yl (4-nitrophenyl) carbonate (0.38 g crude), which was used for next step without further purification.

### Step-2: Preparation of (7R,8R,9S,13S,14S,17S)-17-methoxy-13-methyl-7-(9-((4,4,5,5,5-pentafluoropentyl)sulfinyl)nonyl)-7,8,9,11,12,13,14,15,16,17-decahydro-6H-cyclopenta[a]phenanthren-3-yl (1,3-dihydroxy-2-(hydroxymethyl)propan-2-yl)carbamate

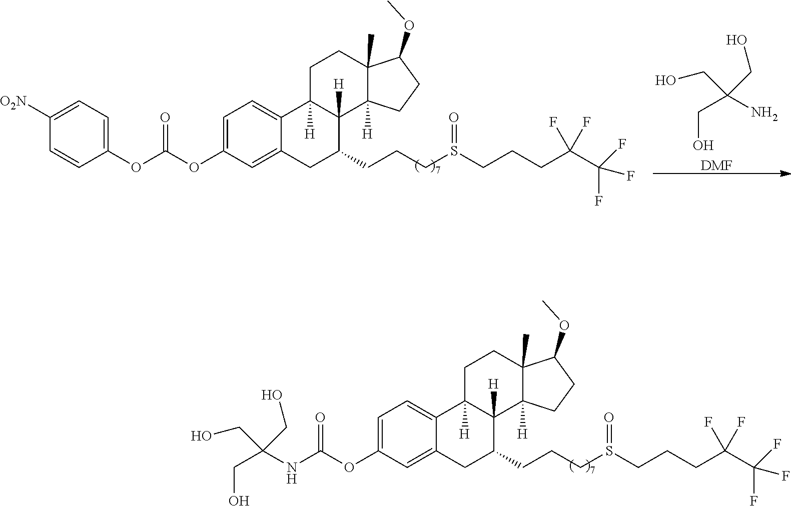

To a stirred solution of (7R,8R,9S,13S,14S,17S)-17-methoxy-13-methyl-7-(9-((4,4,5,5,5-pentafluoropentyl)sulfinyl)nonyl)-7,8,9,11,12,13,14,15,16,17-decahydro-6H-cyclopenta[a]phenanthren-3-yl (4-nitrophenyl) carbonate (0.380 g) in DMF (4 mL) was added 2-amino-2-(hydroxymethyl)propane-1,3-diol (0.175 g). Resulting mixture was stirred at room temperature for 2 h. After confirmation of reaction completion by TLC, the mixture was diluted with water (2 mL). The organic compound was extracted with ethyl acetate (3×5 mL), washed with water (2×20 mL), dried the organic layer over anhydrous sodium sulfate and concentrated under reduced pressure to obtain the crude material. The crude was purified by preparative HPLC using trifluoroacetic acid as a buffer to afford 0.040 g of 7R,8R,9S,13S,14S,17S)-17-methoxy-13-methyl-7-(9-((4,4,5,5,5-pentafluoro pentyl)sulfinyl)nonyl)-7,8,9,11,12,13,14,15,16,17-decahydro-6H-cyclopenta[a]phenanthren-3-yl (1,3-dihydroxy-2-(hydroxymethyl)propan-2-yl)carbamate as a white solid.

LCMS purity: 99.71%

¹H-NMR (DMSO-d6): δ 7.25 (d, 1H), 6.82 (dd, 1H), 6.78 (d, 1H), 6.68 (br s, 1H), 4.53-4.50 (t, 3H), 3.56 (d, 6H), 3.30-3.28 (m, 2H), 3.26 (s, 3H), 2.88-2.61 (m, 6H), 2.43-2.23 (m, 3H), 2.06-1.88 (m, 4H), 1.72-1.52 (m, 5H), 1.39-1.15 (m, 17H), 0.91-0.89 (m, 1H), 0.72 (s, 3H).

### Example-16: Preparation of ((7R,8R,9S,13S,14S)-13-methyl-17-oxo-7-(9-((4,4,5,5,5-pentafluoropentyl)sulfinyl)nonyl)-7,8,9,11,12,13,14,15,16,17-decahydro-6H-cyclopenta[a]phenanthren-3-yl)boronic acid

### Step-1: Preparation of (7R,8R,9S,13S,14S,17S)-13-methyl-7-(9-((4,4,5,5,5-pentafluoropentyl)thio)nonyl)-3-(((trifluoromethyl)sulfonyl)oxy)-7,8,9,11,12,13,14,15,16,17-decahydro-6H-cyclopenta[a]phenanthren-17-yl acetate

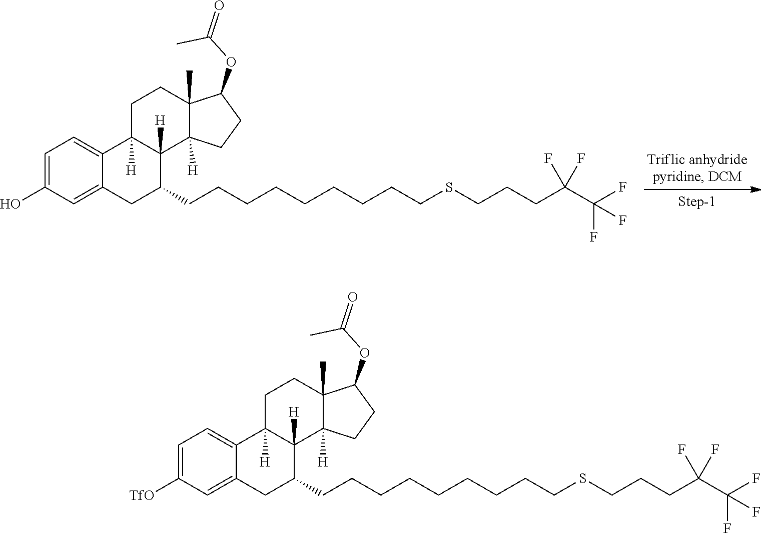

To a stirred solution of (7R,8R,9S,13S,14S,17S)-3-hydroxy-13-methyl-7-(9-((4,4,5,5,5-pentafluoropentyl)thio)nonyl)-7,8,9,11,12,13,14,15,16,17-decahydro-6H-cyclopenta[a]phenanthren-17-yl acetate (8 g) in dichloromethane (80 mL) were added pyridine (18 mL) and trifluoromethanesulfonic anhydride (5.67 g) at 0° C. temperature and resultant reaction mixture was stirred at room temperature for 16 h. After completion of the reaction (monitored by TLC), the mixture was diluted with water and extracted with dichloromethane. Collected organics, dried the organic layer over anhydrous sodium sulfate and concentrated under reduced pressure to obtain the crude. The crude material was purified by flash chromatography (10% ethyl acetate in n-heptane) to afford (7R,8R,9S,13S,14S,17S)-13-methyl-7-(9-((4,4,5,5,5-pentafluoropentyl)thio)nonyl)-3-(((trifluoromethyl)sulfonyl)oxy)-7,8,9,11,12,13,14,15,16,17-decahydro-6H-cyclopenta[a]phenanthren-17-yl acetate (6.88 g, 71.66%) as a pale yellow semi solid.

¹H-NMR (400 MHz, DMSO-d₆): δ 7.46 (d, J=8.40 Hz, 1H), 7.17-7.19 (m, 2H), 4.63 (t, J=8.40 Hz, 1H), 2.91-2.79 (m, 2H), 2.55-2.57 (m, 2H), 2.33-2.24 (m, 3H), 2.12-2.11 (m, 1H), 2.00 (s, 2H), 1.74-1.73 (m, 3H), 1.65-1.68 (m, 1H), 1.57-1.48 (m, 4H), 1.36-1.22 (m, 19H), 0.85-0.79 (m, 8H).

### Step-2: Preparation of (7R,8R,9S,13S,14S,17S)-13-methyl-7-(9-((4,4,5,5,5-pentafluoropentyl)thio)nonyl)-3-(4,4,5,5-tetramethyl-1,3,2-dioxaborolan-2-yl)-7,8,9,11,12,13,14,15,16,17-decahydro-6H-cyclopenta[a]phenanthren-17-yl acetate

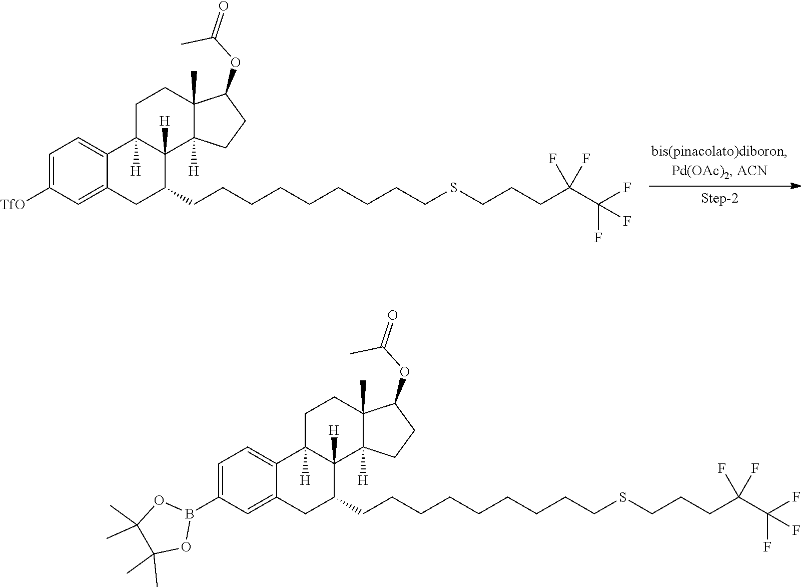

To a stirred solution of (7R,8R,9S,13S,14S,17S)-13-methyl-7-(9-((4,4,5,5,5-pentafluoropentyl)thio)nonyl)-3-(((trifluoromethyl)sulfonyl)oxy)-7,8,9,11,12,13,14,15,16,17-decahydro-6H-cyclopenta[a]phenanthren-17-yl acetate (6.8 g) in 1,4-dioxane under nitrogen atmosphere (60 mL) were added 4,4,4′,4′,5,5,5′,5′-octamethyl-2,2′-bi(1,3,2-dioxaborolane) (3.4 g) and potassium acetate (1.3 g). This reaction mixture was purged with nitrogen gas for 10 min and then added Palladium (II) acetate (0.398 g) followed by addition of tricyclohexylphosphine (1.2 g). Resultant reaction mixture was stirred at 80° C. for 16 h. After completion of the reaction (monitored by TLC), the reaction mixture was concentrated under reduced pressure to obtain crude. The crude was purified by Combi-flash chromatography to obtain (7R,8R,9S,13S,14S,17S)-13-methyl-7-(9-((4,4,5,5,5-pentafluoropentyl)thio)nonyl)-3-(4,4,5,5-tetramethyl-1,3,2-dioxaborolan-2-yl)-7,8,9,11,12,13,14,15,16,17-decahydro-6H-cyclopenta[a]phenanthren-17-yl acetate (5.5 g, 83%) as a colorless semi solid.

¹H-NMR (400 MHz, DMSO-d₆): δ 7.58 (d, J=8.00 Hz, 1H), 7.54 (s, 1H), 7.30 (d, J=8.00 Hz, 1H), 4.70 (t, J=8.80 Hz, 1H), 2.93-2.80 (m, 2H), 2.58 (t, J=7.20 Hz, 2H), 2.50 (t, J=7.60 Hz, 2H), 2.44-2.34 (m, 2H), 2.26-2.16 (m, 3H), 2.16 (s, 3H), 1.92-1.84 (m, 3H), 1.77-1.75 (m, 1H), 1.69-1.64 (m, 2H), 1.58-1.60 (m, 1H), 1.49-1.41 (m, 3H), 1.39-1.30 (m, 15H), 1.27-1.17 (m, 10H), 1.19-1.17 (m, 2H), 0.99-0.97 (m, 1H), 0.88-0.06 (m, 1H), 0.80 (s, 3H).

### Step-3: Preparation of (7R,8R,9S,13S,14S,17S)-13-methyl-7-(9-((4,4,5,5,5-pentafluoropentyl)sulfinyl)nonyl)-3-(4,4,5,5-tetramethyl-1,3,2-dioxaborolan-2-yl)-7,8,9,11,12,13,14,15,16,17-decahydro-6H-cyclopenta[a]phenanthren-17-yl acetate (4-(2-(trifluoromethyl)morpholino)phenyl)carbamate

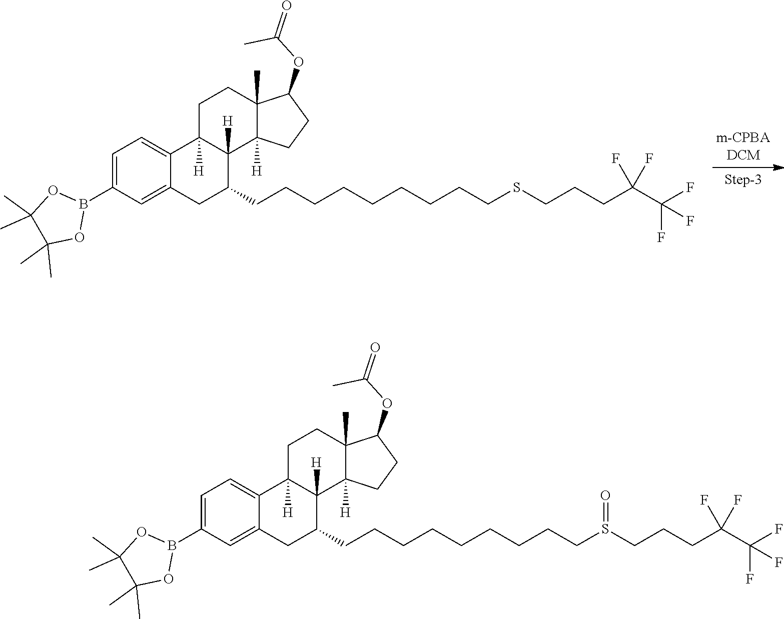

To a stirred solution of (7R,8R,9S,13S,14S,17S)-13-methyl-7-(9-((4,4,5,5,5-pentafluoropentyl)thio)nonyl)-3-(4,4,5,5-tetramethyl-1,3,2-dioxaborolan-2-yl)-7,8,9,11,12,13,14,15,16,17-decahydro-6H-cyclopenta[a]phenanthren-17-yl acetate (5.5 g) in dichloromethane (20 mL) wase added m-chloroperbenzoic acid (1.27 g) at 0° C. temperature and the mixture was stirred at room temperature for 2.5 h. After completion of the reaction (monitored by TLC), reaction mixture was quenched with saturated solution sodium carbonate and extracted with dichloromethane (3×200 mL), dried the organic layer over anhydrous sodium sulfate and concentrated under reduced pressure to obtain the crude material. The crude was purified by washings of n-pentane to obtain (7R,8R,9S,13S,14S,17S)-13-methyl-7-(9-((4,4,5,5,5-pentafluoropentyl)sulfinyl)nonyl)-3-(4,4,5,5-tetramethyl-1,3,2-dioxaborolan-2-yl)-7,8,9,11,12,13,14,15,16,17-decahydro-6H-cyclopenta[a]phenanthren-17-yl acetate (4.0 g, 71%) as a sticky solid.

LCMS: 94.30% (m/z: 759.52, [M+1]⁺, 3.29 min (4 min run), 214 nm).

### Step-4: Preparation of (7R,8R,9S,13S,14S,17S)-13-methyl-7-(9-((4,4,5,5,5-pentafluoropentyl)sulfinyl)nonyl)-3-(4,4,5,5-tetramethyl-1,3,2-dioxaborolan-2-yl)-7,8,9,11,12,13,14,15,16,17-decahydro-6H-cyclopenta[a]phenanthren-17-ol

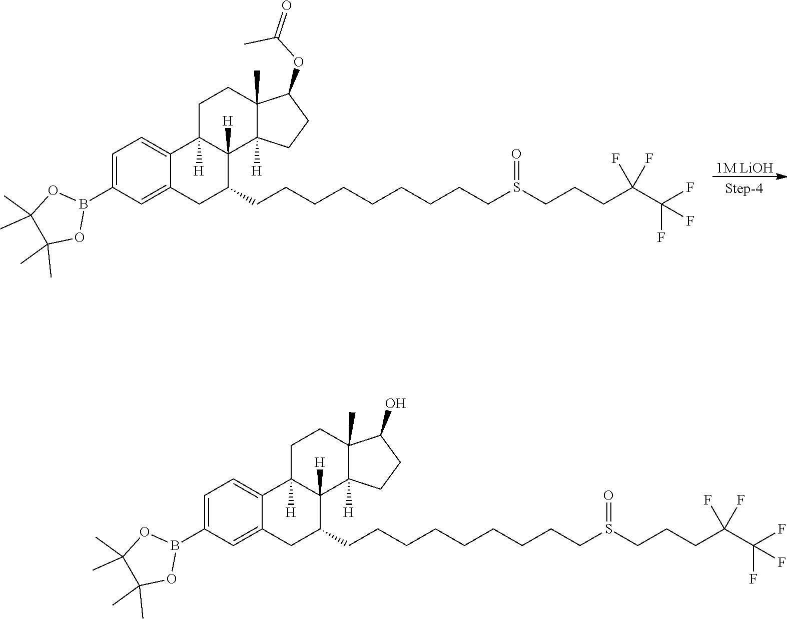

To a stirred solution of (7R,8R,9S,13S,14S,17S)-13-methyl-7-(9-((4,4,5,5,5-pentafluoropentyl)sulfinyl)nonyl)-3-(4,4,5,5-tetramethyl-1,3,2-dioxaborolan-2-yl)-7,8,9,11,12,13,14,15,16,17-decahydro-6H-cyclopenta[a]phenanthren-17-yl acetate (0.5 g) in methanol (7.5 mL) was added 1M lithium hydroxide (3 mL) at 0° C. and resulting solution was stirred at room temperature for 16 h. After completion of the reaction (monitored by TLC), the reaction mixture was quenched with saturated NH₄Cl solution (2 mL). The organic compound was extracted with dichloromethane (3×20 mL), dried the organic layer over anhydrous sodium sulfate and concentrated under reduced pressure to obtain (7R,8R,9S,13S,14S,17S)-13-methyl-7-(9-((4,4,5,5,5-pentafluoropentyl)sulfinyl)nonyl)-3-(4,4,5,5-tetramethyl-1,3,2-dioxaborolan-2-yl)-7,8,9,11,12,13,14,15,16,17-decahydro-6H-cyclopenta[a]phenanthren-17-ol (5, 0.380 g, crude) as a semi solid, which was used for next step without any purification.

LCMS: 68.72% (m/z: 638.34 [M+1]⁺, 4.36 min (6 min run), 214 nm).

### Step-5: Preparation of (7R,8R,9S,13S,14S)-13-methyl-7-(9-((4,4,5,5,5-pentafluoropentyl)sulfinyl)nonyl)-3-(4,4,5,5-tetramethyl-1,3,2-dioxaborolan-2-yl)-6,7,8,9,11,12,13,14,15,16-decahydro-17H-cyclopenta[a]phenanthren-17-one

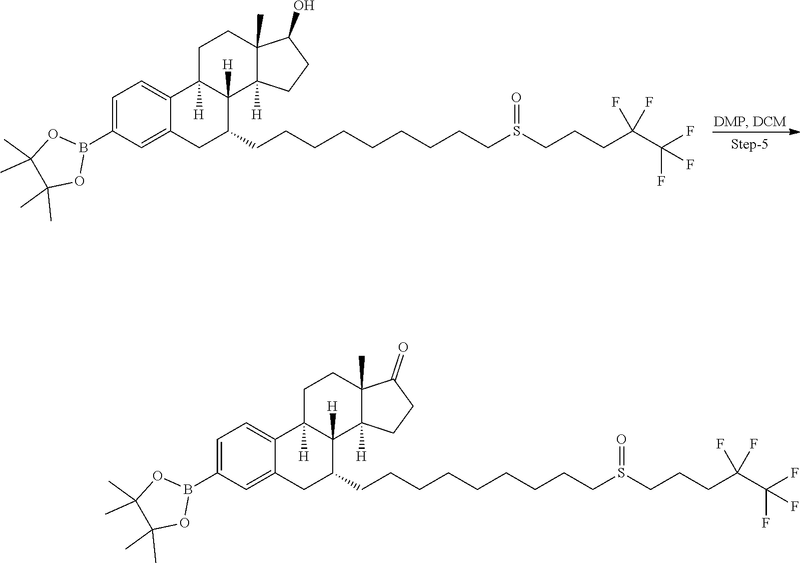

To a stirred solution of as (7R,8R,9S,13S,14S,17S)-13-methyl-7-(9-((4,4,5,5,5-pentafluoropentyl)sulfinyl)nonyl)-3-(4,4,5,5-tetramethyl-1,3,2-dioxaborolan-2-yl)-7,8,9,11,12,13,14,15,16,17-decahydro-6H-cyclopenta[a]phenanthren-17-ol (0.380 g) in dichloromethane (8 mL) was added Dess-Martin periodinane (0.270 g) and resulting mixture was stirred at room temperature for 8 h. After completion of the reaction (monitored by TLC), the reaction mixture was quenched with water (5 mL). The reaction mass was extracted with dichloromethane (3×50 mL), washed with water (2×200 mL), dried the organic layer over anhydrous sodium sulfate and concentrated under reduced pressure to obtain (7R,8R,9S,13S,14S)-13-methyl-7-(9-((4,4,5,5,5-pentafluoropentyl)sulfinyl)nonyl)-3-(4,4,5,5-tetramethyl-1,3,2-dioxaborolan-2-yl)-6,7,8,9,11,12,13,14,15,16-decahydro-17H-cyclopenta[a]phenanthren-17-one (0.390 g, crude) as yellowish sticky liquid, which was used for next step without any purification.

LCMS: 40.65% (m/z: 715.33 [M+1]⁺, 2.96 min (4 min run), 214 nm).

### Step-6: Preparation of ((7R,8R,9S,13S,14S)-13-methyl-17-oxo-7-(9-((4,4,5,5,5-pentafluoropentyl)sulfinyl)nonyl)-7,8,9,11,12,13,14,15,16,17-decahydro-6H-cyclopenta[a]phenanthren-3-yl)boronic acid

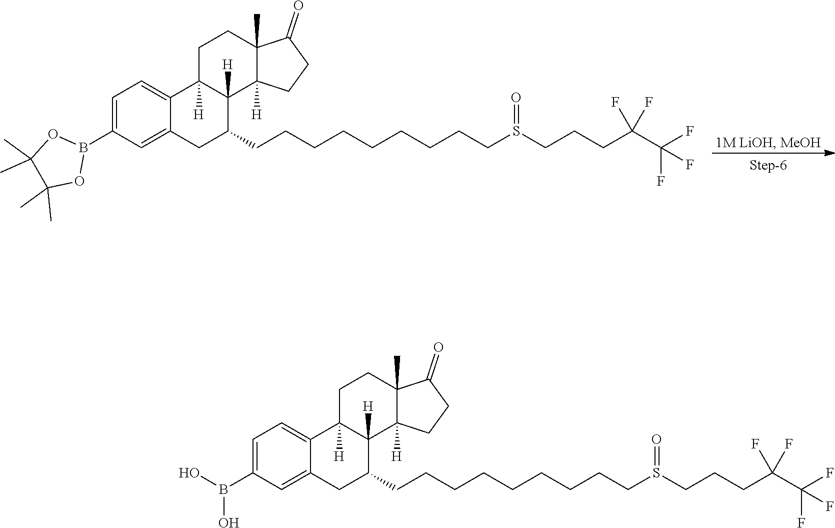

To a stirred solution of (7R,8R,9S,13S,14S)-13-methyl-7-(9-((4,4,5,5,5-pentafluoropentyl)sulfinyl)nonyl)-3-(4,4,5,5-tetramethyl-1,3,2-dioxaborolan-2-yl)-6,7,8,9,11,12,13,14,15,16-decahydro-17H-cyclopenta[a]phenanthren-17-one (0.390 g) in methanol (4.5 mL) was added 1M lithium hydroxide (2.5 mL) at 0° C. and resulting solution was stirred at room temperature for 24 h. After completion of the reaction (monitored by TLC), the reaction mixture was quenched with saturated ammonium chloride solution (3 mL). The organic compound was extracted with DCM (3×50 mL), washed with water (2×100 mL), dried the organic layer over anhydrous sodium sulfate and concentrated under reduced pressure to obtain the crude. Crude was purified by preparative HPLC using trifluoroacetic acid buffer to afford ((7R,8R,9S,13S,14S)-13-methyl-17-oxo-7-(9-((4,4,5,5,5-pentafluoropentyl)sulfinyl)nonyl)-7,8,9,11,12,13,14,15,16,17-decahydro-6H-cyclopenta[a]phenanthren-3-yl)boronic acid (0.038 g, 11%) as a white solid.

LCMS: 99.24% (m/z: 633.40, [M+1]⁺, 6.82 min (10 min run), 214 nm).

¹H-NMR (400 MHz, DMSO-d6-D₂O): δ 7.49 (d, J=7.60 Hz, 1H), 7.45 (s, 1H), 7.23 (d, J=7.60 Hz, 1H), 2.89-2.79 (m, 3H), 2.76-2.60 (m, 5H), 2.45-2.28 (m, 4H), 2.10-2.03 (m, 1H), 1.91-1.83 (m, 4H), 1.77-1.62 (m, 3H), 1.60-1.53 (m, 3H), 1.37-1.32 (m, 6H), 1.21-111.00 (m, 8H), 0.88-0.91 (m, 1H), 0.81 (s, 3H).

### Example-17: Preparation of (7R,8R,9S,13S,14S)-13-methyl-17-oxo-7-(9-((4,4,5,5,5-pentafluoropentyl)sulfinyl)nonyl)-7,8,9,11,12,13,14,15,16,17-decahydro-6H-cyclopenta[a]phenanthren-3-yl 2-(trifluoromethyl)morpholine-4-carboxylate (compound 108)

### Step-1: Preparation of (7R,8R,9S,13S,14S)-3-hydroxy-13-methyl-7-(9-((4,4,5,5,5-pentafluoropentyl)sulfinyl)nonyl)-6,7,8,9,11,12,13,14,15,16-decahydro-17H-cyclopenta[a]phenanthren-17-one

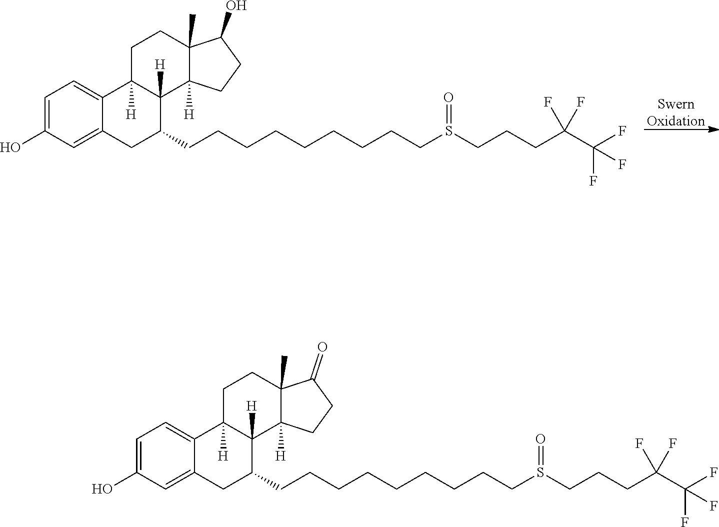

Oxalyl dichloride (0.8 g) was stirred in dry dichloromethane (48 mL) at −70° C. under nitrogen. A solution of dimethyl sulfoxide (1.65 mL) in dry dichloromethane (50 mL) was added drop wisely to above solution over a period of 10-15 min, keeping the internal temperature below −60° C. The mixture was stirred for 5 min, then a solution of fulvestrant (5 g) in dry dichloromethane (50 mL) was added dropwise over a period of 15 min, maintaining the same temperature. After stirring of the mixture at −70° C. for 2 h, N,N′-diisopropylamine (8.3 mL) was added over a period of 5 min and the mixture was stirred at −70° C. for a further 30 min, then allowed to warm to room temperature. Reaction was monitored by TLC (70% ethyl acetate in n-heptane). After completion of the reaction, water (80 mL) and dichloromethane (80 mL) were successively added to the reaction mass and the mixture was stirred for 25 min before the addition of further water (160 mL) and dichloromethane (160 mL). The phases were separated and the organic phase was washed with water (500 mL), then dried over anhydrous sodium sulfate for 16 h and evaporated to afford (7R,8R,9S,13S,14S)-3-hydroxy-13-methyl-7-(9-((4,4,5,5,5-pentafluoropentyl)sulfinyl)nonyl)-6,7,8,9,11,12,13,14,15,16-decahydro-17H-cyclopenta[a]phenanthren-17-one (4.5 g, 91.8%) as an off white solid.

LCMS: 93.93%, (m/z: 605.25) [M+1]⁺, 2.52 min (4 min run), 214 nm).

¹H NMR (400 MHz, DMSO-d₆): δ 9.01 (s, 1H), 7.05 (d, J=8.40 Hz, 1H), 6.51 (d, J=8.80 Hz, 1H), 6.44 (s, 1H), 2.86-2.61 (m, 8H), 2.43-2.25 (m, 6H), 2.11-1.98 (m, 1H), 1.92-1.81 (m, 3H), 1.68-1.35 (m, 6H), 1.33-1.24 (m, 12H), 0.95-0.96 (m, 2H), 0.82 (s, 3H).

### Step-2: Preparation of (7R,8R,9S,13S,14S)-13-methyl-17-oxo-7-(9-((4,4,5,5,5-pentafluoropentyl)sulfinyl)nonyl)-7,8,9,11,12,13,14,15,16,17-decahydro-6H-cyclopenta[a]phenanthren-3-yl 2-(trifluoromethyl)morpholine-4-carboxylate

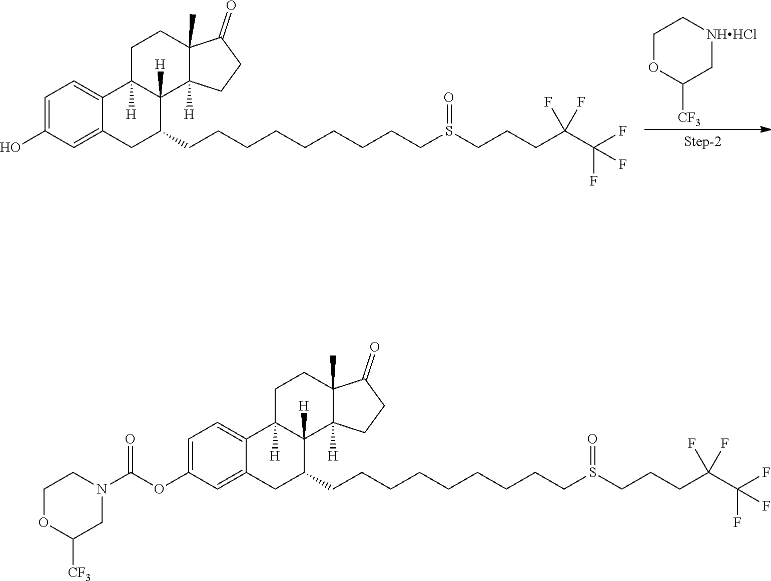

To a stirred solution of (7R,8R,9S,13S,14S)-3-hydroxy-13-methyl-7-(9-((4,4,5,5,5-pentafluoropentyl)sulfinyl)nonyl)-6,7,8,9,11,12,13,14,15,16-decahydro-17H-cyclopenta[a]phenanthren-17-one (0.5 g) in dichloromethane (10 mL) were added DIPEA (0.21 mL) and triphosgene (0.122 g) at 0° C. and resulting mixture was stirred for 10 min at the same temperature. After 10 min, 2-(trifluoromethyl)morpholine hydrochloride (0.237 g) was added in the reaction mixture at 0° C. and the reaction mixture was stirred at room temperature for 16 h. After completion of the reaction (monitored by TLC), the reaction mass was diluted with water (20 mL) and extracted with dichloromethane (3×20 mL). The combined organic layer was dried over anhydrous sodium sulfate and concentrated under reduced pressure. The crude was purified by preparative HPLC using trifluoroacetic acid as a buffer to give (7R,8R,9S,13S,14S)-13-methyl-17-oxo-7-(9-((4,4,5,5,5-pentafluoropentyl)sulfinyl)nonyl)-7,8,9,11,12,13,14,15,16,17-decahydro-6H-cyclopenta[a]phenanthren-3-yl 2-(trifluoromethyl)morpholine-4-carboxylate (0.078 g, 11%) as a pale yellow solid.

LCMS: 97.36%, (m/z: 786.35) [M+1]⁺, 7.27 min (10 min run), 214 nm).

¹H-NMR (400 MHz, DMSO-d₆-D₂O): δ 7.28 (d, J=8.80 Hz, 1H), 6.86 (d, J=8.40 Hz, 1H), 6.83 (s, 1H), 4.30-4.07 (m, 2H), 3.97-3.99 (m, 2H), 3.67-3.65 (m, 1H), 3.25-3.10 (m, 2H), 2.87-2.79 (m, 2H), 2.75-2.69 (m, 2H), 2.67-2.61 (m, 4H), 2.44-2.27 (m, 4H), 2.15-2.02 (m, 1H), 1.93-1.82 (m, 4H), 1.76-1.53 (m, 5H), 1.41-1.29 (m, 6H), 1.29-1.10 (m, 9H), 0.90-0.88 (m, 1H), 0.81 (s, 3H).

### Example-18: Preparation of (7R,8R,9S,13S,14S)-13-methyl-17-oxo-7-(9-((4,4,5,5,5-pentafluoropentyl)sulfinyl)nonyl)-7,8,9,11,12,13,14,15,16,17-decahydro-6H-cyclopenta[a]phenanthren-3-yl (2-(dimethylamino)ethyl)carbamate (compound 115)

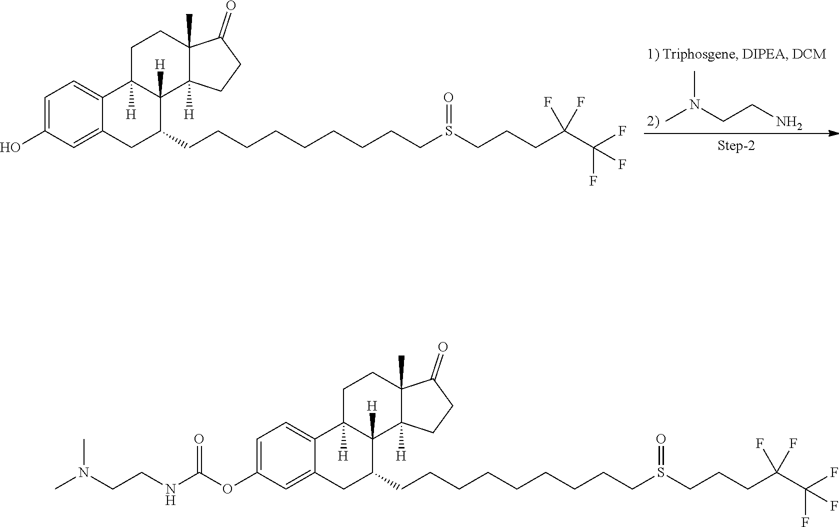

To a stirred solution of (7R,8R,9S,13S,14S)-3-hydroxy-13-methyl-7-(9-((4,4,5,5,5-pentafluoropentyl)sulfinyl)nonyl)-6,7,8,9,11,12,13,14,15,16-decahydro-17H-cyclopenta[a]phenanthren-17-one (1 g, as prepared in example-17, step-1) in dichloromethane (16 mL) were added DIPEA (0.42 mL) and triphosgene (0.24 g) at 0° C. and resultant reaction mixture was stirred for 10 min at the same temperature. After 10 min, N,N′-dimethylethane-1,2-diamine (0.21 g) was added to above reaction mixture at 0° C. and the reaction mixture was stirred at room temperature for 16 h. After completion of the reaction (monitored by TLC), the reaction mass was diluted with water (25 mL) and extracted with dichloromethane (2×25 mL). The combined organic layer was dried over anhydrous sodium sulfate and concentrated under reduced pressure to give 0.8 g crude material.

LCMS: 42.55%, (m/z: 719.15) [M+1]⁺, 3.24 min (4 min run), 214 nm).

### Example-19: Preparation of (7R,8R,9S,13S,14S)-13-methyl-17-oxo-7-(9-((4,4,5,5,5-pentafluoropentyl)sulfinyl)nonyl)-7,8,9,11,12,13,14,15,16,17-decahydro-6H-cyclopenta[a]phenanthren-3-yl 2-(4-methylpiperazin-1-yl)acetate (compound 250)

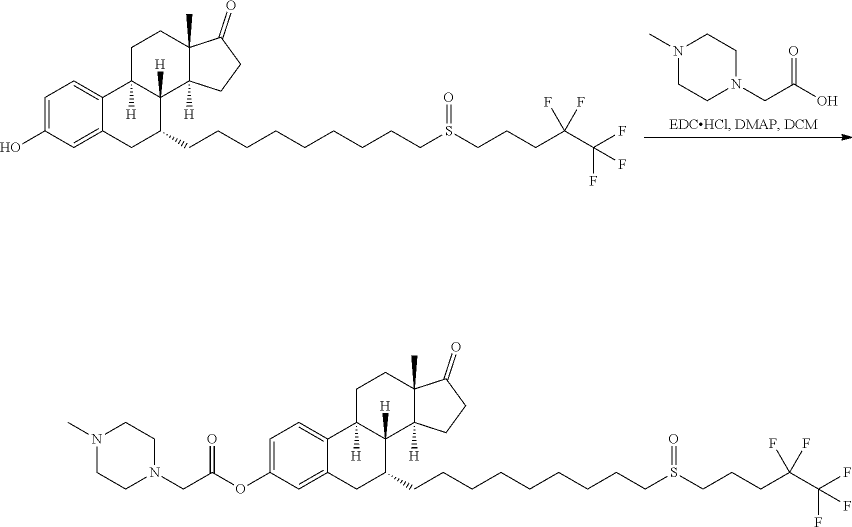

To a stirred solution of 2-(4-methylpiperazin-1-yl)acetic acid (0.16 g) in dichloromethane (8 mL) were added 4-dimethylaminopyridine (0.02 g), 1-(3-dimethylaminopropyl)-3-ethylcarbodiimide hydrochloride (0.20 g) at 0° C. and resultant reaction mixture was stirred at 0° C. for 10 min. After that, (7R,8R,9S,13S,14S)-3-hydroxy-13-methyl-7-(9-((4,4,5,5,5-pentafluoropentyl) sulfinyl)nonyl)-6,7,8,9,11,12,13,14,15,16-decahydro-17H-cyclopenta[a]phenanthren-17-one (0.5 g, as prepared in example-17, step-1) was added and reaction mass was stirred at room temperature for 16 h. After completion of the reaction (monitored by TLC), reaction mass was diluted with ice water (30 mL) and extracted with dichloromethane (3×30 ML). The combined organic layers were washed with water (2×40 mL), dried over anhydrous sodium sulfate and concentrated under reduced pressure to give 0.316 g crude material. The crude material was purified by reverse phase HPLC to furnish (7R,8R,9S,13S,14S)-13-methyl-17-oxo-7-(9-((4,4,5,5,5-pentafluoropentyl) sulfinyl)nonyl)-7,8,9,11,12,13,14,15,16,17-decahydro-6H-cyclopenta[a]phenanthren-3-yl 2-(4-methylpiperazin-1-yl) acetate (0.073 g, 11.8%) as a white color solid.

LCMS: 81.91% (m/z: 744.40, [M+1]⁺, 2.33 min, 214 nm).

### Example-20: Preparation of (7R,8R,9S,13S,14S)-13-methyl-17-oxo-7-(9-((4,4,5,5,5-pentafluoropentyl) sulfinyl)nonyl)-7,8,9,11,12,13,14,15,16,17-decahydro-6H-cyclopenta[a]phenanthren-3-yl 5,7-dihydro-6H-pyrrolo[3,4-b]pyridine-6-carboxylate (compound 117)

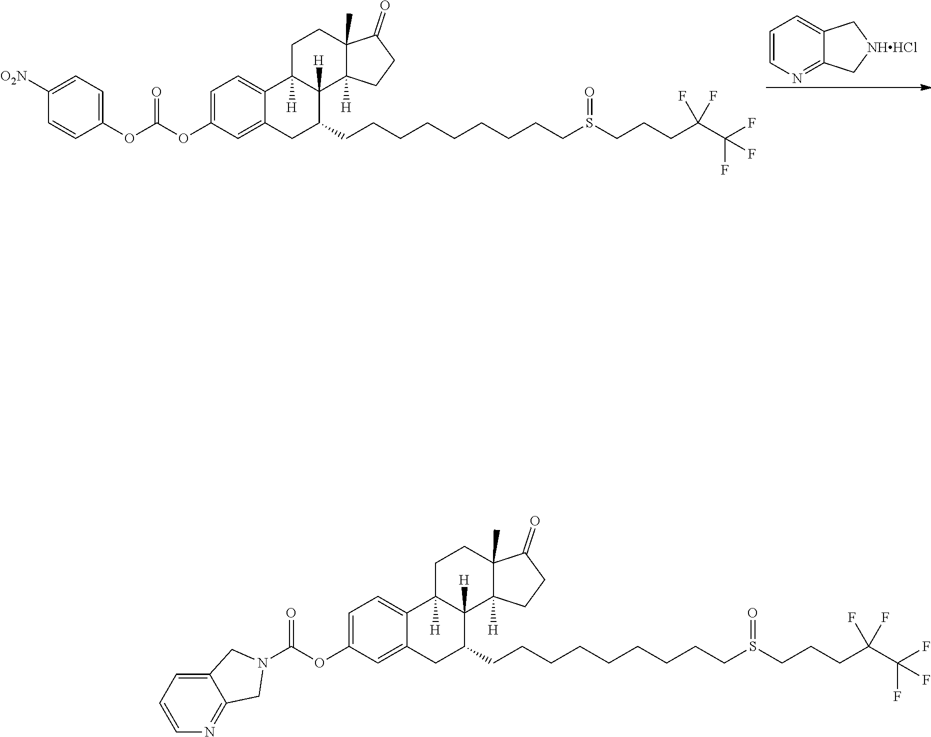

To a stirred solution of 6,7-dihydro-5H-pyrrolo[3,4-b]pyridine hydrochloride (0.15 g) in acetonitrile (10 mL) was added potassium carbonate (0.21 g) and resultant reaction mixture was stirred at room temperature for 2 h. After that (7R,8R,9S,13S,14S)-13-methyl-17-oxo-7-(9-((4,4,5,5,5-pentafluoropentyl) sulfinyl)nonyl)-7,8,9,11,12,13,14,15,16,17-decahydro-6H-cyclopenta[a]phenanthren-3-yl (4-nitrophenyl) carbonate (0.4 g, as prepared in example-6, step-1) was added to the above stirring solution and reaction mass was stirred at room temperature for 16 h. After completion of the reaction (monitored by TLC), reaction mass was diluted with ice water (30 mL) and extracted with ethyl acetate (3×30 mL). The combined organic layers were washed with water (2×40 ml), dried over anhydrous sodium sulfate and concentrated under reduced pressure to give 0.5 g crude material. The crude material was purified by preparative HPLC to afford (7R,8R,9S,13S,14S)-13-methyl-17-oxo-7-(9-((4,4,5,5,5-pentafluoropentyl)sulfinyl)nonyl)-7,8,9,11,12,13,14,15,16,17-decahydro-6H-cyclopenta[a]phenanthren-3-yl 5,7-dihydro-6H-pyrrolo[3,4-b]pyridine-6-carboxylate (0.081 g, 20%) as a white color solid.

LCMS: 96.79% (m/z: 751.50, [M+1]⁺, 7.24 min, 214 nm).

¹H NMR (400 MHz, DMSO-d₆): δ 8.49 (d, J=4.40 Hz, 1H), 7.82 (d, J=7.60 Hz, 1H), 7.36-7.31 (m, 2H), 6.94 (d, J=8.80 Hz, 1H), 6.91 (s, 1H), 4.88 (d, J=28.80 Hz, 2H), 4.71 (d, J=27.60 Hz, 2H), 2.94-2.80 (m, 3H), 2.76-2.61 (m, 4H), 2.44-2.30 (m, 5H), 2.13-2.06 (m, 1H), 1.94-1.79 (m, 4H), 1.76-1.55 (m, 6H), 1.41-1.22 (m, 14H), 0.96-0.91 (m, 1H), 0.85 (s, 3H).

### Example-21: Preparation of (7R,8R,9S,13S,14S)-13-methyl-17-oxo-7-(9-((4,4,5,5,5-pentafluoropentyl)sulfinyl)nonyl)-7,8,9,11,12,13,14,15,16,17-decahydro-6H-cyclopenta[a]phenanthren-3-yl tetrahydro-1H-furo[3,4-c]pyrrole-5(3H)-carboxylate (compound 110)

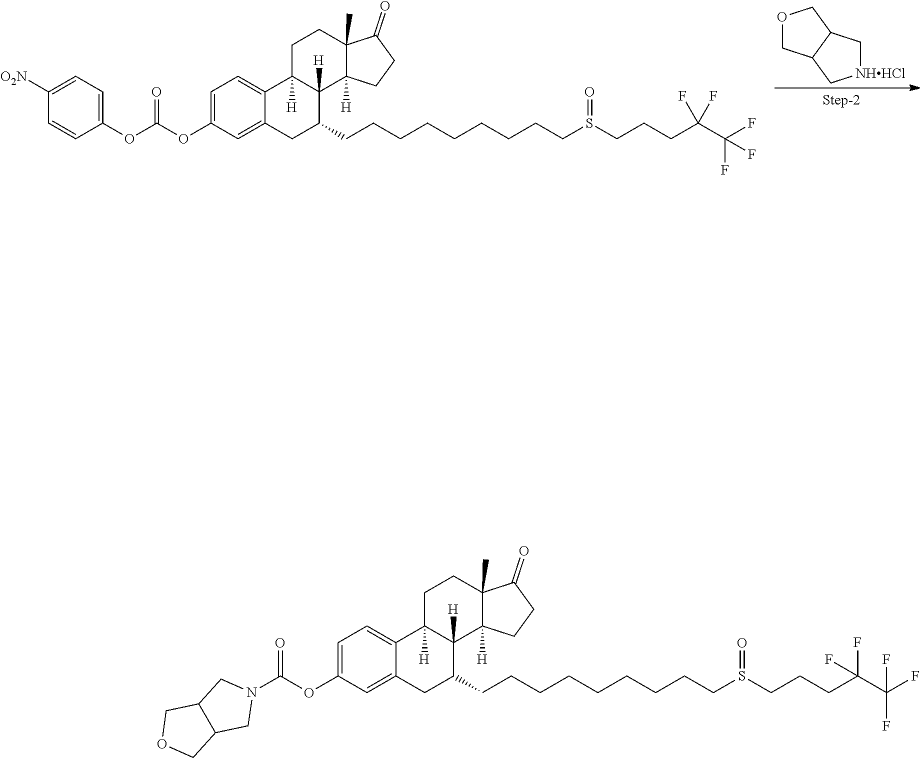

To a stirred solution of (7R,8R,9S,13S,14S,17S)-17-hydroxy-13-methyl-7-(9-((4,4,5,5,5-pentafluoropentyl)sulfinyl)nonyl)-7,8,9,11,12,13,14,15,16,17-decahydro-6H-cyclopenta[a]phenanthren-3-yl (4-nitrophenyl) carbonate (0.45 g, as prepared in example-6, step-1) in acetonitrile (10 mL) was added potassium carbonate (0.80 g) followed by addition of 1′-methyl-[1,4′-bipiperidin]-4-amine (0.130 g). Resultant reaction mixture was stirred at room temperature for 1 h. The reaction mixture was monitored by TLC (mobile phase: 70% ethyl acetate in n-hexane). After completion of the reaction (monitored by TLC), the mixture was diluted with water (30 mL) and extracted with ethyl acetate (3×150 mL). The combined organic layer was dried over anhydrous sodium sulfate and evaporated under reduced pressure to obtain crude material. The crude was purified by reverse phase HPLC [method: (A) 65% ammonium bicarbonate (B) 35% Acetonitrile] and the product fractions were lyophilized to give (7R,8R,9S,13S,14S)-13-methyl-17-oxo-7-(9-((4,4,5,5,5-pentafluoropentyl)sulfinyl)nonyl)-7,8,9,11,12,13,14,15,16,17-decahydro-6H-cyclopenta[a]phenanthren-3-yl tetrahydro-1H-furo[3,4-c]pyrrole-5(3H)-carboxylate (93 mg, 21.4%) as a white solid.

LCMS: 99.28% (m/z: 744.30, [M+1]⁺, 6.73 min, 214 nm).

¹H NMR (400 MHz, DMSO-d₆): δ 7.28 (d, J=8.40 Hz, 1H), 6.85 (d, J=8.40 Hz, 1H), 6.82 (s, 1H), 3.80-3.69 (m, 3H), 3.65-3.39 (m, 5H), 3.25-3.27 (m, 1H), 3.05-2.61 (m, 9H), 2.46-2.34 (m, 4H), 2.12-2.05 (m, 1H), 1.94-1.83 (m, 4H), 1.79-1.67 (m, 3H), 1.66-1.54 (m, 3H), 1.44-1.14 (m, 14H), 0.99-0.90 (m, 1H), 0.84 (s, 3H).

### Example-22: Preparation of (7R,8R,9S,13S,14S)-13-methyl-17-oxo-7-(9-((4,4,5,5,5-pentafluoropentyl)sulfinyl)nonyl)-7,8,9,11,12,13,14,15,16,17-decahydro-6H-cyclopenta[a]phenanthren-3-yl 4-(pyridin-4-yl)piperidine-1-carboxylate (compound 116)

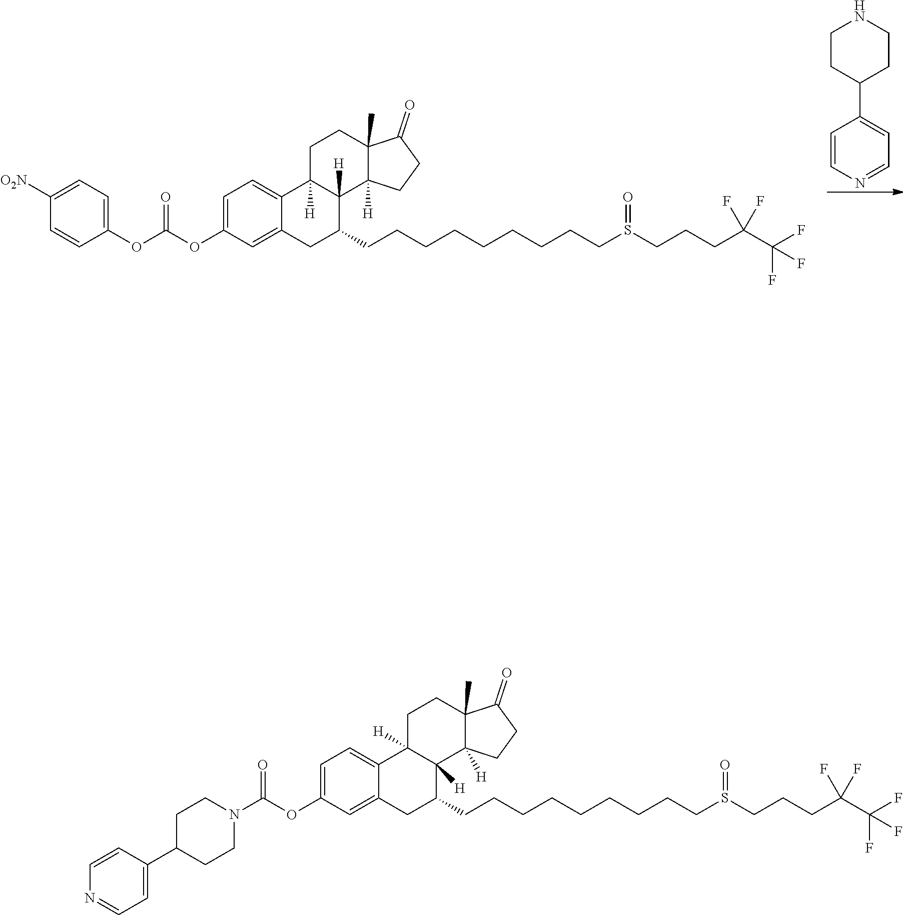

To a stirred solution of 4-(piperidin-4-yl)pyridine (0.12 g) in acetonitrile (10 mL) was added potassium carbonate (0.21 g) and resultant reaction mixture was stirred at room temperature for 2 h. After that (7R,8R,9S,13S,14S)-13-methyl-17-oxo-7-(9-((4,4,5,5,5-pentafluoropentyl)sulfinyl)nonyl)-7,8,9,11,12,13,14,15,16,17-decahydro-6H-cyclopenta[a]phenanthren-3-yl-(4-nitrophenyl) carbonate (0.40 g, as prepared in example-6, step-1) was added and reaction mass was stirred at room temperature for 16 h. After completion of the reaction (monitored by TLC), reaction mass was diluted with ice water (30 mL) and extracted with ethyl acetate (3×30 mL). The combined organic layers were washed with water (2×40 mL), dried over anhydrous sodium sulfate and concentrated under reduced pressure to give 0.5 g crude material. The crude material was purified by preparative HPLC using ammonium acetate as a buffer to afford (7R,8R,9S,13S,14S)-13-methyl-17-oxo-7-(9-((4,4,5,5,5-pentafluoropentyl) sulfinyl)nonyl)-7,8,9,11,12,13,14,15,16,17-decahydro-6H-cyclopenta[a]phenanthren-3-yl 4-(pyridin-4-yl)piperidine-1-carboxylate (0.087 g, 21%) as a white color solid.

LCMS: 95.25% (m/z: 793.55, [M+1]⁺, 6.41 min, 214 nm).

¹H NMR (400 MHz, DMSO-d₆): δ 8.49 (dd, J=1.20, 4.60 Hz, 2H), 7.32 (d, J=4.40 Hz, 2H), 7.29 (d, J=8.80 Hz, 1H), 6.88 (d, J=6.00 Hz, 1H), 6.85 (s, 1H), 4.24-4.14 (m, 2H), 3.11-2.86 (m, 2H), 2.84-2.59 (m, 8H), 2.50-2.32 (m, 5H), 2.15-2.06 (m, 1H), 1.94-1.80 (m, 6H), 1.78-1.48 (m, 8H), 1.39-1.32 (m, 6H), 1.25-1.17 (m, 8H), 0.95-0.90 (m, 1H), 0.84 (s, 3H).

### Example-23: Preparation of (7R,8R,9S,13S,14S,Z)-17-(hydroxyimino)-13-methyl-7-(9-((4,4,5,5,5-pentafluoropentyl)sulfinyl)nonyl)-7,8,9,11,12,13,14,15,16,17-decahydro-6H-cyclopenta[a]phenanthren-3-yl[1,4′-bipiperidine]-1′-carboxylate (compound 126)

### Step-1: Preparation of (7R,8R,9S,13S,14S)-13-methyl-17-oxo-7-(9-((4,4,5,5,5-pentafluoropentyl)sulfinyl)nonyl)-7,8,9,11,12,13,14,15,16,17-decahydro-6H-cyclopenta[a]phenanthren-3-yl[1,4′-bipiperidine]-1′-carboxylate (P1179-J03892-147)

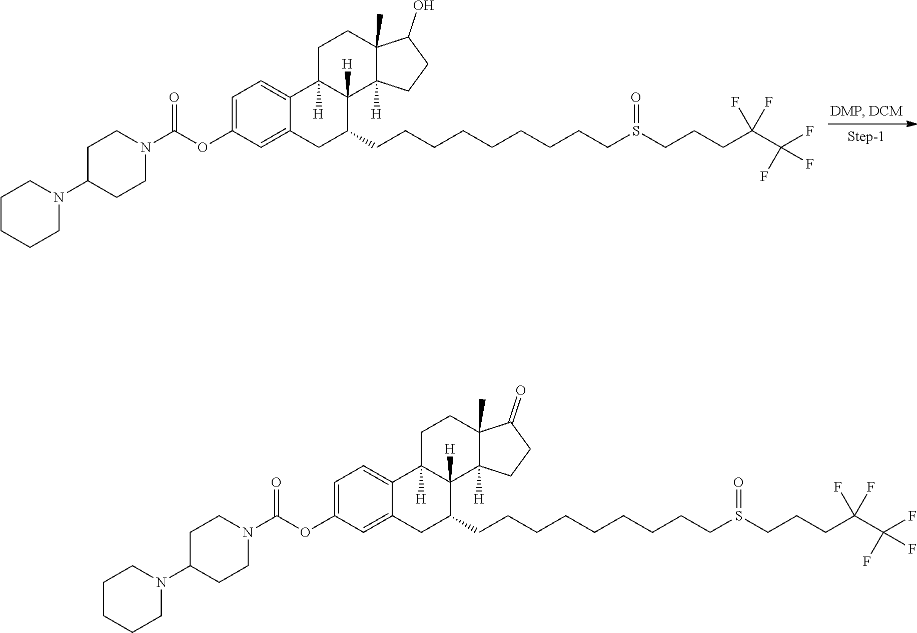

To a stirred solution of (7R,8R,9S,13S,14S)-17-hydroxy-13-methyl-7-(9-((4,4,5,5,5-pentafluoropentyl)sulfinyl)nonyl)-7,8,9,11,12,13,14,15,16,17-decahydro-6H-cyclopenta[a]phenanthren-3-yl[1,4′-bipiperidine]-1′-carboxylate (1.0 g) in dichloromethane (20 mL) was added Dess-Martin Periodinane (0.635 g) and resulting suspension was stirred for 6 h at room temperature. After completion of reaction (monitored by TLC), the mixture was diluted with water (10 mL). The organic compound was extracted with dichloromethane (3×70 mL), washed with water (2×200 mL), dried the organic layer over anhydrous sodium sulfate and concentrated under reduced pressure to afford (7R,8R,9S,13S,14S)-13-methyl-17-oxo-7-(9-((4,4,5,5,5-pentafluoropentyl)sulfinyl)nonyl)-7,8,9,11,12,13,14,15,16,17-decahydro-6H-cyclopenta[a]phenanthren-3-yl[1,4′-bipiperidine]-1′-carboxylate (1.2 g, crude) as a colorless semi solid.

LCMS: 80.82% (m/z: 799.14, [M+1]⁺, 2.48 min (4 min run), 214 nm).

### Step-2: Preparation of (7R,8R,9S,13S,14S,Z)-17-(hydroxyimino)-13-methyl-7-(9-((4,4,5,5,5-pentafluoropentyl)sulfinyl)nonyl)-7,8,9,11,12,13,14,15,16,17-decahydro-6H-cyclopenta[a]phenanthren-3-yl[1,4′-bipiperidine]-1′-carboxylate

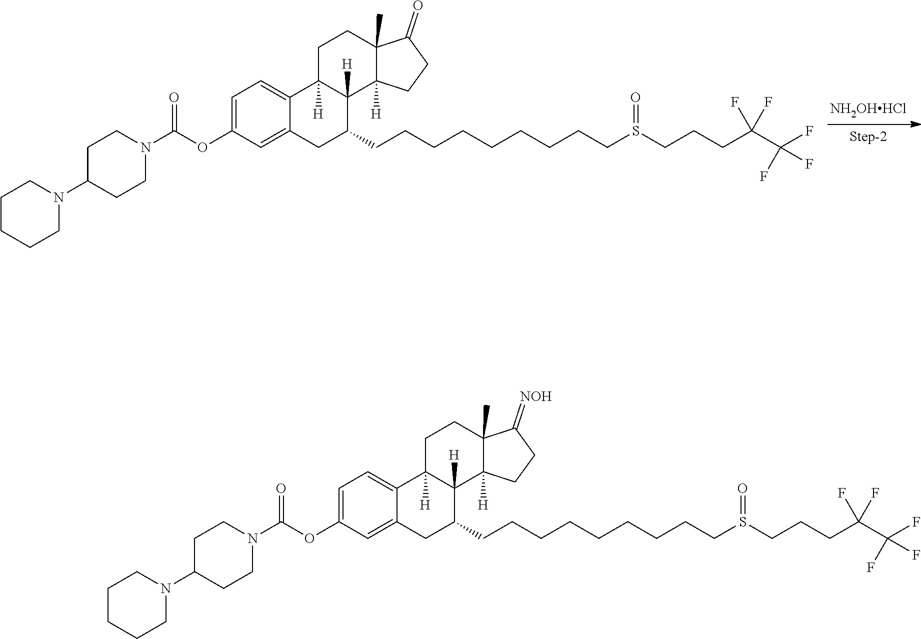

To a stirred solution of (7R,8R,9S,13S,14S)-13-methyl-17-oxo-7-(9-((4,4,5,5,5-pentafluoropentyl)sulfinyl)nonyl)-7,8,9,11,12,13,14,15,16,17-decahydro-6H-cyclopenta[a]phenanthren-3-yl[1,4′-bipiperidine]-1′-carboxylate (1.2 g) in methanol (10 mL) was added sodium acetate (0.30 g) followed by addition of hydroxylamine hydrochloride (0.25 g) and resultant mixture was stirred at 60° C. for 2 h. After completion of the reaction, (monitored by TLC), the mixture was cooled to room temperature and diluted with ethyl acetate (50 mL). The combined organic layers were washed with water (2×200 mL), dried over anhydrous sodium sulfate and evaporated under reduced pressure to obtain crude. The crude was purified by prep HPLC [method: (A) 25% ammonium bicarbonate (B) 70% Acetonitrile] and the product fractions were lyophilized to give (7R,8R,9S,13S,14S,Z)-17-(hydroxyimino)-13-methyl-7-(9-((4,4,5,5,5-pentafluoropentyl)sulfinyl)nonyl)-7,8,9,11,12,13,14,15,16,17-decahydro-6H-cyclopenta[a]phenanthren-3-yl[1,4′-bipiperidine]-1′-carboxylate (compound 126, 270 mg, 22.5%) as white solid.

LCMS: 95.96% (m/z: 814.55, [M+1]⁺, 6.13 min, (10 min run, 214 nm).

¹H NMR (400 MHz, DMSO-d₆): δ 7.27 (d, J=8.40 Hz, 1H), 6.82 (d, J=8.80 Hz, 1H), 6.77 (s, 1H), 4.21-4.12 (m, 2H), 3.72-3.35 (m, 3H), 3.10-2.81 (m, 5H), 2.74-2.67 (m, 3H), 2.37-2.26 (m, 4H), 2.04-2.07 (m, 2H), 1.94-1.84 (m, 5H), 1.76-1.55 (m, 10H), 1.53-1.10 (m, 20H), 0.95-0.80 (m, 4H).

### Example-24: Preparation of (7R,8R,9S,13S,14S,Z)-17-(hydroxyimino)-13-methyl-7-(9-((4,4,5,5,5-pentafluoropentyl)sulfinyl)nonyl)-7,8,9,11,12,13,14,15,16,17-decahydro-6H-cyclopenta[a]phenanthren-3-yl 4-morpholinopiperidine-1-carboxylate (compound 138)

### Step-1 Preparation of (7R,8R,9S,13S,14S)-13-methyl-17-oxo-7-(9-((4,4,5,5,5-pentafluoropentyl)sulfinyl)nonyl)-7,8,9,11,12,13,14,15,16,17-decahydro-6H-cyclopenta[a]phenanthren-3-yl 4-morpholinopiperidine-1-carboxylate

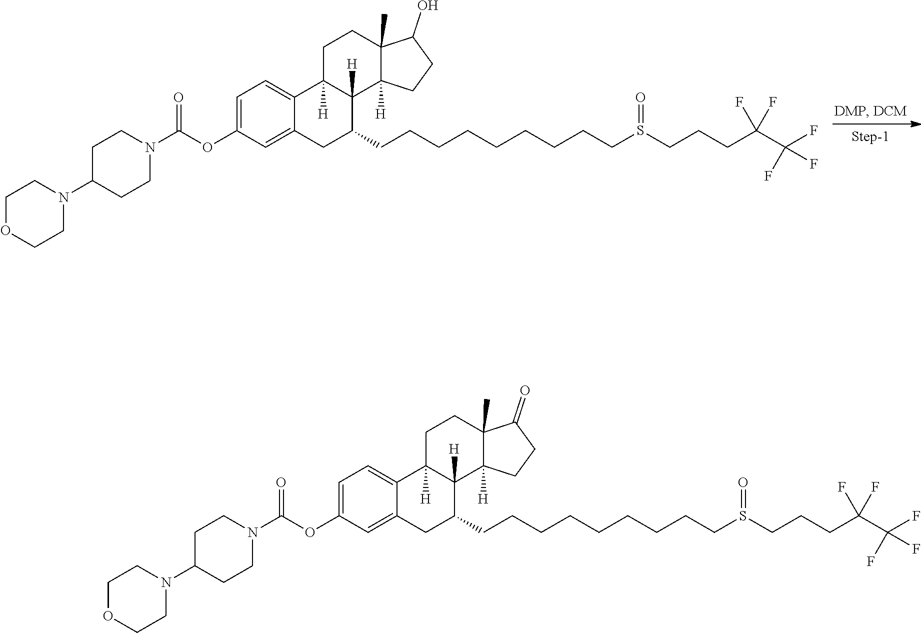

To a stirred solution of (7R,8R,9S,13S,14S)-17-hydroxy-13-methyl-7-(9-((4,4,5,5,5-pentafluoropentyl)sulfinyl)nonyl)-7,8,9,11,12,13,14,15,16,17-decahydro-6H-cyclopenta[a]phenanthren-3-yl 4-morpholinopiperidine-1-carboxylate (0.70 g) in dichloromethane (70 mL) was added Dess-Martin periodinane (0.44 g) and resulting suspension was stirred for 6 h at room temperature. After completion of the reaction (monitored by TLC), the mixture was diluted with water (10 mL). The organic compound was extracted with dichloromethane (3×70 mL), washed with water (2×200 mL), dried the organic layer over anhydrous sodium sulfate and concentrated under reduced pressure to furnish (7R,8R,9S,13S,14S)-13-methyl-17-oxo-7-(9-((4,4,5,5,5-pentafluoropentyl) sulfinyl)nonyl)-7,8,9,11,12,13,14,15,16,17-decahydro-6H-cyclopenta[a]phenanthren-3-yl 4-morpholinopiperidine-1-carboxylate (0.60 g, crude) as a yellowish semi-solid.

LCMS: 38.61% (m/z: 801.50) [M+1]⁺, 2.41 min (4 min run), 214 nm).

### Step-2: Preparation of (7R,8R,9S,13S,14S,Z)-17-(hydroxyimino)-13-methyl-7-(9-((4,4,5,5,5-pentafluoropentyl)sulfinyl)nonyl)-7,8,9,11,12,13,14,15,16,17-decahydro-6H-cyclopenta[a]phenanthren-3-yl 4-morpholinopiperidine-1-carboxylate

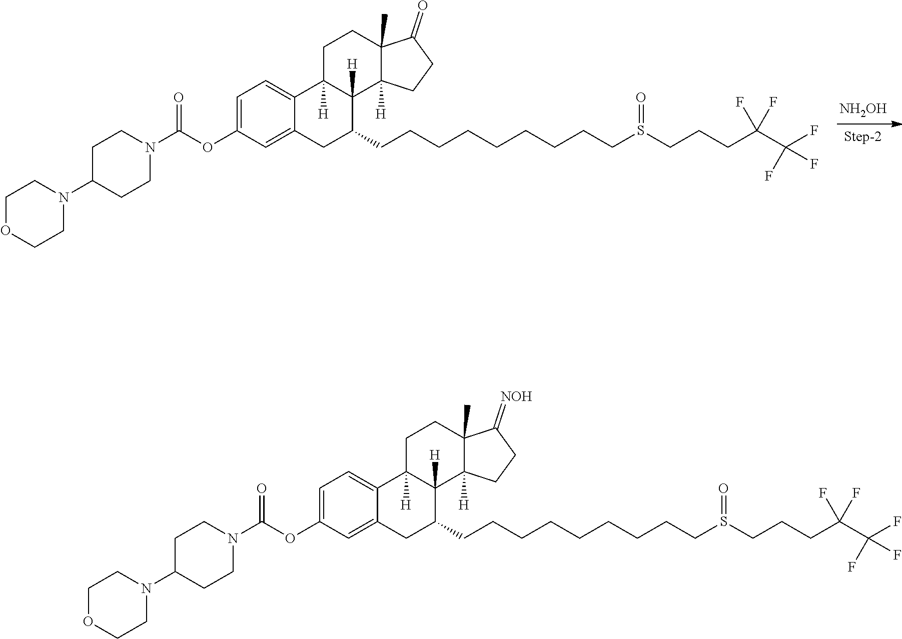

To a stirred solution of (7R,8R,9S,13S,14S)-13-methyl-17-oxo-7-(9-((4,4,5,5,5-pentafluoropentyl)sulfinyl)nonyl)-7,8,9,11,12,13,14,15,16,17-decahydro-6H-cyclopenta[a]phenanthren-3-yl 4-morpholinopiperidine-1-carboxylate (0.60 g) in methanol (10 mL) was added sodium acetate (0.153 g) followed by addition of hydroxylamine hydrochloride (0.124 g) and resultant reaction mixture was stirred for 2 h at 60° C. After completion of the reaction (monitored by TLC), the mixture was cooled to room temperature and diluted with ethyl acetate (30 mL). The combined organic layers were washed with water (2×200 mL), dried over anhydrous sodium sulfate and evaporated under reduced pressure to obtain crude. The crude was purified by preparative HPLC using ammonium bicarbonate as a buffer and the product fractions were lyophilized to give (7R,8R,9S,13S,14S,Z)-17-(hydroxyimino)-13-methyl-7-(9-((4,4,5,5,5-pentafluoropentyl)sulfinyl)nonyl)-7,8,9,11,12,13,14,15,16,17-decahydro-6H-cyclopenta[a]phenanthren-3-yl 4-morpholinopiperidine-1-carboxylate (155 mg, 25.4%) as a white solid.

LCMS: 99.37% (m/z: 816.60, [M+1]⁺, 6.04 min, 214 nm).

¹H NMR (400 MHz, DMSO-d₆-D₂O): δ 7.27 (d, J=8.40 Hz, 1H), 6.82 (d, J=8.40 Hz, 1H), 6.77 (s, 1H), 3.99-4.09 (m, 2H), 3.57-3.55 (m, 4H), 3.10-2.90 (m, 1H), 2.87-2.61 (m, 7H), 2.50-2.45 (m, 4H), 2.39-2.35 (m, 7H), 1.94-1.75 (m, 6H), 1.71-1.68 (m, 2H), 1.65-1.43 (m, 4H), 1.39-1.10 (m, 17H), 0.93-0.89 (m, 1H), 0.85 (s, 3H).

### Example-25: Preparation of (7R,8R,9S,13S,14S)-17-(hydroxyamino)-13-methyl-7-(9-((4,4,5,5,5-pentafluoropentyl)sulfinyl)nonyl)-7,8,9,11,12,13,14,15,16,17-decahydro-6H-cyclopenta[a]phenanthren-3-yl[1,4′-bipiperidine]-1′-carboxylate (compound 151)

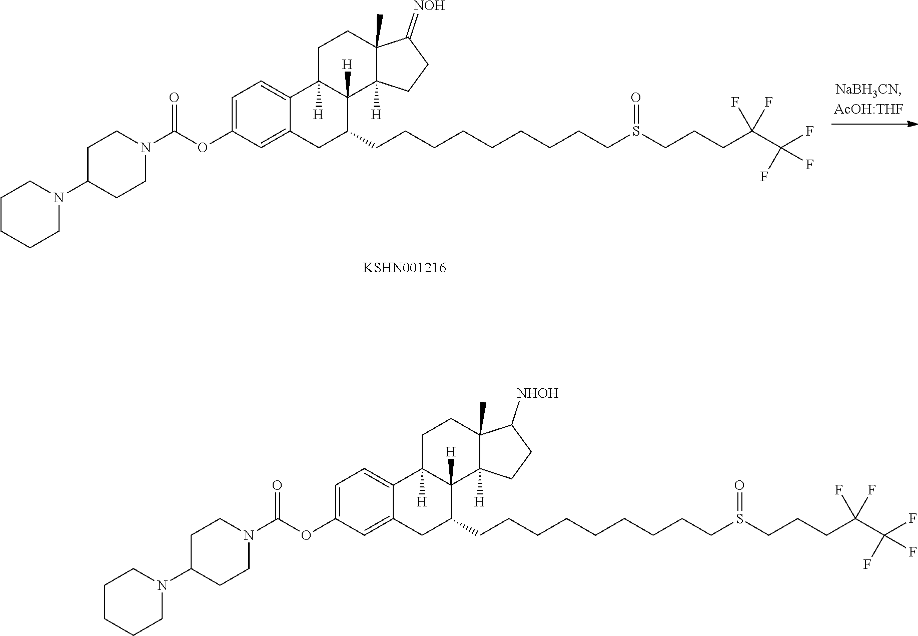

To a stirred solution of (7R,8R,9S,13S,14S,Z)-17-(hydroxyimino)-13-methyl-7-(9-((4,4,5,5,5-pentafluoropentyl)sulfinyl)nonyl)-7,8,9,11,12,13,14,15,16,17-decahydro-6H-cyclopenta[a]phenanthren-3-yl[1,4′-bipiperidine]-1′-carboxylate (0.43 g) in 50% volume of acetic acid and tetrahydrofuran (5 mL) was added NaBH₃CN (0.081 g) and reaction mixture was stirred at room temperature for additional 8 h. After completion of the reaction (monitored by TLC), the mixture was and diluted with ethyl acetate (50 mL). The combined organic layers were washed with water (2×200 mL), dried over anhydrous sodium sulfate and evaporated under reduced pressure to obtain crude. The crude was purified by preparative HPLC using trifluoroacetic acid to furnish (7R,8R,9S,13S,14S)-17-(hydroxyamino)-13-methyl-7-(9-((4,4,5,5,5-pentafluoropentyl)sulfinyl)nonyl)-7,8,9,11,12,13,14,15,16,17-decahydro-6H-cyclopenta[a]phenanthren-3-yl[1,4′-bipiperidine]-1′-carboxylate (75 mg, 17.4%) as a white solid.

LCMS: 92.69% (m/z: 816.60 [M+1]⁺, 5.47 min, (10 min run, 214 nm).

¹H NMR (400 MHz, DMSO-d₆): δ 7.26 (d, J=8.80 Hz, 1H), 6.82 (d, J=8.00 Hz, 1H), 6.77 (s, 1H), 4.10-4.21 (m, 2H), 3.39-3.26 (m, 4H), 3.01-2.79 (m, 6H), 2.76-2.62 (m, 4H), 2.39-2.27 (m, 5H), 2.03-2.05 (m, 4H), 1.93-1.83 (m, 4H), 1.69-1.56 (m, 12H), 1.45-1.10 (m, 16H), 0.84-0.87 (m, 1H), 0.80 (s, 3H).

### Example-26: Preparation of (7R,8R,9S,13S,14S)-17-(hydroxyamino)-13-methyl-7-(9-((4,4,5,5,5-pentafluoropentyl)sulfinyl)nonyl)-7,8,9,11,12,13,14,15,16,17-decahydro-6H-cyclopenta[a]phenanthren-3-yl 4-morpholinopiperidine-1-carboxylate (compound 163)

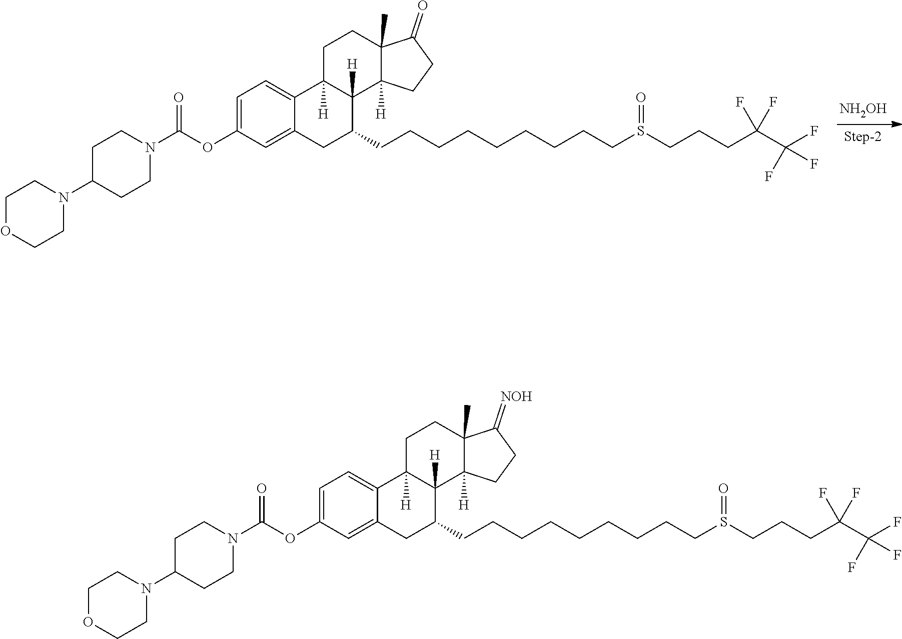

To a stirred solution of (7R,8R,9S,13S,14S)-13-methyl-17-oxo-7-(9-((4,4,5,5,5-pentafluoropentyl)sulfinyl)nonyl)-7,8,9,11,12,13,14,15,16,17-decahydro-6H-cyclopenta[a]phenanthren-3-yl 4-morpholinopiperidine-1-carboxylate (0.6 g, as prepared in example-24, step-1) in methanol (10 mL) was added sodium acetate (0.153 g) followed by addition of hydroxylamine hydrochloride (0.124 g). Resultant reaction mixture was stirred at 60° C. for 2 h. After completion of the reaction (monitored by TLC), the mixture was cooled to room temperature and reaction mixture was diluted with ethyl acetate (30 mL). The combined organic layers were washed with water (2×200 mL), dried over anhydrous sodium sulfate and evaporated under reduced pressure to obtain crude material as (7R,8R,9S,13S,14S,Z)-17-(hydroxyimino)-13-methyl-7-(9-((4,4,5,5,5-pentafluoropentyl)sulfinyl)nonyl)-7,8,9,11,12,13,14,15,16,17-decahydro-6H-cyclopenta[a]phenanthren-3-yl 4-morpholinopiperidine-1-carboxylate (0.600 mg, crude) as semi-solid. Crude was carried forward without any further purification.

LCMS: 56.93% (m/z: 816.10, [M+1]⁺, 2.48 min, 214 nm).

### Step-3: Preparation of (7R,8R,9S,13S,14S)-17-(hydroxyamino)-13-methyl-7-(9-((4,4,5,5,5-pentafluoropentyl)sulfinyl)nonyl)-7,8,9,11,12,13,14,15,16,17-decahydro-6H-cyclopenta[a]phenanthren-3-yl 4-morpholinopiperidine-1-carboxylate

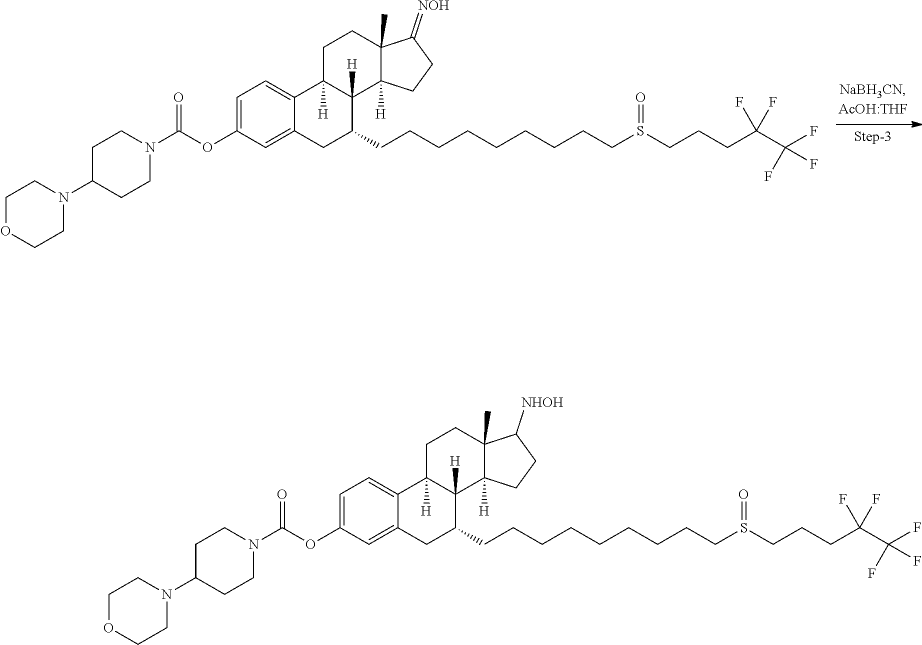

To a stirred solution of (7R,8R,9S,13S,14S,Z)-17-(hydroxyimino)-13-methyl-7-(9-((4,4,5,5,5-pentafluoropentyl)sulfinyl)nonyl)-7,8,9,11,12,13,14,15,16,17-decahydro-6H-cyclopenta[a]phenanthren-3-yl 4-morpholinopiperidine-1-carboxylate (0.6 g) in mixture of acetic acid and tetrahydrofuran (5 mL and 5 mL) was added sodium triacetoxyborohydride (0.115 g) and resultant reaction mixture was stirred at room temperature for 8 h. After completion of the reaction (monitored by TLC), the mixture was extracted with ethyl acetate (50 mL). The combined organic layers were washed with water (2×200 mL), dried over anhydrous sodium sulfate and evaporated under reduced pressure to obtain crude. The crude was purified by preparative HPLC to afford (7R,8R,9S,13S,14S)-17-(hydroxyamino)-13-methyl-7-(9-((4,4,5,5,5-pentafluoropentyl)sulfinyl)nonyl)-7,8,9,11,12,13,14,15,16,17-decahydro-6H-cyclopenta[a]phenanthren-3-yl 4-morpholinopiperidine-1-carboxylate (500 mg, crude) as colourless semi-solid.

LCMS: 45.35% (m/z: 818.03 [M+1]⁺, 2.49 min, (4 min run, 214 nm).

### Example 27: Metabolic Stability Study of in Liver Microsomes

The metabolic stability study was performed in Rat Liver Microsomes (RLM) and Human Liver Microsomes (HLM) with the following test conditions—Microsomal protein concentration—0.5 mg/mL, Test compound concentration—1 μM, NADPH concentration—1 mM, Time points—0, 5, 15, 30, 60 min. The assay was performed in duplicate.

Briefly, the vials containing rat/human liver microsomes were withdrawn from −80±10° C. deep freezer and thawed on the surface of ice bath and then suspended in 66.7 mM potassium phosphate buffer (pH 7.4). To these microsomes, test compound/reference compounds was added to prepare incubation mixture. Aliquots were taken from incubation mixture in tubes labeled as TO (0 min), T5 (5 min), T15 (15 min), T30 (30 min), T60 (60 min) and TC (control-without NADPH). Then, Incubation mixture containing tubes were pre-incubated at 37±1° C. for 5 min in shaking water bath. Simultaneously, NADPH solution (10 mM) was also pre-incubated separately at 37±1° C. for 5 min in shaking water bath.

After pre-incubation, 20 μL NADPH solution (10 mM) was added to the T5, T15, T30 and T60 tubes. The reaction in TO tube was stopped by adding 200 μL quenching solution immediately post addition of NADPH. To the TC (control-without NADPH), After pre-incubation, 20 μL of potassium phosphate buffer (instead of NADPH) was added and incubated for 60 min.

At the end of the incubation period of respective tubes (T5-T60), 200 μL of quenching solution was added to each tube to stop the reaction. Resulting samples were centrifuged at 3220 (relative centrifugal force) x g for 20 min. Supernatant (200 μL) from each reaction tube was taken for LC-MS/MS analysis. Verapamil and Ketoconazole were used as reference compounds during the assay.

Bioanalysis was done using LC-MS/MS.

Results—Compound 121 showed half-life of 106 min in RLM and 44 min in HLM at the end of 60 min incubation. Reference compounds-Verapamil showed half-life of 10 min in RLM and 11 min in HLM and Ketoconazole showed half-life of 45 min in RLM and >120 min in HLM demonstrating the validity of the study.

TABLE 2
Comparison of metabolic stability of compound
121 in Rat and Human Liver microsomes-

| | Half-life (min) |
|---|---|
| Assay Matrix | Compound 121 |
| Rat Liver microsomes | 106 |
| Human Liver microsomes | 44 |

### Example 28: CACO-2 Permeability Assay

Permeability assay was performed using the CACO-2 cell line obtained from ATCC. In the assay, the cells were seeded onto polycarbonate Transwell filter membranes at a density of 60,000 cells/well. After 24 h post seeding, medium was changed and cultured for another 21 days before the treatment of test compounds. On day of treatment, donor solutions were prepared by diluting the stock solutions of test compounds in cell culture medium i.e., HBSS buffer with 10 mM HEPES, pH 7.4. Receiver solutions contain blank 3% BSA (bovine serum albumin) in HBSS buffer with 10 mM HEPES, pH 7.4.

The transport of test compounds (15 μM) was measured in duplicate in two directions [apical to basolateral (A→B) and basolateral to apical (B→A)].

The permeability coefficient for membrane transport of test compounds was determined using the following equation:

$$Papp ( cm / sec ) = ( Vr / C 0 ) ( 1 / S ) ( dC / dt )$$

(Papp=apparent permeability, Vr=volume of medium in the receiver chamber, C0=peak area ratio of the test drug in the receiver chamber, S=surface area of monolayer, dC/dt=peak area ratio of test drug in the receiver chamber with time).
Area of 24-well=0.7 cm² Peak area ratio=Analyte peak area/IS peak area Efflux ratio was defined as Papp B-A/Papp A-B For receiver samples, an aliquot of blank 50 μL HBSS buffer with 10 mM HEPES, pH 7.4 was added in wells containing 50 μL 3% BSA (bovine serum albumin) in HBSS buffer with 10 mM HEPES, pH 7.4 with test compound to match the matrix. For donor samples, an aliquot of 50 μL blank 3% BSA (bovine serum albumin) in HBSS buffer with 10 mM HEPES, pH 7.4 was added in wells containing 50 μL HBSS buffer with 10 mM HEPES, pH 7.4 with test compound to match the matrix.

At the end of time point (120 min for compound 121), resulting samples were centrifuged at 3220 (relative centrifugal force) x g for 20 min and supernatant (200 μL) from each well were taken for LC-MS/MS analysis.

The monolayer integrity was tested post experiment and the movement of lucifer yellow was less than 3% across membrane establishing membrane integrity. Digoxin, Atenolol and Propanolol were used as standard compounds at final assay concentration of 5 μM.

Bioanalysis was done using LC-MS/MS.

Result—compound 121 showed A-B permeability 7.26×10⁻⁶ cm/sec, B-A permeability 4.20×10⁻⁶ cm/sec and B-A/A-B ratio of 0.58.

Reference compounds, atenolol showed A-B permeability 2.18×10⁻⁶ cm/sec, Propranolol showed A-B permeability 62.15×10⁻⁶ cm/sec and Digoxin showed B-A/A-B ratio of 19.7 demonstrated the validation of the assay.

TABLE 3
Comparison of CACO-2 permeability of compound 121-

| Detail | Apparent Permeability (×10⁻⁶ cm/sec) |
|---|---|
| Compound 121 | 7.26 |

### Example 29: Pharmacokinetic Study in Mice

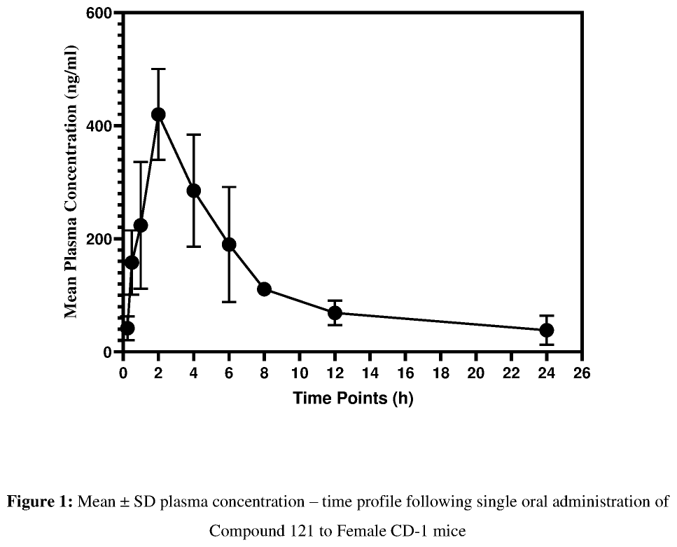

The objective of the study was to evaluate pharmacokinetic exposure of compound 121 after per oral dosing at 26 mg/kg dose in female CD-1 mice as shown in FIG. 1. This study was performed in 6 h fasted female CD-1 mice. Dose formulation was prepared freshly on the day of dosing. 20% PG+10% Solutol HS-15+70% PBS (pH 6.8) was used as vehicle for the preparation of dose formulation.

Blood samples were collected through Retro orbital plexus puncture at 0.25, 0.5, 1, 2, 4, 6, 8, 12 and 24 h post-dose (Total 9 time points/group: n=3 mice/timepoint/group). At each time point, −0.150 mL of blood was withdrawn and transferred into a pre-labeled 0.5 mL of micro centrifuge tubes containing 20 μL of 200 mM K2EDTA solution as anticoagulant and Protease inhibitor cocktail (10% of blood volume collected). Tube was mixed gently by inverting the tube to facilitate mixing of anticoagulant with the blood. Blood samples were kept on gel packs and were centrifuged immediately. The collected blood samples were centrifuged at 4000 rpm for 10 min at 4° C. Plasma was separated after centrifugation. All plasma samples were transferred into pre-labeled tubes and stored at −70±10° C. until bioanalysis. Blank plasma from naïve mice was also collected using same procedure mentioned above and labelled as stabilized mice blank plasma which was used for preparation of calibration standard/quality control samples/blank/zero standard during the bioanalysis.

Bioanalysis was performed using fit-for-purpose LC-MS/MS method for the quantification of compound 121 in plasma samples. The calibration curve was prepared in stabilized blank mice plasma over 0.5-1000 ng/mL range. Samples were cleaned up using the protein precipitation technique and analysed by LC-MS/MS.

The plasma pharmacokinetic parameters for were calculated using standard non-compartmental analysis (using linear trapezoidal method with linear interpolation.

TABLE 4
Arithmetic Mean plasma pharmacokinetic parameters
of compound 121following a single oral route
of administration to Female CD-1 mice

| PK parameters | Compound 121 |
|---|---|
| Cₘₐₓ (ng/mL) | 420 |
| Tₘₐₓ (h) | 2 |
| AUCₗₐₛₜ (h · ng/mL) | 2930 |
| AUC_{inf}_{_{—}}_{obs} (h · ng/mL) | 3540 |
| AUC_{% Extrap}_{_{—}}_{obs} (%) | 17.4 |

TABLE 5
Arithmetic mean plasma concentrations of compound 121following
a single oral administration to Female CD-1 mice

| Time | compound 121 |
|---|---|
| 0.25 | 41.9 ± 21 |
| 0.5 | 158 ± 57 |
| 1 | 224 ± 112 |
| 2 | 420 ± 80.5 |
| 4 | 285 ± 99.3 |
| 6 | 190 ± 102 |
| 8 | 111 ± 10.7 |
| 12 | 68.9 ± 21.7 |
| 24 | 38.3 ± 25.5 |

Note:
Values are expressed as Mean ± SD.

### Example 30: Cytotoxic Effect of Compound 121 on the Human Breast Cancer MCF-7 Cells

MCF-7 human breast cancer cells were procured from the American Type Culture Collection (ATCC, USA) and, were cultured and maintained in Dulbecco's modified Eagle's medium supplemented with 10% fetal bovine serum, and 1% penicillin streptomycin at 37° C. in a humidified 5% CO₂ incubator. Cells were harvested when they reach 70-80% confluence using 0.25% Trypsin-EDTA and were seeded in 96-well tissue culture treated plates@5000 cells/200 μL/well and were maintained in phenol-red Dulbecco's modified Eagle's medium supplemented with 10% fetal bovine serum, and 1% penicillin streptomycin (Growth factor deprived condition). Approximately 24 h after seeding the cells in plate were treated with respective 9 concentrations of compound 121 (30000, 10000, 3000, 1000, 300, 100, 30, 10, 3 nM) in triplicates. Thereafter plates were incubated at 37° C., 5% C02 for 5 days with media replacement on day #3.

For drug formulations, vehicle (0.1% DMSO conc.) was used. Negative control (MAX Viability) was media (with cells) in the presence of 0.1% DMSO. Positive control (MIN Viability) was media (with cells) in presence of highest concentration of compound paclitaxel (1 μM) as reference. After 5 days of treatment, cells were terminated/lysed with Cell titer Glo (CTG) reagent. 40 μl of the content was Transferred in luminescent Plate to record luminescence.

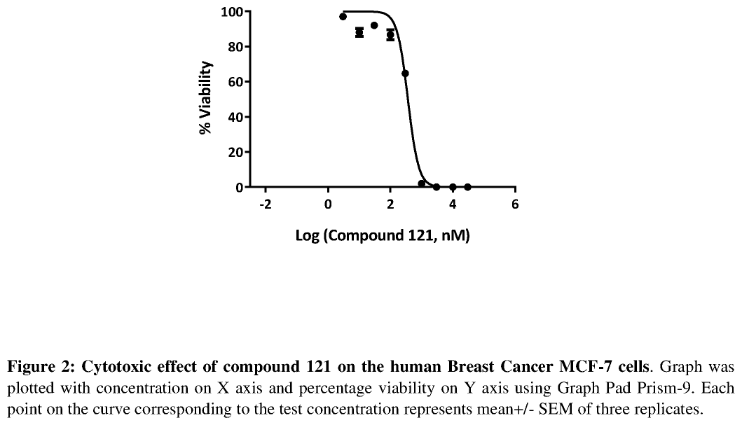

All statistical analysis were performed by Graph Pad Prism-9 software using four-parametric non-linear regression analysis as shown in FIG. 2. The IC50 value of compound 121 was reported to be 362.3 nM.

### Example 31. Effect of Compound 121 on the Uterotrophic Activity of Juvenile Sprague Dawley Rats

This study was carried out on female immature Sprague Dawley rat. At the time of randomization animal age was 18 Days. Rats were housed 3-4 per cage and maintained at controlled environmental conditions. All experimental animals used in this study were under a protocol approved by the Institutional Animal Ethics Committee (IAEC), Approval 057/Jan-2020 No EAL-184_Utero_bioassay. Steam sterilized corn cob (phytoestrogen-free) was used as bedding material and changed along with the cage at least once a week during quarantine, acclimatization and study period. Special rodent maintenance diet, Altromin (1334 P phytoestrogen-poor) was provided ad libitum to all animals.

compound 121 and vehicle treatments were administered orally. Experiment was initiated when animal age was Day 18, and it was considered as Day 1 of treatment. Animals were dosed with respective treatment group as from Day 18 of age to Day 20 of age that is for 3 days period. All animals were observed daily post dosing for any abnormal clinical signs and mortality, observation for individual animals were recorded during the treatment period.

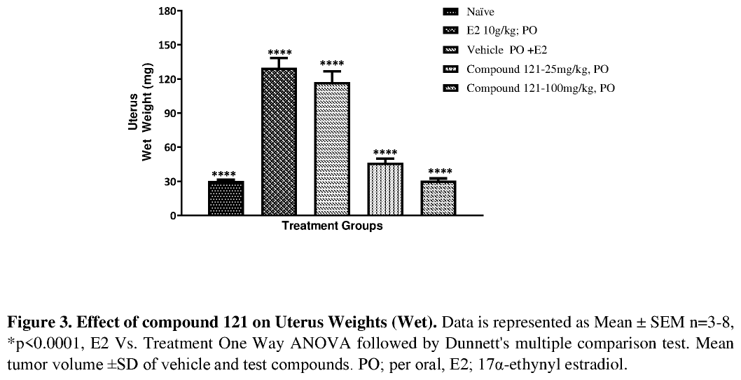

Animals were sacrificed with overdose of gaseous anesthesia; 24 h post last dose of compound administration that is on Day 4. Uterus was removed carefully and weighed for both wet weight and dry weight as shown in FIG. 3.

### Example 32: Anticancer Efficacy of Compound 121 in Nude Mice Bearing MCF7 Tumor

For this purpose, MCF7 cancer cells were procured from ATCC and grown in DMEM Medium supplemented with 10% FBS and 1% penicillin streptomycin. Cells were harvested when they reach 70-80% confluence for tumor implantation. 24 h prior to MCF7 cells implantation, the skin area around the injection site of nude mice were disinfected by mildly swabbing with surgical spirit. 170-estradiol pellet (0.18 mg/pellet 60-day release, Innovative Research of America) was subcutaneously implanted into in the left flank region of the animals. For cell inoculation, cells were mixed with equal volume of Matrigel in a 1:1 ratio followed by injection of 5×10⁶ MCF7 cells subcutaneously on the right flank region using a 1 mL syringe attached to a 24 G needle. On the subsequent days the injection site was monitored for tumor palpability. Tumor grafts were measured within 7-8 days of cell inoculation for tumor palpability. When tumor volume of the implanted animals reaches 200±50 mm³, animals were randomized based on the tumor volume into different groups according to the study design.

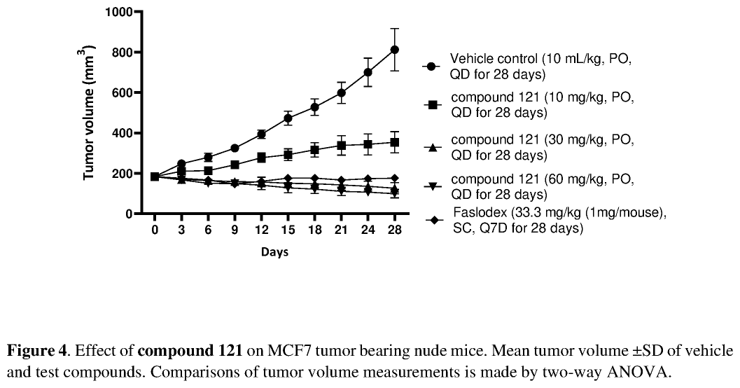

Randomization was carried out in such a way that the mean tumor volume of the individual group remain similar or not significantly different from each other. After randomization, treatment was initiated when the average tumor volume reaches −200±50 mm³. Tumor volume and mouse body weight were measured on the first day of treatment (Day 1) and then three times per week till 28 days as shown in FIG. 4.

Dose formulations of compound 121 (10, 30 and 60 mg/kg) and vehicle (0.5% MC) was administered by oral gavage daily for 28 days. Faslodex(Fulvestrant) was administered subcutaneously.

Tumor volume (mm³) was calculated using the formula, tumor length×(tumor width)2/2. Tumor growth inhibition will be calculated after normalizing the values to that on day 1. % TGI was calculated based on the formula below.

$$\% T G I = [ 1 - ( Mean T V of treatment ) / Mean T V of Control ] \times 100$$

TABLE 6
% Tumor Growth Inhibition (% TGI) of different concentrations
of compound 121 on day 28 in MCF-7 xenograft mice model.

| | | % Tumor Growth |
|---|---|---|
| | | Inhibition (% |
| Group | Treatment | TGI) on day 28 |
| 1 | Vehicle | 0 |
| 2 | compound 121: (10 mg/kg) | 73 |
| 3 | compound 121: (30 mg/kg) | 109 |
| 4 | compound 121: (60 mg/kg) | 113 |
| 5 | Faslodex: 1 mg/mouse (~33.3 mg/kg) | 101 |

### Example 33: Pharmacokinetic Study in Monkey

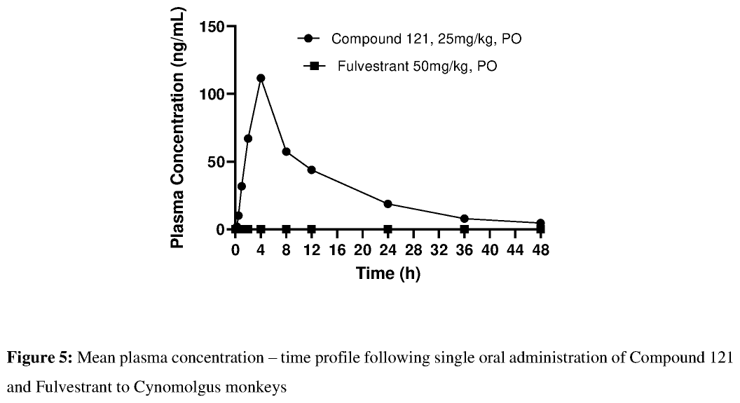

The objective of the study was to evaluate pharmacokinetic exposure of compound 121 after per oral dosing at 25 mg/kg dose and Fulvestrant after per oral dosing at 50 mg/kg dose in Cynomolgus monkey. The study was performed in overnight fasted Cynomolgus monkeys. 0.5% MC was used as vehicle for the preparation of dose formulation as shown in FIG. 5.

Blood samples were collected at each time points like 0 h (pre-dose) and 0.25, 0.5, 1, 2, 4, 8, 12, 24, 36 and 48 h post-dose via the cephalic or saphenous veins into polyethylene microcentrifuge tubes containing potassium (K2) EDTA. There were total 11 time points, n=1 monkey. All blood samples were mixed well by inversion and placed on wet ice until processed for plasma. Blood samples were centrifuged at 2˜8° C. for 10 min at 3000 g to collect plasma. Plasma samples of approximately 200 L at each time point were used for the bioanalysis. The plasma samples were stored frozen in a freezer set to maintain −60° C. to −70° C. until LC/MS/MS analysis.

Bioanalysis was performed using fit-for-purpose LC-MS/MS method for the quantification of compound 121 or Fulvestrant in plasma samples. The calibration curve was prepared in stabilized blank monkey plasma over 1-1000 ng/mL range for compound 121 and 1-3000 ng/mL range for Fulvestrant. Samples were cleaned up using the protein precipitation technique and analysed by LC-MS/MS.

The plasma pharmacokinetic parameters were calculated using standard non-compartmental analysis (using linear trapezoidal method with linear interpolation.)

TABLE 7
Arithmetic Mean plasma pharmacokinetic parameters
of compound 121 and Fulvestrant following a single
oral route of administration to Cynomolgus monkey

| PK parameters | Compound 121 | Fulvestrant |
|---|---|---|
| Cₘₐₓ (ng/mL) | 111.7 | Inestimable |
| Tₘₐₓ (h) | 4.00 | Inestimable |
| AUC_0-t(h*ng/mL) | 1349.89 | Inestimable |
| AUC_0-∞(h*ng/mL) | 1422.37 | Inestimable |
| AUC % Extrap (%) | 5.096 | Inestimable |

### Example-34: Preparation of (7R,8R,9S,13S,14S,17S)-17-hydroxy-13-methyl-7-(9-((4,4,5,5,5-pentafluoropentyl)sulfinyl)nonyl)-7,8,9,11,12,13,14,15,16,17-decahydro-6H-cyclopenta[a]phenanthren-3-yl (4aR,7aS)-4-methylhexahydropyrrolo[3,4-b][1,4]oxazine-6(2H)-carboxylate

### Step-1: Preparation of (7R,8R,9S,13S,14S,17S)-17-hydroxy-13-methyl-7-(9-((4,4,5,5,5-pentafluoropentyl)sulfinyl)nonyl)-7,8,9,11,12,13,14,15,16,17-decahydro-6H-cyclopenta[a]phenanthren-3-yl (4-nitrophenyl) carbonate

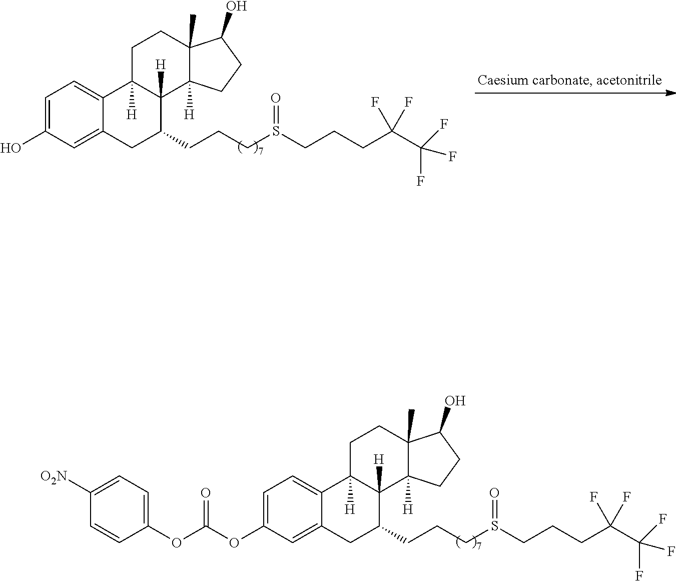

To a stirred solution of Fulvestrant (2.0 g) in acetonitrile (20 mL) were added caesium carbonate (3.24 g) and 4-nitrophenyl chloroformate (0.99 g) at room temperature and stirred the reaction mixture for 15 min at the same temperature. After completion of the reaction on TLC, the reaction mass was diluted with water (15 mL) and extracted with ethyl acetate (2×30 mL), combined organic layer was dried over anhydrous sodium sulfate, and concentrated the organics under reduced pressure to give 7R,8R,9S,13S,14S,17S)-17-hydroxy-13-methyl-7-(9-((4,4,5,5,5-pentafluoropentyl)sulfinyl)nonyl)-7,8,9,11,12,13,14,15,16,17-decahydro-6H-cyclopenta[a]phenanthren-3-yl (4-nitrophenyl) carbonate (2.3 g crude, LCMS 60%), which was used for next step without further purification.

LCMS purity: 60.00%

### Step-2: Preparation of (7R,8R,9S,13S,14S,17S)-17-hydroxy-13-methyl-7-(9-((4,4,5,5,5-pentafluoropentyl)sulfinyl)nonyl)-7,8,9,11,12,13,14,15,16,17-decahydro-6H-cyclopenta[a]phenanthren-3-yl (4aR,7aS)-4-methylhexahydropyrrolo[3,4-b][1,4]oxazine-6(2H)-carboxylate

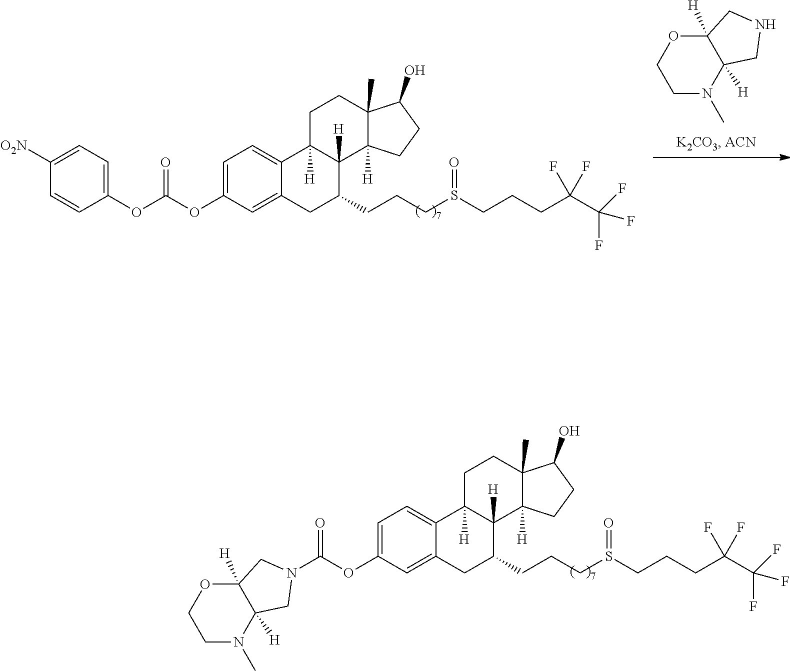

To a stirred solution of (4aR,7aS)-4-methyloctahydropyrrolo[3,4-b][1,4]oxazine hydrochloride (0.110 g) in acetonitrile (10 mL) was added potassium carbonate (0.26 g) and stirred at room temperature for 2 h. After that (7R,8R,9S,13S,14S,17S)-17-hydroxy-13-methyl-7-(9-((4,4,5,5,5-pentafluoropentyl)sulfinyl)nonyl)-7,8,9,11,12,13,14,15,16,17-decahydro-6H-cyclopenta[a]phenanthren-3-yl-(4-nitrophenyl) carbonate (0.3 g) was added and reaction mass was stirred at room temperature for 16 h. After completion of reaction by TLC, reaction mass was diluted with ice water (15 mL) and extracted with ethyl acetate (3×30 mL), The combined organic layers were washed with water (2×100 mL), dried over anhydrous sodium sulfate, and concentrated under reduced pressure which gave crude material. The crude material was purified by preparative HPLC to afford 0.28 g of (7R,8R,9S,13S,14S,17S)-17-hydroxy-13-methyl-7-(9-((4,4,5,5,5-pentafluoropentyl)sulfinyl)nonyl)-7,8,9,11,12,13,14,15,16,17-decahydro-6H-cyclopenta[a]phenanthren-3-yl (4aR,7aS)-4-methylhexahydropyrrolo[3,4-b][1,4]oxazine-6(2H)-carboxylate as a white colour solid.

LCMS purity: 99.83%

¹H NMR (DMSO-d₆): δ 7.27-7.23 (m, 1H), 6.87-6.79 (m, 2H), 4.59-4.53 (bs, 1H), 4.25 (b s, 2H), 415-4.08 (m, 2H), 3.94-3.85 (m, 1H), 3.80-3.68 (m, 3H), 3.61-3.55 (m, 6H), 2.87-2.80 (m, 4H), 2.76-2.68 (m, 3H), 2.65-2.62 (m, 1H), 2.43-2.27 (m, 4H), 1.94-1.88 (m, 3H), 1.81 (d, 1H), 1.70 (d, 1H), 1.63-1.56 (m, 3H), 1.58-1.48 (m, 1H), 1.38-1.19 (m, 18H), 0.90 (bs, 1H), 0.67 (s, 3H).

### Example-35: Preparation of (7R,8R,9S,13S,14S,17S)-17-hydroxy-13-methyl-7-(9-((4,4,5,5,5-pentafluoropentyl)sulfinyl)nonyl)-7,8,9,11,12,13,14,15,16,17-decahydro-6H-cyclopenta[a]phenanthren-3-yl 2-(trifluoromethyl)morpholine-4-carboxylate

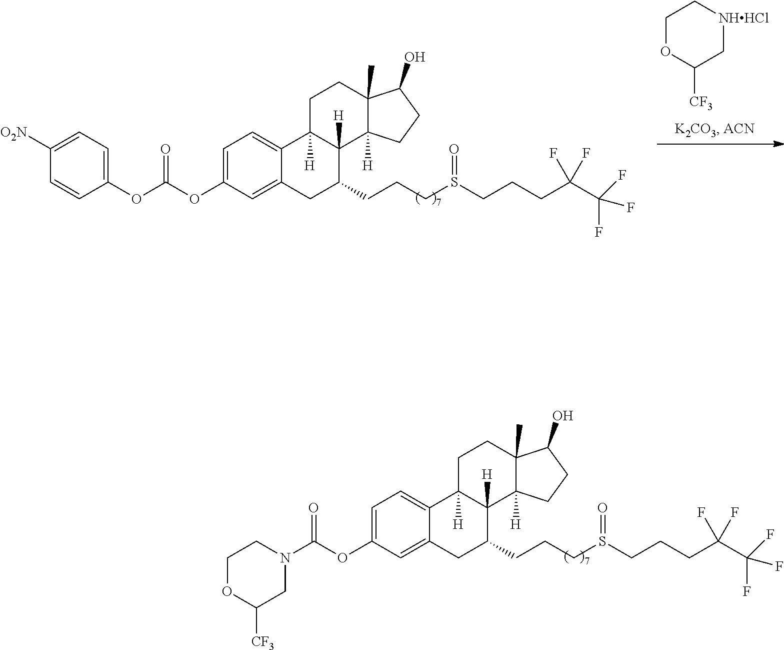

To a stirred solution of 2-(trifluoromethyl)morpholine hydrochloride (0.110 g) in acetonitrile (10 mL) was added potassium carbonate (0.26 g) and stirred at room temperature for 2 h. After that (7R,8R,9S,13S,14S,17S)-17-hydroxy-13-methyl-7-(9-((4,4,5,5,5-pentafluoropentyl)sulfinyl)nonyl)-7,8,9,11,12,13,14,15,16,17-decahydro-6H-cyclopenta[a]phenanthren-3-yl (4-nitrophenyl) carbonate (0.3 g, as prepared in example-34, step-1) was added and reaction mass was stirred at room temperature for 16 h. After completion of reaction monitored by TLC, reaction mass was diluted with ice water (15 mL) and extracted with ethyl acetate (3×30 mL). The combined organic layers were washed with water (2×100 mL), dried over anhydrous sodium sulfate, and concentrated under reduced pressure which gave crude material. The crude material was purified by preparative HPLC to afford 0.10 g of (7R,8R,9S,13S,14S,17S)-17-hydroxy-13-methyl-7-(9-((4,4,5,5,5-pentafluoropentyl)sulfinyl)nonyl)-7,8,9,11,12,13,14,15,16,17-decahydro-6H-cyclopenta[a]phenanthren-3-yl 2-(trifluoromethyl)morpholine-4-carboxylate as a white color solid.

LCMS purity: 99.52%

¹H NMR (DMSO-d₆): δ 7.29 (d, 1H), 6.87 (d, 1H), 6.82 (bs, 1H), 4.36 (bs, 1H), 4.08-3.82 (m, 3H), 3.71-3.65 (t, 1H), 3.57-3.52 (t, 1H), 3.13-3.10 (bs, 2H), 2.90-2.80 (m, 2H), 2.76-2.60 (m, 4H), 2.40-2.25 (m, 5H), 1.93-1.87 (m, 3H), 1.79 (d, 1H), 1.67 (bs, 1H), 1.60-1.51 (m, 3H), 1.47 (bs, 1H), 1.39-1.11 (m, 18H), 0.86 (bs, 1H), 0.65 (s, 3H).

### Example-36: Preparation of (7R,8R,9S,13S,14S,17S)-17-hydroxy-13-methyl-7-(9-((4,4,5,5,5-pentafluoropentyl)sulfinyl)nonyl)-7,8,9,11,12,13,14,15,16,17-decahydro-6H-cyclopenta[a]phenanthren-3-yl tetrahydro-1H-furo[3,4-c]pyrrole-5(3H)-carboxylate

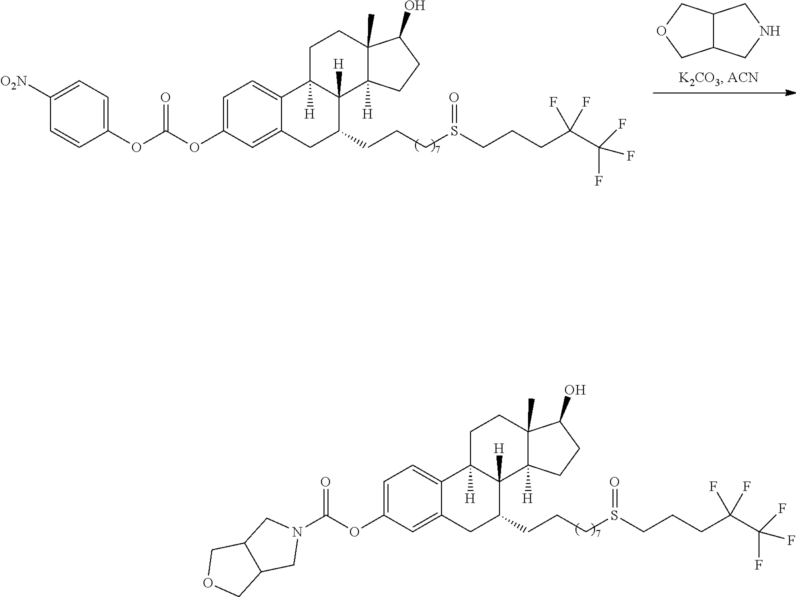

To a stirred solution of hexahydro-1H-furo[3,4-c]pyrrole hydrochloride (0.086 g) in acetonitrile (10 mL) was added potassium carbonate (0.26 g) and stirred at room temperature for 2 h. After that (7R,8R,9S,13S,14S,17S)-17-hydroxy-13-methyl-7-(9-((4,4,5,5,5-pentafluoropentyl)sulfinyl) nonyl)-7,8,9,11,12,13,14,15,16,17-decahydro-6H-cyclopenta[a]phenanthren-3-yl (4-nitrophenyl) carbonate (0.3 g, as prepared in example-34, step-1) was added and reaction mass was stirred at room temperature for 16 h. After completion of reaction by TLC, reaction mass was diluted with ice water (15 mL) and extracted with ethyl acetate (3×30 mL). The combined organic layers were washed with water (2×100 ml), dried over anhydrous sodium sulfate, and concentrated under reduced pressure which gave crude material. The crude material was purified by preparative HPLC to afford 0.065 g of (7R,8R,9S,13S,14S,17S)-17-hydroxy-13-methyl-7-(9-((4,4,5,5,5-pentafluoropentyl)sulfinyl)nonyl)-7,8,9,11,12,13,14,15,16,17-decahydro-6H-cyclopenta[a]phenanthren-3-yl tetrahydro-1H-furo[3,4-c]pyrrole-5(3H)-carboxylate as a white colour solid.

LCMS purity: 99.52%

¹H-NMR (DMSO-d₆): δ 7.26 (d, 1H), 6.83 (d, 1H), 6.78 (bs, 1H), 2.95 (bs, 2H), 2.87-2.80 (m, 2H), 2.76-2.60 (m, 4H), 2.43-2.37 (m, 2H), 2.32-2.24 (m, 2H), 1.94-1.86 (m, 3H), 1.80 (d, 1H), 1.70 (d, 1H), 1.63-1.58 (m, 3H), 1.56-1.53 (m, 1H), 1.38-1.17 (m, 19H), 0.90-0.89 (bs, 1H), 0.67 (s, 3H).

### Example-37: Preparation of (7R,8R,9S,13S,14S,17S)-17-hydroxy-13-methyl-7-(3-((4,4,5,5,5-pentafluoropentyl)sulfinyl)propyl)-7,8,9,11,12,13,14,15,16,17-decahydro-6H-cyclopenta[a]phenanthren-3-yl 4-(2-oxopyrrolidin-1-yl)piperidine-1-carboxylate

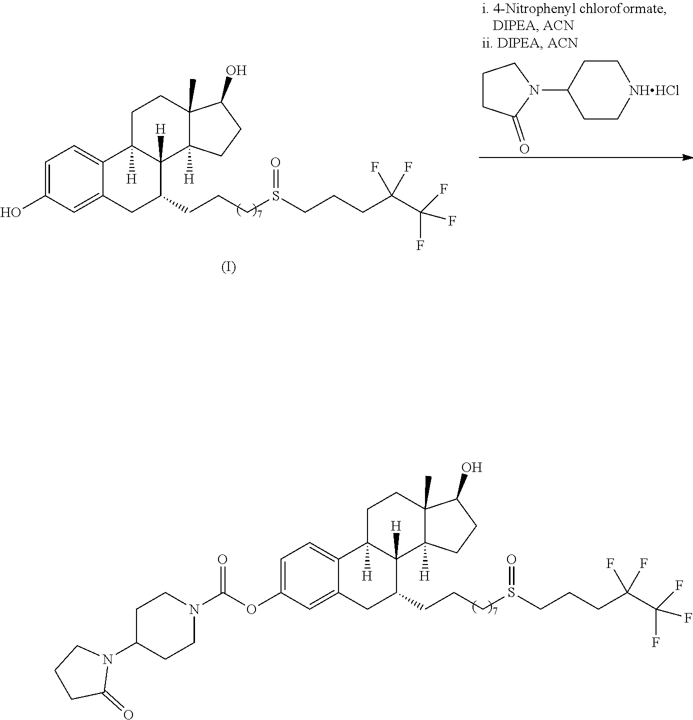

In a 25 mL round bottom flask, (7R,8R,9S,13S,14S,17S)-13-methyl-7-(9-((4,4,5,5,5-pentafluoro pentyl)sulfinyl)nonyl)-7,8,9,11,12,13,14,15,16,17-decahydro-6H-cyclopenta[a]phenanthren-3,17-diol (0.7 g) was dissolved in acetonitrile (7 mL) followed by addition of DIPEA (0.3 mL) and 4-nitrophenyl chloroformate (0.418 g) at 0° C. temperature. In another 25 mL round bottom flask, 1-(piperidin-4-yl)pyrrolidin-2-one hydrochloride (0.353 g) was dissolved in dichloromethane (7 mL) followed by addition of DIPEA (0.2 mL) at room temperature. Both mixtures were stirred for 30 min at the respective temperatures. After 30 minute of stirring, second reaction mixture was added to the first reaction mixture at 0° C. and continued stirring for 3.5 h. After completion of the reaction by TLC and, the reaction mass was diluted with water (25 mL) and extracted with dichloromethane (3×100 mL). Combined organic layer was dried over anhydrous sodium sulfate and then concentrated under reduced pressure to obtain a crude material. The crude was purified by preparative HPLC to afford 0.29 g of (7R,8R,9S,13S,14S,17S)-17-hydroxy-13-methyl-7-(9-((4,4,5,5,5-pentafluoropentyl)sulfinyl)nonyl)-7,8,9,11,12,13,14,15,16,17-decahydro-6H-cyclopenta[a]phenanthren-3-yl-4-(2-oxo pyrrolidin-1-yl)piperidine-1-carboxylate as a white solid.

LCMS purity: 99.00%

¹H NMR (DMSO-d₆-D₂O): δ 7.25 (d, 1H), 6.80 (d, 1H), 6.75 (s, 1H), 4.18-4.03 (m, 2H), 3.93-3.95 (m, 1H), 3.52 (t, 1H), 3.30 (t, 2H), 2.97-3.09 (m, 1H), 2.92-2.63 (m, 7H), 2.39-2.21 (m, 7H), 1.92-1.77 (m, 6H), 1.66-1.45 (m, 9H), 1.39-1.11 (m, 17H), 0.86-0.80 (m, 1H), 0.64 (s, 3H).

### Example-38: Preparation of (7R,8R,9S,13S,14S)-13-methyl-17-oxo-7-(9-((4,4,5,5,5-pentafluoropentyl)sulfinyl)nonyl)-7,8,9,11,12,13,14,15,16,17-decahydro-6H-cyclopenta[a]phenanthren-3-yl 4-(dimethylamino)piperidine-1-carboxylate

### Step-1: Preparation of tert-butyl 4-((2-hydroxyethyl)amino)piperidine-1-carboxylate

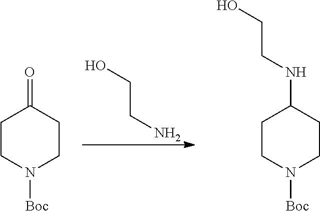

To a stirred solution of tert-butyl 4-oxopiperidine-1-carboxylate (10 g) in dichloromethane (100 mL) were added acetic acid (3.01 g) and 2-aminoethan-1-ol (3 g) and resulting reaction mixture was stirred for 30 min. After that, sodium triacetoxyborohydride (26.5 g) was added to above solution and resultant reaction mixture was stirred at room temperature for 4 h. After completion of the reaction as monitored by TLC, the mixture was quenched with ice water (20 mL). The reaction mass was extracted with dichloromethane (3×200 mL) followed by washings with water (2×500 mL). Collected organics, dried over anhydrous sodium sulfate, and concentrated under reduced pressure to afford 7.5 g of tert-butyl 4-((2-hydroxyethyl)amino)piperidine-1-carboxylate as a brown semi-solid.

¹H NMR (DMSO-d₆): δ 4.41 (bs, 1H), 3.80 (d, 2H), 3.42 (t, 2H), 2.89-2.59 (m, 2H), 2.56-2.59 (m, 2H), 1.75-1.71 (m, 2H), 1.38 (s, 9H), 1.12-1.02 (m, 2H).

### Step-2: Preparation of tert-butyl 4-(2-bromo-N-(2-hydroxyethyl)acetamido)piperidine-1-carboxylate

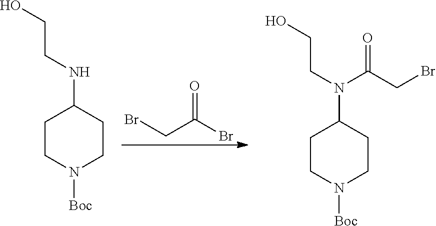

To a stirred solution of (tert-butyl 4-((2-hydroxyethyl)amino)piperidine-1-carboxylate (5.0 g) in tetrahydrofuran (50 mL) was added 2-bromoacetyl bromide (3.29 g) and resultant mixture was stirred at −30° C. for 5 min. After completion of the reaction as monitored by TLC, reaction mixture was quenched with cold water (10 mL). The reaction mass was extracted with ethyl acetate (3×80 mL) followed by washings with water (2×500 mL). Collected organics, dried over anhydrous sodium sulfate, and concentrated under reduced pressure to obtain the crude material. The crude was purified by combi-flash chromatography to afford 3.5 g of tert-butyl 4-(2-bromo-N-(2-hydroxyethyl)acetamido)piperidine-1-carboxylate as a yellow semi solid.

LCMS purity: 77.23%

### Step-3: Preparation of tert-butyl 4-(3-oxomorpholino)piperidine-1-carboxylate

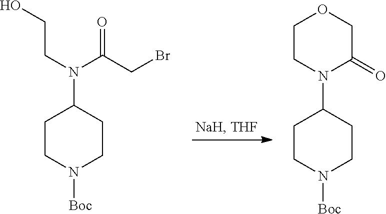 [SMILES: CC(C)(C)OC(=O)N1CCC(N(CCO)C(=O)CBr)CC1 ; CC(C)(C)OC(=O)N1CCC(N2CCOCC2=O)CC1]

To a stirred solution of tert-butyl 4-(2-bromo-N-(2-hydroxyethyl) acetamido)piperidine-1-carboxylate (3.5 g) in tetrahydrofuran (50 mL) was added sodium hydride (0.276 g) and resulting reaction mixture was stirred at room temperature for 1 h. After completion of the reaction as monitored by TLC, reaction mass was quenched with cold water (10 mL). The reaction mass was extracted with ethyl acetate (3×100 mL) followed by washings with water (2×200 mL). Collected organics, dried over anhydrous sodium sulfate and concentrated under reduced pressure to furnish 2.7 g of tert-butyl 4-(3-oxomorpholino) piperidine-1-carboxylate as an off-white semi solid.

LCMS purity: 96.88%

¹H NMR (DMSO-d₆): δ 4.36 (s, 1H), 4.12-4.02 (m, 4H), 3.78 (t, 2H), 3.23 (t, 2H), 2.67-2.77 (m, 2H), 1.58-1.50 (m, 4H), 1.40 (s, 9H).

### Step-4: Preparation of 4-(piperidin-4-yl)morpholin-3-one

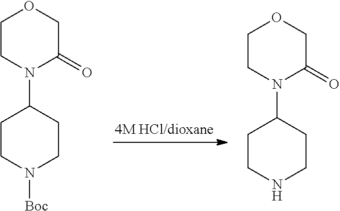 [SMILES: CC(C)(C)OC(=O)N1CCC(N2CCOCC2=O)CC1 ; O=C1COCCN1C1CCNCC1]

To a stirred solution of tert-butyl 4-(3-oxomorpholino)piperidine-1-carboxylate (2.6 g) in dichloromethane (5 mL) was added 4M HCl in dioxane (5 ml) at 0° C. temperature and resulting solution was stirred at room temperature for 2 h. After completion of the reaction as monitored by TLC, the mixture was concentrated under reduced pressure to obtain the crude material. The crude was washed with diethyl ether and n-pentane to afford 1.3 g of 4-(piperidin-4-yl)morpholin-3-one as a white solid.

LCMS purity: 99.62%

¹H NMR (DMSO-d₆): δ 4.48-4.46 (m, 1H), 3.84-3.81 (m, 4H), 3.31 (d, 2H), 3.23 (t, 2H), 3.03-2.97 (m, 2H), 2.00-1.96 (m, 2H), 1.67-1.70 (m, 2H).

### Step-5: Preparation of (7R,8R,9S,13S,14S,17S)-17-hydroxy-13-methyl-7-(9-((4,4,5,5,5-pentafluoropentyl)sulfinyl)nonyl)-7,8,9,11,12,13,14,15,16,17-decahydro-6H-cyclopenta[a]phenanthren-3-yl 4-(3-oxomorpholino)piperidine-1-carboxylate

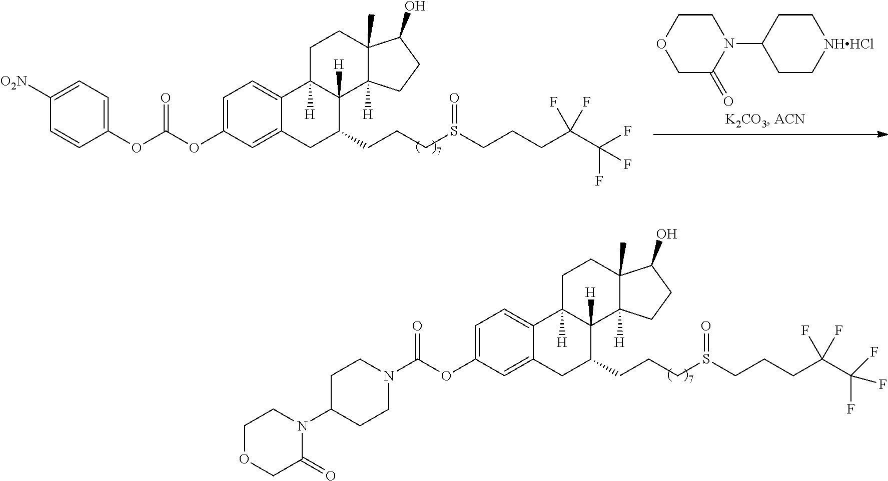

To a stirred solution of 4-(piperidin-4-yl)morpholin-3-one (0.437 g) in acetonitrile (15 mL) was added potassium carbonate (1.7 g) and resulting reaction mixture was stirred at room temperature for 30 min. After that, (7R,8R,9S,13S,14S,17S)-17-hydroxy-13-methyl-7-(9-((4,4,5,5,5-pentafluoropentyl)sulfinyl)nonyl)-7,8,9,11,12,13,14,15,16,17-decahydro-6H-cyclopenta[a]phenanthren-3-yl (4-nitrophenyl)carbonate (1 g, as prepared in example-34, step-1) was added and resulting mixture was stirred at room temperature for 4 h. After completion of the reaction as monitored by TLC, the mixture was quenched with water (10 ml). The organic compound was extracted using ethyl acetate (3×50 mL), washed with water (2×200 mL), dried the organic layer over anhydrous sodium sulfate and concentrated under reduced pressure to obtain the crude material. The crude was purified using preparative HPLC to afford 0.13 g of (7R,8R,9S,13S,14S,17S)-17-hydroxy-13-methyl-7-(9-((4,4,5,5,5-pentafluoropentyl)sulfinyl)nonyl)-7,8,9,11,12,13,14,15,16,17-decahydro-6H-cyclopenta[a]phenanthren-3-yl 4-(3-oxomorpholino)piperidine-1-carboxylate as a white solid.

LCMS purity: 99.30%

¹H-NMR (DMSO-d₆-D₂O): δ 7.26 (d, 1H), 6.81 (d, 1H), 6.76 (s, 1H), 4.44-4.37 (m, 1H), 4.20-4.18 (m, 2H), 4.02 (s, 2H), 3.52 (t, 1H), 3.26 (t, 2H), 3.09-2.88 (m, 2H), 2.86-2.61 (m, 6H), 2.40-2.21 (m, 3H), 1.93-1.78 (m, 4H), 1.67-1.45 (m, 9H), 1.37-1.13 (m, 19H), 1.10-0.75 (m, 1H), 0.64 (s, 3H).

### Example-39: Preparation of (7R,8R,9S,13S,14S,17S)-17-hydroxy-13-methyl-7-(9-((4,4,5,5,5-pentafluoropentyl)sulfinyl)nonyl)-7,8,9,11,12,13,14,15,16,17-decahydro-6H-cyclopenta[a]phenanthren-3-yl-3-(trifluoromethyl)-5,6-dihydro-[1,2,4]triazolo[4,3-a]pyrazine-7(8H)-carboxylate

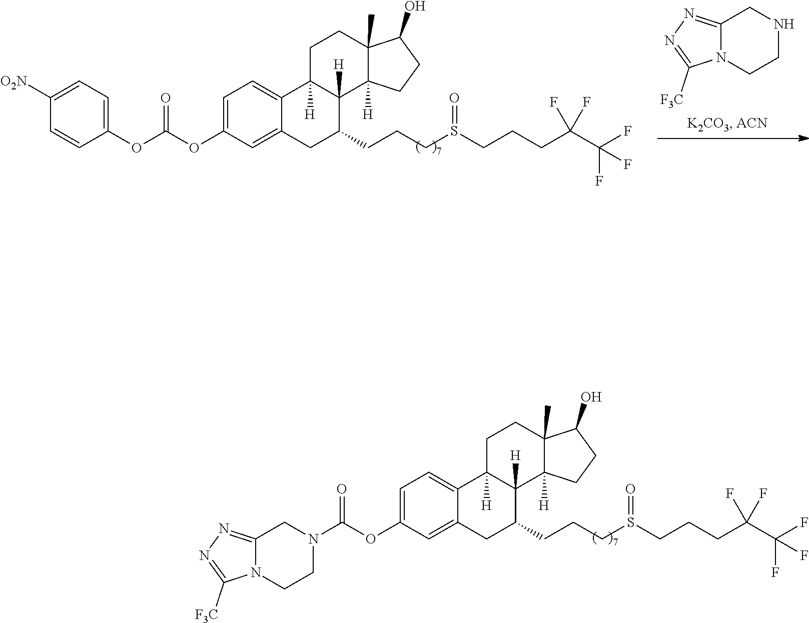

To a stirred solution of 3-(trifluoromethyl)-5,6,7,8-tetrahydro-[1,2,4]triazolo[4,3-a]pyrazine (0.622 g) in acetonitrile (12.5 mL) was added potassium carbonate (0.895 g) at 0° C. and resultant reaction mixture was stirred at 0° C. for 15 min. After that, (7R,8R,9S,13S,14S,17S)-17-hydroxy-13-methyl-7-(9-((4,4,5,5,5-pentafluoropentyl)sulfinyl)nonyl)-7,8,9,11,12,13,14,15,16,17-decahydro-6H-cyclopenta[a]phenanthren-3-yl (4-nitrophenyl) carbonate (0.5 g, as prepared in example-34, step-1) was added to above reaction mixture and resultant reaction mixture was stirred at room temperature for 16 h. After completion of the reaction as monitored by TLC, the mixture was diluted with water (10 mL). The reaction mass was extracted with ethyl acetate (3×10 mL) followed by washings with water (2×10 mL). Collected organics, dried over anhydrous sodium sulfate, and concentrated under reduced pressure to obtain the crude. The crude was further purified by preparative HPLC to afford 0.32 g of (7R,8R,9S,13S,14S,17S)-17-hydroxy-13-methyl-7-(9-((4,4,5,5,5-pentafluoropentyl)sulfinyl)nonyl)-7,8,9,11,12,13,14,15,16,17-decahydro-6H-cyclopenta[a]phenanthren-3-yl 3-(trifluoromethyl)-5,6-dihydro-[1,2,4]triazolo[4,3-a]pyrazine-7(8H)-carboxylate as a white semi solid.

LCMS purity: 99.98%

¹H NMR (DMSO-d6-D₂O): δ 7.28 (d, 1H), 6.89 (d, 1H), 6.84 (s, 1H), 4.85-5.02 (m, 2H), 4.24-4.27 (m, 2H), 3.90-4.03 (m, 2H), 3.52 (t, 1H), 2.86-2.61 (m, 6H), 2.40-2.22 (m, 3H), 1.93-1.76 (m, 4H), 1.69-1.45 (m, 5H), 1.34-1.13 (m, 19H), 0.84-0.86 (m, 1H), 0.65 (s, 3H).

### Example-40: Preparation of (7R,8R,9S,13S,14S,17S)-17-hydroxy-13-methyl-7-(9-((4,4,5,5,5-pentafluoropentyl)sulfinyl)nonyl)-7,8,9,11,12,13,14,15,16,17-decahydro-6H-cyclopenta[a]phenanthren-3-yl 6,7-dihydrothieno[3,2-c]pyridine-5(4H)-carboxylate

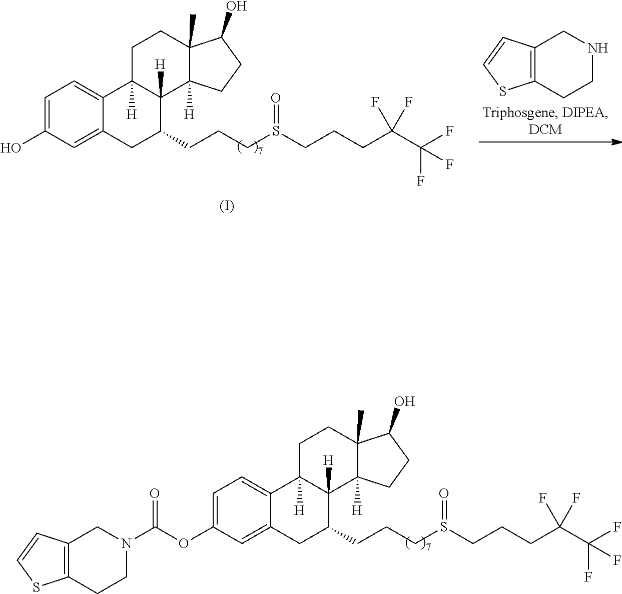

To a stirred solution of compound-I (0.3 g) in dichloromethane (4 mL) were added triphosgene (0.073 g) and DIPEA (6.84 g) at 0° C. temperature. Resulting reaction mixture was stirred at 0° C. for 15 min. After that, tetrahydrothieno[3,2-c]pyridine (0.103 g) was added to above solution and resulted reaction mixture stirred at room temperature for 16 h. After completion of the reaction as monitored by TLC, the mixture was diluted with water (10 mL). The reaction mass was extracted with dichloromethane (3×10 mL), washed with water (2×10 mL). Collected organics, dried over anhydrous sodium sulfate, and concentrated under reduced pressure to obtain the crude material. The crude was purified by preparative HPLC to afford 0.10 g of (7R,8R,9S,13S,14S,17S)-17-hydroxy-13-methyl-7-(9-((4,4,5,5,5-pentafluoropentyl)sulfinyl) nonyl)-7,8,9,11,12,13,14,15,16,17-decahydro-6H-cyclopenta[a]phenanthren-3-yl-6,7-dihydro thieno[3,2-c]pyridine-5(4H)-carboxylate as a white solid.

LCMS purity: 98.01%

¹H-NMR (DMSO-d₆-D₂O): δ 7.29 (d, 1H), 7.25 (d, 1H), 6.88 (d, 1H), 6.85 (dd, 1H), 6.78 (d, 1H), 4.68-4.50 (m, 2H), 3.88-3.69 (m, 2H), 3.55 (t, 1H), 2.89-2.79 (m, 4H), 2.75-2.60 (m, 4H), 2.40-2.25 (m, 3H), 1.95-1.81 (m, 4H), 1.70-1.43 (m, 5H), 1.28 (s, 3H).

### Example-41: Preparation of (7R,8R,9S,13S,14S,17S)-17-hydroxy-13-methyl-7-(9-((4,4,5,5,5-pentafluoropentyl)sulfinyl)nonyl)-7,8,9,11,12,13,14,15,16,17-decahydro-6H-cyclopenta[a]phenanthren-3-yl (4-(2-(trifluoromethyl)morpholino)phenyl)carbamate

### Step-1: Preparation of 4-(4-nitrophenyl)-2-(trifluoromethyl)morpholine

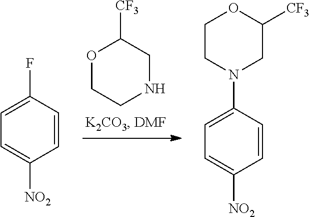

A stirred solution of 1-fluoro-4-nitrobenzene (1.0 g) in dimethylformamide were added potassium carbonate (2.19 g) and 2-(trifluoromethyl)morpholine (1.95 g) at room temperature under nitrogen.

Resulting reaction mixture was stirred at 50° C. After completion of the reaction as monitored by TLC, the mixture was diluted with water and extracted using ethyl acetate. Collected organics, dried the organic layer over anhydrous sodium sulfate and concentrated under reduced pressure to obtain the crude. The crude material was purified by flash chromatography to afford 0.70 g of 4-(4-nitrophenyl)-2-(trifluoromethyl)morpholine as a yellow solid.

LCMS purity: 97.03%

### Step-2: Preparation of 4-(2-(trifluoromethyl)morpholino)aniline

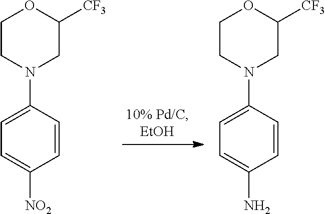 [SMILES: O=[N+]([O-])c1ccc(N2CCOC(C(F)(F)F)C2)cc1 ; Nc1ccc(N2CCOC(C(F)(F)F)C2)cc1]

To a stirred solution of 4-(4-nitrophenyl)-2-(trifluoromethyl)morpholine (0.7 g) in methanol (10 mL) was added 10% palladium on carbon (0.450 g, 50% wet basis) and the reaction mixture was stirred at room temperature under hydrogen atmosphere for 2.5 h. After completion of the reaction as monitored by TLC, the reaction mixture was filtered through celite, and filtrate was concentrated under reduced pressure to afford 0.6 g of 4-(2-(trifluoromethyl)morpholino)aniline as a brown sticky solid.

LCMS purity: 79.16%

### Step-3: Preparation of (7R,8R,9S,13S,14S,17S)-17-hydroxy-13-methyl-7-(9-((4,4,5,5,5-pentafluoropentyl)sulfinyl)nonyl)-7,8,9,11,12,13,14,15,16,17-decahydro-6H-cyclopenta[a]phenanthren-3-yl (4-(2-(trifluoromethyl)morpholino)phenyl)carbamate

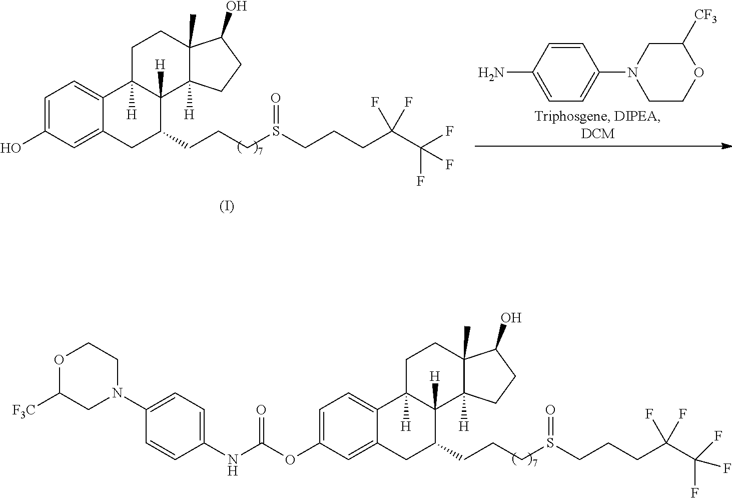

To a stirred solution of (7R,8R,9S,13S,14S,17S)-13-methyl-7-(9-((4,4,5,5,5-pentafluoropentyl) sulfinyl)nonyl)-7,8,9,11,12,13,14,15,16,17-decahydro-6H-cyclopenta[a]phenanthrene-3,17-diol (1.2 g) in dichloromethane (20 mL) were added DIPEA (0.52 mL) and triphosgene (0.293 g) at 0° C. temperature under nitrogen. Resultant reaction mixture was stirred at same temperature for another 10 min. After that, 4-(2-(trifluoromethyl)morpholino)aniline (0.585 g) was added to above stirring solution and the mixture was stirred for 16 h at room temperature. After completion of the reaction as monitored by TLC, the reaction mixture was diluted with water and extracted with dichloromethane (2×100 mL). Collected organics, dried the organics over anhydrous sodium sulfate and concentrated under reduced pressure to obtain the crude material. The crude was purified by preparative HPLC to afford 0.70 g of (7R,8R,9S,13S,14S,17S)-17-hydroxy-13-methyl-7-(9-((4,4,5,5,5-pentafluoropentyl)sulfinyl)nonyl)-7,8,9,11,12,13,14,15,16,17-decahydro-6H-cyclopenta[a]phenanthren-3-yl-(4-(2-(trifluoromethyl)morpholino)phenyl) carbamate as a white solid.

LCMS purity: 95.01%

¹H-NMR (DMSO-d₆-D₂O): δ 7.32 (d, 2H), 7.28 (d, 1H), 6.95 (d, 2H), 6.86 (d, 1H), 6.80 (s, 1H), 4.34-4.14 (m, 1H), 3.72 (t, 2H), 3.59 (d, 1H), 3.52 (t, 4H), 3.40 (d, 4H), 2.78-2.59 (m, 7H), 2.50-2.24 (m, 5H), 1.88-1.77 (m, 4H), 1.66-1.46 (m, 5H), 1.32-113 (m, 18H), 0.87-0.84 (m, 1H), 0.64 (s, 3H).

### Example-42: Preparation of (7R,8R,9S,13S,14S,17S)-17-hydroxy-13-methyl-7-(9-((4,4,5,5,5-pentafluoropentyl)sulfinyl)nonyl)-7,8,9,11,12,13,14,15,16,17-decahydro-6H-cyclopenta[a]phenanthren-3-yl 2-oxo-[1,4′-bipiperidine]-1′-carboxylate

### Step-1: Preparation of tert-butyl 2-oxo-[1,4′-bipiperidine]-1′-carboxylate

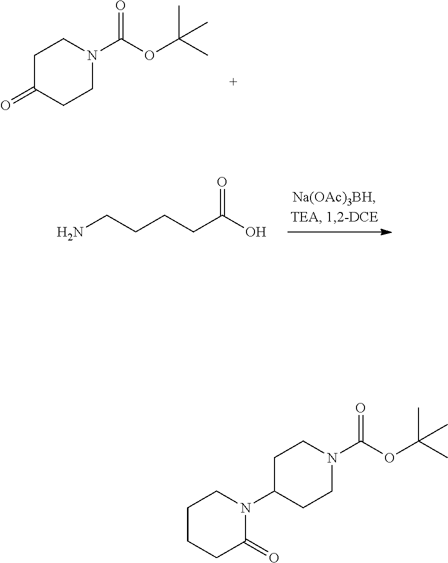 [SMILES: CC(C)(C)OC(=O)N1CCC(=O)CC1 ; NCCCCC(=O)O ; CC(C)(C)OC(=O)N1CCC(N2CCCCC2=O)CC1]

To a stirred solution of tert-butyl 4-oxopiperidine-1-carboxylate (2 g) in dichloroethane (40 mL) was added 5-aminopentanoic acid (2.92 g) and resulting reaction mixture is stirred at room temperature for 16 h. Formation of imine was confirmed by TLC and then sodium triacetoxyborohydride (3.18 g) was added to above stirring solution. Resultant reaction mixture was stirred at room temperature for additional 1 h at room temperature. After 1 h, triethylamine was added drop wisely and the resulting reaction mixture was stirred for additional 14 h at room temperature, followed by heating at 60° C. for 60 h. After completion of the reaction (monitored by TLC), reaction mass was diluted with water (50 mL) followed by extraction with dichloromethane (2×150 mL). The organic layer was washed with water (3×50 mL), dried over anhydrous sodium sulfate, and concentrated under reduced pressure to afford tert-butyl 2-oxo-[1,4′-bipiperidine]-1′-carboxylate (2.5 g, crude) as a light-yellow gum. Product was used for next step without any purification.

LCMS: 65.14% (m/z: 227.31, [M-^{t}Bu], 1.84 min (4 min run), 214 nm).

### Step-2: Preparation of [1,4′-bipiperidin]-2-one hydrochloride

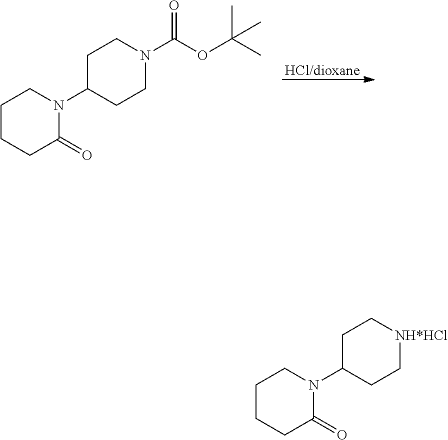

To a stirred solution of tert-butyl 2-oxo-[1,4′-bipiperidine]-1′-carboxylate (3.5 g) in dichloromethane (52 mL) at 0° C. was added 4 M hydrochloric acid in 1,4-dioxane (17.5 mL) under nitrogen atmosphere. Resulting reaction mixture was stirred at room temperature for 1 h. After completion of the reaction (monitored by TLC), reaction mass was evaporated under reduced pressure to obtain crude material. Crude was triturated with n-pentane to afford [1,4′-bipiperidin]-2-one*hydrochloride (2.8 g) as an off-white solid. Crude was carried forward to next step without any further purification.

¹H NMR (400 MHz, DMSO-d₆-D₂O): δ 4.43 (t, 1H), 3.30 (d, 2H), 3.12 (t, 2H), 2.96-2.89 (m, 3H), 2.22 (t, 2H), 1.95-1.86 (m, 2H), 1.66-1.62 (m, 6H).

### Step-3: Preparation of (7R,8R,9S,13S,14S,17S)-17-hydroxy-13-methyl-7-(9-((4,4,5,5,5-penta fluoropentyl)sulfinyl)nonyl)-7,8,9,11,12,13,14,15,16,17-decahydro-6H-cyclopenta[a]phenanthren-3-yl 2-oxo-[1,4′-bipiperidine]-1′-carboxylate

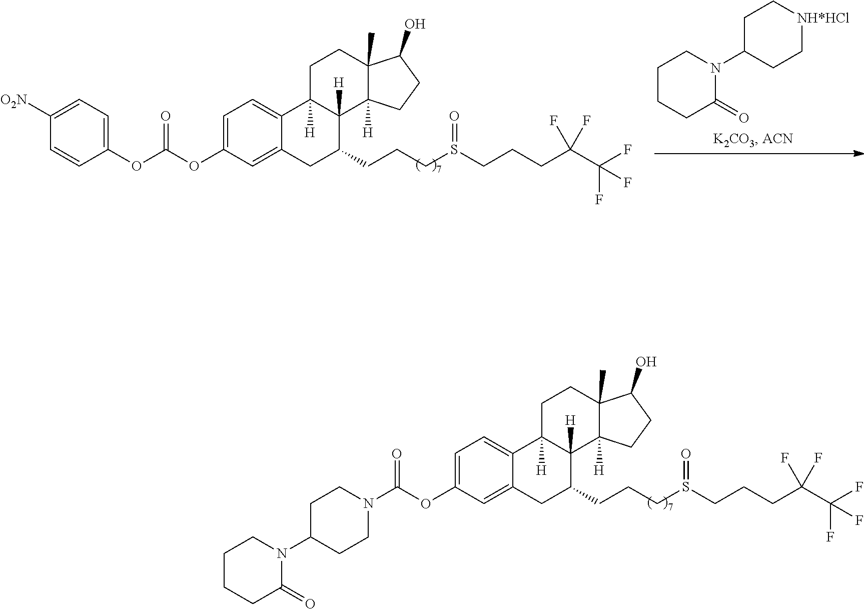

To a stirred solution of (7R,8R,9S,13S,14S,17S)-17-hydroxy-13-methyl-7-(9-((4,4,5,5,5-penta fluoropentyl)sulfinyl)nonyl)-7,8,9,11,12,13,14,15,16,17-decahydro-6H-cyclopenta[a]phenanthren-3-yl (4-nitrophenyl) carbonate (1.5 g, as prepared in example-34, step-1) in acetonitrile (20 mL) were added potassium carbonate (2.6 g) and [1,4′-bipiperidin]-2-one hydrochloride (0.635 g) at room temperature and the resulting reaction mixture was stirred at room temperature for 1 h. After completion of the reaction (monitored by TLC), reaction mass was quenched with ice-cold water followed by extraction using ethyl acetate (3×75 mL). Collected organics were dried over anhydrous sodium sulfate, evaporated under reduced pressure to obtain the crude which was purified by preparative HPLC to afford (7R,8R,9S,13S,14S,17S)-17-hydroxy-13-methyl-7-(9-((4,4,5,5,5-pentafluoropentyl)sulfinyl)nonyl)-7,8,9,11,12,13,14,15,16,17-decahydro-6H-cyclopenta[a]phenanthren-3-yl 2-oxo-[1,4′-bipiperidine]-1′-carboxylate (0.257 g, 16.2%) as a white solid.

LCMS: 99.38% (m/z: 815.31, [M+1]⁺, 6.63 min (10 min run), 214 nm).

¹H NMR (400 MHz, DMSO-d₆-D₂O): δ 7.25 (d, 1H), 6.81 (d, 1H), 6.76 (s, 1H), 4.44 (t, 1H), 4.07-4.16 (m, 1H), 3.52 (t, 1H), 3.16-3.13 (m, 2H), 3.11-3.01 (m, 1H), 2.93-2.62 (m, 7H), 2.43-2.28 (m, 3H), 2.22 (t, 3H), 1.93-1.78 (m, 4H), 1.66-1.40 (m, 13H), 1.34-1.14 (m, 19H).

### Example-43: Preparation of (7R,8R,9S,13S,14S,17S)-17-hydroxy-13-methyl-7-(9-((4,4,5,5,5-pentafluoropentyl)sulfinyl)nonyl)-7,8,9,11,12,13,14,15,16,17-decahydro-6H-cyclopenta[a]phenanthren-3-yl (2-ethylpyridin-4-yl)carbamate

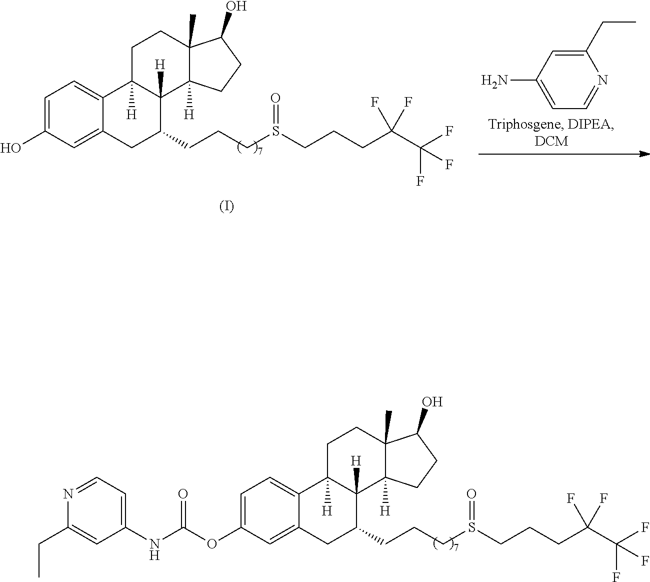

To a stirred solution of compound-I (0.5 g) in dichloromethane (8 mL) were added DIPEA (0.52 mL) and triphosgene (0.12 g) at 0° C. temperature and the resulting mixture was stirred at same temperature for 10 min. After that, 2-ethylpyridin-4-amine (0.151 g) was added, and the mixture was stirred for 2.5 h at room temperature. After completion of the reaction (monitored by TLC), the reaction mixture was diluted with water (300 mL) and then extracted with dichloromethane (2×500 mL). Collected organics, dried over anhydrous sodium sulfate, and concentrated under reduced pressure to obtain the crude material. The crude was purified by preparative HPLC to afford (7R,8R,9S,13S,14S,17S)-17-hydroxy-13-methyl-7-(9-((4,4,5,5,5-pentafluoropentyl)sulfinyl)nonyl)-7,8,9,11,12,13,14,15,16,17-decahydro-6H-cyclopenta[a]phenanthren-3-yl (2-ethylpyridin-4-yl)carbamate (0.145 g, 23.31%) as a white solid.

LCMS: 98.38% (m/z: 755.26, [M+1]⁺, 6.29 min (10 min run), 214 nm).

¹H NMR (400 MHz, DMSO-d₆-D₂O): δ 8.49 (d, 1H), 7.76-7.77 (m, 2H), 7.35 (d, 1H), 6.97 (d, 1H), 6.93 (s, 1H), 3.53 (t, 1H), 2.92-2.63 (m, 8H), 2.40-2.28 (m, 3H), 1.93-1.79 (m, 4H), 1.69-1.71 (m, 1H), 1.60-1.47 (m, 4H), 1.36-1.14 (m, 22H), 0.85-0.87 (m, 1H), 0.66 (s, 3H).

### Example-44: Preparation of (7R,8R,9S,13S,14S,17S)-17-hydroxy-13-methyl-7-(9-((4,4,5,5,5-pentafluoropentyl)sulfinyl)nonyl)-7,8,9,11,12,13,14,15,16,17-decahydro-6H-cyclopenta[a]phenanthren-3-yl (2-(trifluoromethyl)pyridin-4-yl)carbamate-9-carboxylate

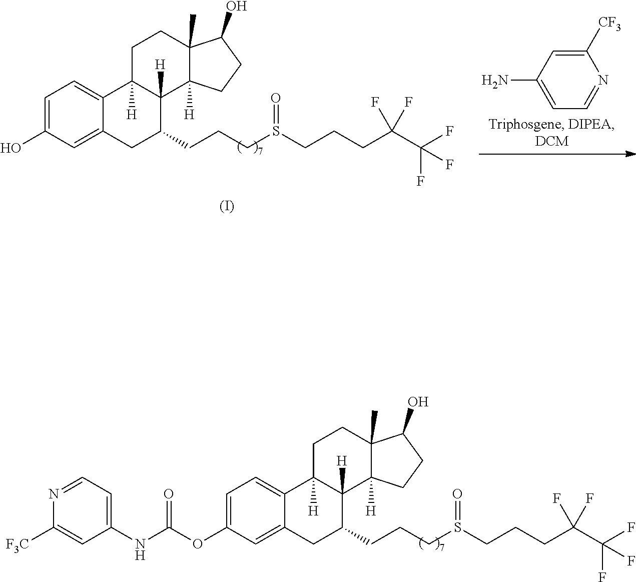

To a stirred solution of compound-I (0.5 g) in dichloromethane (10 mL) were added DIPEA (0.21 mL) and triphosgene (0.12 g) at 0° C. temperature and resultant reaction mixture was stirred at the same temperature for 10 min. 2-(trifluoromethyl)pyridin-4-amine (0.2 g) was added to the reaction mixture at 0° C. temperature and the reaction mixture was stirred at room temperature for 16 h.

After completion of reaction (monitored by TLC), the reaction mass was diluted with water (25 mL) and extracted with dichloromethane (2×25 mL). The combined organic layer was dried over anhydrous sodium sulfate and concentrated under reduced pressure to give crude material, which was purified by preparative HPLC to afford (7R,8R,9S,13S,14S,17S)-17-hydroxy-13-methyl-7-(9-((4,4,5,5,5-pentafluoropentyl)sulfinyl)nonyl)-7,8,9,11,12,13,14,15,16,17-decahydro-6H-cyclopenta[a]phenanthren-3-yl (2-(trifluoromethyl)pyridin-4-yl)carbamate-9-carboxylate (35 mg, 5.4%) as a white solid.

LCMS: 49.81+48.97%% (m/z: 795.22, [M+1]⁺, 5.41 and 5.47 min (10 min run), 214 nm).

¹H NMR (400 MHz, DMSO-d₆-D₂O, mix. of isomers): δ 8.58 (d, 1H), 8.07 (d, 1H), 7.94 (s, 1H), 7.67 (d, 1H), 7.33 (d, 1H), 7.04 (d, 1H), 6.95 (d, 1H), 6.90-6.89 (m, 2H), 6.66-6.65 (m, 1H), 6.48-6.50 (m, 1H), 6.41 (s, 1H), 3.65-3.42 (m, 2H), 2.86-2.61 (m, 11H), 2.38-2.10 (m, 6H), 1.93-1.75 (m, 6H), 1.75-1.46 (m, 8H), 1.45-1.30 (m, 8H), 1.25-1.13 (m, 20H), 0.88-0.86 (m, 1H), 0.63-0.66 (m, 4H).

### Example 45: Cytotoxic Effect of Compound (I-a) and Compound (I-Bh) on the Human Breast Cancer MCF-7 Cells

MCF-7 human breast cancer cells were procured from the American Type Culture Collection (ATCC, USA) and, were cultured and maintained in Dulbecco's modified Eagle's medium supplemented with 10% fetal bovine serum, and 1% penicillin streptomycin at 37° C. in a humidified 5% C02 incubator. Cells were harvested when they reach 70-80% confluence using 0.25% Trypsin-EDTA and were seeded in 96-well tissue culture treated plates @5000 cells/200 μL/well and were maintained in phenol-red Dulbecco's modified Eagle's medium supplemented with 10% fetal bovine serum, and 1% penicillin streptomycin (Growth factor deprived condition). Approximately 24 h after seeding the cells in plate were treated with respective 9 concentrations of compound (I-a) and compound (I-bh) (10000, 1000, 300, 100, 30, 10, 3, 1, 0.1 nM) in triplicates. Thereafter the plates were incubated at 37° C., 5% CO₂ for 5 days with media replacement on day #3.

For drug formulations, vehicle (0.1% DMSO conc.) was used. Negative control (MAX Viability) was media (with cells) in the presence of 0.1% DMSO. Positive control (MIN Viability) was media (with cells) in presence of highest concentration of compound paclitaxel (1 μM) as reference. After 5 days of treatment, cells were terminated/lysed with Cell titer Glo (CTG) reagent. 40 μl of the content was Transferred in luminescent Plate to record luminescence.

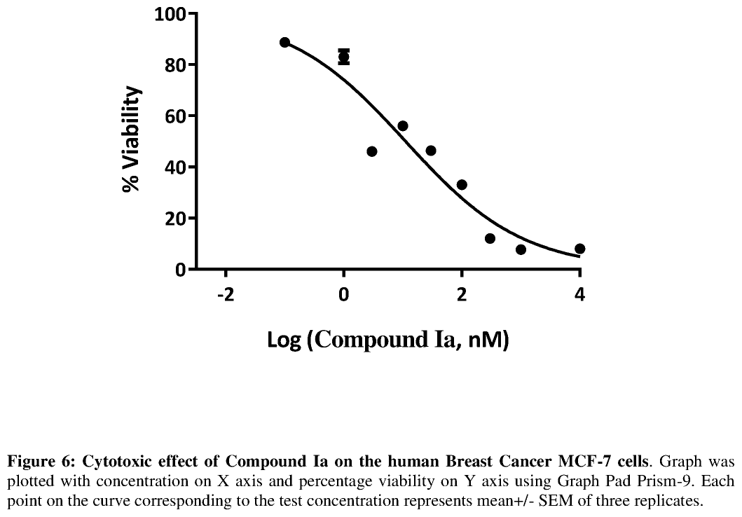

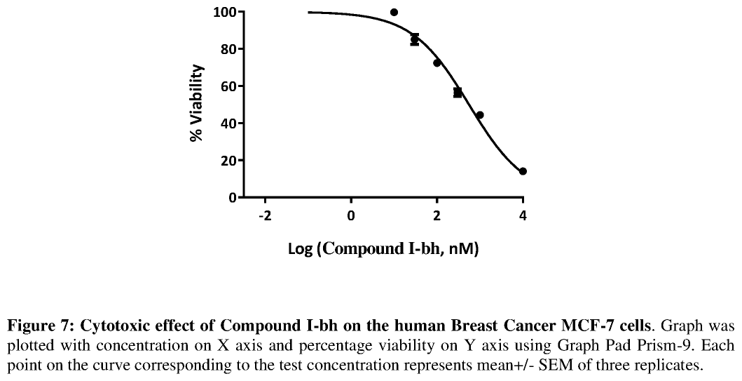

All statistical analysis were performed by Graph Pad Prism-9 software using four-parametric non-linear regression analysis as shown in FIG. 6 and FIG. 7. The IC50 values of compound (I-a) and compound (I-bh) were reported to be 11.15 nM and 555.4 nM respectively.

### Example 46: Effect of Compound (I-a) and Compound (I-Bh) on the ER-Alpha Expression of Human Breast Cancer MCF-7 Cell Line

MCF-7 human breast cancer cells were procured from the American Type Culture Collection (ATCC, USA) and, were cultured and maintained in Dulbecco's modified Eagle's medium supplemented with 10% fetal bovine serum, and 1% penicillin streptomycin at 37° C. in a humidified 5% CO₂ incubator. Cells were harvested when they reach 70-80% confluence using 0.25% Trypsin-EDTA. 3-4 days' prior drug treatment, the cells were maintained in Growth factor deprived condition at 37° C. in a humidified 5% C02 incubator. Post 3-4 days, cells were trypsinized and thereafter, ˜0.20*10⁶ cells/well/mL were seeded in a 24 well culture plates.

Approximately 24 h after seeding, the cells were treated with respective 5 concentrations of compound (I-a) (100, 10, 1, 0.1 & 0.01 μM) and compound (I-bh) (30, 10, 1, 0.1 & 0.01 μM) at 37° C., 5% C02 for another 24 h. For drug formulations, vehicle (0.1% DMSO conc.) was used.

The human MCF-7 cells were used as negative or Vehicle control. After 24 h of treatment, plates washed with PBS, and were lysed with −100 μL of RIPA buffer. Thereafter, cell lysates was collected and supernatant were assayed for protein estimation by Bradford assay and 0.4 μg/μL (ie., 0.4 mg/mL) of protein was used for simple western (WES) with primary antibody (ER-α, 1:50 conc.) and housekeeping gene (GAPDH, 1:2000 conc.). WES were run at one-time point in duplicates. After incubation of samples with primary and secondary antibody, resulting Chemiluminescence were measured by the instrument.

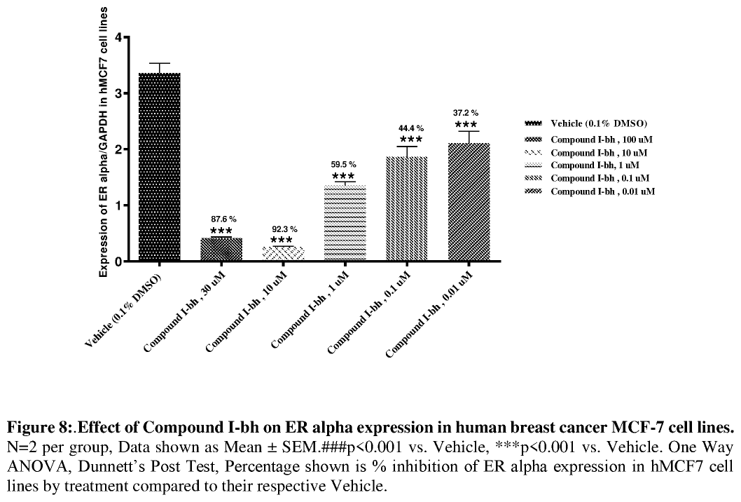

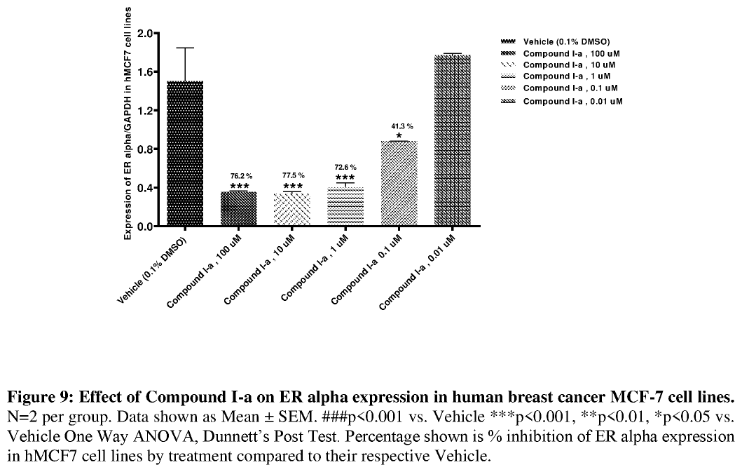

The band area from the software calculation for Vehicle and ER-alpha were considered for analysis. Ratio of ER-alpha to housing keeping protein (GAPDH) were analyzed. The fold over Vehicle control were calculated for plotting graphs as shown in FIG. 8 and FIG. 9.

### Example 47: Pharmacokinetic Data in Rat

The objective of the study was to evaluate pharmacokinetic exposure of compound (I-a) or compound (I-bh) after per oral dosing at 10 mg/kg of compound (I-a) or compound (I-bh) in female Sprague Dawley rat. This study was performed in overnight fasted female Sprague Dawley rat. Dose formulation was prepared freshly on the day of dosing. 10% solutol+20% PG+70% PBS was used as vehicle for the preparation of dose formulation.

Blood samples were collected through retro orbital plexus puncture at pre-dose, 0.25, 0.5, 1, 2, 4, 8, 12 and 24 h post-dose. At each time point, blood was withdrawn and transferred into a pre-labeled micro centrifuge tubes containing anti-coagulant. Tube was mixed gently by inverting the tube to facilitate mixing of anticoagulant with the blood. All blood/plasma samples were analysed using fit-for purpose LC-MS/MS method.

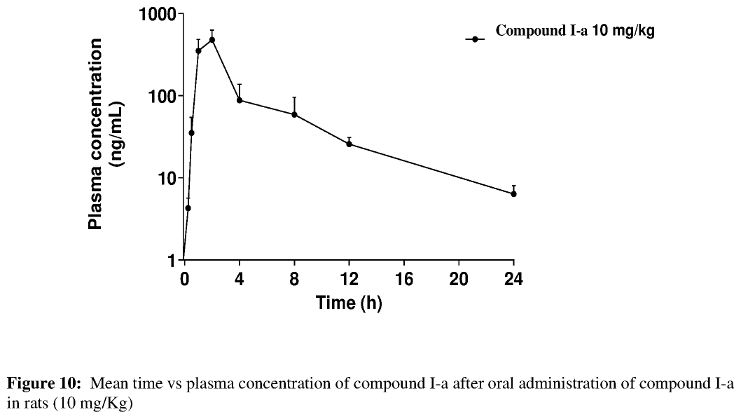

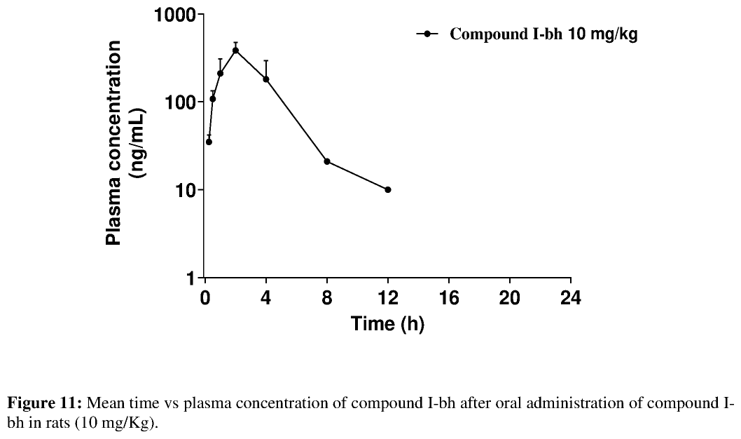

The pharmacokinetic parameters were calculated using standard non-compartmental analysis (Phoenix® software, version 8.3, Pharsight Corporation, Mountain View, California 94040/USA) using linear trapezoidal method with linear interpolation. Refer FIG. 10 and FIG. 11.

TABLE 8
Arithmetic Mean pharmacokinetic parameters of compound (I-a)
or compound (I-bh) after per oral dosing at 10 mg/kg of compound
(I-a) or compound (I-bh) in female Sprague Dawley rat.

| | PK parameters | | | |
|---|---|---|---|---|
| | Cmax | Tₘₐₓ | AUCₗₐₛₜ | AUC_{inf}_{_{—}}_{obs} |
| Analytes | (ng/mL) | (h) | (h · ng/mL) | (h · ng/mL) |
| compound (I-a) | 503 | 1.42 | 1741 | 1793 |
| compound (I-bh) | 384 | 2 | 1430 | 1460 |

### Example 48: Anticancer Efficacy of Compound (I-a) in Nude Mice Bearing MCF7 Tumor

For this purpose, MCF7 cancer cells were procured from ATCC and grown in DMEM Medium supplemented with 10% FBS and 1% penicillin streptomycin. Cells were harvested when they reach 70-80% confluence for tumor implantation. 24 h prior to MCF7 cells implantation, the skin area around the injection site of nude mice were disinfected by mildly swabbing with surgical spirit. 170-estradiol pellet (0.18 mg/pellet 60-day release, Innovative Research of America) was subcutaneously implanted into in the left flank region of the animals. For cell inoculation, cells were mixed with equal volume of Matrigel in a 1:1 ratio followed by injection of 5×10⁶ MCF7 cells subcutaneously on the right flank region using a 1 mL syringe attached to a 24 G needle. On the subsequent days the injection site was monitored for tumor palpability. Tumor grafts were measured within 7-8 days of cell inoculation for tumor palpability. When tumor volume of the implanted animals reaches 200±50 mm³, animals were randomized based on the tumor volume into different groups according to the study design.

Randomization was carried out in such a way that the mean tumor volume of the individual group remain similar or not significantly different from each other. After randomization, treatment was initiated when the average tumor volume reaches −200±50 mm³. Tumor volume and mouse body weight were measured on the first day of treatment (Day 1) and then three times per week till 17 days.

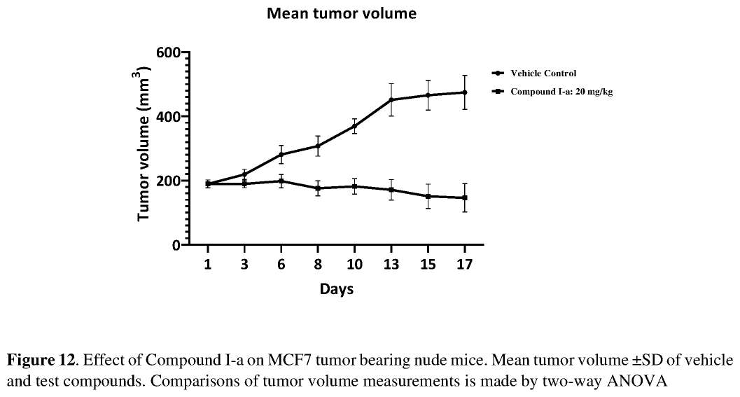

Dose formulations of compound (I-a) (20 mg/kg) and vehicle (0.5% MC) was administered by oral gavage daily for 17 days. Refer FIG. 12.

Tumor volume (mm³) was calculated using the formula, [tumor length×tumor width(2)]/2. Tumor growth inhibition will be calculated after normalizing the values to that on day 1. % TGI was calculated based on the formula below.

$$\% T G I = [ 1 - ( Mean T V of treatment ) / Mean T V of Control ] \times 100$$

TABLE 9
% Tumor Growth Inhibition (% TGI) of different concentrations
of compound(I-a) on day 28 in MCF-7 xenograft mice model.

| | | % Tumor Growth Inhibition |
|---|---|---|
| Group | Treatment | (% TGI) on day 17 |
| 1 | Vehicle | 0 |
| 2 | Compound(I-a) 20 mg/kg | 115 |

1. A compound of formula (I) 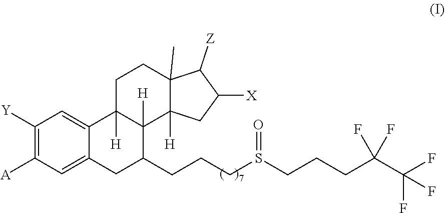
wherein;
X is halogen or hydrogen;
Y is alkyl, —O-alkyl or hydrogen;
Z is selected from ═O, ═N—OH, —O-alkyl, —O-haloalkyl, —NH—OH or —O-alkyl-OH wherein the bond between the Z substituent attached to carbon can be single or double bond depend on the substituent selected;
A is selected from 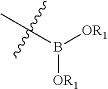
or —O—R₂, wherein R₁ is selected from hydrogen or alkyl; R₂ is selected independently from group consisting of
i) 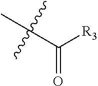
wherein R₃ is selected from NH₂, NHR₄, or NR₅R₆; R₄ is selected from group comprising optionally substituted alkyl, alkenyl, alkynyl, cycloalkyl, heterocycloalkyl, aryl, or heteroaryl; R₅ and R₆ are selected independently from group comprising optionally substituted alkyl, aryl, heteroaryl, heterocycloalkyl, cycloalkyl; or R₅ and R₆ are taken together along with the nitrogen to which they are attached to form a three to seven membered heterocyclic ring and may optionally be substituted at a substitutable position with one or more R₇ radicals, wherein the one or more R₇ radicals are independently selected at each occurrence from the group consisting of optionally substituted alkyl, alkylcarbonyl, formyl, halo, haloalkyl, alkylphosphate, phosphate, oxo, cyano, nitro, amino, hydroxyalkylamino, alkoxy, alkoxycarbonyl, carboxyalkyl, hydroxyalkyl, alkenyl, alkynyl, alkylthio, cycloalkyl, aryl, heteroaryl, aralkyl, heterocycloalkyl, alkylthioalkyl, arylcarbonyl, aralkylcarbonyl, alkoxyalkyl;
ii) 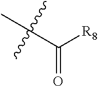
wherein R₈ is selected from group comprising optionally substituted alkyl, alkenyl, alkynyl, cycloalkyl, aryl, heteroaryl; or —CR₉R₁₀; where R₉ and R₁₀ are taken together along with the carbon to which they are attached to form a three to seven membered saturated, partially unsaturated or unsaturated heterocyclic ring in which up to 4 carbon atoms are replaced by heteroatoms chosen from the group consisting of O, S or N and may optionally be substituted at a substitutable position with one or more R₁₁ radicals, wherein one or more R₁₁ radicals are independently selected at each occurrence from the group consisting of optionally substituted alkyl, cycloalkyl, heterocycloalkyl, aryl, heteroaryl, alkylcarbonyl, formyl, halo, alkylphosphate, phosphate, cyano, nitro, alkoxy, amino, alkoxycarbonyl, alkenyl, alkynyl, alkylthio arylcarbonyl;
iii) 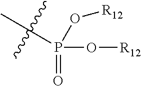
wherein each R₁₂ is selected independently from hydrogen; optionally substituted optionally substituted alkyl, aryl, acyl, heterocycloalkyl or heteroaryl;
iv) 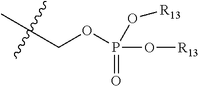
wherein each R₁₃ is selected independently from hydrogen; optionally substituted alkyl, cycloalkyl, heterocycloalkyl, aryl, or heteroaryl;
v) amino acids, wherein the amino acid is linked via ester linkage at the point of attachment;
vi) 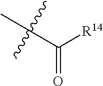
wherein R¹⁴ is 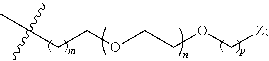
wherein m and p are independently selected from 0 to 5, n refers to degree of polymerization and is selected from 1 to 250 and Z is optionally substituted alkyl or cycloalkyl;
vii) 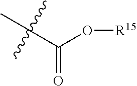
wherein R¹⁵ is selected from group comprising of optionally substituted alkyl, alkenyl, alkynyl, cycloalkyl, heterocycloalkyl, aryl and heteroaryl;
viii) 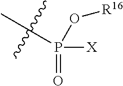
wherein R¹⁶ is selected from group comprising of hydrogen; optionally substituted alkyl, aryl, acyl, heteroaryl; and X is optionally substituted heterocycloalkyl;
ix) 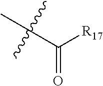
wherein R₁₇ is selected from optionally substituted bicyclic compounds, spirocyclic compounds or bridged bicyclic compounds; pharmaceutically acceptable salts, enantiomers, diastereomers, racemates, or solvates thereof.

2. A compound of formula (I-A) 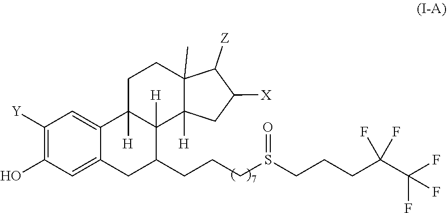
wherein;
X is halogen or hydrogen;
Y is alkyl, —O-alkyl or hydrogen;
Z is selected from ═O, ═N—OH, —O-alkyl, O-haloalkyl, —NH—OH or —O-alkyl-OH wherein the bond between the Z substituent attached to carbon can be single or double bond depend on the substituent selected;
pharmaceutically acceptable salts, enantiomers, diastereomers, racemates, or solvates thereof.

3. The compound according to claim 1 comprising the compound (II) represented by following formulae: 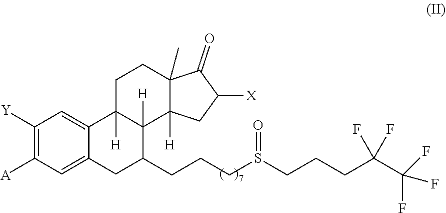
wherein;
X is halogen or hydrogen;
Y is alkyl, —O-alkyl or hydrogen;
A is selected from 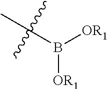
or —O—R₂, wherein R₁ is selected from hydrogen or alkyl; R₂ is selected independently from group consisting of
i) 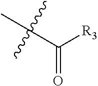
wherein R₃ is selected from NH₂, NHR₄, or NR₅R₆; R₄ is selected from group comprising optionally substituted alkyl, alkenyl, alkynyl, cycloalkyl, heterocycloalkyl, aryl, or heteroaryl; R₅ and R₆ are selected independently from group comprising optionally substituted alkyl, aryl, heteroaryl, heterocycloalkyl, cycloalkyl; or R₅ and R₆ are taken together along with the nitrogen to which they are attached to form a three to seven membered heterocyclic ring and may optionally be substituted at a substitutable position with one or more R₇ radicals, wherein the one or more R₇ radicals are independently selected at each occurrence from the group consisting of optionally substituted alkyl, alkylcarbonyl, formyl, halo, haloalkyl, alkylphosphate, phosphate, oxo, cyano, nitro, amino, hydroxyalkylamino, alkoxy, alkoxycarbonyl, carboxyalkyl, hydroxyalkyl, alkenyl, alkynyl, alkylthio, cycloalkyl, aryl, heteroaryl, aralkyl, heterocycloalkyl, alkylthioalkyl, arylcarbonyl, aralkylcarbonyl, alkoxyalkyl;
ii) 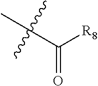
wherein R₈ is selected from group comprising optionally substituted alkyl, alkenyl, alkynyl, cycloalkyl, aryl, heteroaryl; or —CR₉R₁₀; where R₉ and R₁₀ are taken together along with the carbon to which they are attached to form a three to seven membered saturated, partially unsaturated or unsaturated heterocyclic ring in which up to 4 carbon atoms are replaced by heteroatoms chosen from the group consisting of O, S or N and may optionally be substituted at a substitutable position with one or more R₁₁ radicals, wherein one or more R₁₁ radicals are independently selected at each occurrence from the group consisting of optionally substituted alkyl, cycloalkyl, heterocycloalkyl, aryl, heteroaryl, alkylcarbonyl, formyl, halo, alkylphosphate, phosphate, cyano, nitro, alkoxy, amino, alkoxycarbonyl, alkenyl, alkynyl, alkylthio
iii) 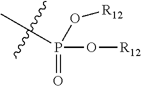
wherein each R₁₂ is selected independently from hydrogen; optionally substituted optionally substituted alkyl, aryl, acyl, heterocycloalkyl or heteroaryl;
iv) 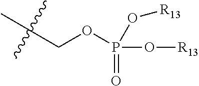
wherein each R₁₃ is selected independently from hydrogen; optionally substituted alkyl, cycloalkyl, heterocycloalkyl, aryl, or heteroaryl;
v) amino acids, wherein the amino acid is linked via ester linkage at the point of attachment;
vi) 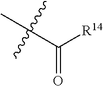
wherein R¹⁴ is 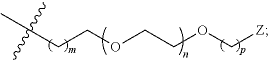
wherein m and p are independently selected from 0 to 5, n refers to degree of polymerization and is selected from 1 to 250 and Z is optionally substituted alkyl or cycloalkyl;
vii) 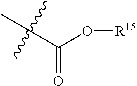
wherein R¹⁵ is selected from group comprising of optionally substituted alkyl, alkenyl, alkynyl cycloalkyl, heterocycloalkyl, aryl and heteroaryl;
viii) 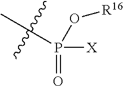
wherein R¹⁶ is selected from group comprising of hydrogen; optionally substituted alkyl, aryl, acyl, heteroaryl; and X is optionally substituted heterocycloalkyl;
ix) 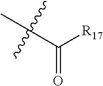
wherein R₁₇ is selected from optionally substituted bicyclic compounds, spirocyclic compounds or bridged bicyclic compounds; pharmaceutically acceptable salts, enantiomers, diastereomers, racemates, or solvates thereof.

4. The compound according to claim 1 comprising the compound (III) represented by following formulae: 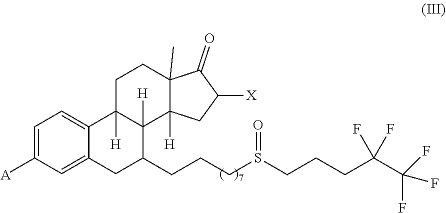
wherein X and A has the same meaning as given in compound of formula (II); pharmaceutically acceptable salts, enantiomers, diastereomers, racemates, or solvates thereof.

5. The compound according to claim 1 comprising the compound (IV) represented by following formulae: 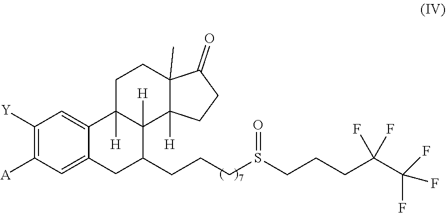
wherein Y and A has the same meaning as given in compound of formula (II); pharmaceutically acceptable salts, enantiomers, diastereomers, racemates, or solvates thereof.

6. The compound according to claim 1 comprising the compound (V) represented by following formulae: 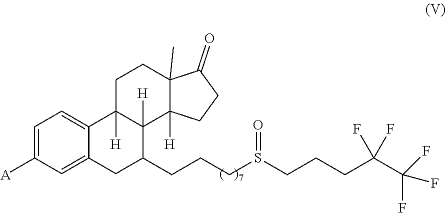
wherein;
A is selected from 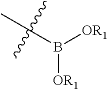
or —O—R₂, wherein R₁ is selected from hydrogen or alkyl; R₂ is selected independently from group consisting of
i) 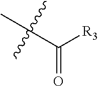
wherein R₃ is selected from NH₂, NHR₄, or NR₅R₆; R₄ is selected from group comprising optionally substituted alkyl, alkenyl, alkynyl, cycloalkyl, heterocycloalkyl, aryl, or heteroaryl; R₅ and R₆ are selected independently from group comprising optionally substituted alkyl, aryl, heteroaryl, heterocycloalkyl, cycloalkyl; or R₅ and R₆ are taken together along with the nitrogen to which they are attached to form a three to seven membered heterocyclic ring and may optionally be substituted at a substitutable position with one or more R₇ radicals, wherein the one or more R₇ radicals are independently selected at each occurrence from the group consisting of optionally substituted alkyl, alkylcarbonyl, formyl, halo, haloalkyl, alkylphosphate, phosphate, oxo, cyano, nitro, amino, hydroxyalkylamino, alkoxy, alkoxycarbonyl, carboxyalkyl, hydroxyalkyl, alkenyl, alkynyl, alkylthio, cycloalkyl, aryl, heteroaryl, aralkyl, heterocycloalkyl, alkylthioalkyl, arylcarbonyl, aralkylcarbonyl, alkoxyalkyl;
ii) 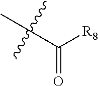
wherein R₈ is selected from group comprising optionally substituted alkyl, alkenyl, alkynyl, cycloalkyl, aryl, heteroaryl; or —CR₉R₁₀; where R₉ and R₁₀ are taken together along with the carbon to which they are attached to form a three to seven membered saturated, partially unsaturated or unsaturated heterocyclic ring in which up to 4 carbon atoms are replaced by heteroatoms chosen from the group consisting of O, S or N and may optionally be substituted at a substitutable position with one or more R₁₁ radicals, wherein one or more R₁₁ radicals are independently selected at each occurrence from the group consisting of optionally substituted alkyl, cycloalkyl, heterocycloalkyl, aryl, heteroaryl, alkylcarbonyl, formyl, halo, alkylphosphate, phosphate, cyano, nitro, alkoxy, amino, alkoxycarbonyl, alkenyl, alkynyl, alkylthio arylcarbonyl;
iii) 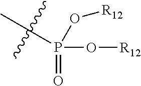
wherein each R₁₂ is selected independently from hydrogen; optionally substituted optionally substituted alkyl, aryl, acyl, heterocycloalkyl or heteroaryl;
iv) 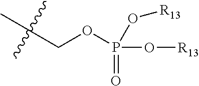
wherein each R₁₃ is selected independently from hydrogen; optionally substituted alkyl, cycloalkyl, heterocycloalkyl, aryl, or heteroaryl;
v) amino acids, wherein the amino acid is linked via ester linkage at the point of attachment;
vi) 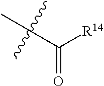
wherein R¹⁴ is 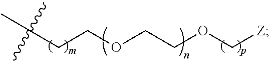
wherein m and p are independently selected from 0 to 5, n refers to degree of polymerization and is selected from 1 to 250 and Z is optionally substituted alkyl or cycloalkyl;
vii) 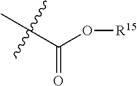
wherein R¹⁵ is selected from group comprising of optionally substituted alkyl, alkenyl, alkynyl, cycloalkyl, heterocycloalkyl, aryl and heteroaryl;
viii) 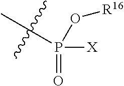
wherein R¹⁶ is selected from group comprising of hydrogen; optionally substituted alkyl, aryl, acyl, heteroaryl; and X is optionally substituted heterocycloalkyl;
ix) 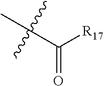
wherein R₁₇ is selected from optionally substituted bicyclic compounds, spirocyclic compounds or bridged bicyclic compounds; pharmaceutically acceptable salts, enantiomers, diastereomers, racemates, or solvates thereof.

7. The compound according to claim 1 comprising the compound (VI) represented by following formulae: 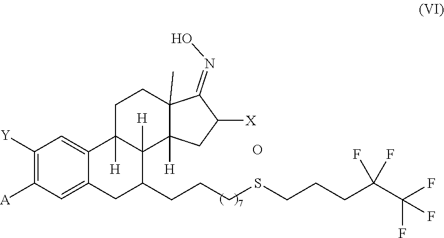
wherein;
X is halogen or hydrogen;
Y is alkyl, —O-alkyl or hydrogen;
A is selected from 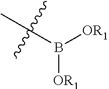
or —O—R₂, wherein R₁ is selected from hydrogen or alkyl; R₂ is selected independently from group consisting of
i) 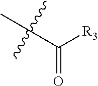
wherein R₃ is selected from NH₂, NHR₄, or NR₅R₆; R₄ is selected from group comprising optionally substituted alkyl, alkenyl, alkynyl, cycloalkyl, heterocycloalkyl, aryl, or heteroaryl; R₅ and R₆ are selected independently from group comprising optionally substituted alkyl, aryl, heteroaryl, heterocycloalkyl, cycloalkyl; or R₅ and R₆ are taken together along with the nitrogen to which they are attached to form a three to seven membered heterocyclic ring and may optionally be substituted at a substitutable position with one or more R₇ radicals, wherein the one or more R₇ radicals are independently selected at each occurrence from the group consisting of optionally substituted alkyl, alkylcarbonyl, formyl, halo, haloalkyl, alkylphosphate, phosphate, oxo, cyano, nitro, amino, hydroxyalkylamino, alkoxy, alkoxycarbonyl, carboxyalkyl, hydroxyalkyl, alkenyl, alkynyl, alkylthio, cycloalkyl, aryl, heteroaryl, aralkyl, heterocycloalkyl, alkylthioalkyl, arylcarbonyl, aralkylcarbonyl, alkoxyalkyl;
ii) 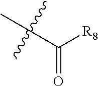
wherein R₈ is selected from group comprising optionally substituted alkyl, alkenyl, alkynyl, cycloalkyl, aryl, heteroaryl; or —CR₉R₁₀; where R₉ and R₁₀ are taken together along with the carbon to which they are attached to form a three to seven membered saturated, partially unsaturated or unsaturated heterocyclic ring in which up to 4 carbon atoms are replaced by heteroatoms chosen from the group consisting of O, S or N and may optionally be substituted at a substitutable position with one or more R₁₁ radicals, wherein one or more R₁₁ radicals are independently selected at each occurrence from the group consisting of optionally substituted alkyl, cycloalkyl, heterocycloalkyl, aryl, heteroaryl, alkylcarbonyl, formyl, halo, alkylphosphate, phosphate, cyano, nitro, alkoxy, amino, alkoxycarbonyl, alkenyl, alkynyl, alkylthio arylcarbonyl;
iii) 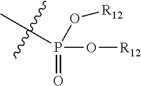
wherein each R₁₂ is selected independently from hydrogen; optionally substituted optionally substituted alkyl, aryl, acyl, heterocycloalkyl or heteroaryl;
iv) 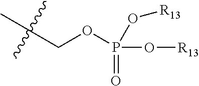
wherein each R₁₃ is selected independently from hydrogen; optionally substituted alkyl, cycloalkyl, heterocycloalkyl, aryl, or heteroaryl;
v) amino acids, wherein the amino acid is linked via ester linkage at the point of attachment;
vi) 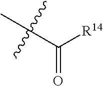
wherein R¹⁴ is 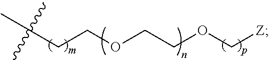
wherein m and p are independently selected from 0 to 5, n refers to degree of polymerization and is selected from 1 to 250 and Z is optionally substituted alkyl or cycloalkyl;
vii) 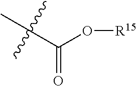
wherein R¹⁵ is selected from group comprising of optionally substituted alkyl, alkenyl, alkynyl, cycloalkyl, heterocycloalkyl, aryl and heteroaryl;
viii) 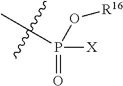
wherein R¹⁶ is selected from group comprising of hydrogen; optionally substituted alkyl, aryl, acyl, heteroaryl; and X is optionally substituted heterocycloalkyl;
ix) 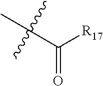
wherein R₁₇ is selected from optionally substituted bicyclic compounds, spirocyclic compounds or bridged bicyclic compounds; pharmaceutically acceptable salts, enantiomers, diastereomers, racemates, or solvates thereof.

8. The compound according to claim 1 comprising the compound (VII) represented by following formulae: 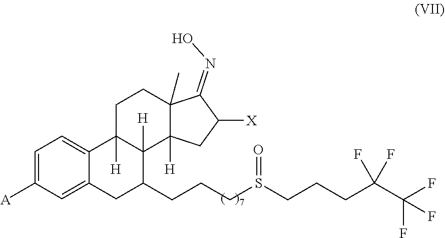
wherein X and A has the same meaning as given in compound of formula (VI); pharmaceutically acceptable salts, enantiomers, diastereomers, racemates, or solvates thereof.

9. The compound according to claim 1 comprising the compound (VIII) represented by following formulae: 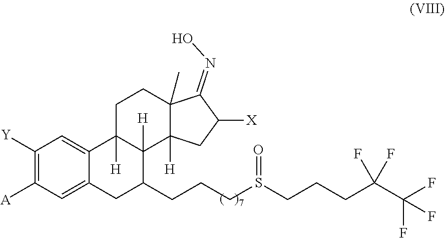
wherein Y and A has the same meaning as given in compound of formula (VI); pharmaceutically acceptable salts, enantiomers, diastereomers, racemates, or solvates thereof.

10. The compound according to claim 1 comprising the compound (IX) represented by following formulae: 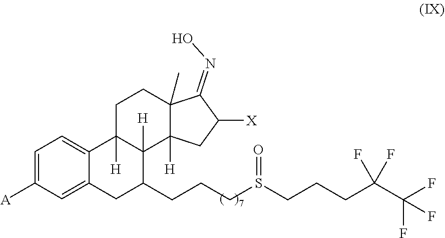
A is selected from 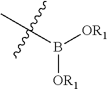
or —O—R₂, wherein R₁ is selected from hydrogen or alkyl; R₂ is selected independently from group consisting of
i) 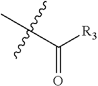
wherein R₃ is selected from NH₂, NHR₄, or NR₅R₆; R₄ is selected from group comprising optionally substituted alkyl, alkenyl, alkynyl, cycloalkyl, heterocycloalkyl, aryl, or heteroaryl; R₅ and R₆ are selected independently from group comprising optionally substituted alkyl, aryl, heteroaryl, heterocycloalkyl, cycloalkyl; or R₅ and R₆ are taken together along with the nitrogen to which they are attached to form a three to seven membered heterocyclic ring and may optionally be substituted at a substitutable position with one or more R₇ radicals, wherein the one or more R₇ radicals are independently selected at each occurrence from the group consisting of optionally substituted alkyl, alkylcarbonyl, formyl, halo, haloalkyl, alkylphosphate, phosphate, oxo, cyano, nitro, amino, hydroxyalkylamino, alkoxy, alkoxycarbonyl, carboxyalkyl, hydroxyalkyl, alkenyl, alkynyl, alkylthio, cycloalkyl, aryl, heteroaryl, aralkyl, heterocycloalkyl, alkylthioalkyl, arylcarbonyl, aralkylcarbonyl, alkoxyalkyl;
ii) 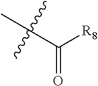
wherein R₈ is selected from group comprising optionally substituted alkyl, alkenyl, alkynyl, cycloalkyl, aryl, heteroaryl; or —CR₉R₁₀; where R₉ and R₁₀ are taken together along with the carbon to which they are attached to form a three to seven membered saturated, partially unsaturated or unsaturated heterocyclic ring in which up to 4 carbon atoms are replaced by heteroatoms chosen from the group consisting of O, S or N and may optionally be substituted at a substitutable position with one or more R₁₁ radicals, wherein one or more R₁₁ radicals are independently selected at each occurrence from the group consisting of optionally substituted alkyl, cycloalkyl, heterocycloalkyl, aryl, heteroaryl, alkylcarbonyl, formyl, halo, alkylphosphate, phosphate, cyano, nitro, alkoxy, amino, alkoxycarbonyl, alkenyl, alkynyl, alkylthio arylcarbonyl;
iii) 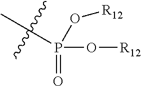
wherein each R₁₂ is selected independently from hydrogen; optionally substituted optionally substituted alkyl, aryl, acyl, heterocycloalkyl or heteroaryl;
iv) 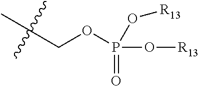
wherein each R₃ is selected independently from hydrogen; optionally substituted alkyl, cycloalkyl, heterocycloalkyl, aryl, or heteroaryl;
v) amino acids, wherein the amino acid is linked via ester linkage at the point of attachment;
vi) 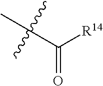
wherein R¹⁴ is 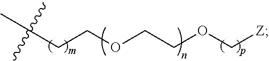
wherein m and p are independently selected from 0 to 5, n refers to degree of polymerization and is selected from 1 to 250 and Z is optionally substituted alkyl or cycloalkyl;
vii) 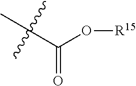
wherein R¹⁵ is selected from group comprising of optionally substituted alkyl, alkenyl, alkynyl, cycloalkyl, heterocycloalkyl, aryl and heteroaryl;
viii) 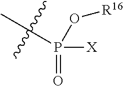
wherein R¹⁶ is selected from group comprising of hydrogen; optionally substituted alkyl, aryl, acyl, heteroaryl; and X is optionally substituted heterocycloalkyl;
ix) 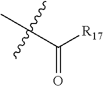
wherein R₁₇ is selected from optionally substituted bicyclic compounds, spirocyclic compounds or bridged bicyclic compounds; pharmaceutically acceptable salts, enantiomers, diastereomers, racemates, or solvates thereof.

11. The compound according to claim 1 comprising the compound (X) represented by following formulae: 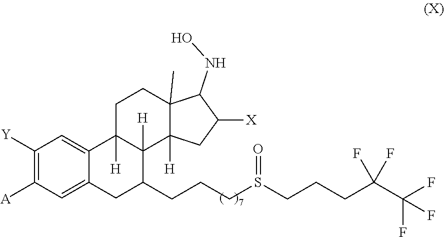
X is halogen or hydrogen;
Y is alkyl, —O-alkyl or hydrogen;
A is selected from 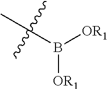
or —O—R₂, wherein R₁ is selected from hydrogen or alkyl; R₂ is selected independently from group consisting of
i) 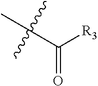
wherein R₃ is selected from NH₂, NHR₄, or NR₅R₆; R₄ is selected from group comprising optionally substituted alkyl, alkenyl, alkynyl, cycloalkyl, heterocycloalkyl, aryl, or heteroaryl; R₅ and R₆ are selected independently from group comprising optionally substituted alkyl, aryl, heteroaryl, heterocycloalkyl, cycloalkyl; or R₅ and R₆ are taken together along with the nitrogen to which they are attached to form a three to seven membered heterocyclic ring and may optionally be substituted at a substitutable position with one or more R₇ radicals, wherein the one or more R₇ radicals are independently selected at each occurrence from the group consisting of optionally substituted alkyl, alkylcarbonyl, formyl, halo, haloalkyl, alkylphosphate, phosphate, oxo, cyano, nitro, amino, hydroxyalkylamino, alkoxy, alkoxycarbonyl, carboxyalkyl, hydroxyalkyl, alkenyl, alkynyl, alkylthio, cycloalkyl, aryl, heteroaryl, aralkyl, heterocycloalkyl, alkylthioalkyl, arylcarbonyl, aralkylcarbonyl, alkoxyalkyl;
ii) 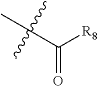
wherein R₈ is selected from group comprising optionally substituted alkyl, alkenyl, alkynyl, cycloalkyl, aryl, heteroaryl; or —CR₉R₁₀; where R₉ and R₁₀ are taken together along with the carbon to which they are attached to form a three to seven membered saturated, partially unsaturated or unsaturated heterocyclic ring in which up to 4 carbon atoms are replaced by heteroatoms chosen from the group consisting of O, S or N and may optionally be substituted at a substitutable position with one or more R₁₁ radicals, wherein one or more R₁₁ radicals are independently selected at each occurrence from the group consisting of optionally substituted alkyl, cycloalkyl, heterocycloalkyl, aryl, heteroaryl, alkylcarbonyl, formyl, halo, alkylphosphate, phosphate, cyano, nitro, alkoxy, amino, alkoxycarbonyl, alkenyl, alkynyl, alkylthio arylcarbonyl;
iii) 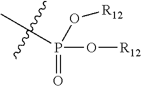
wherein each R₁₂ is selected independently from hydrogen; optionally substituted optionally substituted alkyl, aryl, acyl, heterocycloalkyl or heteroaryl;
iv) 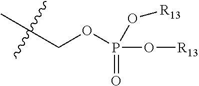
wherein each R₃ is selected independently from hydrogen; optionally substituted alkyl, cycloalkyl, heterocycloalkyl, aryl, or heteroaryl;
v) amino acids, wherein the amino acid is linked via ester linkage at the point of attachment;
vi) 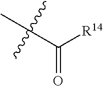
wherein R¹⁴ is 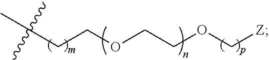
wherein m and p are independently selected from 0 to 5, n refers to degree of polymerization and is selected from 1 to 250 and Z is optionally substituted alkyl or cycloalkyl;
vii) 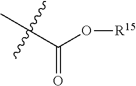
wherein R¹⁵ is selected from group comprising of optionally substituted alkyl, alkenyl, alkynyl, cycloalkyl, heterocycloalkyl, aryl and heteroaryl;
viii) 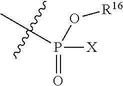
wherein R¹⁶ is selected from group comprising of hydrogen; optionally substituted alkyl, aryl, acyl, heteroaryl; and X is optionally substituted heterocycloalkyl;
ix) 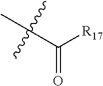
wherein R₁₇ is selected from optionally substituted bicyclic compounds, spirocyclic compounds or bridged bicyclic compounds; pharmaceutically acceptable salts, enantiomers, diastereomers, racemates, or solvates thereof.

12. The compound according to claim 1 comprising the compound (XI) represented by following formulae: 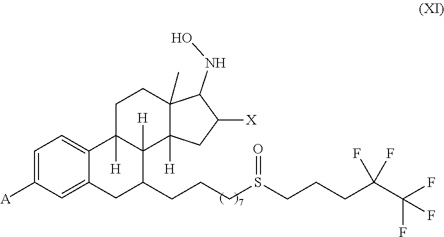
wherein X and A has the same meaning as given in compound of formula (X); pharmaceutically acceptable salts, enantiomers, diastereomers, racemates, or solvates thereof.

13. The compound according to claim 1 comprising the compound (XII) represented by following formulae: 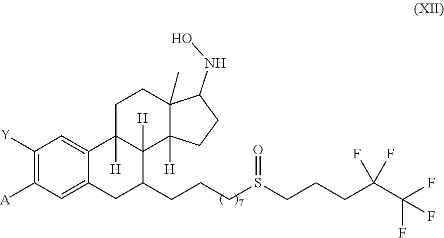
wherein Y and A has the same meaning as given in compound of formula (X); pharmaceutically acceptable salts, enantiomers, diastereomers, racemates, or solvates thereof.

14. The compound according to claim 1 comprising the compound (XIII) represented by following formulae: 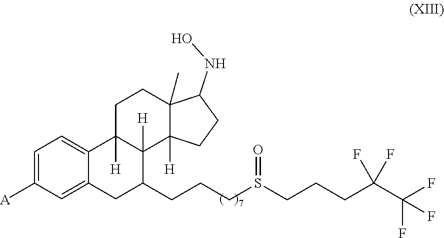
wherein;
A is selected from 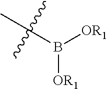
or —O—R₂, wherein R₁ is selected from hydrogen or alkyl; R₂ is selected independently from group consisting of
i) 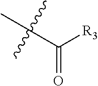
wherein R₃ is selected from NH₂, NHR₄, or NR₅R₆; R₄ is selected from group comprising optionally substituted alkyl, alkenyl, alkynyl, cycloalkyl, heterocycloalkyl, aryl, or heteroaryl; R₅ and R₆ are selected independently from group comprising optionally substituted alkyl, aryl, heteroaryl, heterocycloalkyl, cycloalkyl; or R₅ and R₆ are taken together along with the nitrogen to which they are attached to form a three to seven membered heterocyclic ring and may optionally be substituted at a substitutable position with one or more R₇ radicals, wherein the one or more R₇ radicals are independently selected at each occurrence from the group consisting of optionally substituted alkyl, alkylcarbonyl, formyl, halo, haloalkyl, alkylphosphate, phosphate, oxo, cyano, nitro, amino, hydroxyalkylamino, alkoxy, alkoxycarbonyl, carboxyalkyl, hydroxyalkyl, alkenyl, alkynyl, alkylthio, cycloalkyl, aryl, heteroaryl, aralkyl, heterocycloalkyl, alkylthioalkyl, arylcarbonyl, aralkylcarbonyl, alkoxyalkyl;
ii) 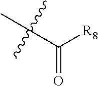
wherein R₈ is selected from group comprising optionally substituted alkyl, alkenyl, alkynyl, cycloalkyl, aryl, heteroaryl; or —CR₉R₁₀; where R₉ and R₁₀ are taken together along with the carbon to which they are attached to form a three to seven membered saturated, partially unsaturated or unsaturated heterocyclic ring in which up to 4 carbon atoms are replaced by heteroatoms chosen from the group consisting of O, S or N and may optionally be substituted at a substitutable position with one or more Ru radicals, wherein one or more Ru radicals are independently selected at each occurrence from the group consisting of optionally substituted alkyl, cycloalkyl, heterocycloalkyl, aryl, heteroaryl, alkylcarbonyl, formyl, halo, alkylphosphate, phosphate, cyano, nitro, alkoxy, amino, alkoxycarbonyl, alkenyl, alkynyl, alkylthio arylcarbonyl;
iii) 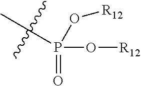
wherein each R₁₂ is selected independently from hydrogen; optionally substituted optionally substituted alkyl, aryl, acyl, heterocycloalkyl or heteroaryl;
iv) 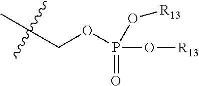
wherein each R₁₃ is selected independently from hydrogen; optionally substituted alkyl, cycloalkyl, heterocycloalkyl, aryl, or heteroaryl;
v) amino acids, wherein the amino acid is linked via ester linkage at the point of attachment;
vi) 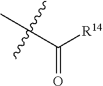
wherein R¹⁴ is 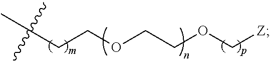
wherein m and p are independently selected from 0 to 5, n refers to degree of polymerization and is selected from 1 to 250 and Z is optionally substituted alkyl or cycloalkyl;
vii) 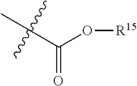
wherein R¹⁵ is selected from group comprising of optionally substituted alkyl, alkenyl, alkynyl, cycloalkyl, heterocycloalkyl, aryl and heteroaryl;
viii) 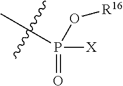
wherein R¹⁶ is selected from group comprising of hydrogen; optionally substituted alkyl, aryl, acyl, heteroaryl; and X is optionally substituted heterocycloalkyl;
ix) 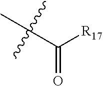
wherein R₁₇ is selected from optionally substituted bicyclic compounds, spirocyclic compounds or bridged bicyclic compounds; pharmaceutically acceptable salts, enantiomers, diastereomers, racemates, or solvates thereof.

15. The compound according to claim 1 comprising the compound (XIV) represented by following formulae: 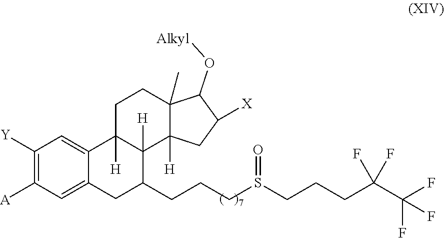
wherein;
X is halogen or hydrogen;
Y is alkyl, —O-alkyl or hydrogen;
A is selected from 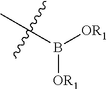
or —O—R₂, wherein R₁ is selected from hydrogen or alkyl; R₂ is selected independently from group consisting of
i) 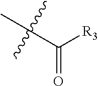
wherein R₃ is selected from NH₂, NHR₄, or NR₅R₆; R₄ is selected from group comprising optionally substituted alkyl, alkenyl, alkynyl, cycloalkyl, heterocycloalkyl, aryl, or heteroaryl; R₅ and R₆ are selected independently from group comprising optionally substituted alkyl, aryl, heteroaryl, heterocycloalkyl, cycloalkyl; or R₅ and R₆ are taken together along with the nitrogen to which they are attached to form a three to seven membered heterocyclic ring and may optionally be substituted at a substitutable position with one or more R₇ radicals, wherein the one or more R₇ radicals are independently selected at each occurrence from the group consisting of optionally substituted alkyl, alkylcarbonyl, formyl, halo, haloalkyl, alkylphosphate, phosphate, oxo, cyano, nitro, amino, hydroxyalkylamino, alkoxy, alkoxycarbonyl, carboxyalkyl, hydroxyalkyl, alkenyl, alkynyl, alkylthio, cycloalkyl, aryl, heteroaryl, aralkyl, heterocycloalkyl, alkylthioalkyl, arylcarbonyl, aralkylcarbonyl, alkoxyalkyl;
ii) 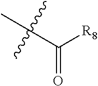
wherein R₈ is selected from group comprising optionally substituted alkyl, alkenyl, alkynyl, cycloalkyl, aryl, heteroaryl; or —CR₉R₁₀; where R₉ and R₁₀ are taken together along with the carbon to which they are attached to form a three to seven membered saturated, partially unsaturated or unsaturated heterocyclic ring in which up to 4 carbon atoms are replaced by heteroatoms chosen from the group consisting of O, S or N and may optionally be substituted at a substitutable position with one or more R₁₁ radicals, wherein one or more R₁₁ radicals are independently selected at each occurrence from the group consisting of optionally substituted alkyl, cycloalkyl, heterocycloalkyl, aryl, heteroaryl, alkylcarbonyl, formyl, halo, alkylphosphate, phosphate, cyano, nitro, alkoxy, amino, alkoxycarbonyl, alkenyl, alkynyl, alkylthio arylcarbonyl;
iii) 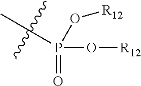
wherein each R₁₂ is selected independently from hydrogen; optionally substituted optionally substituted alkyl, aryl, acyl, heterocycloalkyl or heteroaryl;
iv) 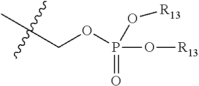
wherein each R₁₃ is selected independently from hydrogen; optionally substituted alkyl, cycloalkyl, heterocycloalkyl, aryl, or heteroaryl;
v) amino acids, wherein the amino acid is linked via ester linkage at the point of attachment;
vi) 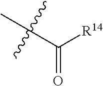
wherein R¹⁴ is 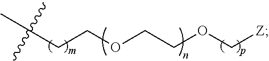
wherein m and p are independently selected from 0 to 5, n refers to degree of polymerization and is selected from 1 to 250 and Z is optionally substituted alkyl or cycloalkyl;
vii) 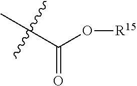
wherein R¹⁵ is selected from group comprising of optionally substituted alkyl, alkenyl, alkynyl, cycloalkyl, heterocycloalkyl, aryl and heteroaryl;
viii) 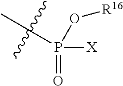
wherein R¹⁶ is selected from group comprising of hydrogen; optionally substituted alkyl, aryl, acyl, heteroaryl; and X is optionally substituted heterocycloalkyl;
ix) 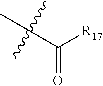
wherein R₁₇ is selected from optionally substituted bicyclic compounds, spirocyclic compounds or bridged bicyclic compounds; pharmaceutically acceptable salts, enantiomers, diastereomers, racemates, or solvates thereof.

16. The compound according to claim 1 comprising the compound (XV) represented by following formulae: 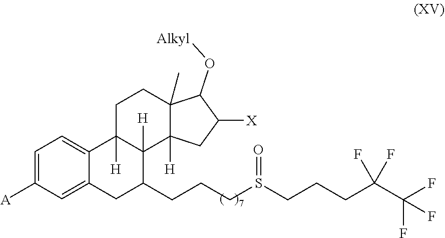
wherein X and A has the same meaning as given in compound of formula (XIV); pharmaceutically acceptable salts, enantiomers, diastereomers, racemates, or solvates thereof.

17. The compound according to claim 1 comprising the compound (XVI) represented by following formulae: 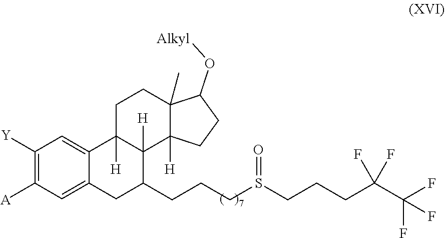
wherein Y and A has the same meaning as given in compound of formula (XIV); pharmaceutically acceptable salts, enantiomers, diastereomers, racemates, or solvates thereof.

18. The compound according to claim 1 comprising the compound (XVII) represented by following formulae: 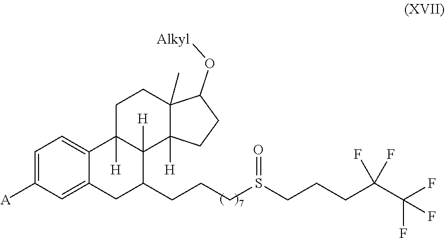
wherein;
A is selected from 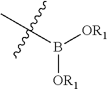
or —O—R₂, wherein R₁ is selected from hydrogen or alkyl; R₂ is selected independently from group consisting of
i) 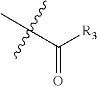
wherein R₃ is selected from NH₂, NHR₄, or NR₅R₆; R₄ is selected from group comprising optionally substituted alkyl, alkenyl, alkynyl, cycloalkyl, heterocycloalkyl, aryl, or heteroaryl; R₅ and R₆ are selected independently from group comprising optionally substituted alkyl, aryl, heteroaryl, heterocycloalkyl, cycloalkyl; or R₅ and R₆ are taken together along with the nitrogen to which they are attached to form a three to seven membered heterocyclic ring and may optionally be substituted at a substitutable position with one or more R₇ radicals, wherein the one or more R₇ radicals are independently selected at each occurrence from the group consisting of optionally substituted alkyl, alkylcarbonyl, formyl, halo, haloalkyl, alkylphosphate, phosphate, oxo, cyano, nitro, amino, hydroxyalkylamino, alkoxy, alkoxycarbonyl, carboxyalkyl, hydroxyalkyl, alkenyl, alkynyl, alkylthio, cycloalkyl, aryl, heteroaryl, aralkyl, heterocycloalkyl, alkylthioalkyl, arylcarbonyl, aralkylcarbonyl, alkoxyalkyl;
ii) 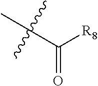
wherein R₈ is selected from group comprising optionally substituted alkyl, alkenyl, alkynyl, cycloalkyl, aryl, heteroaryl; or —CR₉R₁₀; where R₉ and R₁₀ are taken together along with the carbon to which they are attached to form a three to seven membered saturated, partially unsaturated or unsaturated heterocyclic ring in which up to 4 carbon atoms are replaced by heteroatoms chosen from the group consisting of O, S or N and may optionally be substituted at a substitutable position with one or more Ru radicals, wherein one or more Ru radicals are independently selected at each occurrence from the group consisting of optionally substituted alkyl, cycloalkyl, heterocycloalkyl, aryl, heteroaryl, alkylcarbonyl, formyl, halo, alkylphosphate, phosphate, cyano, nitro, alkoxy, amino, alkoxycarbonyl, alkenyl, alkynyl, alkylthio arylcarbonyl;
iii) 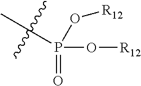
wherein each R₁₂ is selected independently from hydrogen; optionally substituted optionally substituted alkyl, aryl, acyl, heterocycloalkyl or heteroaryl;
iv) 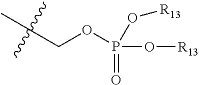
wherein each R₁₃ is selected independently from hydrogen; optionally substituted alkyl, cycloalkyl, heterocycloalkyl, aryl, or heteroaryl;
v) amino acids, wherein the amino acid is linked via ester linkage at the point of attachment;
vi) 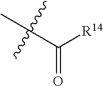
wherein R¹⁴ is 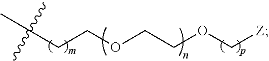
wherein m and p are independently selected from 0 to 5, n refers to degree of polymerization and is selected from 1 to 250 and Z is optionally substituted alkyl or cycloalkyl;
vii) 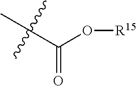
wherein R¹⁵ is selected from group comprising of optionally substituted alkyl, alkenyl, alkynyl, cycloalkyl, heterocycloalkyl, aryl and heteroaryl;
viii) 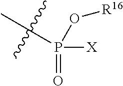
wherein R¹⁶ is selected from group comprising of hydrogen; optionally substituted alkyl, aryl, acyl, heteroaryl; and X is optionally substituted heterocycloalkyl;
ix) 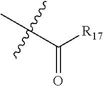
wherein R₁₇ is selected from optionally substituted bicyclic compounds, spirocyclic compounds or bridged bicyclic compounds; pharmaceutically acceptable salts, enantiomers, diastereomers, racemates, or solvates thereof.

19. The compound according to claim 1 comprising the compound (XVIII) represented by following formulae: 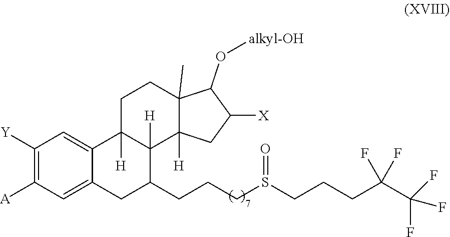
wherein;
X is halogen or hydrogen;
Y is alkyl, —O-alkyl or hydrogen;
A is selected from 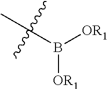
or —O—R₂, wherein R₁ is selected from hydrogen or alkyl; R₂ is selected independently from group consisting of
i) 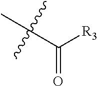
wherein R₃ is selected from NH₂, NHR₄, or NR₅R₆; R₄ is selected from group comprising optionally substituted alkyl, alkenyl, alkynyl, cycloalkyl, heterocycloalkyl, aryl, or heteroaryl; R₅ and R₆ are selected independently from group comprising optionally substituted alkyl, aryl, heteroaryl, heterocycloalkyl, cycloalkyl; or R₅ and R₆ are taken together along with the nitrogen to which they are attached to form a three to seven membered heterocyclic ring and may optionally be substituted at a substitutable position with one or more R₇ radicals, wherein the one or more R₇ radicals are independently selected at each occurrence from the group consisting of optionally substituted alkyl, alkylcarbonyl, formyl, halo, haloalkyl, alkylphosphate, phosphate, oxo, cyano, nitro, amino, hydroxyalkylamino, alkoxy, alkoxycarbonyl, carboxyalkyl, hydroxyalkyl, alkenyl, alkynyl, alkylthio, cycloalkyl, aryl, heteroaryl, aralkyl, heterocycloalkyl, alkylthioalkyl, arylcarbonyl, aralkylcarbonyl, alkoxyalkyl;
ii) 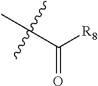
wherein R₈ is selected from group comprising optionally substituted alkyl, alkenyl, alkynyl, cycloalkyl, aryl, heteroaryl; or —CR₉R₁₀; where R₉ and R₁₀ are taken together along with the carbon to which they are attached to form a three to seven membered saturated, partially unsaturated or unsaturated heterocyclic ring in which up to 4 carbon atoms are replaced by heteroatoms chosen from the group consisting of O, S or N and may optionally be substituted at a substitutable position with one or more R₁₁ radicals, wherein one or more R₁₁ radicals are independently selected at each occurrence from the group consisting of optionally substituted alkyl, cycloalkyl, heterocycloalkyl, aryl, heteroaryl, alkylcarbonyl, formyl, halo, alkylphosphate, phosphate, cyano, nitro, alkoxy, amino, alkoxycarbonyl, alkenyl, alkynyl, alkylthio arylcarbonyl;
iii) 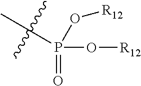
wherein each R₁₂ is selected independently from hydrogen; optionally substituted optionally substituted alkyl, aryl, acyl, heterocycloalkyl or heteroaryl;
iv) 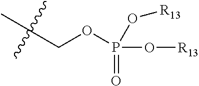
wherein each R₃ is selected independently from hydrogen; optionally substituted alkyl, cycloalkyl, heterocycloalkyl, aryl, or heteroaryl;
v) amino acids, wherein the amino acid is linked via ester linkage at the point of attachment;
vi) 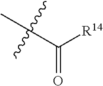
wherein R¹⁴ is 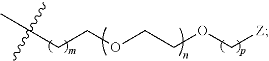
wherein m and p are independently selected from 0 to 5, n refers to degree of polymerization and is selected from 1 to 250 and Z is optionally substituted alkyl or cycloalkyl;
vii) 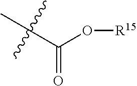
wherein R¹⁵ is selected from group comprising of optionally substituted alkyl, alkenyl, alkynyl, cycloalkyl, heterocycloalkyl, aryl and heteroaryl;
viii) 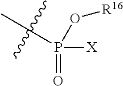
wherein R¹⁶ is selected from group comprising of hydrogen; optionally substituted alkyl, aryl, acyl, heteroaryl; and X is optionally substituted heterocycloalkyl;
ix) 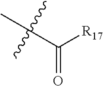
wherein R₁₇ is selected from optionally substituted bicyclic compounds, spirocyclic compounds or bridged bicyclic compounds; pharmaceutically acceptable salts, enantiomers, diastereomers, racemates, or solvates thereof.

20. The compound according to claim 1 comprising the compound (XIX) represented by following formulae: 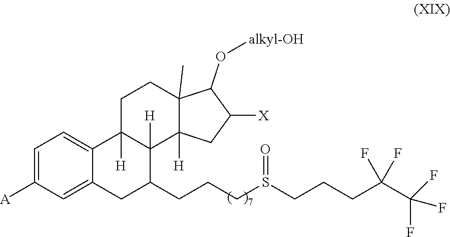
wherein X and A has the same meaning as given in compound of formula (XVIII); pharmaceutically acceptable salts, enantiomers, diastereomers, racemates, or solvates thereof.

21. The compound according to claim 1 comprising the compound (XX) represented by following formulae: 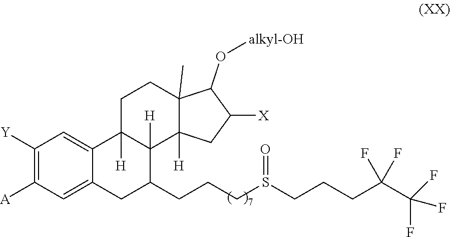
wherein Y and A has the same meaning as given in compound of formula XVIII; pharmaceutically acceptable salts, enantiomers, diastereomers, racemates, or solvates thereof.

22. The compound according to claim 1 comprising the compound (XXI) represented by following formulae: 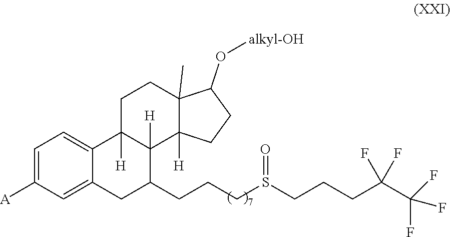
wherein;
A is selected from 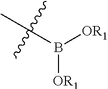
or —O—R₂, wherein R₁ is selected from hydrogen or alkyl; R₂ is selected independently from group consisting of
i) 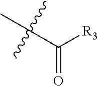
wherein R₃ is selected from NH₂, NHR₄, or NR₅R₆; R₄ is selected from group comprising optionally substituted alkyl, alkenyl, alkynyl, cycloalkyl, heterocycloalkyl, aryl, or heteroaryl; R₅ and R₆ are selected independently from group comprising optionally substituted alkyl, aryl, heteroaryl, heterocycloalkyl, cycloalkyl; or R₅ and R₆ are taken together along with the nitrogen to which they are attached to form a three to seven membered heterocyclic ring and may optionally be substituted at a substitutable position with one or more R₇ radicals, wherein the one or more R₇ radicals are independently selected at each occurrence from the group consisting of optionally substituted alkyl, alkylcarbonyl, formyl, halo, haloalkyl, alkylphosphate, phosphate, oxo, cyano, nitro, amino, hydroxyalkylamino, alkoxy, alkoxycarbonyl, carboxyalkyl, hydroxyalkyl, alkenyl, alkynyl, alkylthio, cycloalkyl, aryl, heteroaryl, aralkyl, heterocycloalkyl, alkylthioalkyl, arylcarbonyl, aralkylcarbonyl, alkoxyalkyl;
ii) 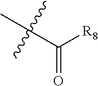
wherein R₈ is selected from group comprising optionally substituted alkyl, alkenyl, alkynyl, cycloalkyl, aryl, heteroaryl; or —CR₉R₁₀; where R₉ and R₁₀ are taken together along with the carbon to which they are attached to form a three to seven membered saturated, partially unsaturated or unsaturated heterocyclic ring in which up to 4 carbon atoms are replaced by heteroatoms chosen from the group consisting of O, S or N and may optionally be substituted at a substitutable position with one or more R₁₁ radicals, wherein one or more R₁₁ radicals are independently selected at each occurrence from the group consisting of optionally substituted alkyl, cycloalkyl, heterocycloalkyl, aryl, heteroaryl, alkylcarbonyl, formyl, halo, alkylphosphate, phosphate, cyano, nitro, alkoxy, amino, alkoxycarbonyl, alkenyl, alkynyl, alkylthio arylcarbonyl;
iii) 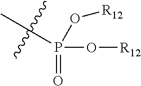
wherein each R₁₂ is selected independently from hydrogen; optionally substituted optionally substituted alkyl, aryl, acyl, heterocycloalkyl or heteroaryl;
iv) 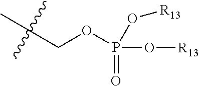
wherein each R₁₃ is selected independently from hydrogen; optionally substituted alkyl, cycloalkyl, heterocycloalkyl, aryl, or heteroaryl;
v) amino acids, wherein the amino acid is linked via ester linkage at the point of attachment;
vi) 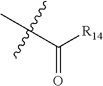
wherein R¹⁴ is 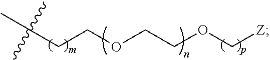
wherein m and p are independently selected from 0 to 5, n refers to degree of polymerization and is selected from 1 to 250 and Z is optionally substituted alkyl or cycloalkyl;
vii) 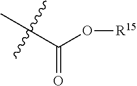
wherein R¹⁵ is selected from group comprising of optionally substituted alkyl, alkenyl, alkynyl, cycloalkyl, heterocycloalkyl, aryl and heteroaryl;
viii) 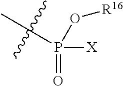
wherein R¹⁶ is selected from group comprising of hydrogen; optionally substituted alkyl, aryl, acyl, heteroaryl; and X is optionally substituted heterocycloalkyl;
ix) 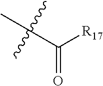
wherein R₁₇ is selected from optionally substituted bicyclic compounds, spirocyclic compounds or bridged bicyclic compounds; pharmaceutically acceptable salts, enantiomers, diastereomers, racemates, or solvates thereof.

23. The compound according to claim 1 comprising the compound (XXII) represented by following formulae: 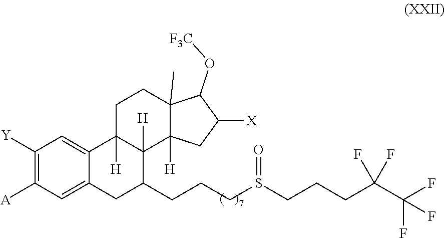
wherein;
X is halogen or hydrogen;
Y is alkyl, —O-alkyl or hydrogen;
A is selected from 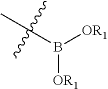
or —O—R₂, wherein R₁ is selected from hydrogen or alkyl; R₂ is selected independently from group consisting of
i) 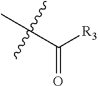
wherein R₃ is selected from NH₂, NHR₄, or NR₅R₆; R₄ is selected from group comprising optionally substituted alkyl, alkenyl, alkynyl, cycloalkyl, heterocycloalkyl, aryl, or heteroaryl; R₅ and R₆ are selected independently from group comprising optionally substituted alkyl, aryl, heteroaryl, heterocycloalkyl, cycloalkyl; or R₅ and R₆ are taken together along with the nitrogen to which they are attached to form a three to seven membered heterocyclic ring and may optionally be substituted at a substitutable position with one or more R₇ radicals, wherein the one or more R₇ radicals are independently selected at each occurrence from the group consisting of optionally substituted alkyl, alkylcarbonyl, formyl, halo, haloalkyl, alkylphosphate, phosphate, oxo, cyano, nitro, amino, hydroxyalkylamino, alkoxy, alkoxycarbonyl, carboxyalkyl, hydroxyalkyl, alkenyl, alkynyl, alkylthio, cycloalkyl, aryl, heteroaryl, aralkyl, heterocycloalkyl, alkylthioalkyl, arylcarbonyl, aralkylcarbonyl, alkoxyalkyl;
ii) 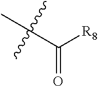
wherein R₈ is selected from group comprising optionally substituted alkyl, alkenyl, alkynyl, cycloalkyl, aryl, heteroaryl; or —CR₉R₁₀; where R₉ and R₁₀ are taken together along with the carbon to which they are attached to form a three to seven membered saturated, partially unsaturated or unsaturated heterocyclic ring in which up to 4 carbon atoms are replaced by heteroatoms chosen from the group consisting of O, S or N and may optionally be substituted at a substitutable position with one or more R₁₁ radicals, wherein one or more R₁₁ radicals are independently selected at each occurrence from the group consisting of optionally substituted alkyl, cycloalkyl, heterocycloalkyl, aryl, heteroaryl, alkylcarbonyl, formyl, halo, alkylphosphate, phosphate, cyano, nitro, alkoxy, amino, alkoxycarbonyl, alkenyl, alkynyl, alkylthio arylcarbonyl;
iii) 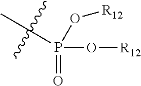
wherein each R₁₂ is selected independently from hydrogen; optionally substituted optionally substituted alkyl, aryl, acyl, heterocycloalkyl or heteroaryl;
iv) 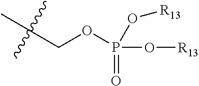
wherein each R₁₃ is selected independently from hydrogen; optionally substituted alkyl, cycloalkyl, heterocycloalkyl, aryl, or heteroaryl;
v) amino acids, wherein the amino acid is linked via ester linkage at the point of attachment;
vi) 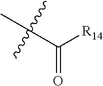
wherein R¹⁴ is 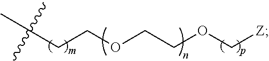
wherein m and p are independently selected from 0 to 5, n refers to degree of polymerization and is selected from 1 to 250 and Z is optionally substituted alkyl or cycloalkyl;
vii) 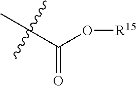
wherein R¹⁵ is selected from group comprising of optionally substituted alkyl, alkenyl, alkynyl, cycloalkyl, heterocycloalkyl, aryl and heteroaryl;
viii) 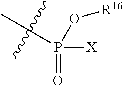
wherein R¹⁶ is selected from group comprising of hydrogen; optionally substituted alkyl, aryl, acyl, heteroaryl; and X is optionally substituted heterocycloalkyl;
ix) 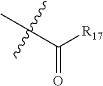
wherein R₁₇ is selected from optionally substituted bicyclic compounds, spirocyclic compounds or bridged bicyclic compounds; pharmaceutically acceptable salts, enantiomers, diastereomers, racemates, or solvates thereof.

24. The compound according to claim 1 comprising the compound (XXIII) represented by following formulae: 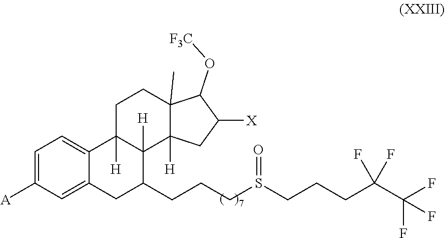
wherein X and A has the same meaning as given in compound of formula XXII; pharmaceutically acceptable salts, enantiomers, diastereomers, racemates, or solvates thereof.

25. The compound according to claim 1 comprising the compound (XXIV) represented by following formulae: 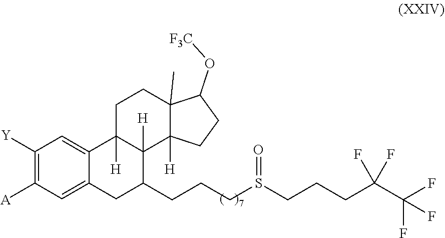
wherein Y and A has the same meaning as given in compound of formula XXII; pharmaceutically acceptable salts, enantiomers, diastereomers, racemates, or solvates thereof.

26. The compound according to claim 1 comprising the compound (XXV) represented by following formulae: 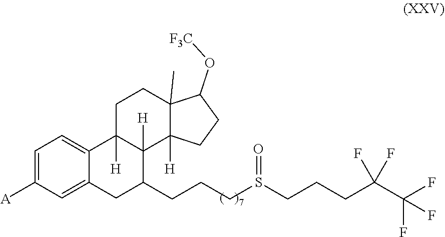
wherein;
A is selected from 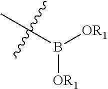
or —O—R₂, wherein R₁ is selected from hydrogen or alkyl; R₂ is selected independently from group consisting of
i) 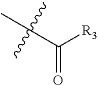
wherein R₃ is selected from NH₂, NHR₄, or NR₅R₆; R₄ is selected from group comprising optionally substituted alkyl, alkenyl, alkynyl, cycloalkyl, heterocycloalkyl, aryl, or heteroaryl; R₅ and R₆ are selected independently from group comprising optionally substituted alkyl, aryl, heteroaryl, heterocycloalkyl, cycloalkyl; or R₅ and R₆ are taken together along with the nitrogen to which they are attached to form a three to seven membered heterocyclic ring and may optionally be substituted at a substitutable position with one or more R₇ radicals, wherein the one or more R₇ radicals are independently selected at each occurrence from the group consisting of optionally substituted alkyl, alkylcarbonyl, formyl, halo, haloalkyl, alkylphosphate, phosphate, oxo, cyano, nitro, amino, hydroxyalkylamino, alkoxy, alkoxycarbonyl, carboxyalkyl, hydroxyalkyl, alkenyl, alkynyl, alkylthio, cycloalkyl, aryl, heteroaryl, aralkyl, heterocycloalkyl, alkylthioalkyl, arylcarbonyl, aralkylcarbonyl, alkoxyalkyl;
ii) 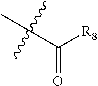
wherein R₈ is selected from group comprising optionally substituted alkyl, alkenyl, alkynyl, cycloalkyl, aryl, heteroaryl; or —CR₉R₁₀; where R₉ and R₁₀ are taken together along with the carbon to which they are attached to form a three to seven membered saturated, partially unsaturated or unsaturated heterocyclic ring in which up to 4 carbon atoms are replaced by heteroatoms chosen from the group consisting of O, S or N and may optionally be substituted at a substitutable position with one or more R₁ radicals, wherein one or more R₁ radicals are independently selected at each occurrence from the group consisting of optionally substituted alkyl, cycloalkyl, heterocycloalkyl, aryl, heteroaryl, alkylcarbonyl, formyl, halo, alkylphosphate, phosphate, cyano, nitro, alkoxy, amino, alkoxycarbonyl, alkenyl, alkynyl, alkylthio arylcarbonyl;
iii) 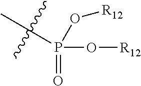
wherein each R₁₂ is selected independently from hydrogen; optionally substituted optionally substituted alkyl, aryl, acyl, heterocycloalkyl or heteroaryl;
iv) 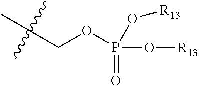
wherein each R₁₃ is selected independently from hydrogen; optionally substituted alkyl, cycloalkyl, heterocycloalkyl, aryl, or heteroaryl;
v) amino acids, wherein the amino acid is linked via ester linkage at the point of attachment;
vi) 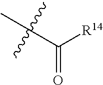
wherein R¹⁴ is 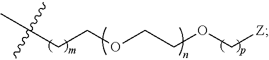
wherein m and p are independently selected from 0 to 5, n refers to degree of polymerization and is selected from 1 to 250 and Z is optionally substituted alkyl or cycloalkyl;
vii) 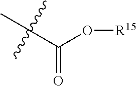
wherein R⁵ is selected from group comprising of optionally substituted alkyl, alkenyl, alkynyl, cycloalkyl, heterocycloalkyl, aryl and heteroaryl;
viii) 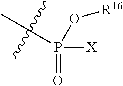
wherein R¹⁶ is selected from group comprising of hydrogen; optionally substituted alkyl, aryl, acyl, heteroaryl; and X is optionally substituted heterocycloalkyl;
ix) 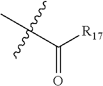
wherein R₁₇ is selected from optionally substituted bicyclic compounds, spirocyclic compounds or bridged bicyclic compounds; pharmaceutically acceptable salts, enantiomers, diastereomers, racemates, or solvates thereof.

27. The compound according to claim 1 comprising the compound (XXVI) represented by following formulae: 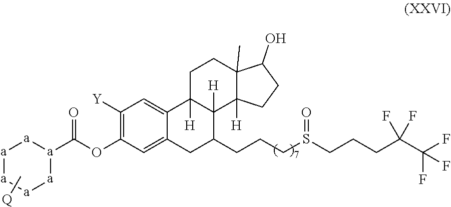
X is halogen or hydrogen;
Y is alkyl, —O-alkyl or hydrogen;
Z is selected from ═O, ═N—OH, —O-alkyl, O-haloalkyl, —NH—OH or —O-alkyl-OH wherein the bond between the Z substituent attached to carbon can be single or double bond depend on the substituent selected,
a is selected from carbon or nitrogen;
Q is selected from group consisting of optionally substituted alkyl, alkylcarbonyl, formyl, halo, haloalkyl, alkylphosphate, phosphate, oxo, cyano, nitro, amino, alkoxy, alkoxycarbonyl, carboxyalkyl, hydroxyalkyl, alkenyl, alkynyl, alkylthio, cycloalkyl, aryl, heteroaryl, aralkyl, heterocycloalkyl, alkylthioalkyl, arylcarbonyl, aralkylcarbonyl, alkoxyalkyl;
pharmaceutically acceptable salts, enantiomers, diastereomers, racemates, or solvates thereof.

28. The compound according to claim 1 comprising the compound (XXVII) represented by following formulae: 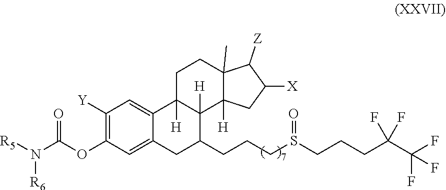
X is halogen or hydrogen;
Y is alkyl, —O-alkyl or hydrogen;
Z is selected from ═O, ═N—OH, —O-alkyl, O-haloalkyl, —NH—OH or —O-alkyl-OH wherein the bond between the Z substituent attached to carbon can be single or double bond depend on the substituent selected;
wherein R₅ and R₆ are taken together along with the nitrogen to which they are attached to form a three to seven membered saturated, partially unsaturated or unsaturated heterocyclic ring and substituted at a substitutable position with one or more R₇ radicals, wherein the one or more R₇ radicals are independently selected at each occurrence from the group consisting of substituted alkyl, alkylcarbonyl, formyl, halo, haloalkyl, alkylphosphate, phosphate, phosphonyl, oxo, cyano, nitro, amino, alkoxy, alkoxycarbonyl, carboxyalkyl, hydroxyalkyl, alkenyl, alkynyl, alkylthio, cycloalkyl, aryl, heteroaryl, aralkyl, heterocycloalkyl, alkylthioalkyl, arylcarbonyl, aralkylcarbonyl, alkoxyalkyl and; pharmaceutically acceptable salts, enantiomers, diastereomers, racemates, or solvates thereof. In one embodiment, one or more substitution on radical R₇ is further substituted with one or more substitution selected from group consisting of alkyl, alkenyl, alkynyl, cycloalkyl, heterocycloalkyl, aryl, heteroaryl, amino, alkylamino, acyl, acyloxy, acylamino, aminocarbonyl, alkoxycarbonyl, alkoxyalkyloxy, alkoxycarbonyloxy, carbamate, sulfinyl, sulfonyl, alkoxy, sulfanyl, halogen, carboxy, trihalomethyl, cyano, hydroxy, mercapto, nitro, phosphate, alkylphosphate; pharmaceutically acceptable salts, enantiomers, diastereomers, racemates, or solvates thereof.

29. The compound according to claim 1 comprising the compound (XVIII) represented by following formulae: 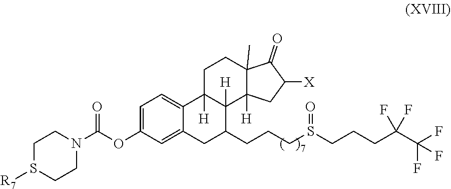
X is halogen or hydrogen;
Y is alkyl, —O-alkyl or hydrogen;
S is selected from O, C, or N; and
wherein R₇ is selected from group consisting of optionally substituted alkyl, alkylcarbonyl, formyl, halo, haloalkyl, alkylphosphate, phosphate, oxo, cyano, nitro, amino, alkoxy, alkoxycarbonyl, carboxyalkyl, hydroxyalkyl, alkenyl, alkynyl, alkylthio, cycloalkyl, aryl, heteroaryl, aralkyl, heterocycloalkyl, alkylthioalkyl, arylcarbonyl, aralkylcarbonyl, alkoxyalkyl;
pharmaceutically acceptable salts, enantiomers, diastereomers, racemates, or solvates thereof.

30. The compound according to claim 1 comprising the compound (XXIX) represented by following formulae: 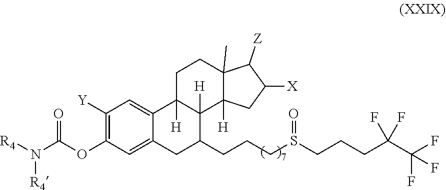
X is halogen or hydrogen;
Y is alkyl, —O-alkyl or hydrogen;
Z is selected from ═O, ═N—OH, —O-alkyl, O-haloalkyl, —NH—OH or —O-alkyl-OH wherein the bond between the Z substituent attached to carbon can be single or double bond depend on the substituent selected;
R_{4′} is selected from group Hydrogen and optionally substituted alkyl.
R₄ is selected from group comprising optionally substituted alkyl, alkenyl, alkynyl, cycloalkyl, heterocycloalkyl, aryl, or heteroaryl; pharmaceutically acceptable salts, enantiomers, diastereomers, racemates, or solvates thereof.

31. The compound according to claim 30 comprising optionally substituted substituents on R₄ and R₄′ is selected from the group consisting of radicals such as C1-C8-alkyl, C2-C8-alkenyl, C2-C8-alkynyl, cycloalkyl, heterocycloalkyl, aryl, heteroaryl, amino, alkylamino, dialkylamino acyl, acyloxy, acylamino, aminocarbonyl, alkoxycarbonyl, alkoxyalkyloxy, alkoxycarbonyloxy, carbamate, sulfinyl, sulfonyl, alkoxy, sulfanyl, halogen, carboxy, haloalkyl, cyano, hydroxy, mercapto, nitro, phosphate, oxo, alkylphosphate.

32. The compound according to claim 1 comprising the compound (XXX) represented by following formulae: 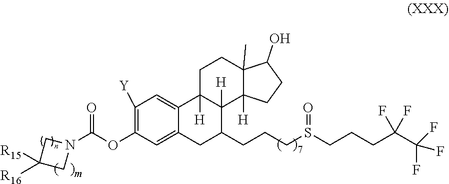
X is halogen or hydrogen;
Y is alkyl, —O-alkyl or hydrogen;
Z is selected from ═O, ═N—OH, —O-alkyl, O-haloalkyl, —NH—OH or —O-alkyl-OH wherein the bond between the Z substituent attached to carbon can be single or double bond depend on the substituent selected;
d is selected from carbon or nitrogen; wherein R₁₅ and R₁₆ together forms a optionally substituted carbocyclic or heterocyclic structure;
m and n can be independently 1 to 3;
pharmaceutically acceptable salts, enantiomers, diastereomers, racemates, or solvates thereof.

33. The compound according to claim 32, the formula XXX is optionally substituted with substituents selected from group consisting of alkyl, alkylcarbonyl, formyl, halo, haloalkyl, alkylphosphate, phosphate, oxo, cyano, nitro, amino, alkoxy, alkoxycarbonyl, carboxyalkyl, hydroxyalkyl, alkenyl, alkynyl, alkylthio, cycloalkyl, aryl, heteroaryl, aralkyl, heterocycloalkyl, alkylthioalkyl, arylcarbonyl, aralkylcarbonyl, alkoxyalkyl.

34. The compound according to claim 1 comprising the compound (XXXI) represented by following formulae: 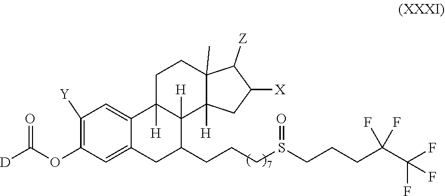
X is halogen or hydrogen;
Y is alkyl, —O-alkyl or hydrogen;
Z is selected from ═O, ═N—OH, —O-alkyl, O-haloalkyl, —NH—OH or —O-alkyl-OH wherein the bond between the Z substituent attached to carbon can be single or double bond depend on the substituent selected;
D is selected from optionally substituted bicyclic compound, spirocyclic compound or bridged bicyclic compounds, wherein the attachment of the bicyclic compound, spirocyclic compound or bridged bicyclic compounds is through either carbon or nitrogen atom; pharmaceutically acceptable salts, enantiomers, diastereomers, racemates, or solvates thereof.

35. The compound according to claim 1, wherein the compound is selected from the group comprising of

| Com- | |
|---|---|
| pound | |
| No. | Structures |
| 1 | 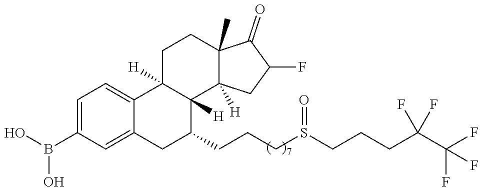 |
| 2 | 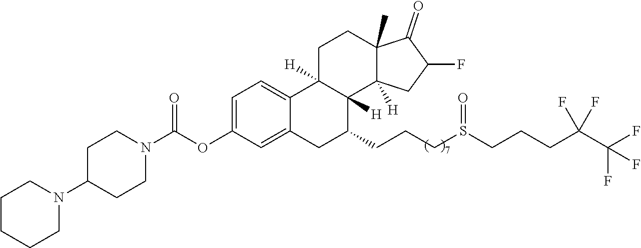 |
| 3 | 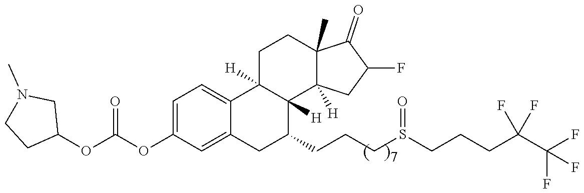 |
| 4 | 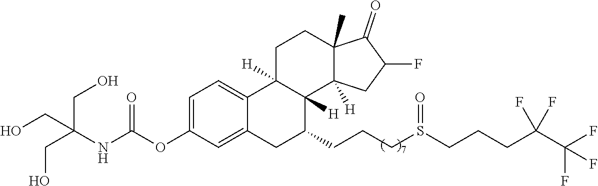 |
| 5 | 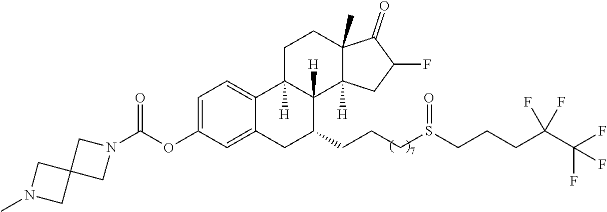 |
| 6 | 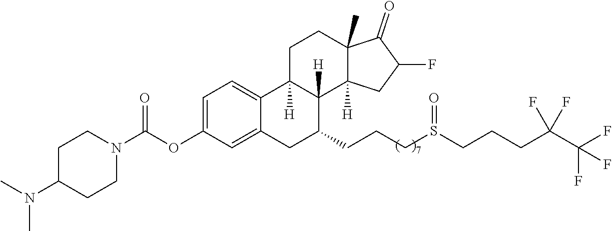 |
| 7 | 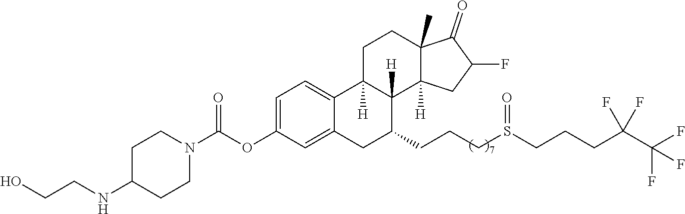 |
| 8 | 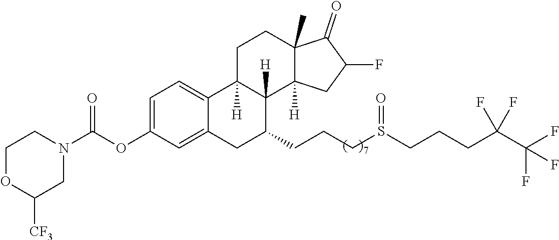 |
| 9 | 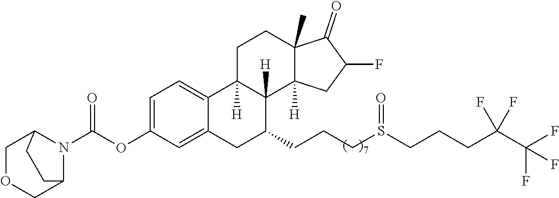 |
| 10 | 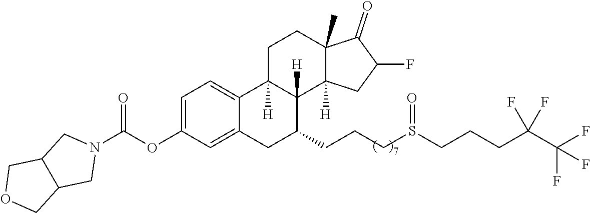 |
| 11 | 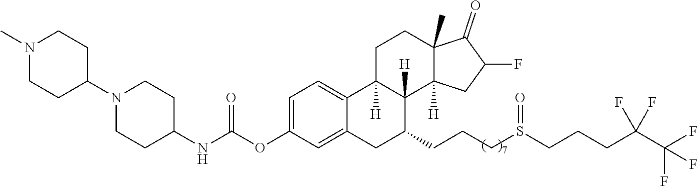 |
| 12 | 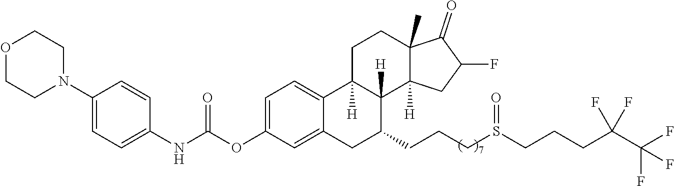 |
| 13 | 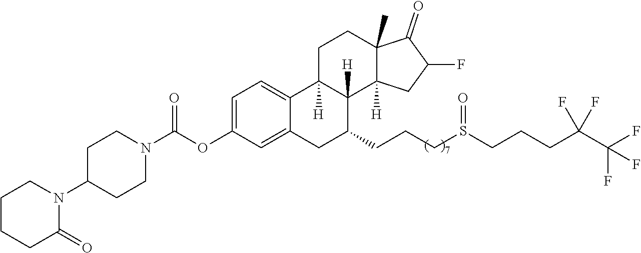 |
| 14 | 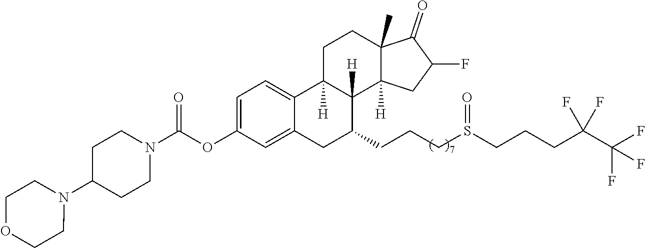 |
| 15 | 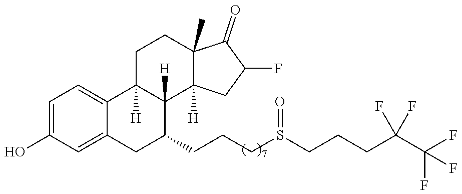 |
| 16 | 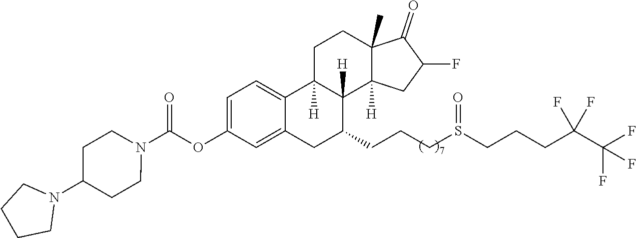 |
| 17 | 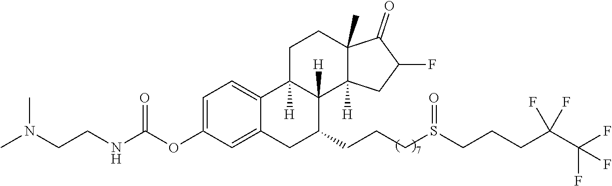 |
| 18 | 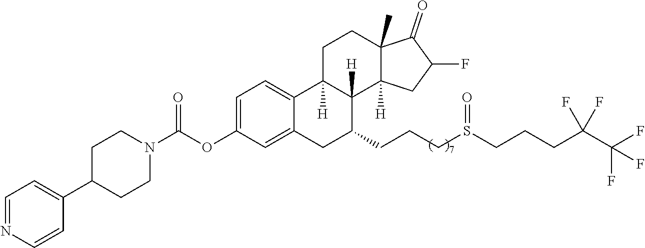 |
| 19 | 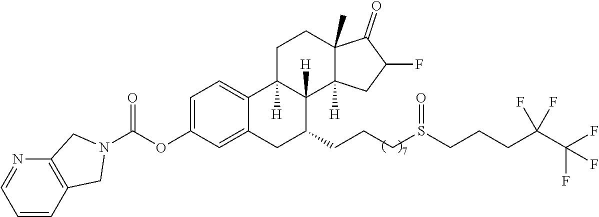 |
| 20 | 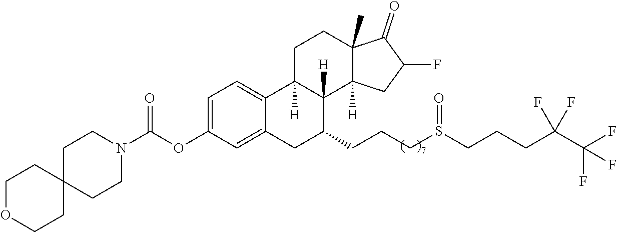 |
| 21 | 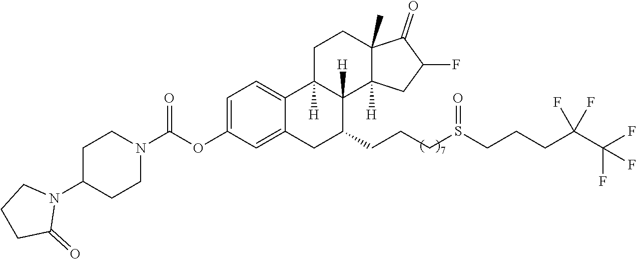 |
| 22 | 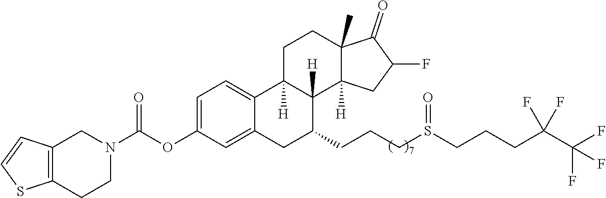 |
| 23 | 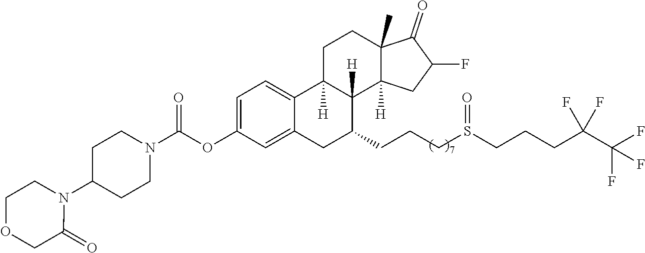 |
| 24 | 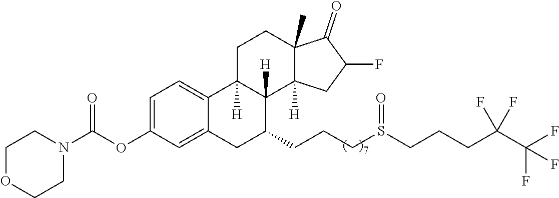 |
| 25 | 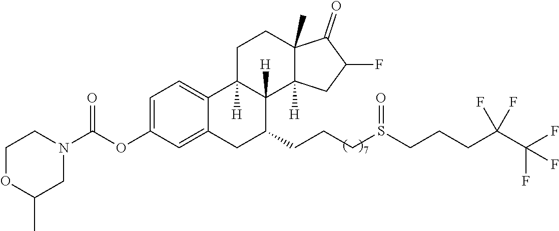 |
| 26 | 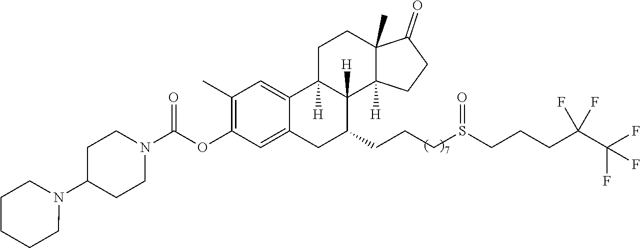 |
| 27 | 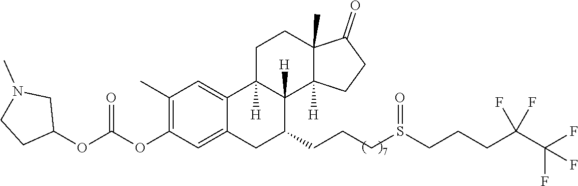 |
| 28 | 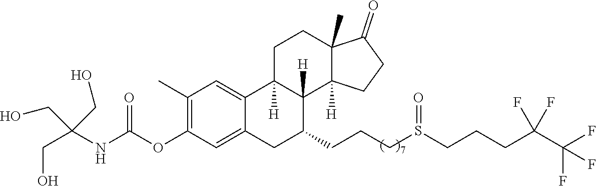 |
| 29 | 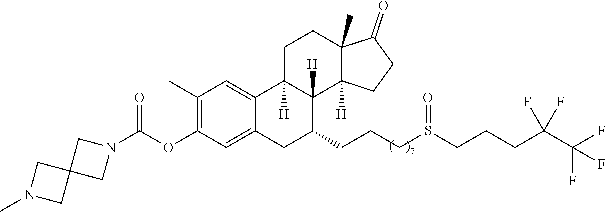 |
| 30 | 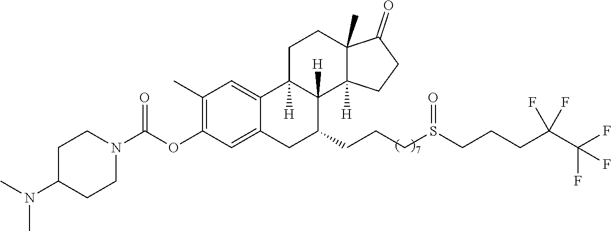 |
| 31 | 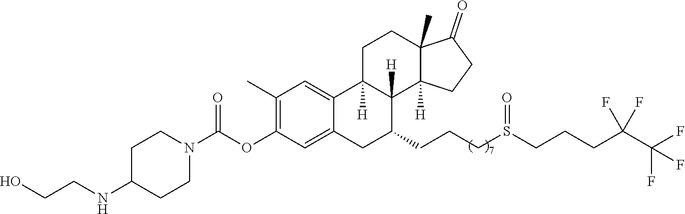 |
| 32 | 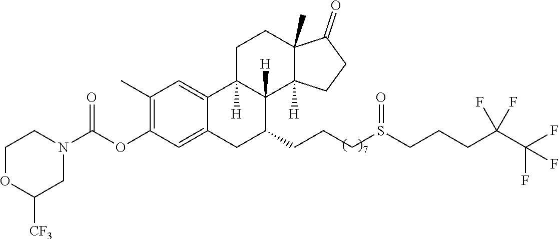 |
| 33 | 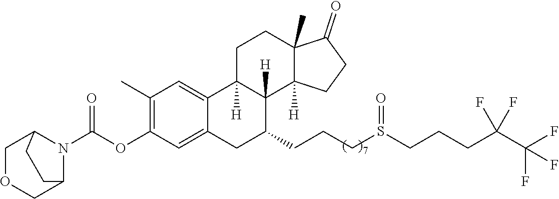 |
| 34 | 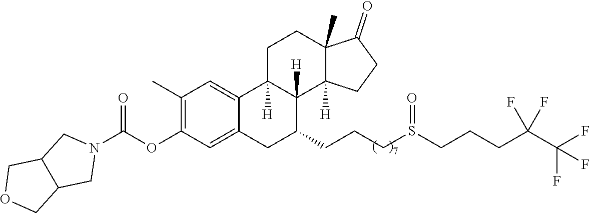 |
| 35 | 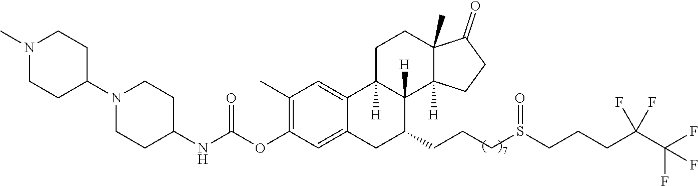 |
| 36 | 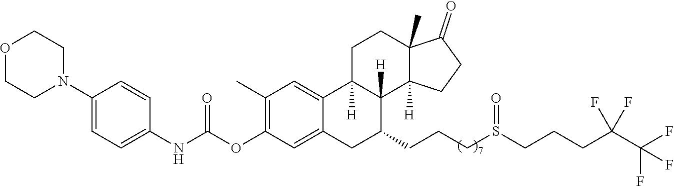 |
| 37 | 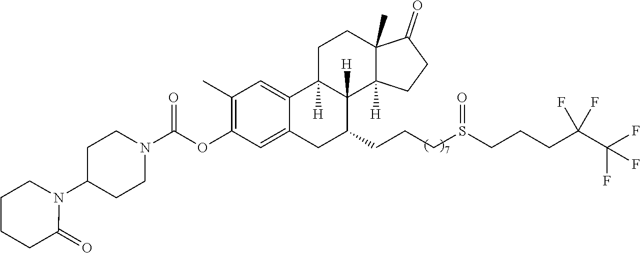 |
| 38 | 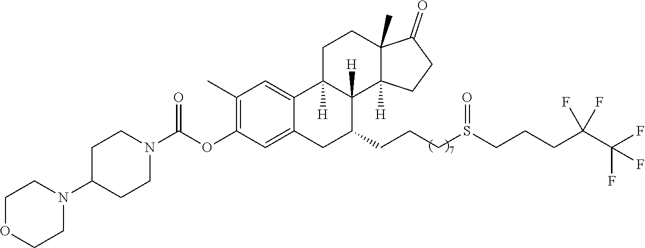 |
| 39 | 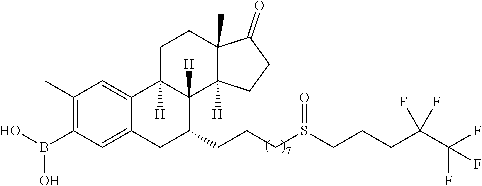 |
| 40 | 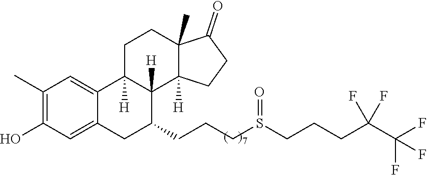 |
| 41 | 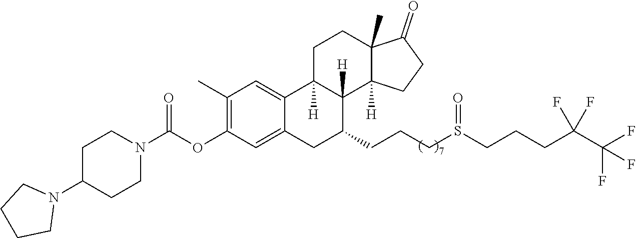 |
| 42 | 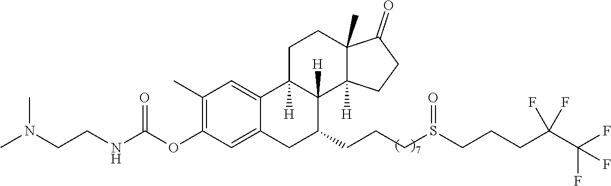 |
| 43 | 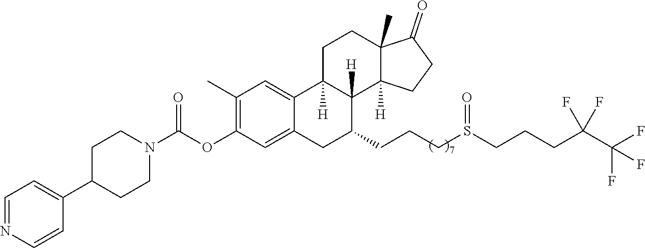 |
| 44 | 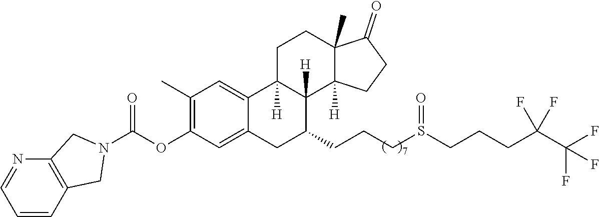 |
| 45 | 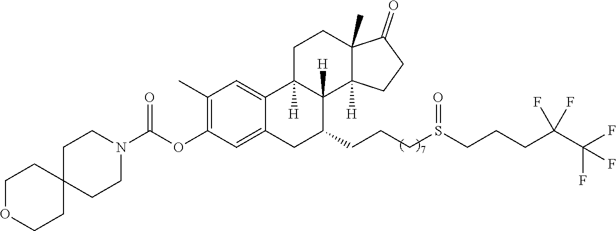 |
| 46 | 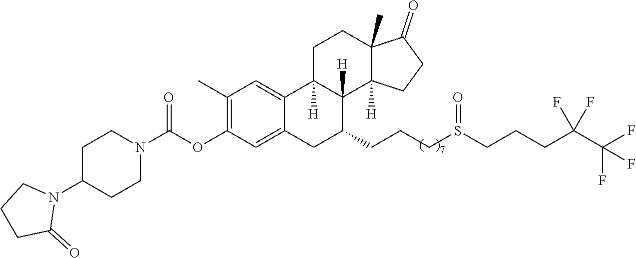 |
| 47 | 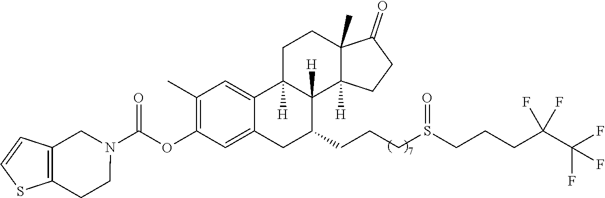 |
| 48 | 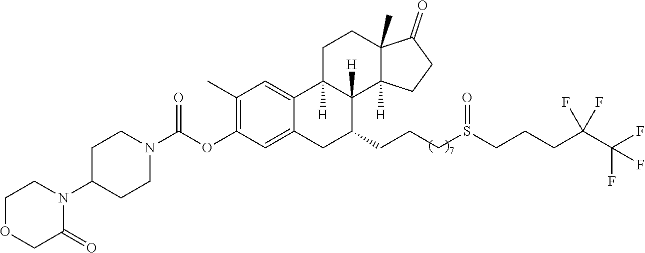 |
| 49 | 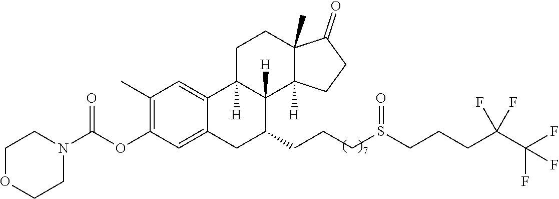 |
| 50 | 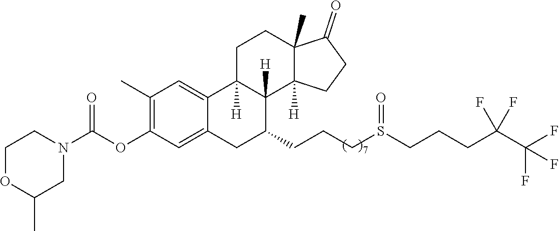 |
| 51 | 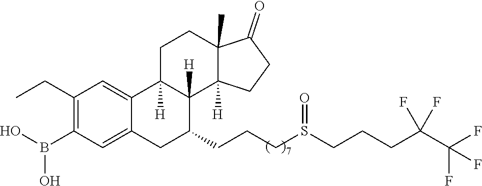 |
| 52 | 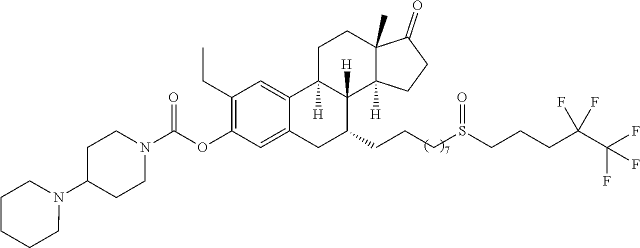 |
| 53 | 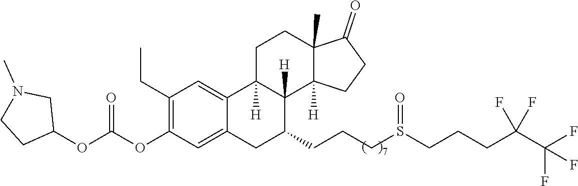 |
| 54 | 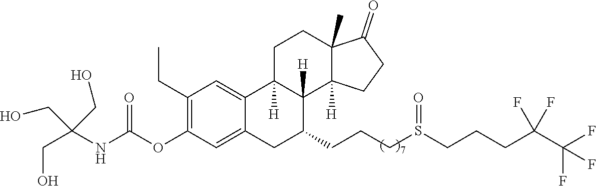 |
| 55 | 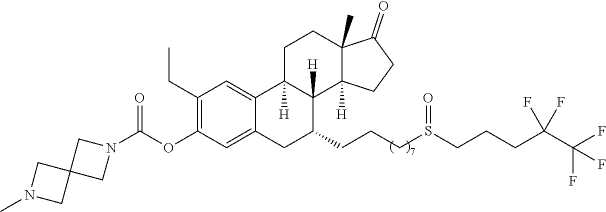 |
| 56 | 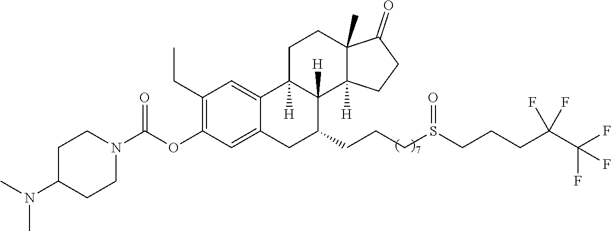 |
| 57 | 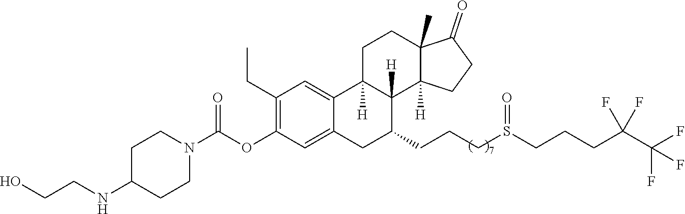 |
| 58 | 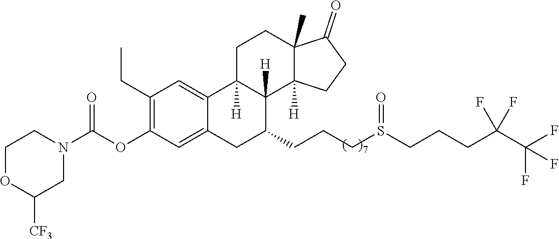 |
| 59 | 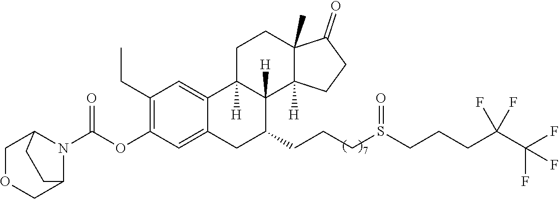 |
| 60 | 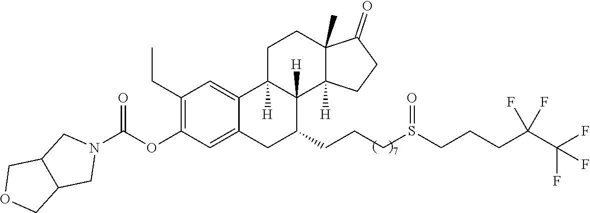 |
| 61 | 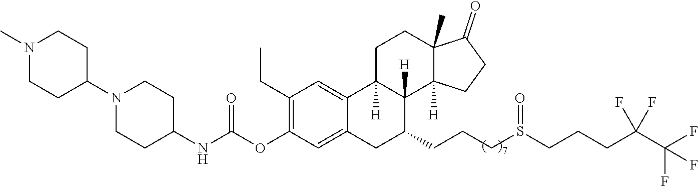 |
| 62 | 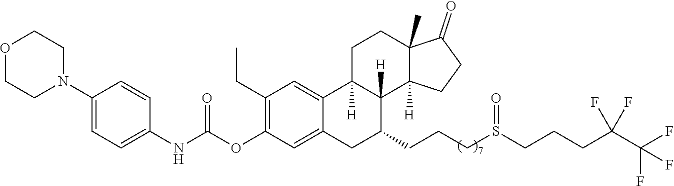 |
| 63 | 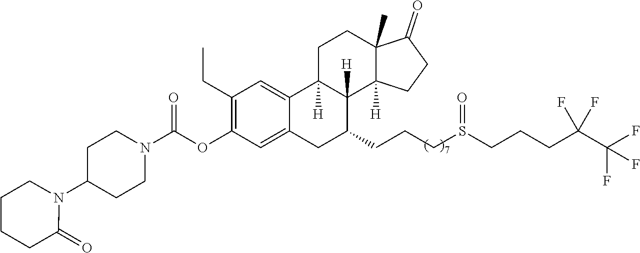 |
| 64 | 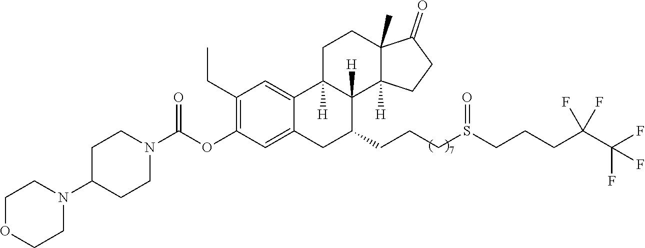 |
| 65 | 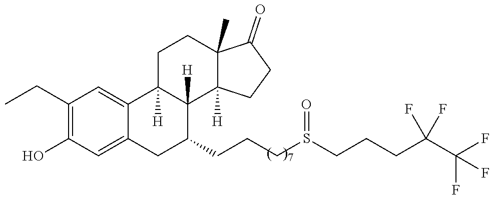 |
| 66 | 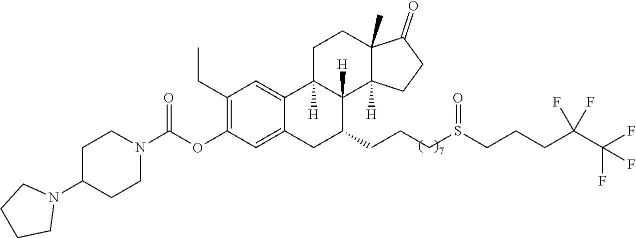 |
| 67 | 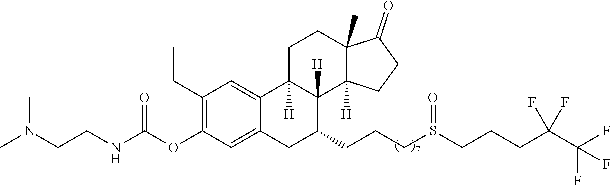 |
| 68 | 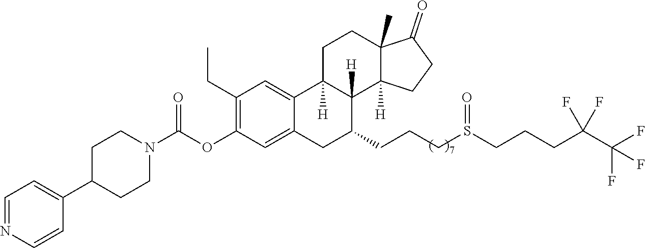 |
| 69 | 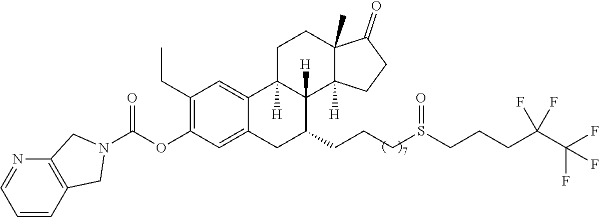 |
| 70 | 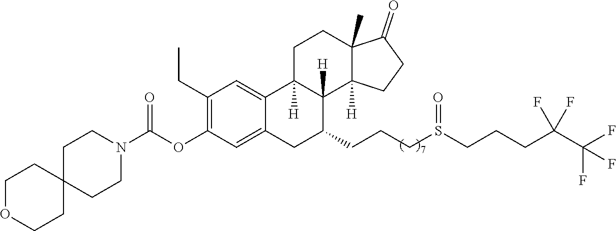 |
| 71 | 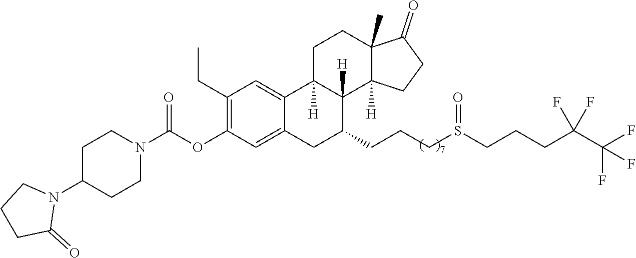 |
| 72 | 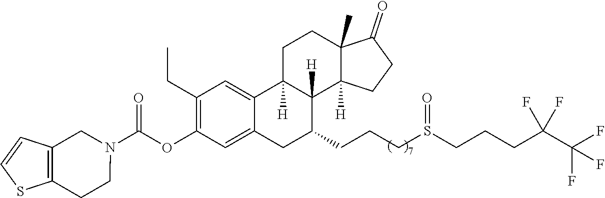 |
| 73 | 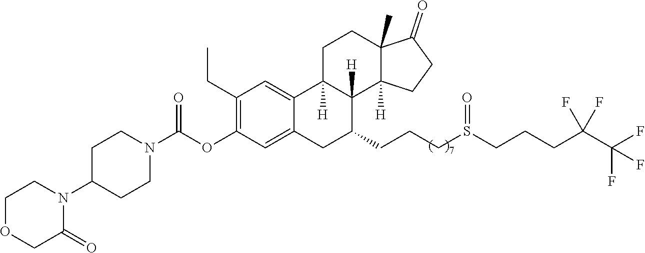 |
| 74 | 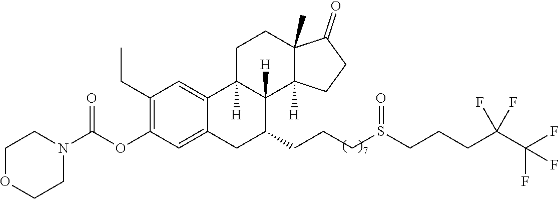 |
| 75 | 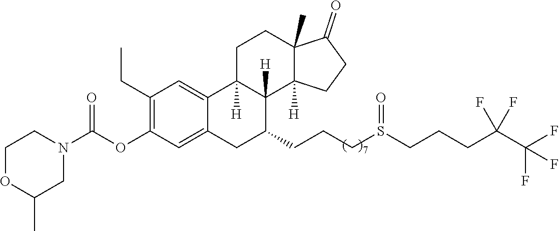 |
| 76 | 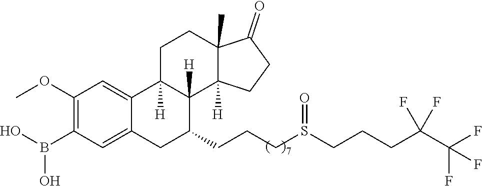 |
| 77 | 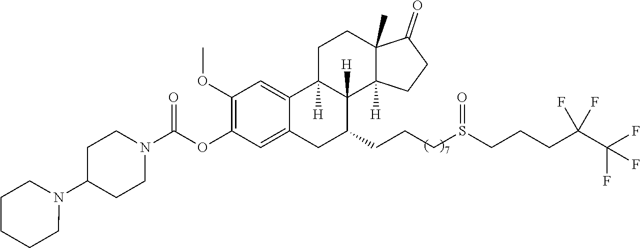 |
| 78 | 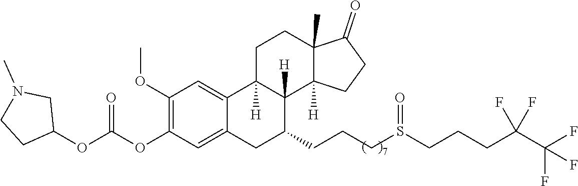 |
| 79 | 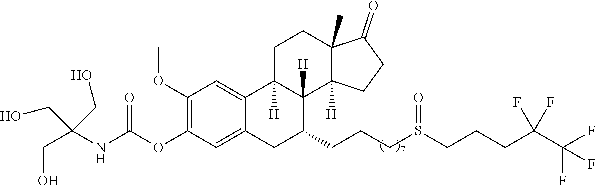 |
| 80 | 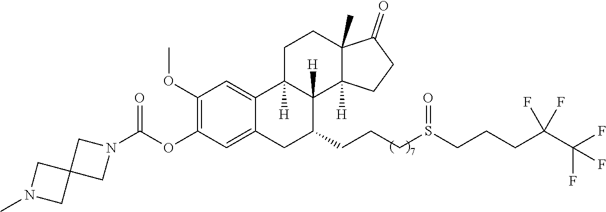 |
| 81 | 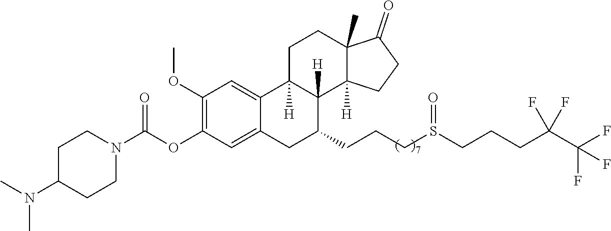 |
| 82 | 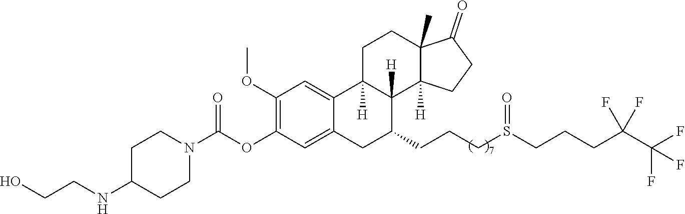 |
| 83 | 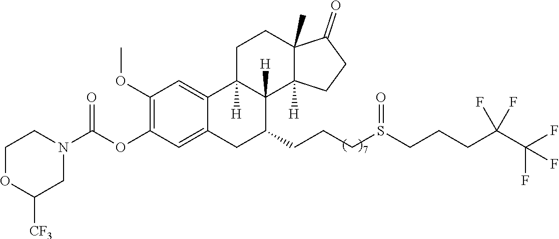 |
| 84 | 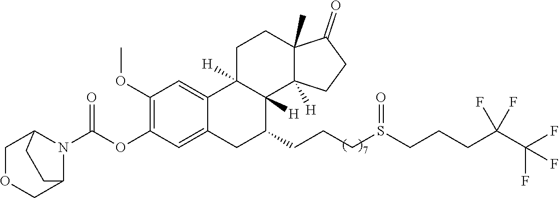 |
| 85 | 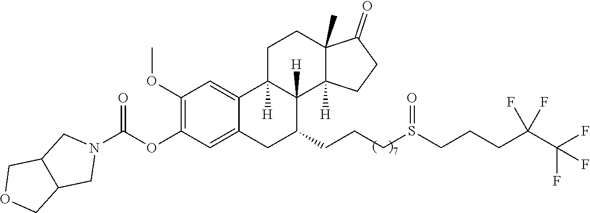 |
| 86 | 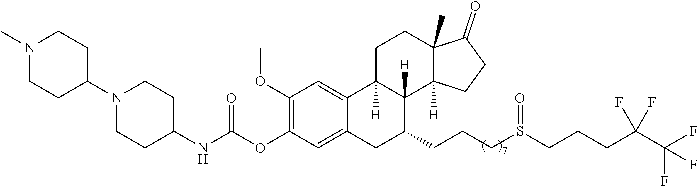 |
| 87 | 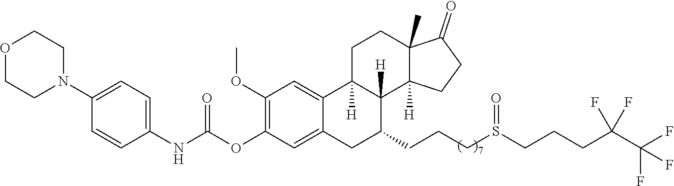 |
| 88 | 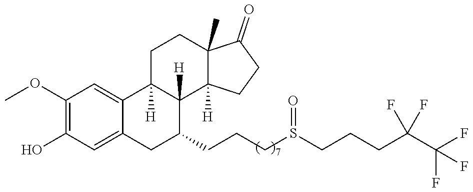 |
| 89 | 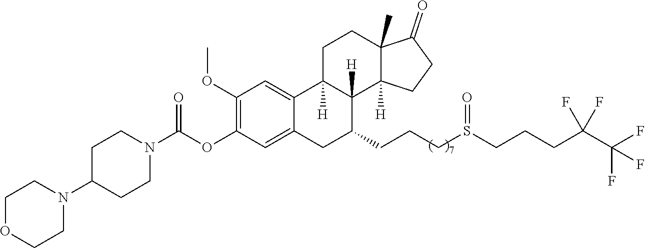 |
| 90 | 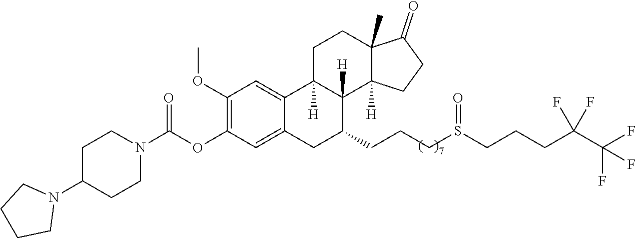 |
| 91 | 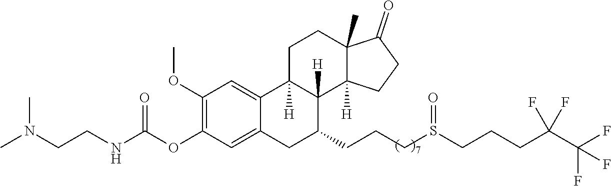 |
| 92 | 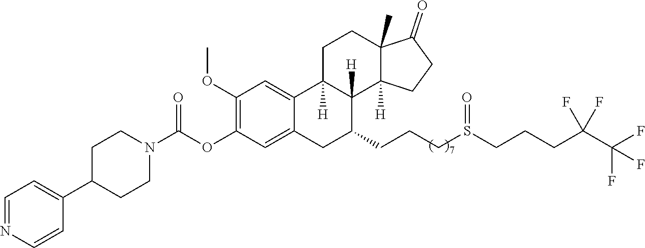 |
| 93 | 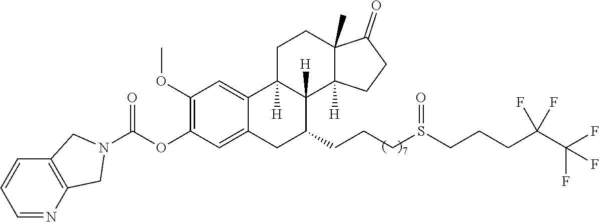 |
| 94 | 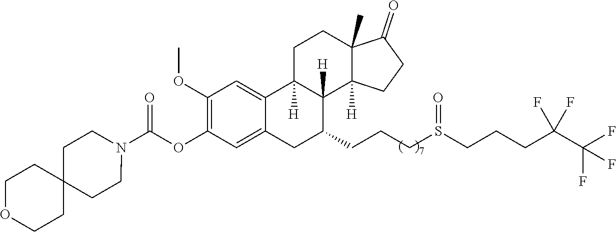 |
| 95 | 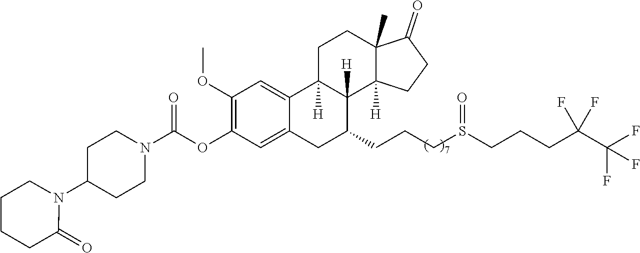 |
| 96 | 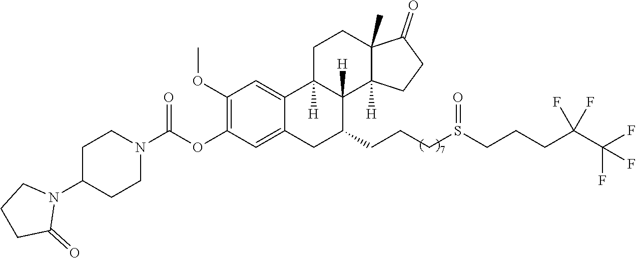 |
| 97 | 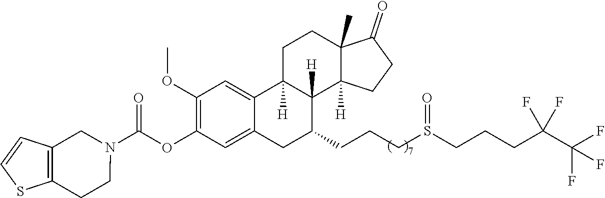 |
| 98 | 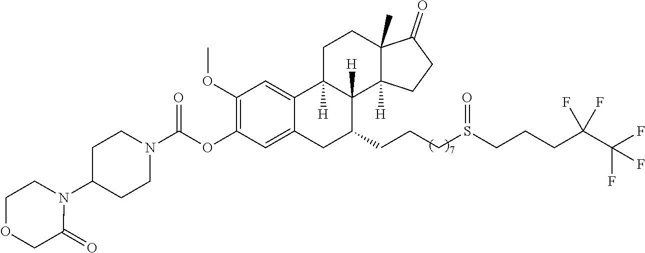 |
| 99 | 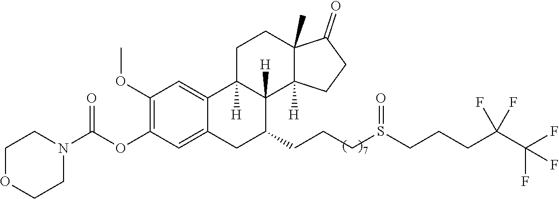 |
| 100 | 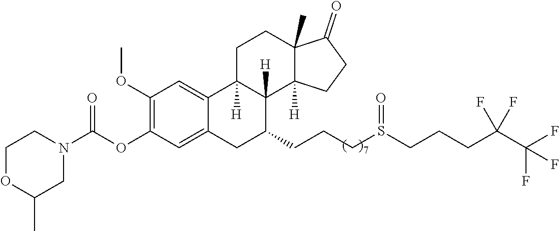 |
| 101 | 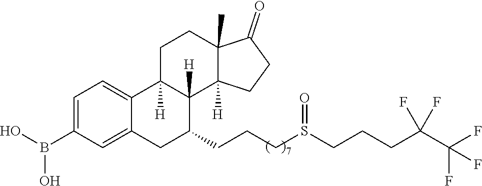 |
| 102 | 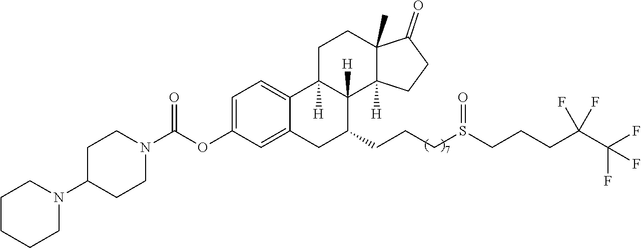 |
| 103 | 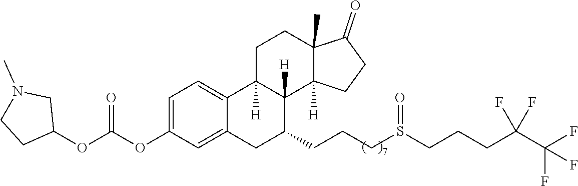 |
| 104 | 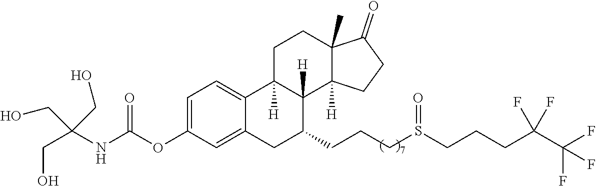 |
| 105 | 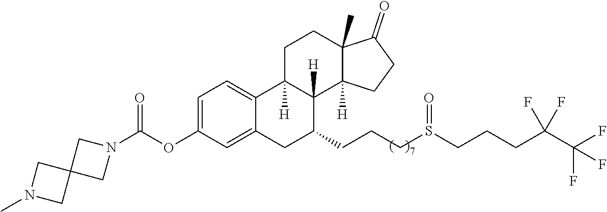 |
| 106 | 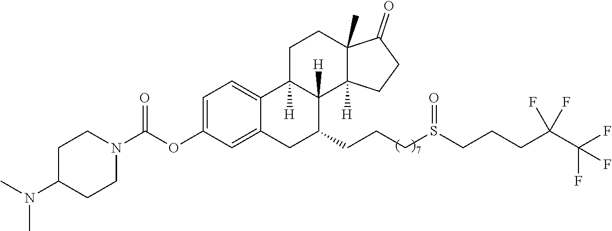 |
| 107 | 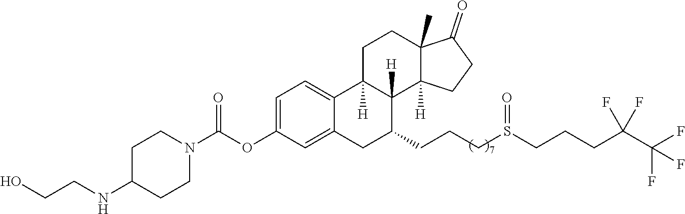 |
| 108 | 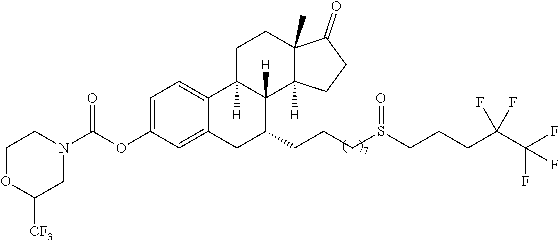 |
| 109 | 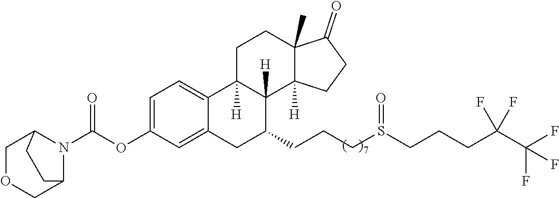 |
| 110 | 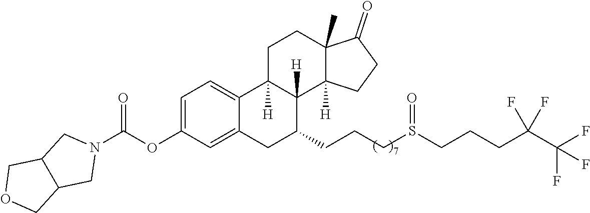 |
| 111 | 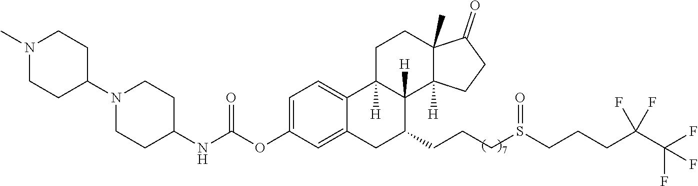 |
| 112 | 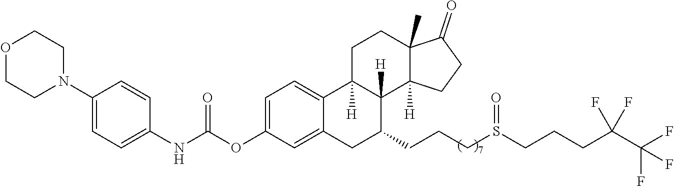 |
| 113 | 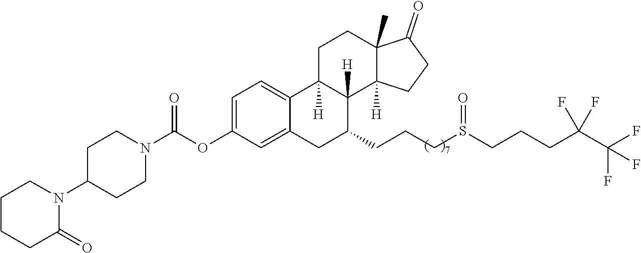 |
| 114 | 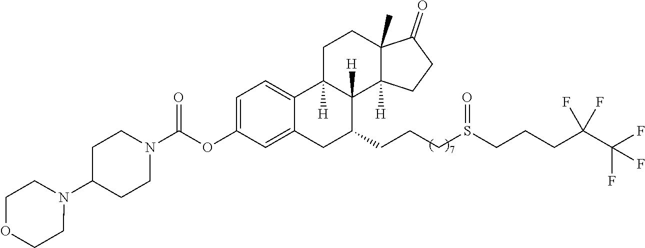 |
| 115 | 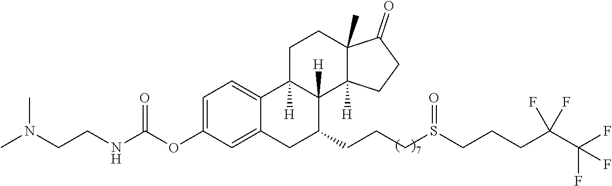 |
| 116 | 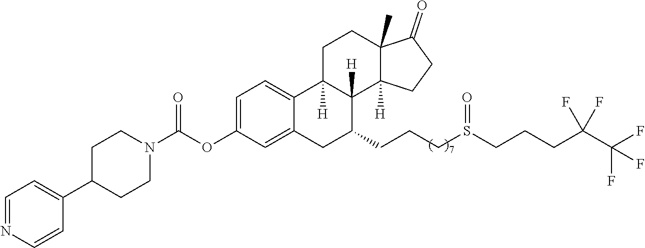 |
| 117 | 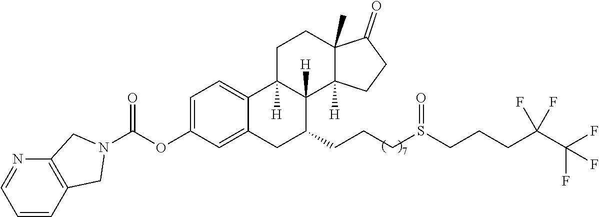 |
| 118 | 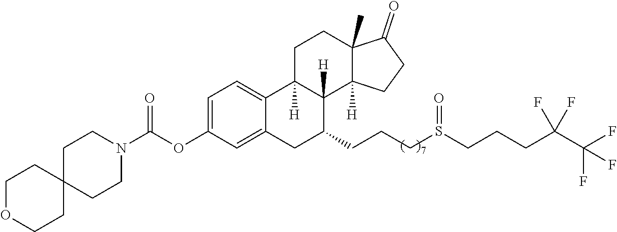 |
| 119 | 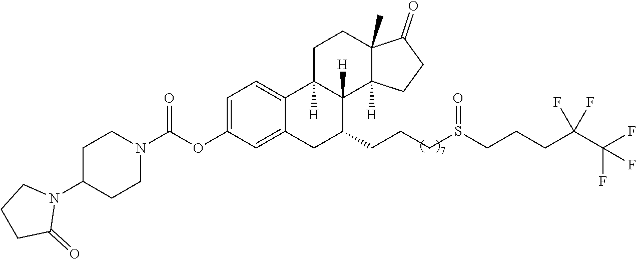 |
| 120 | 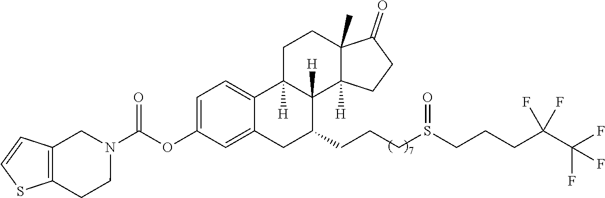 |
| 121 | 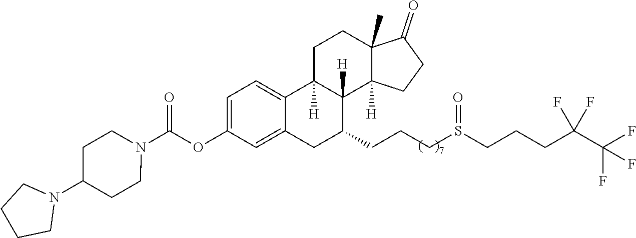 |
| 122 | 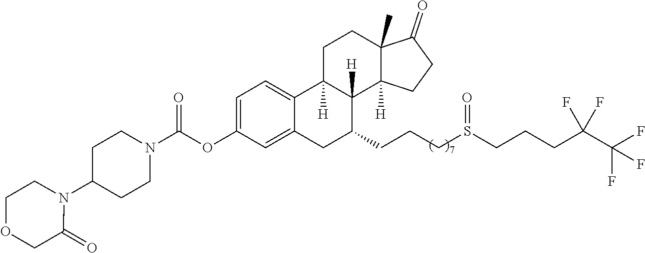 |
| 123 | 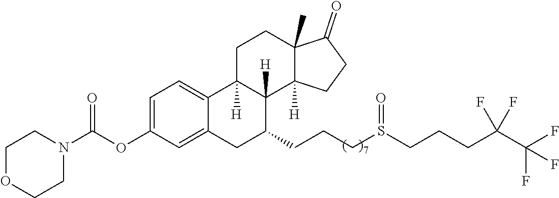 |
| 124 | 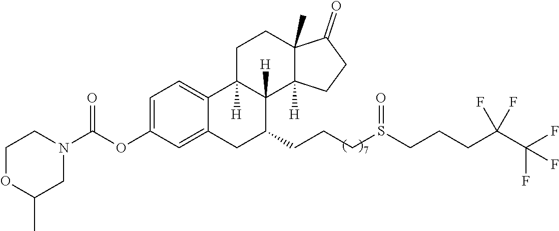 |
| 125 | 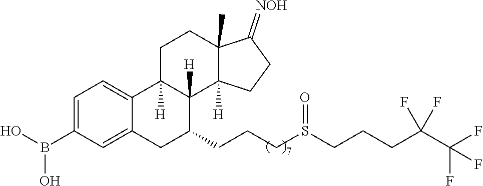 |
| 126 | 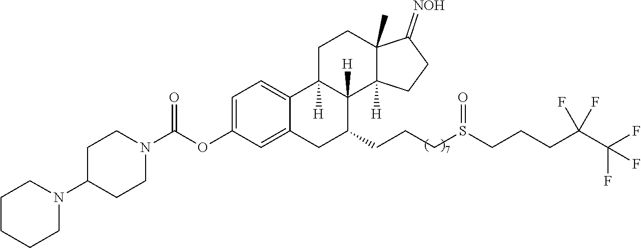 |
| 127 | 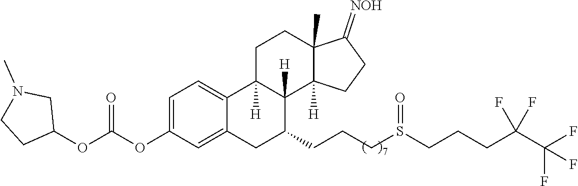 |
| 128 | 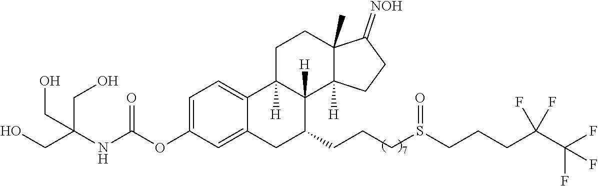 |
| 129 | 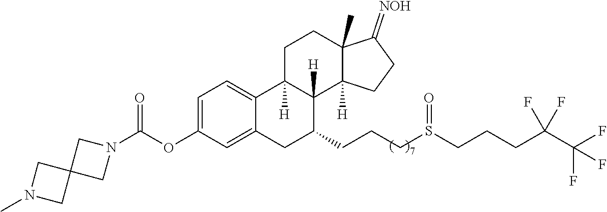 |
| 130 | 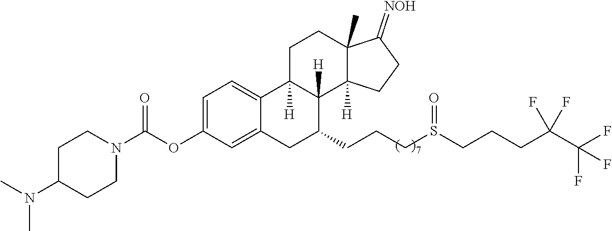 |
| 131 | 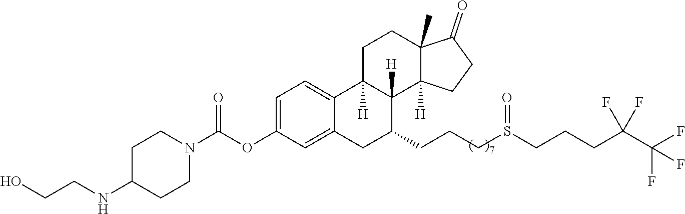 |
| 132 | 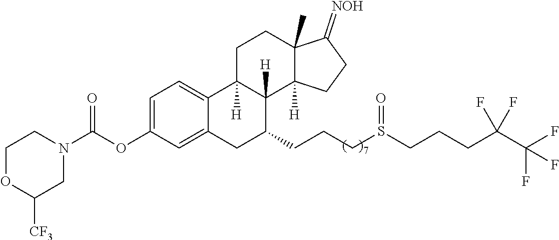 |
| 133 | 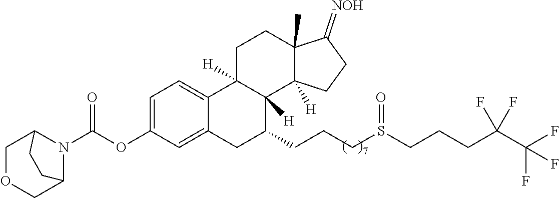 |
| 134 | 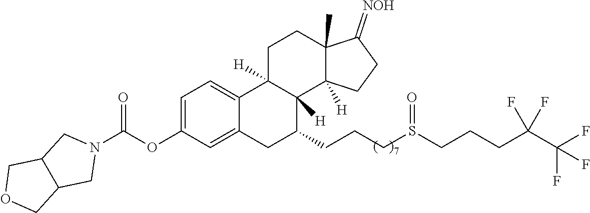 |
| 135 | 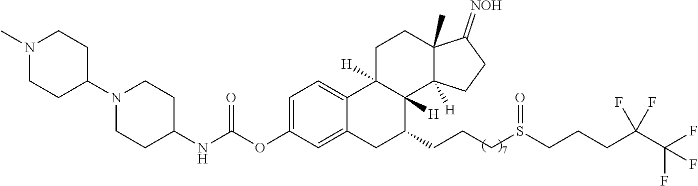 |
| 136 | 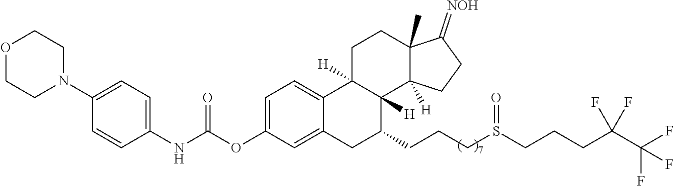 |
| 137 | 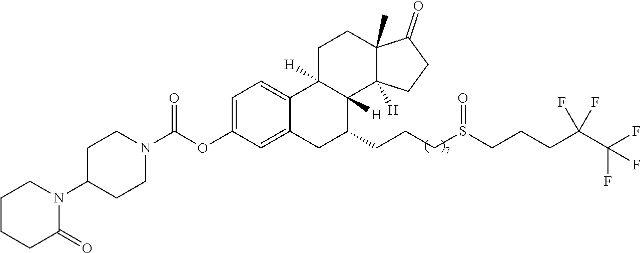 |
| 138 | 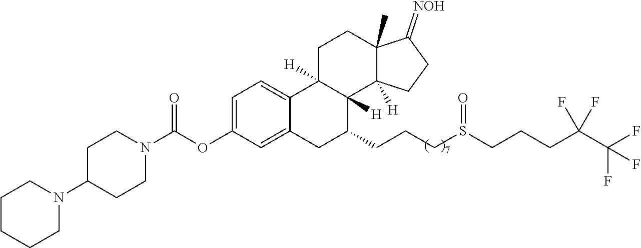 |
| 139 | 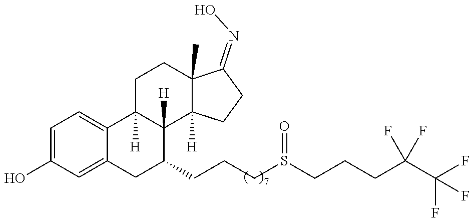 |
| 140 | 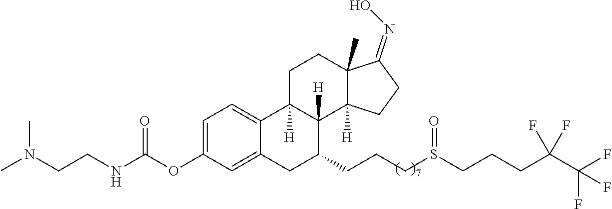 |
| 141 | 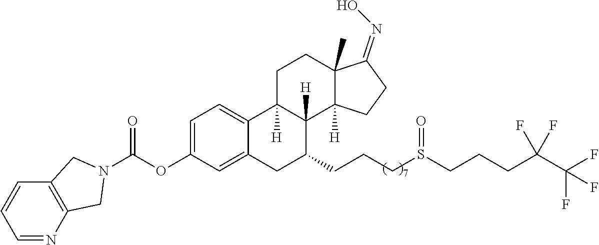 |
| 142 | 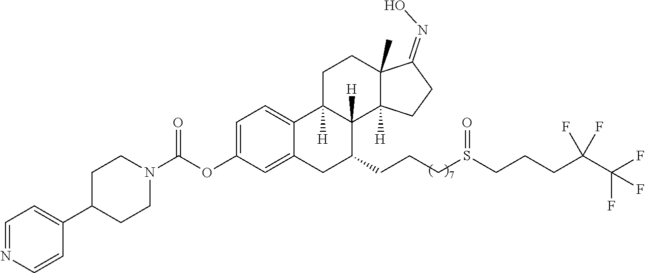 |
| 143 | 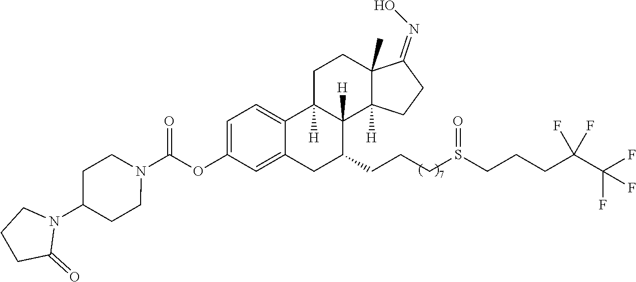 |
| 144 | 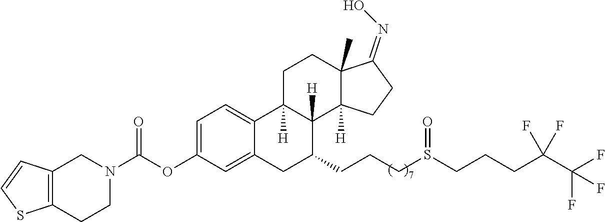 |
| 145 | 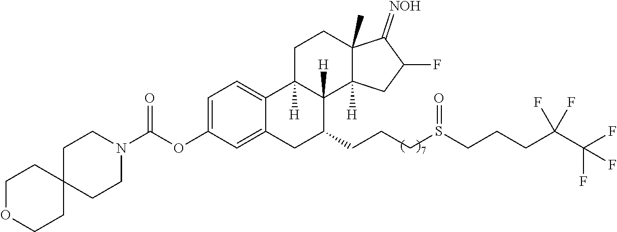 |
| 146 | 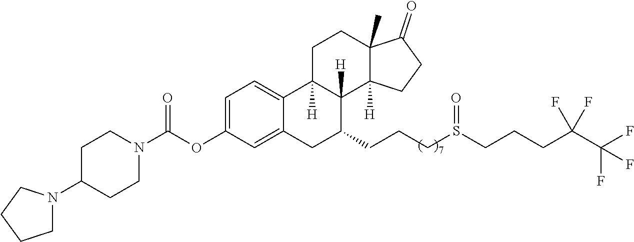 |
| 147 | 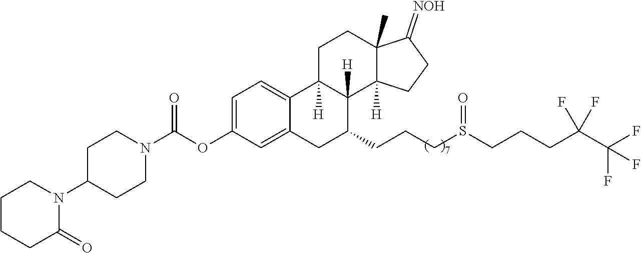 |
| 148 | 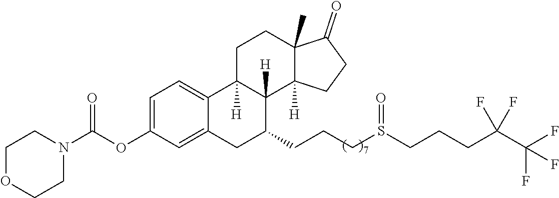 |
| 149 | 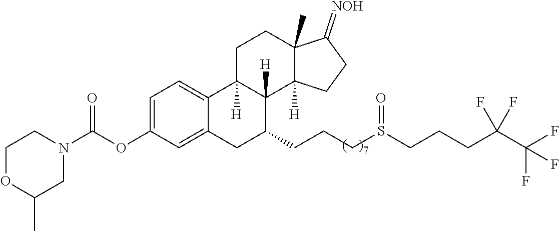 |
| 150 | 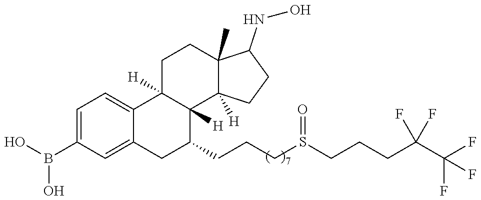 |
| 151 | 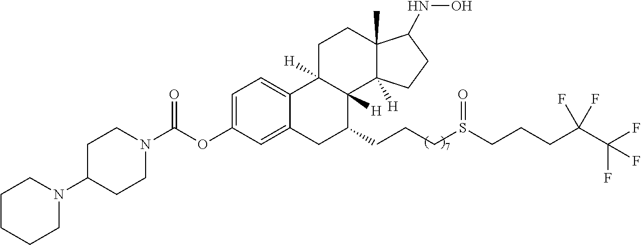 |
| 152 | 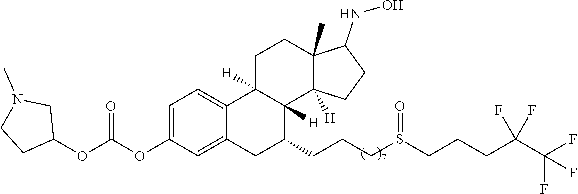 |
| 153 | 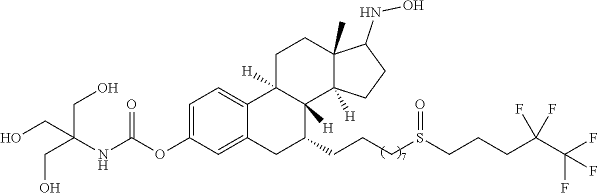 |
| 154 | 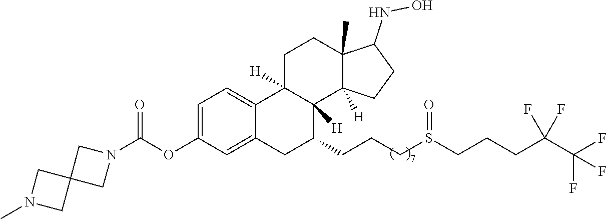 |
| 155 | 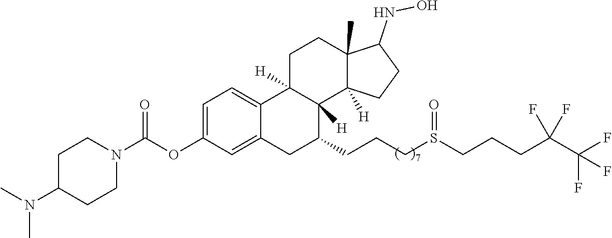 |
| 156 | 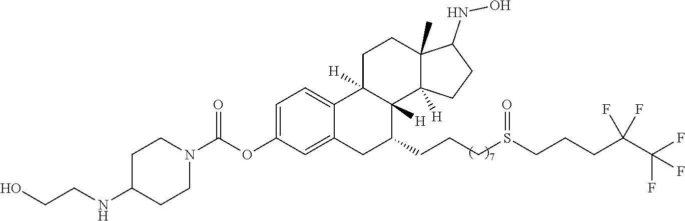 |
| 157 | 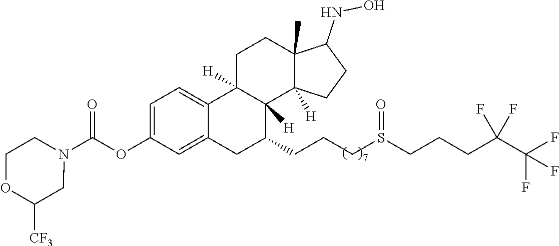 |
| 158 | 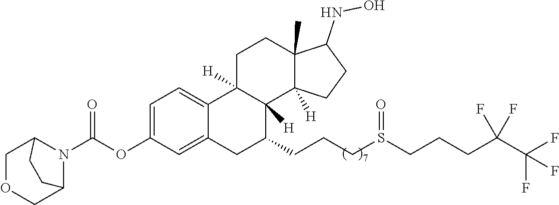 |
| 159 | 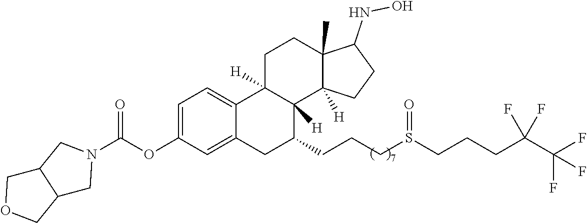 |
| 160 | 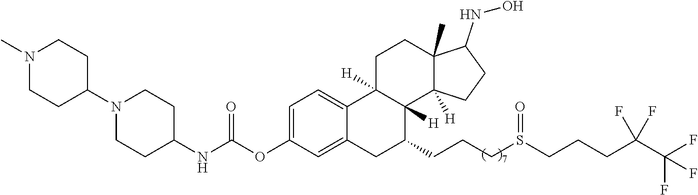 |
| 161 | 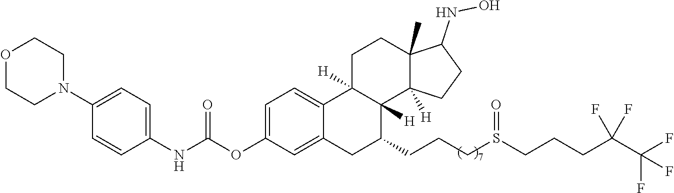 |
| 162 | 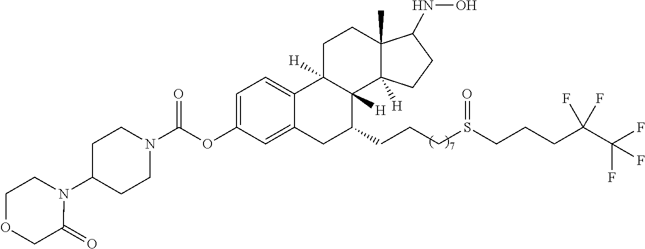 |
| 163 | 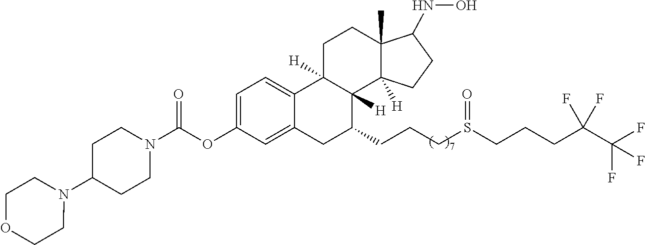 |
| 164 | 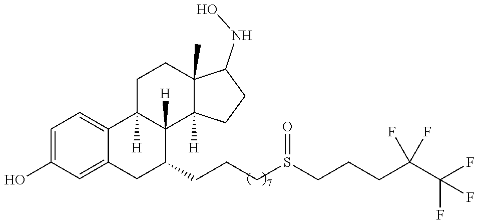 |
| 165 | 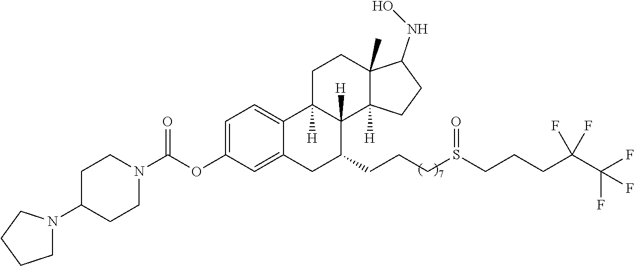 |
| 166 | 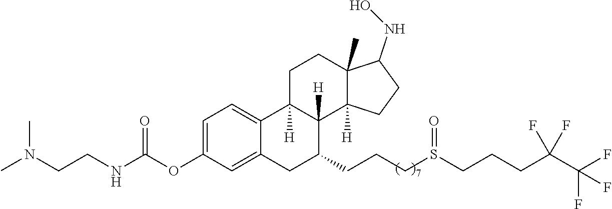 |
| 167 | 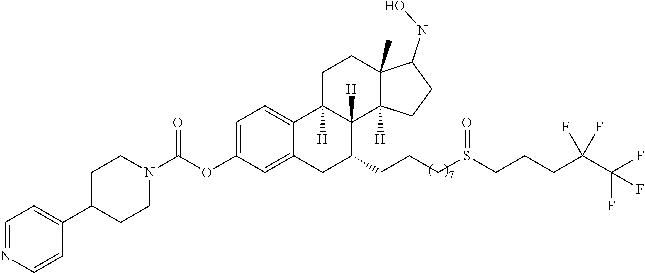 |
| 168 | 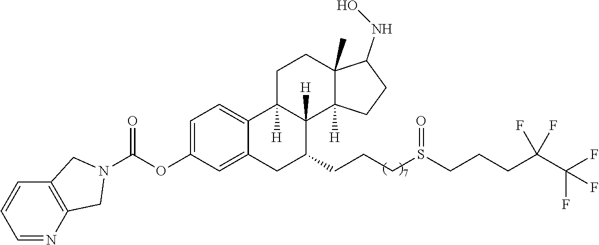 |
| 169 | 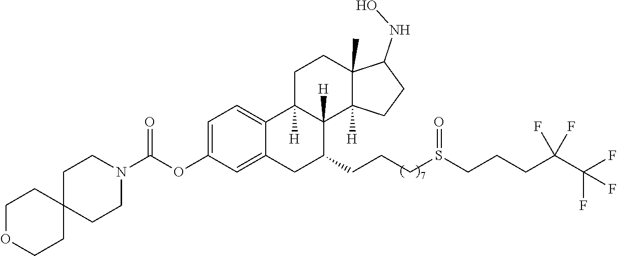 |
| 170 | 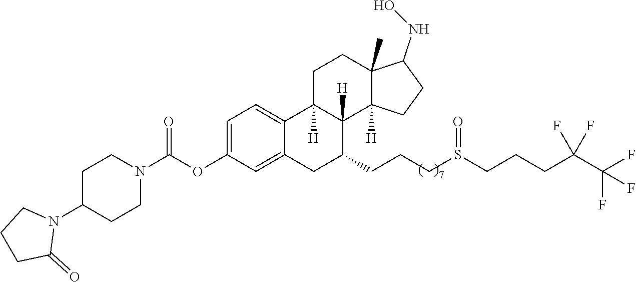 |
| 171 | 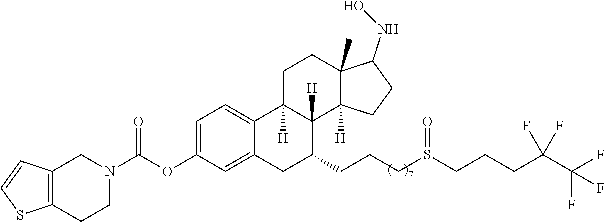 |
| 172 | 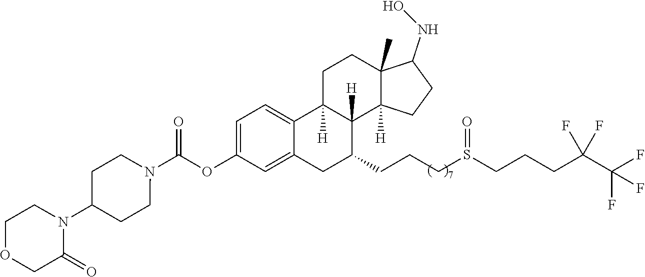 |
| 173 | 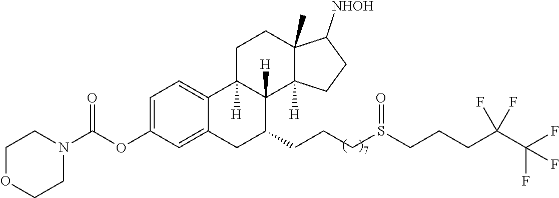 |
| 174 | 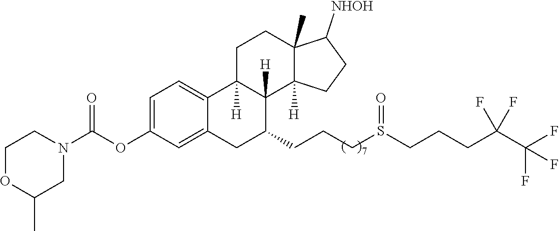 |
| 175 | 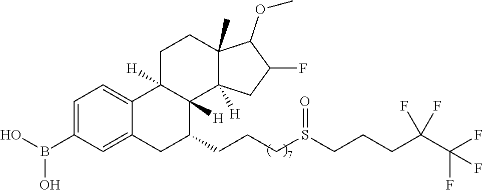 |
| 176 | 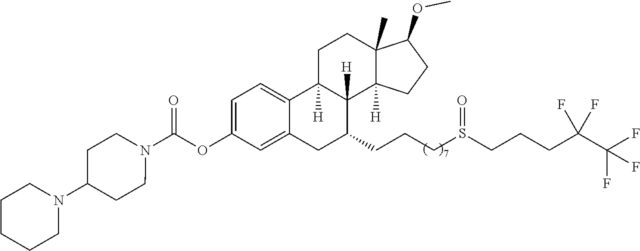 |
| 177 | 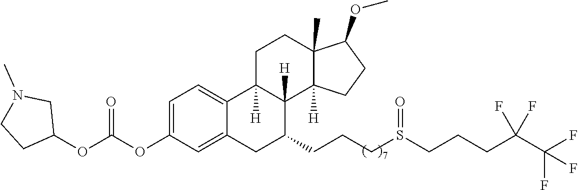 |
| 178 | 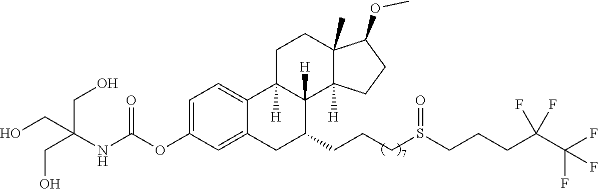 |
| 179 | 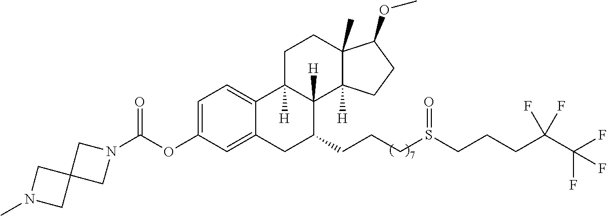 |
| 180 | 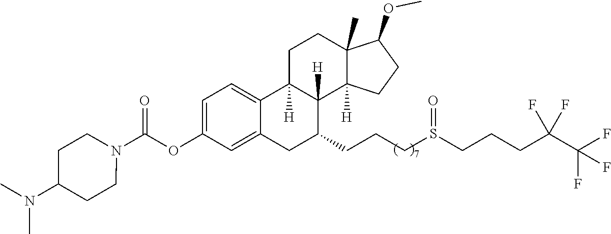 |
| 181 | 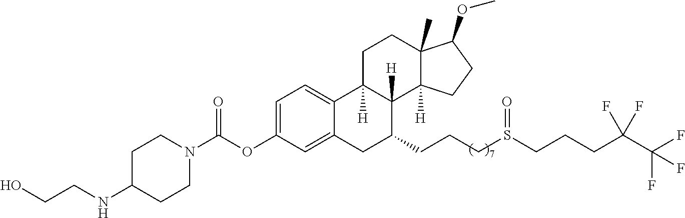 |
| 182 | 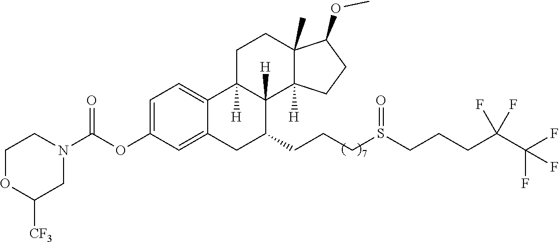 |
| 183 | 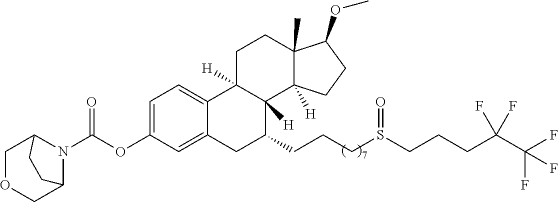 |
| 184 | 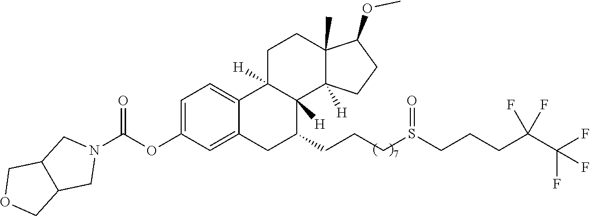 |
| 185 | 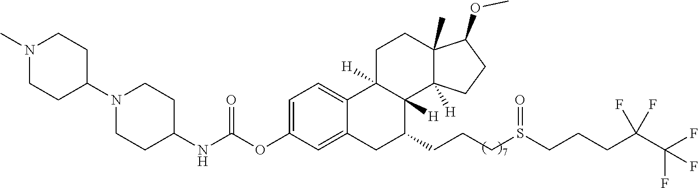 |
| 186 | 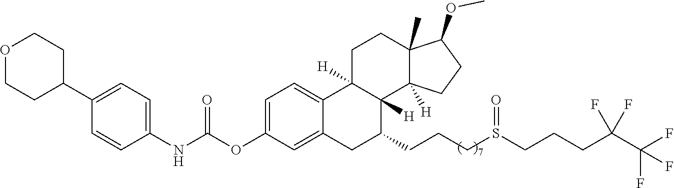 |
| 187 | 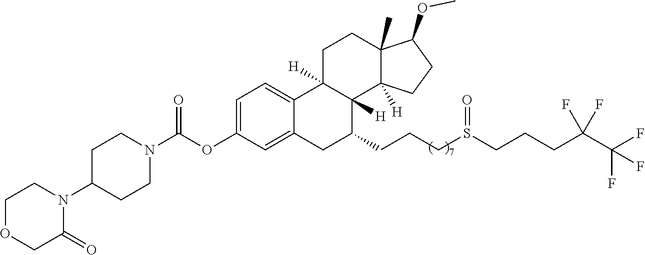 |
| 188 | 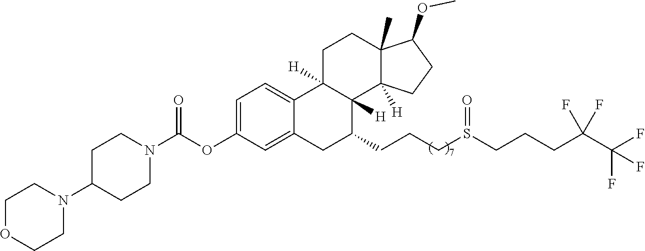 |
| 189 | 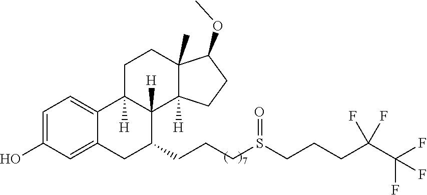 |
| 190 | 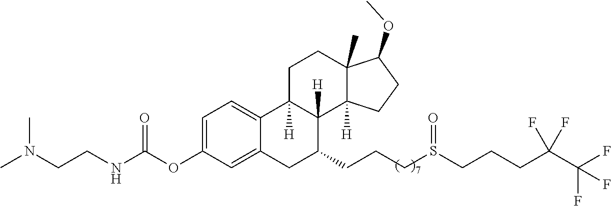 |
| 191 | 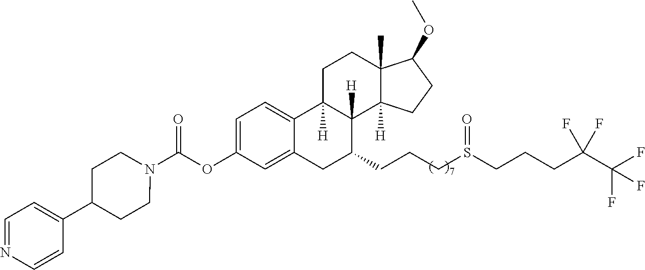 |
| 192 | 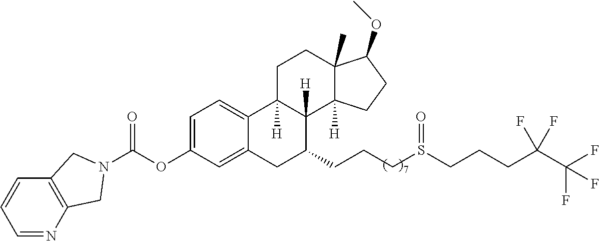 |
| 193 | 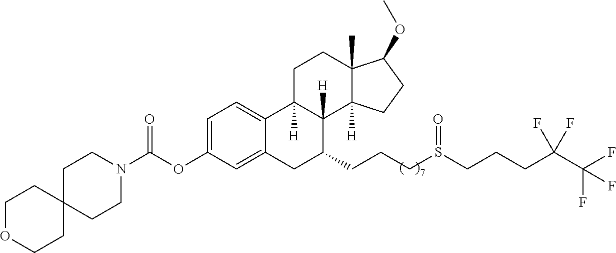 |
| 194 | 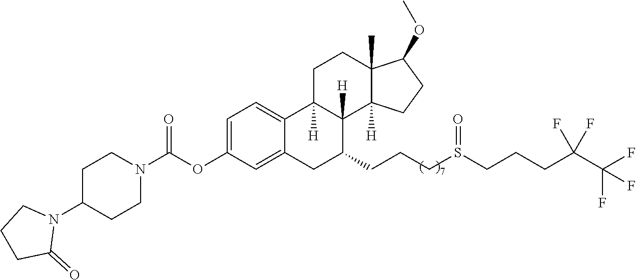 |
| 195 | 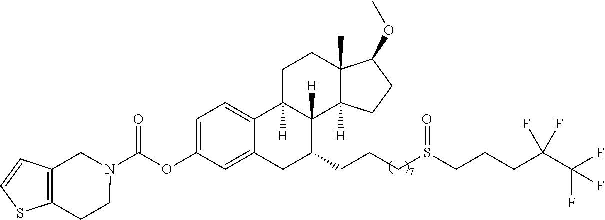 |
| 196 | 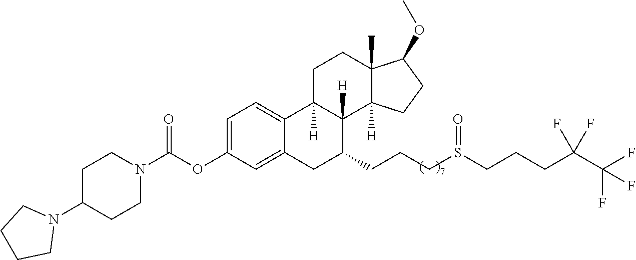 |
| 197 | 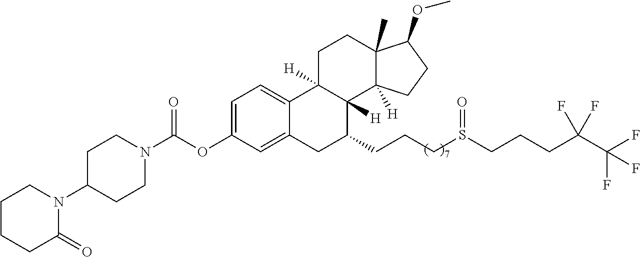 |
| 198 | 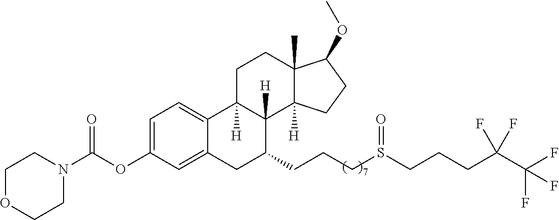 |
| 199 | 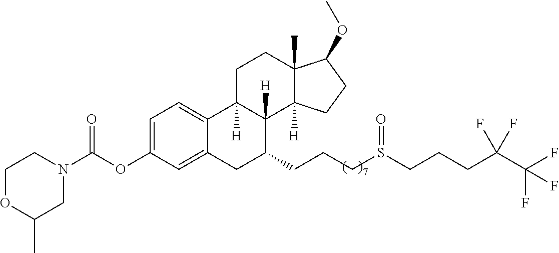 |
| 200 | 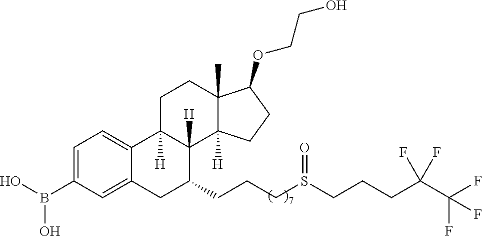 |
| 201 | 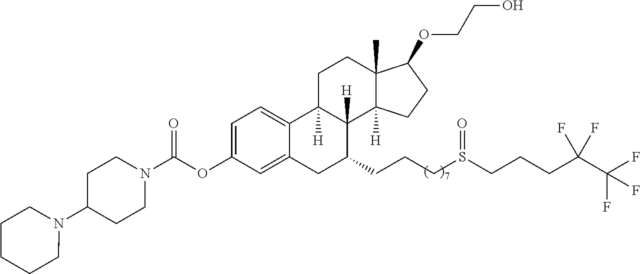 |
| 202 | 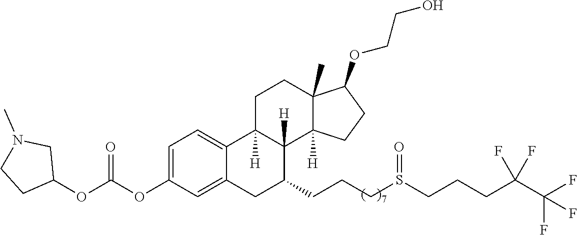 |
| 203 | 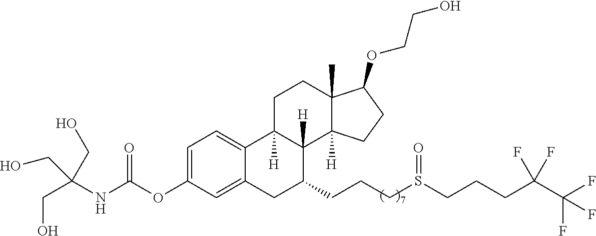 |
| 204 | 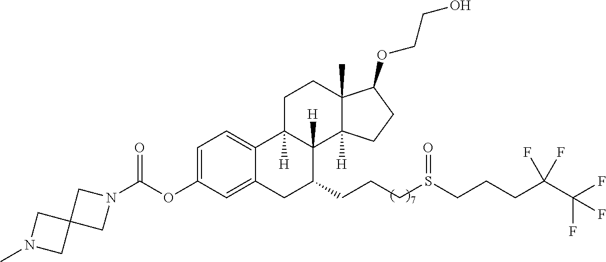 |
| 205 | 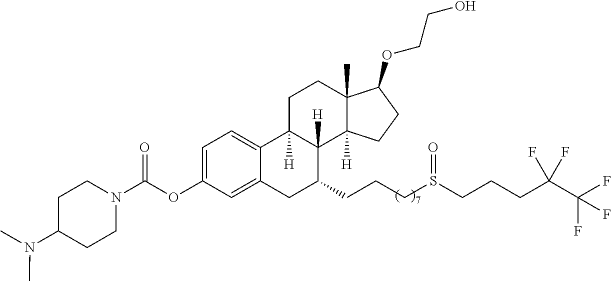 |
| 206 | 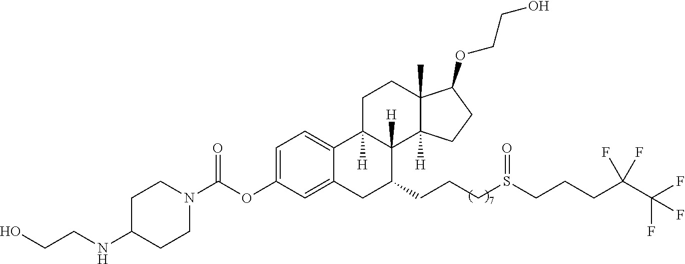 |
| 207 | 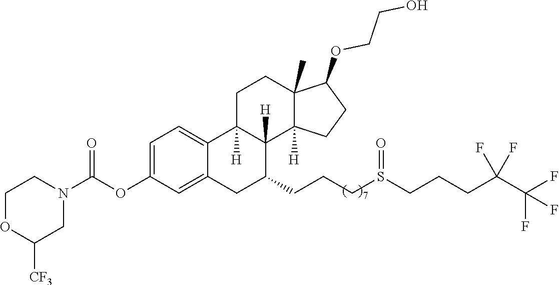 |
| 208 | 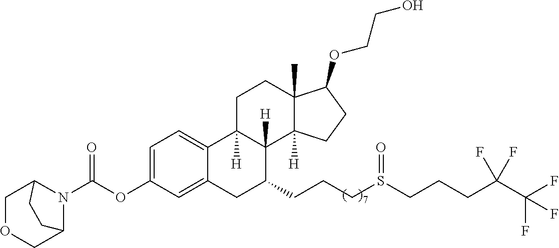 |
| 209 | 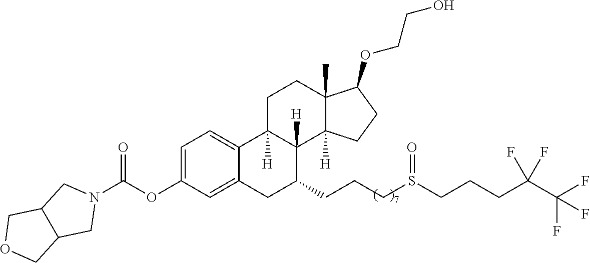 |
| 210 | 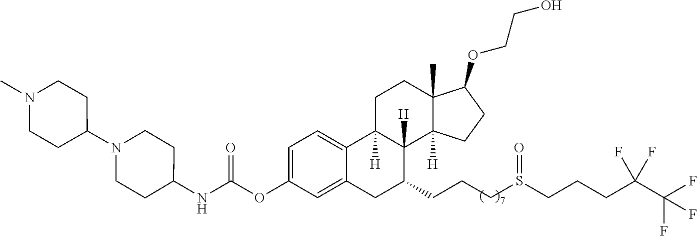 |
| 211 | 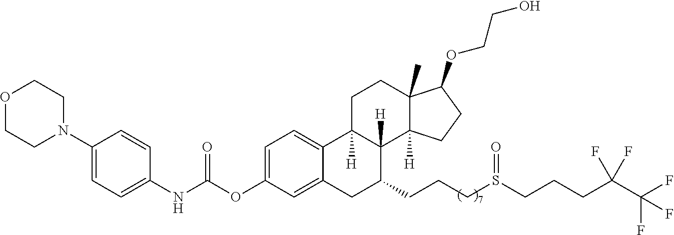 |
| 212 | 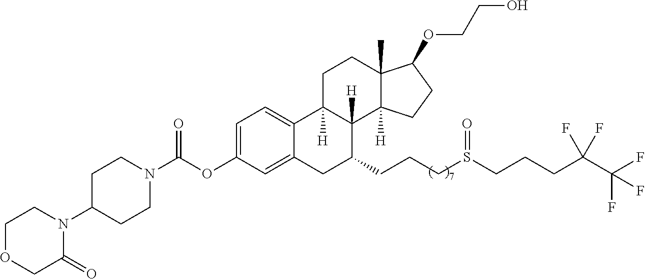 |
| 213 | 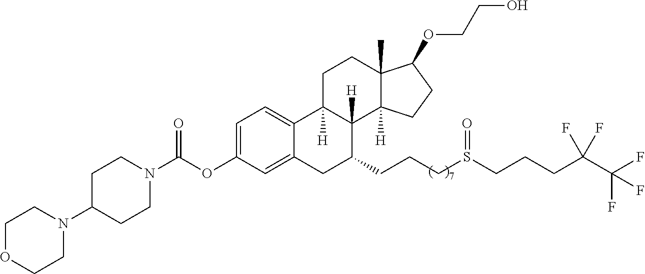 |
| 214 | 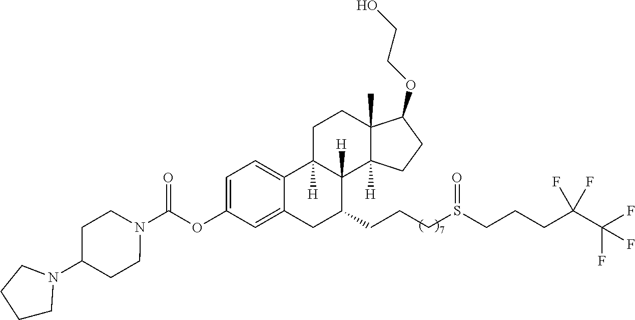 |
| 215 | 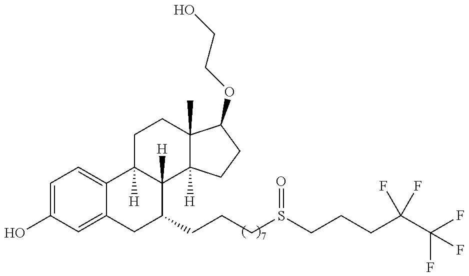 |
| 216 | 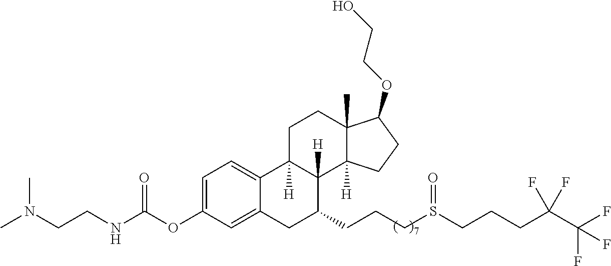 |
| 217 | 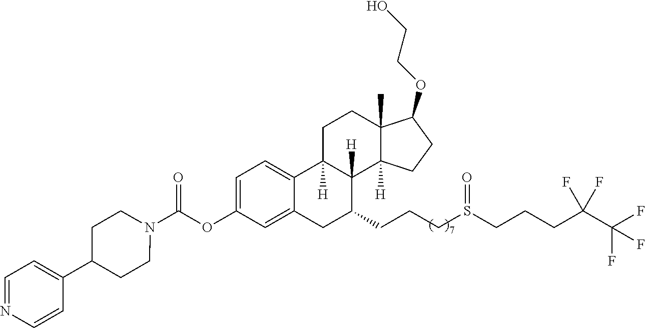 |
| 218 | 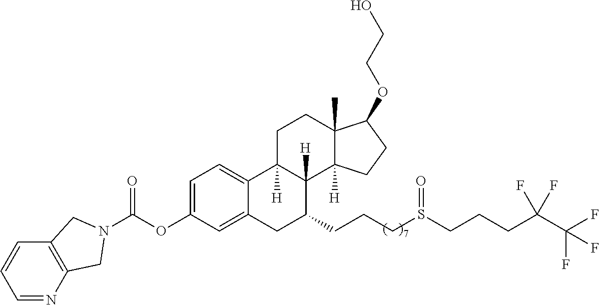 |
| 219 | 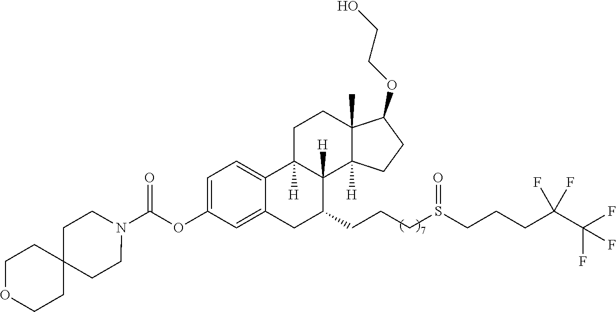 |
| 220 | 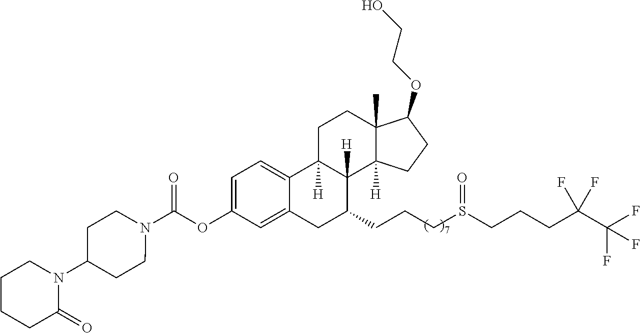 |
| 221 | 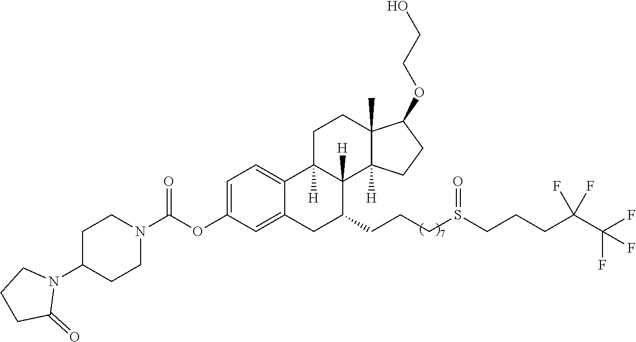 |
| 222 | 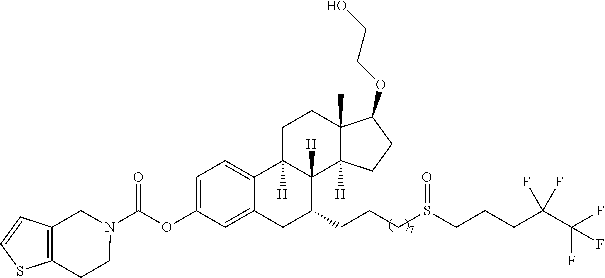 |
| 223 | 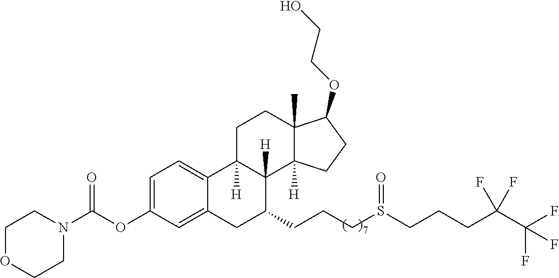 |
| 224 | 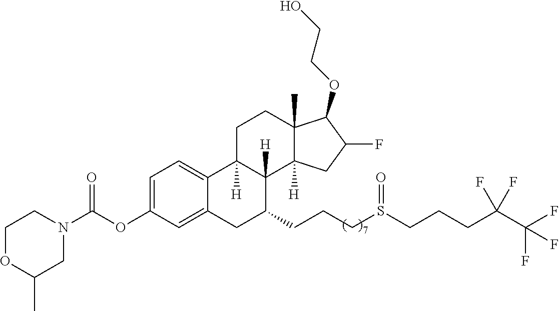 |
| 225 | 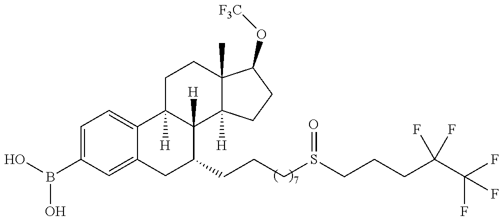 |
| 226 | 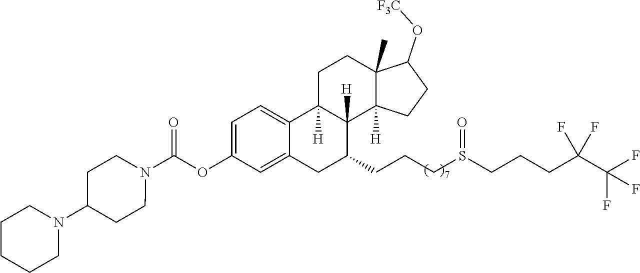 |
| 227 | 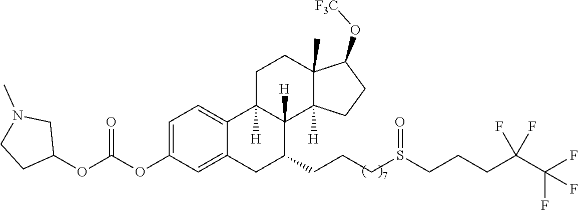 |
| 228 | 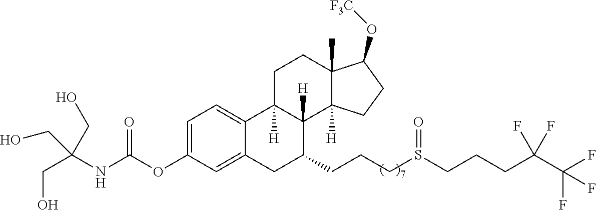 |
| 229 | 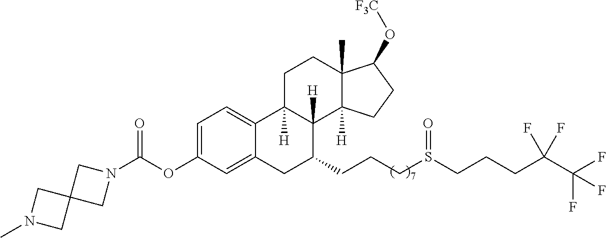 |
| 230 | 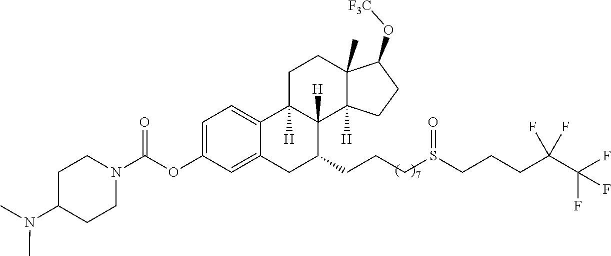 |
| 231 | 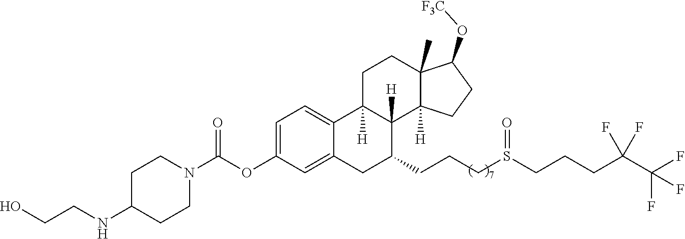 |
| 232 | 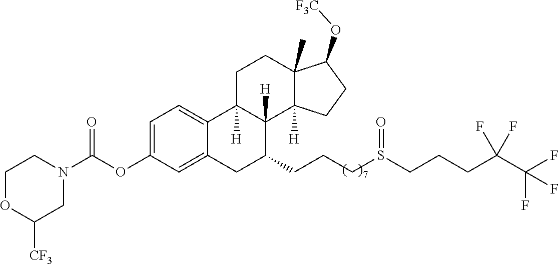 |
| 233 | 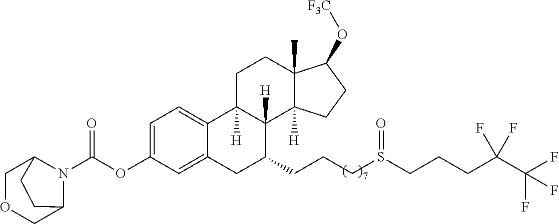 |
| 234 | 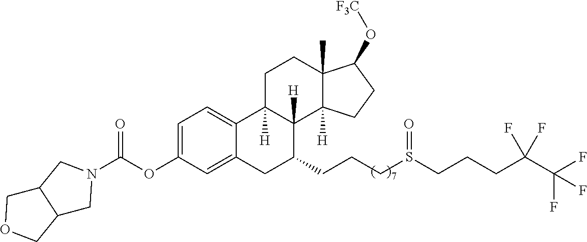 |
| 235 | 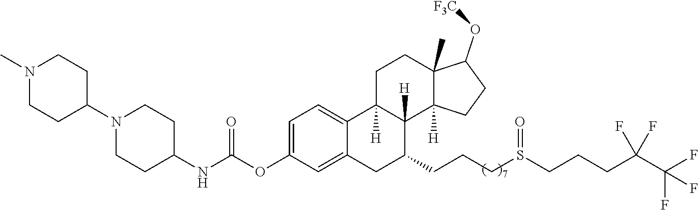 |
| 236 | 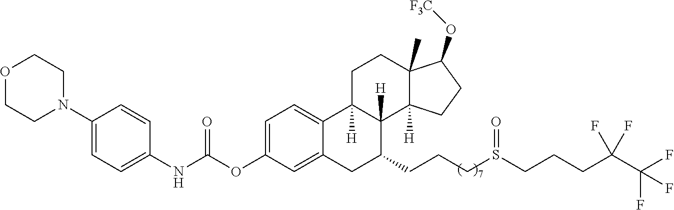 |
| 237 | 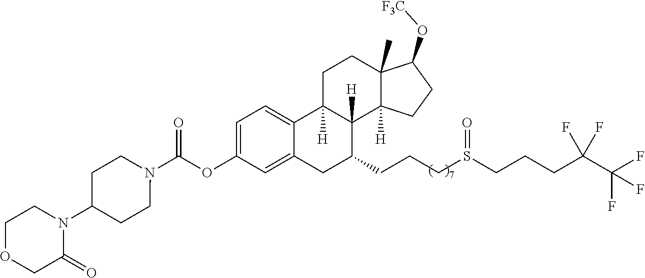 |
| 238 | 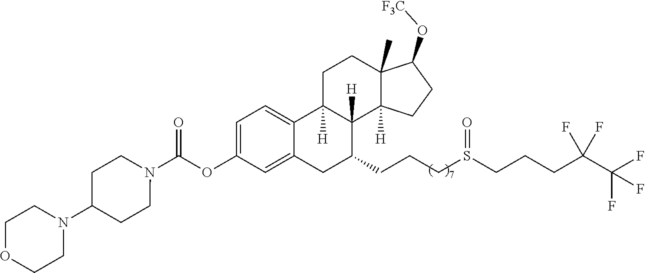 |
| 239 | 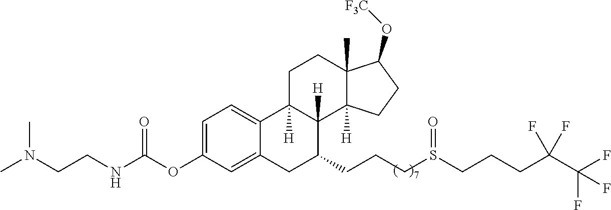 |
| 240 | 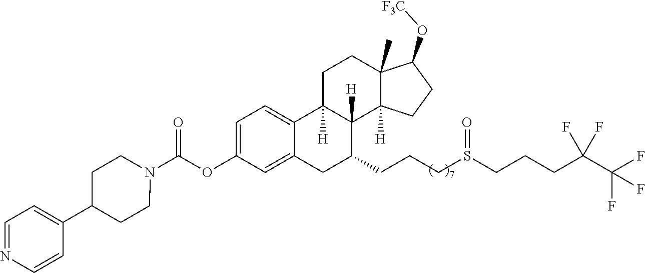 |
| 241 | 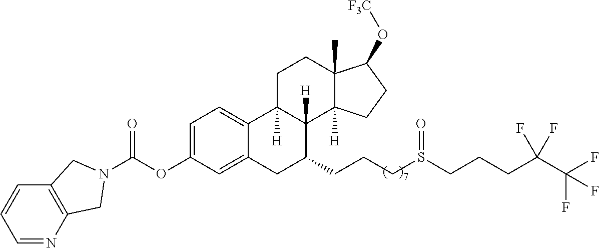 |
| 242 | 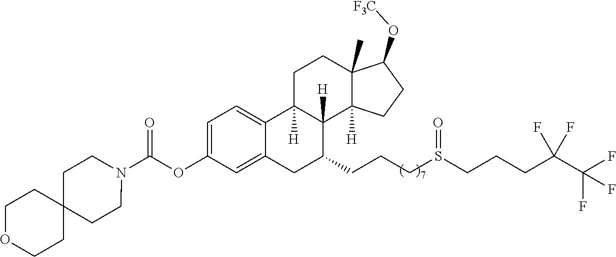 |
| 243 | 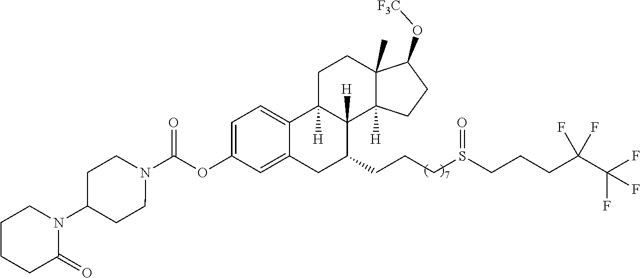 |
| 244 | 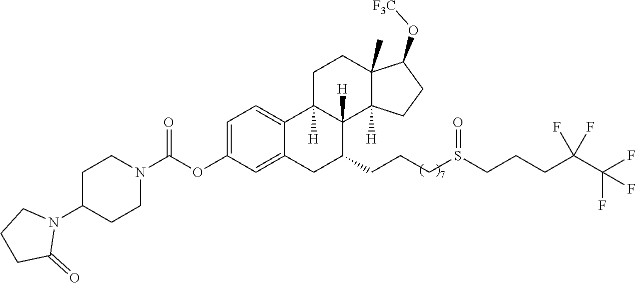 |
| 245 | 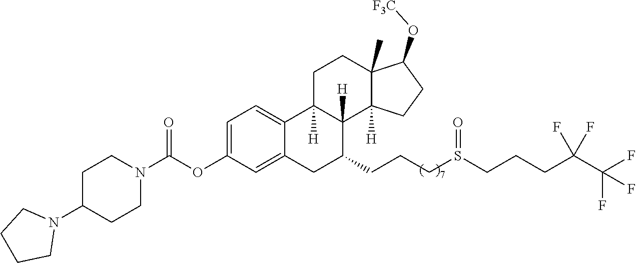 |
| 246 | 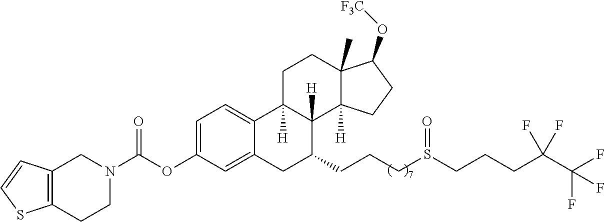 |
| 247 | 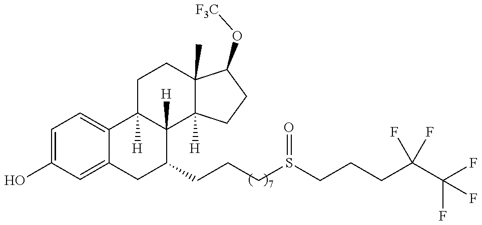 |
| 248 | 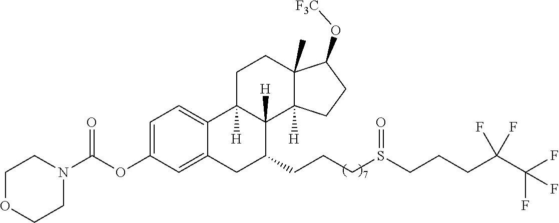 |
| 249 | 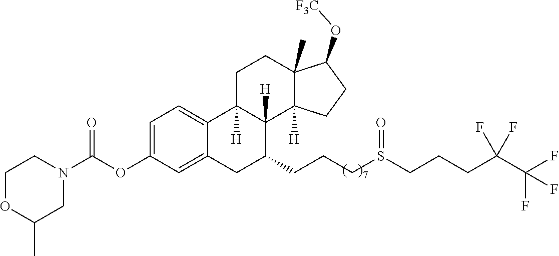 |
| 250 | 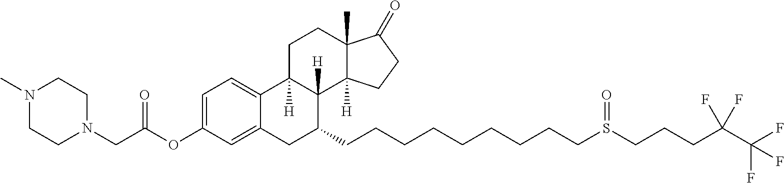 |

36. A pharmaceutical composition comprising a compound of formula (I) of claim 1 or a compound (I-A) of claim 2; enantiomers, diastereomers, racemates, pharmaceutically acceptable salts or solvates thereof, and a pharmaceutically acceptable excipients.

37. (canceled)

38. A method of treating a benign or malignant diseases of the breast or reproductive tract, comprising administration of a compound of formula (I) 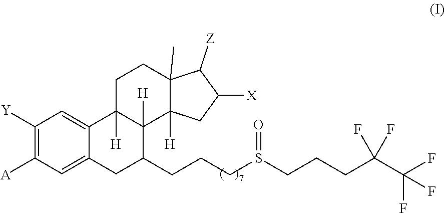
wherein;
X is halogen or hydrogen;
Y is alkyl, —O-alkyl or hydrogen;
Z is selected from ═O, ═N—OH, —O-alkyl, —O-haloalkyl, —NH—OH or —O-alkyl-OH wherein the bond between the Z substituent attached to carbon can be single or double bond depend on the substituent selected;
A is selected from 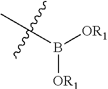
or —O—R₂, wherein R₁ is selected from hydrogen or alkyl; R₂ is selected independently from group consisting of
i) 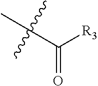
wherein R₃ is selected from NH₂, NHR₄, or NR₅R₆; R₄ is selected from group comprising optionally substituted alkyl, alkenyl, alkynyl, cycloalkyl, heterocycloalkyl, aryl, or heteroaryl; R₅ and R₆ are selected independently from group comprising optionally substituted alkyl, aryl, heteroaryl, heterocycloalkyl, cycloalkyl; or R₅ and R₆ are taken together along with the nitrogen to which they are attached to form a three to seven membered heterocyclic ring and may optionally be substituted at a substitutable position with one or more R₇ radicals, wherein the one or more R₇ radicals are independently selected at each occurrence from the group consisting of optionally substituted alkyl, alkylcarbonyl, formyl, halo, haloalkyl, alkylphosphate, phosphate, oxo, cyano, nitro, amino, hydroxyalkylamino, alkoxy, alkoxycarbonyl, carboxyalkyl, hydroxyalkyl, alkenyl, alkynyl, alkylthio, cycloalkyl, aryl, heteroaryl, aralkyl, heterocycloalkyl, alkylthioalkyl, arylcarbonyl, aralkylcarbonyl, alkoxyalkyl;
ii) 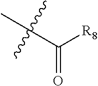
wherein R₈ is selected from group comprising optionally substituted alkyl, alkenyl, alkynyl, cycloalkyl, aryl, heteroaryl; or —CR₉R₁₀; where R₉ and R₁₀ are taken together along with the carbon to which they are attached to form a three to seven membered saturated, partially unsaturated or unsaturated heterocyclic ring in which up to 4 carbon atoms are replaced by heteroatoms chosen from the group consisting of O, S or N and may optionally be substituted at a substitutable position with one or more Ru radicals, wherein one or more Ru radicals are independently selected at each occurrence from the group consisting of optionally substituted alkyl, cycloalkyl, heterocycloalkyl, aryl, heteroaryl, alkylcarbonyl, formyl, halo, alkylphosphate, phosphate, cyano, nitro, alkoxy, amino, alkoxycarbonyl, alkenyl, alkynyl, alkylthio arylcarbonyl;
iii) 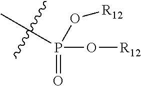
wherein each R₁₂ is selected independently from hydrogen; optionally substituted optionally substituted alkyl, aryl, acyl, heterocycloalkyl or heteroaryl;
iv) 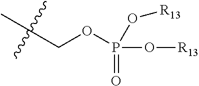
wherein each R₁₃ is selected independently from hydrogen; optionally substituted alkyl, cycloalkyl, heterocycloalkyl, aryl, or heteroaryl;
v) amino acids, wherein the amino acid is linked via ester linkage at the point of attachment;
vi) 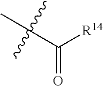
wherein R¹⁴ is 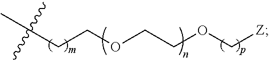
wherein m and p are independently selected from 0 to 5, n refers to degree of polymerization and is selected from 1 to 250 and Z is optionally substituted alkyl or cycloalkyl;
vii) 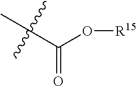
wherein R¹⁵ is selected from group comprising of optionally substituted alkyl, alkenyl, alkynyl, cycloalkyl, heterocycloalkyl, aryl and heteroaryl;
viii) 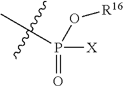
wherein R¹⁶ is selected from group comprising of hydrogen; optionally substituted alkyl, aryl, acyl, heteroaryl; and X is optionally substituted heterocycloalkyl;
ix) 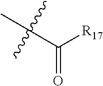
wherein R₁₇ is selected from optionally substituted bicyclic compounds, spirocyclic compounds or bridged bicyclic compounds; pharmaceutically acceptable salts, enantiomers, diastereomers, racemates, or solvates thereof.

39.-80. (canceled)

81. The pharmaceutical composition according to claim 36, comprising at least one formula of compound selected from formula II, formula III, formula IV, formula V, formula VI, formula VII, formula VIII, formula IX, formula X, formula XI, formula XII, formula XIII, formula XIV, formula XV, formula XVI, formula XVII, formula XVIII, formula XIX, formula XX, formula XXI, formula XXII, formula XXIII, formula XXIV, formula XXV, formula XXVI, formula XXVII, formula XXVIII, formula XXIX, formula XXX and formula XXXI pharmaceutically acceptable salts, enantiomers, diastereomers, racemates or solvates thereof; and at least one pharmaceutically acceptable excipient.

82. The method of treating cancer according to claim 38, comprising administering at least one formula of compound selected from formula II, formula III, formula IV, formula V, formula VI, formula VII, formula VIII, formula IX, formula X, formula XI, formula XII, formula XIII, formula XIV, formula XV, formula XVI, formula XVII, formula XVIII, formula XIX, formula XX, formula XXI, formula XXII, formula XXIII, formula XXIV, formula XXV, formula XXVI, formula XXVII, formula XXVIII, formula XXIX, formula XXX and formula XXXI; pharmaceutically acceptable salts, enantiomers, diastereomers, racemates, or solvates thereof.
